SEQUENCE LISTING (204 sequences)

SEQ ID NO: 1 - DNA, 1677 bp, Homo sapiens
atggagctccgggcccgaggctggtggctgctatgtgcggccgcagcgctggtcgcctgcgcccgcggggacccggccagcaagagccggagctgcggcgaggtccgccagatctacggagccaagggcttcagcctgagcgacgtgccccaggcggagatctcgggtgagcacctgcggatctgtccccagggctacacctgctgcaccagcgagatggaggagaacctggccaaccgcagccatgccgagctggagaccgcgctccgggacagcagccgcgtcctgcaggccatgcttgccacccagctgcgcagcttcgatgaccacttccagcacctgctgaacgactcggagcggacgctgcaggccaccttccccggcgccttcggagagctgtacacgcagaacgcgagggccttccgggacctgtactcagagctgcgcctgtactaccgcggtgccaacctgcacctggaggagacgctggccgagttctgggcccgcctgctcgagcgcctcttcaagcagctgcacccccagctgctgctgcctgatgactacctggactgcctgggcaagcaggccgaggcgctgcggcccttcggggaggccccgagagagctgcgcctgcgggccacccgtgccttcgtggctgctcgctcctttgtgcagggcctgggcgtggccagcgacgtggtccggaaagtggctcaggtccccctgggcccggagtgctcgagagctgtcatgaagctggtctactgtgctcactgcctgggagtccccggcgccaggccctgccctgactattgccgaaatgtgctcaagggctgccttgccaaccaggccgacctggacgccgagtggaggaacctcctggactccatggtgctcatcaccgacaagttctggggtacatcgggtgtggagagtgtcatcggcagcgtgcacacgtggctggcggaggccatcaacgccctccaggacaacagggacacgctcacggccaaggtcatccagggctgcgggaaccccaaggtcaacccccagggccccgggcctgaggagaagcggcgccggggcaagctggccccgcgggagaggccaccttcaggcacgctggagaagctggtctccgaagccaaggcccagctccgcgacgtccaggacttctggatcagcctcccagggacactgtgcagtgagaagatggccctgagcactgccagtgatgaccgctgctggaacgggatggccagaggccggtacctccccgaggtcatgggtgacggcctggccaaccagatcaacaaccccgaggtggaggtggacatcaccaagccggacatgaccatccggcagcagatcatgcagctgaagatcatgaccaaccggctgcgcagcgcctacaacggcaacgacgtggacttccaggacgccagtgacgacggcagcggctcgggcagcggtgatggctgtctggatgacctctgcagccggaaggtcagcaggaagagctccagctcccggacgcccttgacccatgccctcccaggcctgtcagagcaggaaggacagaagacctcggctgccagctgcccccagcccccgaccttcctcctgcccctcctcctcttcctggcccttacagtagccaggccccggtggcggtaa

SEQ ID NO: 2 - protein, 558 aa, Homo sapiens
MELRARGWWLLCAAAALVACARGDPASKSRSCGEVRQIYGAKGFSLSDVPQAEISGEHLRICPQGYTCCTSEMEENLANRSHAELETALRDSSRVLQAMLATQLRSFDDHFQHLLNDSERTLQATFPGAFGELYTQNARAFRDLYSELRLYYRGANLHLEETLAEFWARLLERLFKQLHPQLLLPDDYLDCLGKQAEALRPFGEAPRELRLRATRAFVAARSFVQGLGVASDVVRKVAQVPLGPECSRAVMKLVYCAHCLGVPGARPCPDYCRNVLKGCLANQADLDAEWRNLLDSMVLITDKFWGTSGVESVIGSVHTWLAEAINALQDNRDTLTAKVIQGCGNPKVNPQGPGPEEKRRRGKLAPRERPPSGTLEKLVSEAKAQLRDVQDFWISLPGTLCSEKMALSTASDDRCWNGMARGRYLPEVMGDGLANQINNPEVEVDITKPDMTIRQQIMQLKIMTNRLRSAYNGNDVDFQDASDDGSGSGSGDGCLDDLCSRKVSRKSSSSRTPLTHALPGLSEQEGQKTSAASCPQPPTFLLPLLLFLALTVARPRWR

SEQ ID NO: 3 - DNA, 1673 bp, Mus musculus
tggaactccggacccgaggctggtggctgctgtgcgcggccgccgcgctggtcgtctgcgcccgcggggaccccgccagcaagagccggagctgcagcgaagtccgccagatctacggggctaagggctttagcctgagcgatgtgcctcaggcagagatctcgggtgagcacctgcggatctgcccccagggctacacttgctgtactagtgagatggaggagaatttggccaaccacagccgaatggagctggagagcgcactccatgacagcagccgcgccctgcaggccacactggccacccagctgcatggcatcgatgaccacttccagcgcctgctgaatgactcggagcgcacactgcaggaggctttccctggggcctttggggacctgtatacgcagaacactcgtgccttccgggacctatatgctgagctgcgcctctactaccgtggggccaacctgcaccttgaggagacgctggccgagttctgggcacggctgctggagcgcctcttcaagcagctgcacccccagctgctgcctgatgactacctggactgcctgggcaagcaggcggaggcactgcggccgtttggagatgcccctcgagaactgcgcctgcgggccacccgtgcctttgtggctgcacgttcctttgtgcagggcctgggtgtggccagtgatgtagtccggaaggtggcccaggtacctctggccccagaatgttctcgggccatcatgaagttggtctactgtgctcattgccggggagtcccgggcgcccggccctgccccgactattgccgaaatgtgctcaaaggctgccttgccaaccaggccgacctggatgccgagtggaggaacctcctggactccatggtgctcatcactgacaagttctggggcccgtcgggtgcggagagtgtcattggcggtgtgcacgtgtggctggcggaggccatcaacgccctccaggacaacaaggacacactcacagctaaggtcatccaggcctgtggaaaccccaaggtcaatccccacggctctgggcccgaggagaagcgtcgccgtggcaaattggcactgcaggagaagccctccacaggtactctggaaaaactggtctctgaggccaaggcccagctccgagacattcaggacttctggatcagcctcccagggacactgtgcagtgagaagatggccatgagtcctgccagtgacgaccgctgctggaatggaatttccaagggccggtacctaccagaggtgatgggtgacgggctggccaaccagatcaacaaccctgaggtggaagtggacatcaccaagccagacatgaccatccgccagcagattatgcagctcaagatcatgaccaaccgtttacgtggcgcctatggcggcaacgacgtggacttccaggatgctagtgatgacggcagtggctccggcagcggtggcggatgcccagatgacacctgtggccggagggtcagcaagaagagttccagctcccggacccccttgacccatgccctccccggcctgtcagaacaggagggacagaagacctcagctgccacctgcccagagccccacagcttcttcctgctcttcctcgtcaccttggtccttgcggcagccaggcccaggtggcggtaa

SEQ ID NO: 4 - protein, 557 aa, Mus musculus
MELRTRGWWLLCAAAALVVCARGDPASKSRSCSEVRQIYGAKGFSLSDVPQAEISGEHLRICPQGYTCCTSEMEENLANHSRMELESALHDSSRALQATLATQLHGIDDHFQRLLNDSERTLQEAFPGAFGDLYTQNTRAFRDLYAELRLYYRGANLHLEETLAEFWARLLERLFKQLHPQLLPDDYLDCLGKQAEALRPFGDAPRELRLRATRAFVAARSFVQGLGVASDVVRKVAQVPLAPECSRAIMKLVYCAHCRGVPGARPCPDYCRNVLKGCLANQADLDAEWRNLLDSMVLITDKFWGPSGAESVIGGVHVWLAEAINALQDNKDTLTAKVIQACGNPKVNPHGSGPEEKRRRGKLALQEKPSTGTLEKLVSEAKAQLRDIQDFWISLPGTLCSEKMAMSPASDDRCWNGISKGRYLPEVMGDGLANQINNPEVEVDITKPDMTIRQQIMQLKIMTNRLRGAYGGNDVDFQDASDDGSGSGSGGGCPDDTCGRRVSKKSSSSRTPLTHALPGLSEQEGQKTSAATCPEPHSFFLLFLVTLVLAAARPRWR

SEQ ID NO: 5 - protein, 5 aa, artificial sequence
  Amino Acid Sequence of Heavy Chain CDR1 of K090-01a002
RYYMH

SEQ ID NO: 6 - protein, 17 aa, artificial sequence
  Amino Acid Sequence of Heavy Chain CDR2 of K090-01a002
EINPSTGDTTYNQKFKA

SEQ ID NO: 7 - protein, 10 aa, artificial sequence
  Amino Acid Sequence of Heavy Chain CDR3 of K090-01a002
EKRDDGVLAY

SEQ ID NO: 8 - protein, 11 aa, artificial sequence
  Amino Acid Sequence of Light Chain CDR1 of K090-01a002
KASQSVGNNVA

SEQ ID NO: 9 - protein, 7 aa, artificial sequence
  Amino Acid Sequence of Light Chain CDR2 of K090-01a002
YASNRYT

SEQ ID NO: 10 - protein, 9 aa, artificial sequence
  Amino Acid Sequence of Light Chain CDR3 of K090-01a002
QQHYSSPLT

SEQ ID NO: 11 - protein, 5 aa, artificial sequence
  Amino Acid Sequence of Heavy Chain CDR1 of K090-01a007
SYWMH

SEQ ID NO: 12 - protein, 17 aa, artificial sequence
  Amino Acid Sequence of Heavy Chain CDR2 of K090-01a007
NIDPYDSETHYNQKFKD

SEQ ID NO: 13 - protein, 9 aa, artificial sequence
  Amino Acid Sequence of Heavy Chain CDR3 of K090-01a007
DSNWGYFDY

SEQ ID NO: 14 - protein, 16 aa, artificial sequence
  Amino Acid Sequence of Light Chain CDR1 of K090-01a007
RSSQSLVHSNGNTYLH

SEQ ID NO: 15 - protein, 7 aa, artificial sequence
  Amino Acid Sequence of Light Chain CDR2 of K090-01a007
KVSNRFS

SEQ ID NO: 16 - protein, 9 aa, artificial sequence
  Amino Acid Sequence of Light Chain CDR3 of K090-01a007
SQSTHVPWT

SEQ ID NO: 17 - protein, 5 aa, artificial sequence
  Amino Acid Sequence of Heavy Chain CDR1 of K090-01a016
DYYIN

SEQ ID NO: 18 - protein, 17 aa, artificial sequence
  Amino Acid Sequence of Heavy Chain CDR2 of K090-01a016
YIDPYNDGSKYNEKFKG

SEQ ID NO: 19 - protein, 15 aa, artificial sequence
  Amino Acid Sequence of Heavy Chain CDR3 of K090-01a016
TPYYSFYSYGFYFDY

SEQ ID NO: 20 - protein, 15 aa, artificial sequence
  Amino Acid Sequence of Light Chain CDR1 of K090-01a016
RASKSVSTSSYSYMH

SEQ ID NO: 21 - protein, 7 aa, artificial sequence
  Amino Acid Sequence of Light Chain CDR2 of K090-01a016
YASYLES

SEQ ID NO: 22 - protein, 9 aa, artificial sequence
  Amino Acid Sequence of Light Chain CDR3 of K090-01a016
QHSREFPLT

SEQ ID NO: 23 - protein, 5 aa, artificial sequence
  Amino Acid Sequence of Heavy Chain CDR1 of K090-01a017
NYWIG

SEQ ID NO: 24 - protein, 17 aa, artificial sequence
  Amino Acid Sequence of Heavy Chain CDR2 of K090-01a017
DLYPGGGYTHYNEKFKG

SEQ ID NO: 25 - protein, 13 aa, artificial sequence
  Amino Acid Sequence of Heavy Chain CDR3 of K090-01a017
ERVYFDGSRYFDV

SEQ ID NO: 26 - protein, 11 aa, artificial sequence
  Amino Acid Sequence of Light Chain CDR1 of K090-01a017
KASQSVGNNVA

SEQ ID NO: 27 - protein, 7 aa, artificial sequence
  Amino Acid Sequence of Light Chain CDR2 of K090-01a017
YASNRYT

SEQ ID NO: 28 - protein, 9 aa, artificial sequence
  Amino Acid Sequence of Light Chain CDR3 of K090-01a017
QQHYSSPWT

SEQ ID NO: 29 - protein, 5 aa, artificial sequence
  Amino Acid Sequence of Heavy Chain CDR1 of K090-01a021
GYYMH

SEQ ID NO: 30 - protein, 17 aa, artificial sequence
  Amino Acid Sequence of Heavy Chain CDR2 of K090-01a021
EINPSTGGTTYNQKFKA

SEQ ID NO: 31 - protein, 10 aa, artificial sequence
  Amino Acid Sequence of Heavy Chain CDR3 of K090-01a021
EKRDDGVFAY

SEQ ID NO: 32 - protein, 11 aa, artificial sequence
  Amino Acid Sequence of Light Chain CDR1 of K090-01a021
KASQSVGNNVA

SEQ ID NO: 33 - protein, 7 aa, artificial sequence
  Amino Acid Sequence of Light Chain CDR2 of K090-01a021
YASNRYT

SEQ ID NO: 34 - protein, 9 aa, artificial sequence
  Amino Acid Sequence of Light Chain CDR3 of K090-01a021
QQHYSSPLT

SEQ ID NO: 35 - protein, 5 aa, artificial sequence
  Amino Acid Sequence of Heavy Chain CDR1 of K090-01a026
NYWIG

SEQ ID NO: 36 - protein, 17 aa, artificial sequence
  Amino Acid Sequence of Heavy Chain CDR2 of K090-01a026
DIYPGGGYTHYNEKFKG

SEQ ID NO: 37 - protein, 13 aa, artificial sequence
  Amino Acid Sequence of Heavy Chain CDR3 of K090-01a026
ERVYYDGSRFFDV

SEQ ID NO: 38 - protein, 11 aa, artificial sequence
  Amino Acid Sequence of Light Chain CDR1 of K090-01a026
KASQSVGNNVA

SEQ ID NO: 39 - protein, 7 aa, artificial sequence
  Amino Acid Sequence of Light Chain CDR2 of K090-01a026
YASNRYT

SEQ ID NO: 40 - protein, 9 aa, artificial sequence
  Amino Acid Sequence of Light Chain CDR3 of K090-01a026
QQHYSSPWT

SEQ ID NO: 41 - protein, 5 aa, artificial sequence
  Amino Acid Sequence of Heavy Chain CDR1 of K090-01a009
GYFMN

SEQ ID NO: 42 - protein, 17 aa, artificial sequence
  Amino Acid Sequence of Heavy Chain CDR2 of K090-01a009
RINPDNGDIFYNQKFKG

SEQ ID NO: 43 - protein, 8 aa, artificial sequence
  Amino Acid Sequence of Heavy Chain CDR3 of K090-01a009
SWNWYFDV

SEQ ID NO: 44 - protein, 11 aa, artificial sequence
  Amino Acid Sequence of Light Chain CDR1 of K090-01a009
RASENIYSYLA

SEQ ID NO: 45 - protein, 7 aa, artificial sequence
  Amino Acid Sequence of Light Chain CDR2 of K090-01a009
NAKTLAE

SEQ ID NO: 46 - protein, 9 aa, artificial sequence
  Amino Acid Sequence of Light Chain CDR3 of K090-01a009
QHHYGTPYT

SEQ ID NO: 47 - protein, 5 aa, artificial sequence
  Amino Acid Sequence of Heavy Chain CDR1 of K090-01a030
NYWIG

SEQ ID NO: 48 - protein, 17 aa, artificial sequence
  Amino Acid Sequence of Heavy Chain CDR2 of K090-01a030
DIYPGGGYTHYNEKFKG

SEQ ID NO: 49 - protein, 13 aa, artificial sequence
  Amino Acid Sequence of Heavy Chain CDR3 of K090-01a030
ERVYYDGSRYFDV

SEQ ID NO: 50 - protein, 11 aa, artificial sequence
  Amino Acid Sequence of Light Chain CDR1 of K090-01a030
KASQSVGNNVA

SEQ ID NO: 51 - protein, 7 aa, artificial sequence
  Amino Acid Sequence of Light Chain CDR2 of K090-01a030
YASNRYT

SEQ ID NO: 52 - protein, 9 aa, artificial sequence
  Amino Acid Sequence of Light Chain CDR3 of K090-01a030
QQHYSSPWT

SEQ ID NO: 53 - protein, 5 aa, artificial sequence
  Amino Acid Sequence of Heavy Chain CDR1 of K090-01a033
GYYMH

SEQ ID NO: 54 - protein, 17 aa, artificial sequence
  Amino Acid Sequence of Heavy Chain CDR2 of K090-01a033
EINPSTGDTTYNQKFKA

SEQ ID NO: 55 - protein, 10 aa, artificial sequence
  Amino Acid Sequence of Heavy Chain CDR3 of K090-01a033
EKRDDGVFAY

SEQ ID NO: 56 - protein, 11 aa, artificial sequence
  Amino Acid Sequence of Light Chain CDR1 of K090-01a033
KASENVGTYVS

SEQ ID NO: 57 - protein, 7 aa, artificial sequence
  Amino Acid Sequence of Light Chain CDR2 of K090-01a033
GASNRYT

SEQ ID NO: 58 - protein, 9 aa, artificial sequence
  Amino Acid Sequence of Light Chain CDR3 of K090-01a033
GQSYSYPLT

SEQ ID NO: 59 - protein, 5 aa, artificial sequence
  Amino Acid Sequence of Heavy Chain CDR1 of K090-01a042
RYWMH

SEQ ID NO: 60 - protein, 17 aa, artificial sequence
  Amino Acid Sequence of Heavy Chain CDR2 of K090-01a042
YINPSSGYTEYNQKFKD

SEQ ID NO: 61 - protein, 10 aa, artificial sequence
  Amino Acid Sequence of Heavy Chain CDR3 of K090-01a042
LGDYSYYFDY

SEQ ID NO: 62 - protein, 16 aa, artificial sequence
  Amino Acid Sequence of Light Chain CDR1 of K090-01a042
RSSQSIVHSNGNTYLE

SEQ ID NO: 63 - protein, 7 aa, artificial sequence
  Amino Acid Sequence of Light Chain CDR2 of K090-01a042
KVSNRFS

SEQ ID NO: 64 - protein, 9 aa, artificial sequence
  Amino Acid Sequence of Light Chain CDR3 of K090-01a042
FQGSHVPWT

SEQ ID NO: 65 - protein, 5 aa, artificial sequence
  Amino Acid Sequence of Heavy Chain CDR1 of K090-02a002
DYGMH

SEQ ID NO: 66 - protein, 17 aa, artificial sequence
  Amino Acid Sequence of Heavy Chain CDR2 of K090-02a002
YISSGSSTIYYADTVKG

SEQ ID NO: 67 - protein, 16 aa, artificial sequence
  Amino Acid Sequence of Heavy Chain CDR3 of K090-02a002
HSYYSYDVGGDYVMDY

SEQ ID NO: 68 - protein, 11 aa, artificial sequence
  Amino Acid Sequence of Light Chain CDR1 of K090-02a002
KASQDVGTAVA

SEQ ID NO: 69 - protein, 7 aa, artificial sequence
  Amino Acid Sequence of Light Chain CDR2 of K090-02a002
WASTRHT

SEQ ID NO: 70 - protein, 9 aa, artificial sequence
  Amino Acid Sequence of Light Chain CDR3 of K090-02a002
QQYSSYPLT

SEQ ID NO: 71 - protein, 5 aa, artificial sequence
  Amino Acid Sequence of Heavy Chain CDR1 of K090-02a006
GYNMN

SEQ ID NO: 72 - protein, 17 aa, artificial sequence
  Amino Acid Sequence of Heavy Chain CDR2 of K090-02a006
NINPYYGTTNYNQKFKG

SEQ ID NO: 73 - protein, 9 aa, artificial sequence
  Amino Acid Sequence of Heavy Chain CDR3 of K090-02a006
DGYYGAMDY

SEQ ID NO: 74 - protein, 11 aa, artificial sequence
  Amino Acid Sequence of Light Chain CDR1 of K090-02a006
KASQNVGTYVA

SEQ ID NO: 75 - protein, 7 aa, artificial sequence
  Amino Acid Sequence of Light Chain CDR2 of K090-02a006
SASYRYS

SEQ ID NO: 76 - protein, 9 aa, artificial sequence
  Amino Acid Sequence of Light Chain CDR3 of K090-02a006
QQYYNYPLT

SEQ ID NO: 77 - protein, 5 aa, artificial sequence
  Amino Acid Sequence of Heavy Chain CDR1 of K090-02a010
DYGMH

SEQ ID NO: 78 - protein, 17 aa, artificial sequence
  Amino Acid Sequence of Heavy Chain CDR2 of K090-02a010
YINSGSSTIYYADTVKG

SEQ ID NO: 79 - protein, 16 aa, artificial sequence
  Amino Acid Sequence of Heavy Chain CDR3 of K090-02a010
HSYYSYDVGGDYVMDY

SEQ ID NO: 80 - protein, 11 aa, artificial sequence
  Amino Acid Sequence of Light Chain CDR1 of K090-02a010
KASQDVGTAVA

SEQ ID NO: 81 - protein, 7 aa, artificial sequence
  Amino Acid Sequence of Light Chain CDR2 of K090-02a010
WASTRHT

SEQ ID NO: 82 - protein, 9 aa, artificial sequence
  Amino Acid Sequence of Light Chain CDR3 of K090-02a010
QQYSSYPLT

SEQ ID NO: 83 - protein, 5 aa, artificial sequence
  Amino Acid Sequence of Heavy Chain CDR1 of K090-02a014
GYYIH

SEQ ID NO: 84 - protein, 17 aa, artificial sequence
  Amino Acid Sequence of Heavy Chain CDR2 of K090-02a014
EINPSTGDITYNQKFKA

SEQ ID NO: 85 - protein, 9 aa, artificial sequence
  Amino Acid Sequence of Heavy Chain CDR3 of K090-02a014
ERNYDRFAY

SEQ ID NO: 86 - protein, 12 aa, artificial sequence
  Amino Acid Sequence of Light Chain CDR1 of K090-02a014
SVSSSISSSNLH

SEQ ID NO: 87 - protein, 7 aa, artificial sequence
  Amino Acid Sequence of Light Chain CDR2 of K090-02a014
GTSNLAS

SEQ ID NO: 88 - protein, 9 aa, artificial sequence
  Amino Acid Sequence of Light Chain CDR3 of K090-02a014
QQWSSYPLT

SEQ ID NO: 89 - protein, 5 aa, artificial sequence
  Amino Acid Sequence of Heavy Chain CDR1 of K090-02a022
DYYMN

SEQ ID NO: 90 - protein, 17 aa, artificial sequence
  Amino Acid Sequence of Heavy Chain CDR2 of K090-02a022
RVNPSNGGTSYNQKFKG

SEQ ID NO: 91 - protein, 10 aa, artificial sequence
  Amino Acid Sequence of Heavy Chain CDR3 of K090-02a022
SGENYYAMDY

SEQ ID NO: 92 - protein, 11 aa, artificial sequence
  Amino Acid Sequence of Light Chain CDR1 of K090-02a022
RASENIYSYLA

SEQ ID NO: 93 - protein, 7 aa, artificial sequence
  Amino Acid Sequence of Light Chain CDR2 of K090-02a022
NGKTLAE

SEQ ID NO: 94 - protein, 9 aa, artificial sequence
  Amino Acid Sequence of Light Chain CDR3 of K090-02a022
QNHYGVPWT

SEQ ID NO: 95 - protein, 5 aa, artificial sequence
  Amino Acid Sequence of Heavy Chain CDR1 of K090-02a034
GYYMH

SEQ ID NO: 96 - protein, 17 aa, artificial sequence
  Amino Acid Sequence of Heavy Chain CDR2 of K090-02a034
EINPSTGDTTYNQKFKA

SEQ ID NO: 97 - protein, 9 aa, artificial sequence
  Amino Acid Sequence of Heavy Chain CDR3 of K090-02a034
ERNYDRFAY

SEQ ID NO: 98 - protein, 10 aa, artificial sequence
  Amino Acid Sequence of Light Chain CDR1 of K090-02a034
SASSSVSYMY

SEQ ID NO: 99 - protein, 7 aa, artificial sequence
  Amino Acid Sequence of Light Chain CDR2 of K090-02a034
DTSNLAS

SEQ ID NO: 100 - protein, 9 aa, artificial sequence
  Amino Acid Sequence of Light Chain CDR3 of K090-02a034
QQWSSYPLT

SEQ ID NO: 101 - protein, 5 aa, artificial sequence
  Amino Acid Sequence of Heavy Chain CDR1 of K090-02a035
SYWMH

SEQ ID NO: 102 - protein, 17 aa, artificial sequence
  Amino Acid Sequence of Heavy Chain CDR2 of K090-02a035
NIDPSDSETHYNQKFKD

SEQ ID NO: 103 - protein, 4 aa, artificial sequence
  Amino Acid Sequence of Heavy Chain CDR3 of K090-02a035
NLGY

SEQ ID NO: 104 - protein, 16 aa, artificial sequence
  Amino Acid Sequence of Light Chain CDR1 of K090-02a035
RSSQSLVHSNGNTYLH

SEQ ID NO: 105 - protein, 7 aa, artificial sequence
  Amino Acid Sequence of Light Chain CDR2 of K090-02a035
KVSNRFS

SEQ ID NO: 106 - protein, 9 aa, artificial sequence
  Amino Acid Sequence of Light Chain CDR3 of K090-02a035
SQSTHVPYT

SEQ ID NO: 107 - protein, 5 aa, artificial sequence
  Amino Acid Sequence of Heavy Chain CDR1 of K090-02b006
DYGMH

SEQ ID NO: 108 - protein, 17 aa, artificial sequence
  Amino Acid Sequence of Heavy Chain CDR2 of K090-02b006
YISSGSSTIYYADTVKG

SEQ ID NO: 109 - protein, 16 aa, artificial sequence
  Amino Acid Sequence of Heavy Chain CDR3 of K090-02b006
HSYYNYDIGGYYAMDY

SEQ ID NO: 110 - protein, 11 aa, artificial sequence
  Amino Acid Sequence of Light Chain CDR1 of K090-02b006
RASQEISGYLS

SEQ ID NO: 111 - protein, 7 aa, artificial sequence
  Amino Acid Sequence of Light Chain CDR2 of K090-02b006
AASTLDS

SEQ ID NO: 112 - protein, 9 aa, artificial sequence
  Amino Acid Sequence of Light Chain CDR3 of K090-02b006
LQYASYPLT

SEQ ID NO: 113 - protein, 5 aa, artificial sequence
  Amino Acid Sequence of Heavy Chain CDR1 of Clone 4
RYAMY

SEQ ID NO: 114 - protein, 16 aa, artificial sequence
  Amino Acid Sequence of Heavy Chain CDR2 of Clone 4
IGNTGRYTGYGSAVKG

SEQ ID NO: 115 - protein, 14 aa, artificial sequence
  Amino Acid Sequence of Heavy Chain CDR3 of Clone 4
SVSPYCCDAADIDA

SEQ ID NO: 116 - protein, 10 aa, artificial sequence
  Amino Acid Sequence of Light Chain CDR1 of Clone 4
SGGSSGYAYG

SEQ ID NO: 117 - protein, 7 aa, artificial sequence
  Amino Acid Sequence of Light Chain CDR2 of Clone 4
SNNNRPS

SEQ ID NO: 118 - protein, 11 aa, artificial sequence
  Amino Acid Sequence of Light Chain CDR3 of Clone 4
GSVDSSSYAGI

SEQ ID NO: 119 - protein, 5 aa, artificial sequence
  Amino Acid Sequence of Heavy Chain CDR1 of Clone 18
SVNMF

SEQ ID NO: 120 - protein, 17 aa, artificial sequence
  Amino Acid Sequence of Heavy Chain CDR2 of Clone 18
GIDNDATFTLYGSAVKG

SEQ ID NO: 121 - protein, 19 aa, artificial sequence
  Amino Acid Sequence of Heavy Chain CDR3 of Clone 18
TLCSTTWGCGAYSAGDIDA

SEQ ID NO: 122 - protein, 12 aa, artificial sequence
  Amino Acid Sequence of Light Chain CDR1 of Clone 18
SGGGSSGYGYYG

SEQ ID NO: 123 - protein, 7 aa, artificial sequence
  Amino Acid Sequence of Light Chain CDR2 of Clone 18
NNNKRPT

SEQ ID NO: 124 - protein, 10 aa, artificial sequence
  Amino Acid Sequence of Light Chain CDR3 of Clone 18
GSRDNTYVGI

SEQ ID NO: 125 - DNA, 1431 bp, artificial sequence
  Nucleic Acid Sequence of Heavy Chain of K090-01a002
  CDS (16)..(1422)
aagcttgccgccaccatgaaacacctgtggttcttcctcctcctggtggcggcccccagatgggtcctgtccgaggttcagctgcagcagtctggacctgaactggtgaagcctggggcttcagtgaagatatcatgcaagtcttctggttactcattcactcgctactacatgcactgggtgaagcacagtcctgaaaagagccttgagtggattggagagattaatcctagcactggtgatactacgtacaaccagaagttcaaggccaaggccacattgactgtagacaaatcctccagcacagcctacatgcagctcaagagcctgacatctgaggactctgcagtctattactgtgcaagagagaagagagatgatggcgtacttgcttactggggccaagggactctggtcactgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 126 - protein, 468 aa, artificial sequence
  Synthetic Construct
MKHLWFFLLLVAAPRWVLSEVQLQQSGPELVKPGASVKISCKSSGYSFTRYYMHWVKHSPEKSLEWIGEINPSTGDTTYNQKFKAKATLTVDKSSSTAYMQLKSLTSEDSAVYYCAREKRDDGVLAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 127 - DNA, 731 bp, artificial sequence
  Nucleic Acid Sequence of Light Chain of K090-01a002
  CDS (16)..(720)
aagcttgccgccaccatgaggctccttgctcagcttctggggctgctaatgctctgggtccctggatccagtggggacatccagatgacccagtctcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgatcgcttcactggcagtggatctgggacagatttcactttcaccatcagcagtgtgcaggttgaagacctggcagtttatttctgtcagcagcattatagctctcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 128 - protein, 234 aa, artificial sequence
  Synthetic Construct
MRLLAQLLGLLMLWVPGSSGDIQMTQSPKFLPVSAGDRVTMTCKASQSVGNNVAWYQQKPGQSPKLLIYYASNRYTGVPDRFTGSGSGTDFTFTISSVQVEDLAVYFCQQHYSSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 129 - DNA, 1428 bp, artificial sequence
  Nucleic Acid Sequence of Heavy Chain of K090-01a007
  CDS (16)..(1419)
aagcttgccgccaccatgaaacacctgtggttcttcctcctcctggtggcggcccccagatgggtcctgtcccaggtccacctgcagcagtctggggctgagctggtgaagcctggggcttcagtgaagctgtcctgcaaggcttctggctacaccttcaccagctactggatgcactgggtgaagcagaggcctggacaaggccttgaatggattggtaatattgacccttatgatagtgaaactcactacaatcaaaagttcaaggacaaggccacattgactgtagacaaatcctccagcacagcctacatgcagctcagcagcctgacatctgaggactctgcggtctattactgtgcaagggatagtaactggggctactttgactactggggccaaggcaccactctcacagtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 130 - protein, 467 aa, artificial sequence
  Synthetic Construct
MKHLWFFLLLVAAPRWVLSQVHLQQSGAELVKPGASVKLSCKASGYTFTSYWMHWVKQRPGQGLEWIGNIDPYDSETHYNQKFKDKATLTVDKSSSTAYMQLSSLTSEDSAVYYCARDSNWGYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 131 - DNA, 746 bp, artificial sequence
  Nucleic Acid Sequence of Light Chain of K090-01a007
  CDS (16)..(735)
aagcttgccgccaccatgaggctccttgctcagcttctggggctgctaatgctctgggtccctggatccagtggggatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 132 - protein, 239 aa, artificial sequence
  Synthetic Construct
MRLLAQLLGLLMLWVPGSSGDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 133 - DNA, 1446 bp, artificial sequence
  Nucleic Acid Sequence of Heavy Chain of K090-01a016
  CDS (16)..(1437)
aagcttgccgccaccatgaaacacctgtggttcttcctcctcctggtggcggcccccagatgggtcctgtcccaggtccacctgcagcagtctggacctgagctggtgaagcctggggcttcagtgaagatatcctgcaaggcttctggctacaccttcactgactactatataaactgggtgaagcagaagcctggacagggccttgagtggattggatatattgatccttacaatgatggttctaagtacaatgagaagttcaaaggcaaggccacactgacttcagacaagtcctccagcacagcctacatggagctcagcagtctgacctctgaggactctgcggtctattactgtgcaagaacgccctactatagtttctatagttacgggttctactttgactactggggccaaggcaccactctcacagtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 134 - protein, 473 aa, artificial sequence
  Synthetic Construct
MKHLWFFLLLVAAPRWVLSQVHLQQSGPELVKPGASVKISCKASGYTFTDYYINWVKQKPGQGLEWIGYIDPYNDGSKYNEKFKGKATLTSDKSSSTAYMELSSLTSEDSAVYYCARTPYYSFYSYGFYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 135 - DNA, 743 bp, artificial sequence
  Nucleic Acid Sequence of Light Chain of K090-01a016
  CDS (16)..(732)
aagcttgccgccaccatgaggctccttgctcagcttctggggctgctaatgctctgggtccctggatccagtggggacattgtgctgacacagtctcctgcttccttagctgtatctctggggcagagggccaccatctcctgcagggccagcaaaagtgtcagtacatctagctatagttacatgcactggtaccaacagaaaccaggacagccacccaaactcctcatcaagtatgcatcctacctagaatctggggttcctgccaggttcagtggcagtgggtctgggacagacttcaccctcaacatccatcctgtggaggaggaggatgctgcaacatattactgtcagcacagtagggagtttccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 136 - protein, 238 aa, artificial sequence
  Synthetic Construct
MRLLAQLLGLLMLWVPGSSGDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 137 - DNA, 1440 bp, artificial sequence
  Nucleic Acid Sequence of Heavy Chain of K090-01a017
  CDS (16)..(1431)
aagcttgccgccaccatgaaacacctgtggttcttcctcctcctggtggcggcccccagatgggtcctgtccgaggttcagctgcagcagtctggaggagagctggtaaggcctgggacttcagtgaagatgtcctgcaaggcagctggatacaccttcactaactactggataggttgggtaaagcagaggcctggacatggccttgagtggattggagatctttaccctggaggtggttatactcactacaatgagaagttcaagggcaaggccacactgactgcagacacatcctccagcacagcctacatgcagctcagcagcctgacatctgaggactctgccatctattactgtgcaagagagagggtttacttcgatggtagccggtacttcgatgtctggggcgcagggaccacggtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 138 - protein, 471 aa, artificial sequence
  Synthetic Construct
MKHLWFFLLLVAAPRWVLSEVQLQQSGGELVRPGTSVKMSCKAAGYTFTNYWIGWVKQRPGHGLEWIGDLYPGGGYTHYNEKFKGKATLTADTSSSTAYMQLSSLTSEDSAIYYCARERVYFDGSRYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 139 - DNA, 731 bp, artificial sequence
  Nucleic Acid Sequence of Light Chain of K090-01a017
  CDS (16)..(720)
aagcttgccgccaccatgaggctccttgctcagcttctggggctgctaatgctctgggtccctggatccagtgggagtattgtgatgacccagactcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgatcgcttcactggtagtggatctgggacagatttcactttcaccatcagcagtgtgcaggttgaagacctggcagtttatttctgtcagcagcattatagctctccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 140 - protein, 234 aa, artificial sequence
  Synthetic Construct
MRLLAQLLGLLMLWVPGSSGSIVMTQTPKFLPVSAGDRVTMTCKASQSVGNNVAWYQQKPGQSPKLLIYYASNRYTGVPDRFTGSGSGTDFTFTISSVQVEDLAVYFCQQHYSSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 141 - DNA, 1431 bp, artificial sequence
  Nucleic Acid Sequence of Heavy Chain of K090-01a021
  CDS (16)..(1422)
aagcttgccgccaccatgaaacacctgtggttcttcctcctcctggtggcggcccccagatgggtcctgtcccaggtgcagctgaagcagtcaggacctgagctggtgaagcctggggcttcagtgaagatatcctgcaaggcttctggttactcattcactggctactacatgcactgggtgaagcaaagtcctgaaaagagccttgagtggattggagagattaatcctagcactggtggtactacctacaaccagaagttcaaggccaaggccactttgactgtagacaaatcctccagtacagcctacatgcagctcaagagcctgacatctgaggactctgcagtctattactgtgcaagagagaagagagatgatggcgtatttgcttactggggccaagggactctggtcactgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 142 - protein, 468 aa, artificial sequence
  Synthetic Construct
MKHLWFFLLLVAAPRWVLSQVQLKQSGPELVKPGASVKISCKASGYSFTGYYMHWVKQSPEKSLEWIGEINPSTGGTTYNQKFKAKATLTVDKSSSTAYMQLKSLTSEDSAVYYCAREKRDDGVFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 143 - DNA, 731 bp, artificial sequence
  Nucleic Acid Sequence of Light Chain of K090-01a021
  CDS (16)..(720)
aagcttgccgccaccatgaggctccttgctcagcttctggggctgctaatgctctgggtccctggatccagtgggagtattgtgatgacccagactcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgatcgcttcactggcagtggatctgggacagatttccctttcaccatcagcagtgtgcaggttgaagacctggcagtttatttctgtcagcagcattatagctctcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 144 - protein, 234 aa, artificial sequence
  Synthetic Construct
MRLLAQLLGLLMLWVPGSSGSIVMTQTPKFLPVSAGDRVTMTCKASQSVGNNVAWYQQKPGQSPKLLIYYASNRYTGVPDRFTGSGSGTDFPFTISSVQVEDLAVYFCQQHYSSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 145 - DNA, 1440 bp, artificial sequence
  Nucleic Acid Sequence of Heavy Chain of K090-01a026
  CDS (16)..(1431)
aagcttgccgccaccatgaaacacctgtggttcttcctcctcctggtggcggcccccagatgggtcctgtcccaggtccacctgcagcagtctggagctgagctggtaaggcctgggacttcagtgaagatgtcctgcaaggctgctggatacaccttcaccaactactggataggttgggtaaaacagaggcctggacatggccttgagtggattggagatatttaccctggaggtggttatactcactacaatgagaagttcaagggcaaggccacactgactgcagacacatcctccagcacagcctacatgcagctcagcagcctgacatctgaggactctgccatctattactgtgcaagagagagggtttactacgatggtagccggttcttcgatgtctggggcgcagggaccacggtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 146 - protein, 471 aa, artificial sequence
  Synthetic Construct
MKHLWFFLLLVAAPRWVLSQVHLQQSGAELVRPGTSVKMSCKAAGYTFTNYWIGWVKQRPGHGLEWIGDIYPGGGYTHYNEKFKGKATLTADTSSSTAYMQLSSLTSEDSAIYYCARERVYYDGSRFFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 147 - DNA, 731 bp, artificial sequence
  Nucleic Acid Sequence of Light Chain of K090-01a026
  CDS (16)..(720)
aagcttgccgccaccatgaggctccttgctcagcttctggggctgctaatgctctgggtccctggatccagtggggacattgtgatgtcacagtctcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgaccgcttcactggcagtggatctgggacagatttcactttcaccatcagcagtgtgcaggttgaagacctggcagtttatttctgtcagcagcattatagctctccgtggacgttcggtggaggcaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 148 - protein, 234 aa, artificial sequence
  Synthetic Construct
MRLLAQLLGLLMLWVPGSSGDIVMSQSPKFLPVSAGDRVTMTCKASQSVGNNVAWYQQKPGQSPKLLIYYASNRYTGVPDRFTGSGSGTDFTFTISSVQVEDLAVYFCQQHYSSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 149 - DNA, 1425 bp, artificial sequence
  Nucleic Acid Sequence of Heavy Chain of K090-01a009
  CDS (16)..(1416)
aagcttgccgccaccatgaaacacctgtggttcttcctcctcctggtggcggcccccagatgggtcctgtccgaggttcagctgcagcagtctggacctgagctggtgaagcctggggcttcagtggagatatcctgcaaggcttctggttactcatttactggctactttatgaactgggtgaagcagagccaaggaaagagccttgagtggattggacgtattaatcctgacaatggtgatattttctacaaccagaagttcaagggcaaggccacattaactgtagacaaatcctctagcacagcccacatggagctccggagcctgacatctgaggactctgcagtctattattgtgcaagatcgtggaactggtacttcgatgtctggggcgcagggaccacggtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 150 - protein, 466 aa, artificial sequence
  Synthetic Construct
MKHLWFFLLLVAAPRWVLSEVQLQQSGPELVKPGASVEISCKASGYSFTGYFMNWVKQSQGKSLEWIGRINPDNGDIFYNQKFKGKATLTVDKSSSTAHMELRSLTSEDSAVYYCARSWNWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 151 - DNA, 731 bp, artificial sequence
  Nucleic Acid Sequence of Light Chain of K090-01a009
  CDS (16)..(720)
aagcttgccgccaccatgaggctccttgctcagcttctggggctgctaatgctctgggtccctggatccagtggggacatccagatgacacagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtactccgtatacgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 152 - protein, 234 aa, artificial sequence
  Synthetic Construct
MRLLAQLLGLLMLWVPGSSGDIQMTQSPASLSASVGETVTITCRASENIYSYLAWYQQKQGKSPQLLVYNAKTLAEGVPSRFSGSGSGTQFSLKINSLQPEDFGSYYCQHHYGTPYTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 153 - DNA, 1440 bp, artificial sequence
  Nucleic Acid Sequence of Heavy Chain of K090-01a030
  CDS (16)..(1431)
aagcttgccgccaccatgaaacacctgtggttcttcctcctcctggtggcggcccccagatgggtcctgtcccaggtccagctgcagcagtctggggctgagttggtaaggcctgggacttcagtgaagatgtcctgcaaggctgctggatacaccttcacaaactactggataggttgggtaaagcagaggcctggacatggccttgagtggattggagatatttaccctggaggtggttatactcactacaatgagaagttcaagggcaaggccacactgactgcagacacatcctccagcacagcctacatgcagctcagcagcctgacatctgaggactctgccatctattactgtgcaagagagagggtttactacgatggtagccggtacttcgatgtctggggcgcagggaccacggtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 154 - protein, 471 aa, artificial sequence
  Synthetic Construct
MKHLWFFLLLVAAPRWVLSQVQLQQSGAELVRPGTSVKMSCKAAGYTFTNYWIGWVKQRPGHGLEWIGDIYPGGGYTHYNEKFKGKATLTADTSSSTAYMQLSSLTSEDSAIYYCARERVYYDGSRYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 155 - DNA, 731 bp, artificial sequence
  Nucleic Acid Sequence of Light Chain of K090-01a030
  CDS (16)..(720)
aagcttgccgccaccatgaggctccttgctcagcttctggggctgctaatgctctgggtccctggatccagtggggacatcaagatgacccagtctcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgatcgcttcactggcagtggatctgggacagatttcactttcaccatcagcagtgtgcaggttgaagacctggcagtttatttctgtcagcagcattatagctctccgtggacgttcggtggaggcacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 156 - protein, 234 aa, artificial sequence
  Synthetic Construct
MRLLAQLLGLLMLWVPGSSGDIKMTQSPKFLPVSAGDRVTMTCKASQSVGNNVAWYQQKPGQSPKLLIYYASNRYTGVPDRFTGSGSGTDFTFTISSVQVEDLAVYFCQQHYSSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 157 - DNA, 1431 bp, artificial sequence
  Nucleic Acid Sequence of Heavy Chain of K090-01a033
  CDS (16)..(1422)
aagcttgccgccaccatgaaacacctgtggttcttcctcctcctggtggcggcccccagatgggtcctgtcccaggtgcagctgaagcagtcaggacctgaactggtgaagcctggggcttcagtgaagatatcatgcaaggcttctggttactcattcactggctactacatgcactgggtgaagcaaagtcctgaaaagagccttgagtggattggagagattaatcctagcactggtgatactacctacaaccagaagttcaaggccaaggccacattgactgtagacaaatcctccagcacagcctacatgcagctcaagagcctgacatctgaggactctgcagtctattactgtgcaagagagaagagagatgatggcgtatttgcttactggggccaagggactctggtcactgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 158 - protein, 468 aa, artificial sequence
  Synthetic Construct
MKHLWFFLLLVAAPRWVLSQVQLKQSGPELVKPGASVKISCKASGYSFTGYYMHWVKQSPEKSLEWIGEINPSTGDTTYNQKFKAKATLTVDKSSSTAYMQLKSLTSEDSAVYYCAREKRDDGVFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 159 - DNA, 731 bp, artificial sequence
  Nucleic Acid Sequence of Light Chain of K090-01a033
  CDS (16)..(720)
aagcttgccgccaccatgaggctccttgctcagcttctggggctgctaatgctctgggtccctggatccagtggggacattgtgatgtcacagtctcccaaatccatgtccatgtcagtaggagagagggtcgccttgagctgcaaggccagtgagaatgtgggtacttatgtatcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattattactgtggacagagttacagctatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 160 - protein, 234 aa, artificial sequence
  Synthetic Construct
MRLLAQLLGLLMLWVPGSSGDIVMSQSPKSMSMSVGERVALSCKASENVGTYVSWYQQKPEQSPKLLIYGASNRYTGVPDRFTGSGSATDFTLTISSVQAEDLADYYCGQSYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 161 - DNA, 1431 bp, artificial sequence
  Nucleic Acid Sequence of Heavy Chain of K090-01a042
  CDS (16)..(1422)
aagcttgccgccaccatgaaacacctgtggttcttcctcctcctggtggcggcccccagatgggtcctgtcccaggtccaactgcagcagcctggggctgaactggcaaaacctggggcctcagtgaagatgtcctgcaaggcttctggctacacctttactaggtactggatgcattgggtaaaacagaggcctggacagggtctggaatggattggatacattaatcctagcagtggttatactgagtacaatcagaagttcaaggacaaggccacattgactgcagacaaatcctccagcacagcctacatgcaactaagcagcctgacatctgaggactctgcagtctattactgtgtaagacttggtgactactcgtactactttgactactggggccaaggcaccactctcacagtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 162 - protein, 468 aa, artificial sequence
  Synthetic Construct
MKHLWFFLLLVAAPRWVLSQVQLQQPGAELAKPGASVKMSCKASGYTFTRYWMHWVKQRPGQGLEWIGYINPSSGYTEYNQKFKDKATLTADKSSSTAYMQLSSLTSEDSAVYYCVRLGDYSYYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 163 - DNA, 746 bp, artificial sequence
  Nucleic Acid Sequence of Light Chain of K090-01a042
  CDS (16)..(735)
aagcttgccgccaccatgaggctccttgctcagcttctggggctgctaatgctctgggtccctggatccagtggggatgttgtgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 164 - protein, 239 aa, artificial sequence
  Synthetic Construct
MRLLAQLLGLLMLWVPGSSGDVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 165 - DNA, 1449 bp, artificial sequence
  Nucleic Acid Sequence of Heavy Chain of K090-02a002
  CDS (16)..(1440)
aagcttgccgccaccatgaaacacctgtggttcttcctcctcctggtggcggcccccagatgggtcctgtccgaggtgaagctggtggagtctgggggaggcttagtgaagcctggagggtcccggaaactctcctgtgcagcctctggattcactttcagtgactatggaatgcactgggtccgtcaggctccagagaaggggctggagtgggttgcatacattagtagtggcagtagtaccatctactatgcagacacagtgaagggccgattcaccatctccagagacaatgccaagaacaccctgttcctgcaaatgaccagtctaaggtctgaggacacagccatgtattactgtgcaaggcatagttactatagttatgacgttgggggtgactatgttatggactactggggtcaaggaacctcagtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 166 - protein, 474 aa, artificial sequence
  Synthetic Construct
MKHLWFFLLLVAAPRWVLSEVKLVESGGGLVKPGGSRKLSCAASGFTFSDYGMHWVRQAPEKGLEWVAYISSGSSTIYYADTVKGRFTISRDNAKNTLFLQMTSLRSEDTAMYYCARHSYYSYDVGGDYVMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 167 - DNA, 731 bp, artificial sequence
  Nucleic Acid Sequence of Light Chain of K090-02a002
  CDS (16)..(720)
aagcttgccgccaccatgaggctccttgctcagcttctggggctgctaatgctctgggtccctggatccagtggggacattgtgatgtcacagtctcccaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatcctctcacgttcggtgctgggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 168 - protein, 234 aa, artificial sequence
  Synthetic Construct
MRLLAQLLGLLMLWVPGSSGDIVMSQSPKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 169 - DNA, 1428 bp, artificial sequence
  Nucleic Acid Sequence of Heavy Chain of K090-02a006
  CDS (16)..(1419)
aagcttgccgccaccatgaaacacctgtggttcttcctcctcctggtggcggcccccagatgggtcctgtccgaggttcagctgcagcagtctggagctgagctggtgaagcctggggcttcagtgaagatatcctgcagggcttctggttactcattcactggctacaacatgaactgggtgaagcagagccatggaaagagccttgagtggattggaaatattaatccttactatggtactactaactacaatcagaagttcaagggcaaggccacattgactgtagacaaatcttccagcacagcctacatgcagctcaacagcctgacatctgaggactctgcagtctattactgtgcaagtgatggttactacggggctatggactactggggtcaaggaacctcagtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 170 - protein, 467 aa, artificial sequence
  Synthetic Construct
MKHLWFFLLLVAAPRWVLSEVQLQQSGAELVKPGASVKISCRASGYSFTGYNMNWVKQSHGKSLEWIGNINPYYGTTNYNQKFKGKATLTVDKSSSTAYMQLNSLTSEDSAVYYCASDGYYGAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 171 - DNA, 731 bp, artificial sequence
  Nucleic Acid Sequence of Light Chain of K090-02a006
  CDS (16)..(720)
aagcttgccgccaccatgaggctccttgctcagcttctggggctgctaatgctctgggtccctggatccagtggggacatcaagatgacccagtctccaaaattcatgtccacatcagtaggagacagggtcagcgtcacctgcaaggccagtcagaatgtgggtacttatgtagcctggtatcaacagaaaccagggcaatctcctaaagcactgatttactcggcatcctaccggtacagtggagtccctgatcgcttcacaggcactggatctgggacagatttcactctcaccatcagcaatgtgcagtctgaagacttggcagagtatttctgtcagcaatattacaactatccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcc

SEQ ID NO: 172 - protein, 234 aa, artificial sequence
  Synthetic Construct
MRLLAQLLGLLMLWVPGSSGDIKMTQSPKFMSTSVGDRVSVTCKASQNVGTYVAWYQQKPGQSPKALIYSASYRYSGVPDRFTGTGSGTDFTLTISNVQSEDLAEYFCQQYYNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 173 - DNA, 1449 bp, artificial sequence
  Nucleic Acid Sequence of Heavy Chain of K090-02a010
  CDS (16)..(1440)
aagcttgccgccaccatgaaacacctgtggttcttcctcctcctggtggcggcccccagatgggtcctgtccgaggtgaagctggtggaatctgggggaggcttagtgaagcctggagggtcccggaaactctcctgtgcagcctctggattcactttcagtgactatggaatgcactgggtccgtcaggctccagagaaggggctggagtgggttgcatacattaatagtggcagtagtaccatctactatgcagacacagtgaagggccgattcaccatctccagagacaatgccaagaacaccctgttcctgcaaatgaccagtctaaggtctggggacacagccatgtattactgtgcaaggcatagttactatagttatgacgttgggggtgactatgttatggactactggggtcaaggaacctcagtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 174 - protein, 474 aa, artificial sequence
  Synthetic Construct
MKHLWFFLLLVAAPRWVLSEVKLVESGGGLVKPGGSRKLSCAASGFTFSDYGMHWVRQAPEKGLEWVAYINSGSSTIYYADTVKGRFTISRDNAKNTLFLQMTSLRSGDTAMYYCARHSYYSYDVGGDYVMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 175 - DNA, 798 bp, artificial sequence
  Nucleic Acid Sequence of Light Chain of K090-02a010
  CDS (16)..(720)
aagcttgccgccaccatgaggctccttgctcagcttctggggctgctaatgctctgggtccctggatccagtggggacattgtgctgacacagtctcccaaattcatgtccacatcagtaggagacagggtcagcatcacctgcaaggccagtcaggatgtgggtactgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatatagcagctatccgctcacgttcggtgctgggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcctctagatgatcattccctttagtgagggttaatgcttcgagcagacatgataagatacattgatgag

SEQ ID NO: 176 - protein, 234 aa, artificial sequence
  Synthetic Construct
MRLLAQLLGLLMLWVPGSSGDIVLTQSPKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 177 - DNA, 1428 bp, artificial sequence
  Nucleic Acid Sequence of Heavy Chain of K090-02a014
  CDS (16)..(1419)
aagcttgccgccaccatgaaacacctgtggttcttcctcctcctggtggcggcccccagatgggtcctgtccgaggttcagcttcagcagtctggacctgaactggtgaagcctggggcttcagtgaagatatcctgcaaggcttctggttactcattcactggctactacatacactgggtgaagcaaagtcctgaaaagagccttgagtggattggagagattaatcctagcactggtgatattacctacaaccagaaattcaaggccaaggccacattgactgtagacaaatcctccagcacagcctacatgcagctcaagagcctgacatctgatgactctgcagtctattactgtgcaagagagcggaattacgaccggtttgcttactggggccaagggactctggtcactgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 178 - protein, 467 aa, artificial sequence
  Synthetic Construct
MKHLWFFLLLVAAPRWVLSEVQLQQSGPELVKPGASVKISCKASGYSFTGYYIHWVKQSPEKSLEWIGEINPSTGDITYNQKFKAKATLTVDKSSSTAYMQLKSLTSDDSAVYYCARERNYDRFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 179 - DNA, 798 bp, artificial sequence
  Nucleic Acid Sequence of Light Chain of K090-02a014
  CDS (16)..(723)
aagcttgccgccaccatgaggctccttgctcagcttctggggctgctaatgctctgggtccctggatccagtgggagtattgtgatgacccagactccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccgctcacgttcggtgctgggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcctctagatgatcattccctttagtgagggttaatgcttcgagcagacatgataagatacattgat

SEQ ID NO: 180 - protein, 235 aa, artificial sequence
  Synthetic Construct
MRLLAQLLGLLMLWVPGSSGSIVMTQTPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 181 - DNA, 1431 bp, artificial sequence
  Nucleic Acid Sequence of Heavy Chain of K090-02a022
  CDS (16)..(1422)
aagcttgccgccaccatgaaacacctgtggttcttcctcctcctggtggcggcccccagatgggtcctgtcccaggtccaactgcagcagcctggacctgagctggtgaagcctggggcttcagtgaagatgtcctgtaaggcttctggatacacattcactgactactacatgaactgggtgaagcagagtcatggaaagagccttgagtggattggacgtgttaatcctagcaatggtggtactagctacaaccagaagttcaagggcaaggccacattgacagtagacaaatccctcagcacagcctacatgcagctcaacagcctgacatctgaggactctgcggtctattactgtgcaagaagtggagagaattactatgctatggactactggggtcaaggaacctcagtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 182 - protein, 468 aa, artificial sequence
  Synthetic Construct
MKHLWFFLLLVAAPRWVLSQVQLQQPGPELVKPGASVKMSCKASGYTFTDYYMNWVKQSHGKSLEWIGRVNPSNGGTSYNQKFKGKATLTVDKSLSTAYMQLNSLTSEDSAVYYCARSGENYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 183 - DNA, 795 bp, artificial sequence
  Nucleic Acid Sequence of Light Chain of K090-02a022
  CDS (16)..(720)
aagcttgccgccaccatgaggctccttgctcagcttctggggctgctaatgctctgggtccctggatccagtggggacatccagatgacccagtctccagcctccctatctgcatctgtgggagaaactgtcaccatcacatgtcgagcaagtgagaatatttacagttatttagcatggtatcagcagaaacagggaaaatctcctcagctcctggtctataatggaaaaaccttagcagaaggtgtgtcatcaaggttcagtggcagtggatcaggcacacagttttctctgaagattaacagcctgcagcctgaagattttgggagttattactgtcaaaatcattatggtgttccgtggacgttcggaggaggcaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcctctagatgatcattccctttagtgagggttaatgcttcgagcagacatgataagatacattgat

SEQ ID NO: 184 - protein, 234 aa, artificial sequence
  Synthetic Construct
MRLLAQLLGLLMLWVPGSSGDIQMTQSPASLSASVGETVTITCRASENIYSYLAWYQQKQGKSPQLLVYNGKTLAEGVSSRFSGSGSGTQFSLKINSLQPEDFGSYYCQNHYGVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 185 - DNA, 1428 bp, artificial sequence
  Nucleic Acid Sequence of Heavy Chain of K090-02a034
  CDS (16)..(1419)
aagcttgccgccaccatgaaacacctgtggttcttcctcctcctggtggcggcccccagatgggtcctgtccgaggttcagcttcagcagtctggacctgagctggtgaagcctggggcttcagtgaagatatcctgcaaggcttctggttactcattcactggctactacatgcactgggtgaagcaaagtcctgaaaagagccttgagtggattggagagattaatcctagcactggtgatactacctacaaccagaagttcaaggccaaggccacattgactgtagacaaatcctccagcacagcctacatacagctcaagagcctgacatctgaggactctgcagtctattactgtgcaagagagcggaattacgaccggtttgcttactggggccaagggactctggtcactgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 186 - protein, 467 aa, artificial sequence
  Synthetic Construct
MKHLWFFLLLVAAPRWVLSEVQLQQSGPELVKPGASVKISCKASGYSFTGYYMHWVKQSPEKSLEWIGEINPSTGDTTYNQKFKAKATLTVDKSSSTAYIQLKSLTSEDSAVYYCARERNYDRFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 187 - DNA, 796 bp, artificial sequence
  Nucleic Acid Sequence of Light Chain of K090-02a034
  CDS (16)..(717)
  misc_feature (775)..(775): n is a, c, g, t or u
aagcttgccgccaccatgaggctccttgctcagcttctggggctgctaatgctctgggtccctggatccagtgggcaaattgttctctcccagtctccagcaatcatgtctgcatctccaggggagaaggtcaccatgacctgcagtgccagctcaagtgtaagttacatgtactggtaccagcagaagccaggctcctcccccagactcctgatttatgacacatccaaccttgcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcctctagatgatcattccctttagtgagggttaatgcttcgagcagacnatgataagatacattgatgag

SEQ ID NO: 188 - protein, 233 aa, artificial sequence
  Synthetic Construct
MRLLAQLLGLLMLWVPGSSGQIVLSQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 189 - DNA, 1413 bp, artificial sequence
  Nucleic Acid Sequence of Heavy Chain of K090-02a035
  CDS (16)..(1404)
aagcttgccgccaccatgaaacacctgtggttcttcctcctcctggtggcggcccccagatgggtcctgtcccaggtccagctgcagcagtctggggccgagctggtgaagcctggggcttcagtgaagctgtcctgcaagacttctggttacaccttcaccagctactggatgcactgggtgaagcagaggcctggacaaggccttgaatggattggtaatattgacccttctgatagtgaaactcactacaatcaaaagttcaaggacaaggccacattgactgttgacaaatcctccagcacagcctacatgcagctcagcggcctgacatctgaggactctgcggtctattactgtgcaagaaacttgggctactggggtcaaggaacctcagtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 190 - protein, 462 aa, artificial sequence
  Synthetic Construct
MKHLWFFLLLVAAPRWVLSQVQLQQSGAELVKPGASVKLSCKTSGYTFTSYWMHWVKQRPGQGLEWIGNIDPSDSETHYNQKFKDKATLTVDKSSSTAYMQLSGLTSEDSAVYYCARNLGYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 191 - DNA, 812 bp, artificial sequence
  Nucleic Acid Sequence of Light Chain of K090-02a035
  CDS (16)..(735)
aagcttgccgccaccatgaggctccttgctcagcttctggggctgctaatgctctgggtccctggatccagtggggacatcaagatgacccagtctccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcctctagatgatcattccctttagtgagggttaatgcttcgagcagacatgataagatacattgatga

SEQ ID NO: 192 - protein, 239 aa, artificial sequence
  Synthetic Construct
MRLLAQLLGLLMLWVPGSSGDIKMTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 193 - DNA, 1449 bp, artificial sequence
  Nucleic Acid Sequence of Heavy Chain of K090-02b006
  CDS (16)..(1440)
aagcttgccgccaccatgaaacacctgtggttcttcctcctcctggtggcggcccccagatgggtcctgtccgaagtgcagctggtggagtctgggggaggcttagtgaagcctggagggtcccggaaactctcctgtgcagcctctggattcactttcagtgactatggaatgcactgggtccgtcaggctccagagaaggggctggagtgggttgcatacattagtagtggcagtagtaccatctactatgcagacacagtgaagggccgattcaccatctccagagacaatgccaagaacaccctgttcctgcaaatgaccagtctaaggtctgaggacacagccatatattactgtgtaaggcatagttactataattacgacattgggggttactatgctatggactactggggtcaaggaacctcagtcaccgtctcgagcgccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagccccggggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgatagtctaga

SEQ ID NO: 194 - protein, 474 aa, artificial sequence
  Synthetic Construct
MKHLWFFLLLVAAPRWVLSEVQLVESGGGLVKPGGSRKLSCAASGFTFSDYGMHWVRQAPEKGLEWVAYISSGSSTIYYADTVKGRFTISRDNAKNTLFLQMTSLRSEDTAIYYCVRHSYYNYDIGGYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 195 - DNA, 797 bp, artificial sequence
  Nucleic Acid Sequence of Light Chain of K090-02b006
  CDS (16)..(720)
aagcttgccgccaccatgaggctccttgctcagcttctggggctgctaatgctctgggtccctggatccagtggggacattgtgctgacacagtctccatcctccttatctgcctctctgggagaaagagtcagtctcacttgtcgggcaagtcaggaaattagtggttacttaagctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactattactgtctacaatatgctagttatcctctcacgttcggtgctgggaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatagggcgcgcctctagatgatcattccctttagtgagggttaatgcttcgagcagacatgataagatacattgatga

SEQ ID NO: 196 - protein, 234 aa, artificial sequence
  Synthetic Construct
MRLLAQLLGLLMLWVPGSSGDIVLTQSPSSLSASLGERVSLTCRASQEISGYLSWLQQKPDGTIKRLIYAASTLDSGVPKRFSGSRSGSDYSLTISSLESEDFADYYCLQYASYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 197 - DNA, 369 bp, artificial sequence
  Nucleic Acid Sequence of Heavy Chain of Clone 4
  CDS (1)..(369)
gcggtgacgttggacgagtccgggggcggcctccagacgcccggaagagcgctcagcctcgtctgtaaggcctccgggttcaccttcagccgttacgccatgtactgggtgcgacaggcgcccggcaaggggctggagttcgtcgctggtattggcaacactggtagatacacaggctacgggtcggcggtgaagggccgtgccaccatctcgagggacagcgggcagagcacagtgaggctgcaactgaacaacctcagggctgaggacaccggcaactactactgcgccaaaagtgttagtccttactgttgtgatgctgctgacatcgacgcatggggccacgggaccgaagtcatcgtctcctcc

SEQ ID NO: 198 - protein, 123 aa, artificial sequence
  Synthetic Construct
AVTLDESGGGLQTPGRALSLVCKASGFTFSRYAMYWVRQAPGKGLEFVAGIGNTGRYTGYGSAVKGRATISRDSGQSTVRLQLNNLRAEDTGNYYCAKSVSPYCCDAADIDAWGHGTEVIVSS

SEQ ID NO: 199 - DNA, 318 bp, artificial sequence
  Nucleic Acid Sequence of Light Chain of Clone4
  CDS (1)..(318)
gctagcactcagccgtcctcggtgtcagcaaacccaggagaaaccgtcaagatcacctgctccgggggtagcagtggctatgcttatggctggtaccagcagaagtctcctggcagtgcccctgtcactctgctctatagcaacaacaacagaccctcggacatcccttcacgattctccggttccaaatccggctccacagccacattaaccatcactggggtccaagccgaggacgaggctgtctatttctgtgggagtgtagacagcagcagttatgctggtatatttggggccgggacaaccctgaccgtccta

SEQ ID NO: 200 - protein, 106 aa, artificial sequence
  Synthetic Construct
ASTQPSSVSANPGETVKITCSGGSSGYAYGWYQQKSPGSAPVTLLYSNNNRPSDIPSRFSGSKSGSTATLTITGVQAEDEAVYFCGSVDSSSYAGIFGAGTTLTVL

SEQ ID NO: 201 - DNA, 384 bp, artificial sequence
  Nucleic Acid Sequence of Heavy Chain of Clone18
  CDS (1)..(384)
gcggtgacgttggacgagtccgggggcggcctccagacgcccggaggaacgctcagcctcgtctgcaaggcctccgggttcgacttcagcagcgtcaacatgttctgggtgcgacaggcgcccggcaaggggctggagttcgtcgctggtattgataacgatgctactttcacactctacgggtcggcggtgaagggccgtgcctccatctcgagggacaacgggcagagcacagtgaggctgcagctgaacaacctcagggctgaggacaccggcacctactactgcgccaaaactctttgtagtactacttggggttgtggtgcttatagtgctggagatatcgacgcatggggccacgggaccgaagtcatcgtctcctcc

SEQ ID NO: 202 - protein, 128 aa, artificial sequence
  Synthetic Construct
AVTLDESGGGLQTPGGTLSLVCKASGFDFSSVNMFWVRQAPGKGLEFVAGIDNDATFTLYGSAVKGRASISRDNGQSTVRLQLNNLRAEDTGTYYCAKTLCSTTWGCGAYSAGDIDAWGHGTEVIVSS

SEQ ID NO: 203 - DNA, 321 bp, artificial sequence
  Nucleic Acid Sequence of Light Chain of Clone 18
  CDS (1)..(321)
gctagcactcagccgtcctcggtgtcagcaaacccaggggaaactgtcaagatcacctgttccgggggtggcagcagtggctatggttactatggctggtatcagcagaagtcacctggcagtgctcctgtcactgtgatctataacaacaacaagagacccacggacatcccttcacgattctccggtgccctctctggctccacagccacattaaccatcactggggtccaagtcgaggacgaggctgtctattactgtgggagtagggacaacacttatgttggtatatttggggccgggacaaccctgaccgtccta

SEQ ID NO: 204 - protein, 107 aa, artificial sequence
  Synthetic Construct
ASTQPSSVSANPGETVKITCSGGGSSGYGYYGWYQQKSPGSAPVTVIYNNNKRPTDIPSRFSGALSGSTATLTITGVQVEDEAVYYCGSRDNTYVGIFGAGTTLTVL

(19) United States
(12) Patent Application Publication
(10) Pub. No.: US 2020/0308299 A1
(43) Pub. Date: Oct. 1, 2020

(54) ANTI-GPC-1 ANTIBODY

(71) Applicant: National University Corporation Kochi University, Kochi-shi, Kochi (JP)

(72) Inventors: Tetsuji Naka, Kochi-shi, Kochi (JP); Satoshi Serada, Kochi-shi, Kochi (JP); Minoru Fujimoto, Kochi-shi, Kochi (JP)

(21) Appl. No.: 16/608,795

(22) PCT Filed: Apr. 27, 2018
(86) PCT No.: PCT/JP2018/017291

§ 371 Date: Oct. 25, 2019

(30) Foreign Application Priority Data
Apr. 28, 2017 (JP) 2017-090054

(51) Int. Cl. C07K 16/30; A61P 35/00

(52) U.S. Cl. CPC C07K 16/303; A61P 35/00; C07K 2317/565; A61K 2039/505; C07K 2317/73; C07K 2317/92; C07K 2317/77

(57) ABSTRACT

Provided is a novel anti-Glypican-1 antibody and a method for using the same. Provided is an anti-Glypican-1 antibody having an intracellular invasion activity which has never been observed in conventional anti-Glypican-1 antibodies. By taking advantage of the intracellular invasion activity of the antibody according to the present invention, the present invention is usable for various therapeutic purposes beyond the scope of the conventional assumption. Also provided is a composition for preventing or treating Glypican-1 positive cancer, said composition comprising a complex of a substance capable of binding to Glypican-1 (for example, an anti-Glypican-1 antibody) with a drug having a cytotoxic activity.

### TECHNICAL FIELD

The present invention relates to an antibody that specifically binds to Glypican-1 (GPC-1), and related technology, method, agent, and the like.

### BACKGROUND ART

It was found that Glypican-1 molecules are significantly more strongly expressed in esophageal cancer cells than in normal cells and can be used as tumor markers (Patent Literature 1). Antibodies that specifically bind to Glypican-1 have also been isolated (Patent Literature 1). However, an effective therapeutic drug that targets Glypican-1 has not yet been found.

### CITATION LIST

### Patent Literature

[PTL 1] International Publication No. WO 2015/098112

### SUMMARY OF INVENTION

### Solution to Problem

In one aspect, the present invention provides an anti-gypican-1 antibody having intracellular invasion activity. Such activity was not found in conventional anti-Glypican-1 antibodies. The present invention can be used in various therapeutic applications that were inconceivable with conventional art by utilizing intracellular invasion activity of the antibody of the invention.

Thus, in another aspect, the present invention also provides a composition for preventing or treating Glypican-1 positive cancer, comprising a complex of a substance that binds to Glypican-1 (e.g., anti-Glypican-1 antibody) and an agent having cytotoxic activity.

In another aspect, the present invention provides an anti-Glypican-1 antibody having a binding activity that is stronger than conventional antibodies, or a fragment thereof. Such an antibody or fragment thereof is useful as a diagnostic agent for cancer or the like.

Such an antibody or a fragment thereof is also applicable as a companion diagnostic drug, companion therapeutic drug, and the like.

In view of the above, for example, the present invention provides the following items.

### <Antibody>

### (Item 1A)

An anti-human Glypican-1 antibody or antigen binding fragment thereof, wherein the antibody is selected from the group consisting of:

(a) an antibody comprising amino acid sequences of heavy chain CDR1, heavy chain CDR2, and heavy chain CDR3 set forth in SEQ ID NOs: 53, 54, and 55, respectively, and light chain CDR1, light chain CDR2, and light chain CDR3 set forth in SEQ ID NOs: 56, 57, and 58, respectively;
(b) an antibody comprising amino acid sequences of heavy chain CDR1, heavy chain CDR2, and heavy chain CDR3 set forth in SEQ ID NOs: 5, 6, and 7, respectively, and light chain CDR1, light chain CDR2, and light chain CDR3 set forth in SEQ ID NOs: 8, 9, and 10, respectively;
(c) an antibody comprising amino acid sequences of heavy chain CDR1, heavy chain CDR2, and heavy chain CDR3 set forth in SEQ ID NOs: 11, 12, and 13, respectively, and light chain CDR1, light chain CDR2, and light chain CDR3 set forth in SEQ ID NOs: 14, 15, and 16, respectively;
(d) an antibody comprising amino acid sequences of heavy chain CDR1, heavy chain CDR2, and heavy chain CDR3 set forth in SEQ ID NOs: 17, 18, and 19, respectively, and light chain CDR1, light chain CDR2, and light chain CDR3 set forth in SEQ ID NOs: 20, 21, and 22, respectively;
(e) an antibody comprising amino acid sequences of heavy chain CDR1, heavy chain CDR2, and heavy chain CDR3 set forth in SEQ ID NOs: 23, 24, and 25, respectively, and light chain CDR1, light chain CDR2, and light chain CDR3 set forth in SEQ ID NOs: 26, 27, and 28, respectively;
(f) an antibody comprising amino acid sequences of heavy chain CDR1, heavy chain CDR2, and heavy chain CDR3 set forth in SEQ ID NOs: 29, 30, and 31, respectively, and light chain CDR1, light chain CDR2, and light chain CDR3 set forth in SEQ ID NOs: 32, 33, and 34, respectively;
(g) an antibody comprising amino acid sequences of heavy chain CDR1, heavy chain CDR2, and heavy chain CDR3 set forth in SEQ ID NOs: 35, 36, and 37, respectively, and light chain CDR1, light chain CDR2, and light chain CDR3 set forth in SEQ ID NOs: 38, 39, and 40, respectively;
(h) an antibody comprising amino acid sequences of heavy chain CDR1, heavy chain CDR2, and heavy chain CDR3 set forth in SEQ ID NOs: 41, 42, and 43, respectively, and light chain CDR1, light chain CDR2, and light chain CDR3 set forth in SEQ ID NOs: 44, 45, and 46, respectively;
(i) an antibody comprising amino acid sequences of heavy chain CDR1, heavy chain CDR2, and heavy chain CDR3 set forth in SEQ ID NOs: 47, 48, and 49, respectively, and light chain CDR1, light chain CDR2, and light chain CDR3 set forth in SEQ ID NOs: 50, 51, and 52, respectively;
(j) an antibody comprising amino acid sequences of heavy chain CDR1, heavy chain CDR2, and heavy chain CDR3 set forth in SEQ ID NOs: 59, 60, and 61, respectively, and light chain CDR1, light chain CDR2, and light chain CDR3 set forth in SEQ ID NOs: 62, 63, and 64, respectively;
(k) an antibody comprising amino acid sequences of heavy chain CDR1, heavy chain CDR2, and heavy chain CDR3 set forth in SEQ ID NOs: 65, 66, and 67, respectively, and light chain CDR1, light chain CDR2, and light chain CDR3 set forth in SEQ ID NOs: 68, 69, and 70, respectively;
(l) an antibody comprising amino acid sequences of heavy chain CDR1, heavy chain CDR2, and heavy chain CDR3 set forth in SEQ ID NOs: 71, 72, and 73, respectively, and light chain CDR1, light chain CDR2, and light chain CDR3 set forth in SEQ ID NOs: 74, 75, and 76, respectively;
(m) an antibody comprising amino acid sequences of heavy chain CDR1, heavy chain CDR2, and heavy chain CDR3 set forth in SEQ ID NOs: 77, 78, and 79, respectively, and light chain CDR1, light chain CDR2, and light chain CDR3 set forth in SEQ ID NOs: 80, 81, and 82, respectively;
(n) an antibody comprising amino acid sequences of heavy chain CDR1, heavy chain CDR2, and heavy chain CDR3 set forth in SEQ ID NOs: 83, 84, and 85, respectively, and light chain CDR1, light chain CDR2, and light chain CDR3 set forth in SEQ ID NOs: 86, 87, and 88, respectively;
(o) an antibody comprising amino acid sequences of heavy chain CDR1, heavy chain CDR2, and heavy chain CDR3 set forth in SEQ ID NOs: 89, 90, and 91, respectively, and light chain CDR1, light chain CDR2, and light chain CDR3 set forth in SEQ ID NOs: 92, 93, and 94, respectively;
(p) an antibody comprising amino acid sequences of heavy chain CDR1, heavy chain CDR2, and heavy chain CDR3 set forth. in SEQ ID NOs: 95, 96, and 97, respectively, and light chain CDR1, light chain CDR2, and light chain CDR3 set forth in SEQ ID NOs: 98, 99, and 100, respectively;
(q) an antibody comprising amino acid sequences of heavy chain CDR1, heavy chain CDR2, and heavy chain CDR3 set forth in SEQ ID NOs: 101, 102, and 103, respectively, and light chain CDR1, light chain CDR2, and light chain CDR3 set forth in SEQ ID NOs: 104, 105, and 106, respectively;
(r) an antibody comprising amino acid sequences of heavy chain CDR1, heavy chain CDR2, and heavy chain CDR3 set forth in SEQ ID NOs: 107, 108, and 109, respectively, and light chain CDR1, light chain CDR2, and light chain CDR3 set forth in SEQ ID NOs: 110, 111, and 112, respectively; and
(s) a mutant of an antibody selected from (a) to (r) comprising at least one substitution, addition, or deletion in a CDR moiety.

### (Item 1A-1)

The antibody or antigen binding fragment thereof of item 1A, wherein the antibody is selected from the group consisting of (a), (b), (d), (e), (g), (h), (i), (j), (k), (l), (m), and (n).

### (Item 2A)

The antibody or antigen binding fragment thereof of item 1A, wherein the antibody is selected from the group consisting of:

(a) an antibody comprising amino acid sequences of a heavy chain set forth in SEQ ID NO: 158 and a light chain set forth in SEQ ID NO: 160;
(b) an antibody comprising amino acid sequences of a heavy chain set forth in SEQ ID NO: 126 and a light chain set forth in SEQ ID NO: 128;
(c) an antibody comprising amino acid sequences of a heavy chain set forth in SEQ ID NO: 130 and a light chain set forth in SEQ ID NO: 132;
(d) an antibody comprising amino acid sequences of a heavy chain set forth in SEQ ID NO: 134 and a light chain set forth in SEQ ID NO: 136;
(e) an antibody comprising amino acid sequences of a heavy chain set forth in SEQ ID NO: 138 and a light chain set forth in SEQ ID NO: 140;
(f) an antibody comprising amino acid sequences of a heavy chain set forth in SEQ ID NO: 142 and a light chain set forth in SEQ ID NO: 144;
(g) an antibody comprising amino acid sequences of a heavy chain set forth in SEQ ID NO: 146 and a light chain set forth in SEQ ID NO: 148;
(h) an antibody comprising amino acid sequences of a heavy chain set forth in SEQ ID NO: 150 and a light chain set forth in SEQ ID NO: 152;
(i) an antibody comprising amino acid sequences of a heavy chain set forth in SEQ ID NO: 154 and a light chain set forth in SEQ ID NO: 156;
(j) an antibody comprising amino acid sequences of a heavy chain set forth in SEQ ID NO: 162 and a light chain set forth in SEQ ID NO: 164;
(k) an antibody comprising amino acid sequences of a heavy chain set forth in SEQ ID NO: 166 and a light chain set forth in SEQ ID NO: 168;
(l) an antibody comprising amino acid sequences of a heavy chain set forth in SEQ ID NO: 170 and a light chain set forth in SEQ ID NO: 172;
(m) an antibody comprising amino acid sequences of a heavy chain set forth in SEQ ID NO: 174 and a light chain set forth in SEQ ID NO: 176;
(n) an antibody comprising amino acid sequences of a heavy chain set forth in SEQ ID NO: 178 and a light chain set forth in SEQ ID NO: 180;
(o) an antibody comprising amino acid sequences of a heavy chain set forth in SEQ ID NO: 182 and a light chain set forth in SEQ ID NO: 184;
(p) an antibody comprising amino acid sequences of a heavy chain set forth in SEQ ID NO: 186 and a light chain set forth in SEQ ID NO: 188;
(q) an antibody comprising amino acid sequences of a heavy chain set forth in SEQ ID NO: 190 and a light chain set forth in SEQ ID NO: 192;
(r) an antibody comprising amino acid sequences of a heavy chain set forth in SEQ ID NO: 194 and a light chain set forth in SEQ ID NO: 196; and
(s) a mutant of an antibody selected from (a) to (r) comprising at least one substitution, addition, or deletion.

### (Item 2A-1)

The antibody or antigen binding fragment thereof of item 2A, wherein the antibody is selected from the group consisting of (a), (b), (d), (e), (g), (h), (i), (j), (k), (l), (m), and (n).

### (Item 3A)

The antibody or antigen binding fragment thereof of item 1A or 2A, wherein the mutant comprises at least one substitution, addition, or deletion in a framework of the antibody.

### (Item 4A)

The antibody or antigen binding fragment thereof of any one of items 1A to 3A, which binds to human Glypican-1 at a binding constant of about 10 nM or less.

### (Item 5A)

The antibody or antigen binding fragment thereof of any one of items 1A to 4A, having an internalization activity of about 30% or greater with respect to Glypican-1 positive cells after 6 hours from starting incubation with the antibody or antigen fragment thereof in an internalization assay.

### (Item 5A-1)

The antibody or antigen binding fragment thereof of any one of items 1A to 4A, having an internalization activity of about 30% or greater with respect to Glypican-1 positive cells after 2 hours from starting incubation with the antibody or antigen fragment thereof in an internalization assay.

### (Item 6A)

The antibody or antigen binding fragment thereof of any one of items 1A to 5A, having an internalization activity of about 50% or greater with respect to Glypican-1 positive cells after 6 hours from starting incubation with the antibody or antigen fragment thereof in an internalization assay.

### (Item 6A-1)

The antibody or antigen binding fragment thereof of any one of items 1A to 4A and 5A-1, having an internalization activity of about 50% or greater with respect to Glypican-1 positive cells after 2 hours from starting incubation with the antibody or antigen fragment thereof in an internalization assay.

### (Item 7A)

The antibody or antigen binding fragment thereof of any one of items 1A to 6A, having an internalization activity of about 60% or greater with respect to Glypican-1 positive cells after 6 hours from starting incubation with the antibody or antigen fragment thereof in an internalization assay.

### (Item 7A-1)

The antibody or antigen binding fragment thereof of any one of items 1A to 4A, 5A-1, and 6A-1 having an internalization activity of about 60% or greater with respect to Glypican-1 positive cells after 2 hours from starting incubation with the antibody or antigen fragment thereof in an internalization assay.

### (Item 8A)

The antibody or antigen binding fragment thereof of any one of items 1A to 7A, wherein the antibody is an antibody selected from a monoclonal antibody, a polyclonal antibody, a chimeric antibody, a humanized antibody, a human antibody, a multifunctional antibody, a bispecific or oligospecific antibody, a single chain antibody, an scFV, a diabody, an sc(Fv)2 (single chain (Fv)2), and an scFv-Fc.

### (Item 9A)

A pharmaceutical composition comprising the antibody or antigen binding fragment thereof of any one of items 1A to 8A.

### (Item 10A)

The pharmaceutical composition of item 9A, wherein the pharmaceutical composition is for intracellular invasion of an active ingredient.

### <Complex>

### (Item 1B)

A complex of an anti-Glypican-1 antibody having activity for intracellular invasion into Glypican-1 positive cells or antigen binding fragment thereof and an agent having cytotoxic activity.

### (Item 2B)

The complex of item 1B, wherein a substance that binds to the Glypican-1 is operably linked to the agent having cytotoxic activity via a linker.

### (Item 3B)

The complex of item 1B or 2B, wherein an epitope of the antibody comprises:

(a) positions 33 to 61 of SEQ ID NO: 2;
(b) positions 339 to 358 and/or 388 to 421 of SEQ ID NO: 2;
(c) positions 430 to 530 of SEQ ID NO: 2;
(d) positions 33 to 61, 339 to 358, and/or 388 to 421 of SEQ ID NO: 2;
(e) positions 339 to 358, 388 to 421, and/or 430 to 530 of SEQ ID NO: 2; or
(f) positions 33 to 61, 339 to 358, 388 to 421, and/or 430 to 530 of SEQ ID NO: 2.

### (Item 4B)

The complex of item 3B, wherein the epitope of the antibody comprises:

(a) positions 33 to 61 of SEQ ID NO: 2;
(b) positions 339 to 358 and 388 to 421 of SEQ ID NO: 2;
(c) positions 33 to 61, 339 to 358, and 388 to 421 of SEQ ID NO: 2; or
(d) positions 33 to 61, 339 to 358, 388 to 421, and 430 to 530 of SEQ ID NO: 2.

### (Item 5B)

The complex of any one of items 1B to 4B, wherein the antibody is selected from the group consisting of:

(a) an antibody comprising amino acid sequences of heavy chain CDR1, heavy chain CDR2, and heavy chain CDR3 set forth in SEQ ID NOs: 53, 54, and 55, respectively, and light chain CDR1, light chain CDR2, and light chain CDR3 set forth in SEQ ID NOs: 56, 57, and 58, respectively;
(b) an antibody comprising amino acid sequences of heavy chain CDR1, heavy chain CDR2, and heavy chain CDR3 set forth in SEQ ID NOs: 5, 6, and 7, respectively, and light chain CDR1, light chain CDR2, and light chain CDR3 set forth in SEQ ID NOs: 8, 9, and 10, respectively;
(c) an antibody comprising amino acid sequences of heavy chain CDR1, heavy chain CDR2, and heavy chain CDR3 set forth in SEQ ID NOs: 11, 12, and 13, respectively, and light chain CDR1, light chain CDR2, and light chain CDR3 set forth in SEQ ID NOs: 14, 15, and 16, respectively;
(d) an antibody comprising amino acid sequences of heavy chain CDR1, heavy chain CDR2, and heavy chain CDR3 set forth in SEQ ID NOs: 17, 18, and 19, respectively, and light chain CDR1, light chain CDR2, and light chain CDR3 set forth in SEQ ID NOs: 20, 21, and 22, respectively;
(e) an antibody comprising amino acid sequences of heavy chain CDR1, heavy chain CDR2, and heavy chain CDR3 set forth in SEQ ID NOs: 23, 24, and 25, respectively, and light chain CDR1, light chain CDR2, and light chain CDR3 set forth in SEQ ID NOs: 26, 27, and 28, respectively;
(f) an antibody comprising amino acid sequences of heavy chain CDR1, heavy chain CDR2, and heavy chain CDR3 set forth in SEQ ID NOs: 29, 30, and 31, respectively, and light chain CDR1, light chain CDR2, and light chain CDR3 set forth in SEQ ID NOs: 32, 33, and 34, respectively;
(g) an antibody comprising amino acid sequences of heavy chain CDR1, heavy chain CDR2, and heavy chain CDR3 set forth in SEQ ID NOs: 35, 36, and 37, respectively, and light chain CDR1, light chain CDR2, and light chain CDR3 set forth in SEQ ID NOs: 38, 39, and 40, respectively;
(h) an antibody comprising amino acid sequences of heavy chain CDR1, heavy chain CDR2, and heavy chain CDR3 set forth in SEQ ID NOs: 41, 42, and 43, respectively, and light chain CDR1, light chain CDR2, and light chain CDR3 set forth in SEQ ID NOs: 44, 45, and 46, respectively;
(i) an antibody comprising amino acid sequences of heavy chain CDR1, heavy chain CDR2, and heavy chain CDR3 set forth in SEQ ID NOs: 47, 48, and 49, respectively, and light chain CDR1, light chain CDR2, and light chain CDR3 set forth in SEQ ID NOs: 50, 51, and 52, respectively;
(j) an antibody comprising amino acid sequences of heavy chain CDR1, heavy chain CDR2, and heavy chain CDR3 set forth in SEQ ID NOs: 59, 60, and 61, respectively, and light chain CDR1, light chain CDR2, and light chain CDR3 set forth in SEQ ID NOs: 62, 63, and 64, respectively;
(k) an antibody comprising amino acid sequences of heavy chain CDR1, heavy chain CDR2, and heavy chain CDR3 set forth in SEQ ID NOs: 65, 66, and 67, respectively, and light chain CDR1, light chain CDR2, and light chain CDR3 set forth in SEQ ID NOs: 68, 69, and 70, respectively;
(l) an antibody comprising amino acid sequences of heavy chain CDR1, heavy chain CDR2, and heavy chain CDR3 set forth in SEQ ID NOs: 71, 72, and 73, respectively, and light chain CDR1, light chain CDR2, and light chain CDR3 set forth in SEQ ID NOs: 74, 75, and 76, respectively;
(m) an antibody comprising amino acid sequences of heavy chain CDR1, heavy chain. CDR2, and heavy chain CDR3 set forth in SEQ ID NOs: 77, 78, and 79, respectively, and light chain CDR1, light chain CDR2, and light chain CDR3 set forth in SEQ ID NOs: 80, 81, and 82, respectively;
(n) an antibody comprising amino acid sequences of heavy chain CDR1, heavy chain CDR2, and heavy chain CDR3 set forth in SEQ ID NOs: 83, 84, and 85, respectively, and light chain CDR1, light chain CDR2, and light chain CDR3 set forth in SEQ ID NOs: 86, 87, and 88, respectively;
(o) an antibody comprising amino acid sequences of heavy chain CDR1, heavy chain CDR2, and heavy chain CDR3 set forth in SEQ ID NOs: 89, 90, and 91, respectively, and light chain CDR1, light chain CDR2, and light chain CDR3 set forth in SEQ ID NOs: 92, 93, and 94, respectively;
(p) an antibody comprising amino acid sequences of heavy chain CDR1, heavy chain CDR2, and heavy chain CDR3 set forth in SEQ ID NOs: 95, 96, and 97, respectively, and light chain CDR1, light chain CDR2, and light chain CDR3 set forth in SEQ ID NOs: 98, 99, and 100, respectively;
(q) an antibody comprising amino acid sequences of heavy chain CDR1, heavy chain CDR2, and heavy chain CDR3 set forth in SEQ ID NOs: 101, 102, and 103, respectively, and light chain CDR1, light chain CDR2, and light chain CDR3 set forth in SEQ ID NOs: 104, 105, and 106, respectively;
(r) an antibody comprising amino acid sequences of heavy chain CDR1, heavy chain CDR2, and heavy chain CDR3 set forth in SEQ ID NOs: 107, 108, and 109, respectively, and light chain CDR1, light chain CDR2, and light chain CDR3 set forth in SEQ ID NOs: 110, 111, and 112, respectively;
(s) an antibody comprising amino acid sequences of heavy chain CDR1, heavy chain CDR2, and heavy chain CDR3 set forth in SEQ ID NOs: 113, 114, and 115, respectively, and light chain CDR1, light chain CDR2, and light chain CDR3 set forth in SEQ ID NOs: 116, 117, and 118, respectively;
(t) an antibody comprising amino acid sequences of heavy chain CDR1, heavy chain CDR2, and heavy chain CDR3 set forth in SEQ ID NOs: 125, 126, and 127, respectively, and light chain CDR1, light chain CDR2, and light chain CDR3 set forth in SEQ ID NOs: 128, 129, and 130, respectively; and
(u) a mutant of an antibody selected from (a) to (t) comprising at least one substitution, addition, or deletion.

### (Item 5B-1)

The complex of any one of item 5B, wherein the antibody is selected from the group consisting of (a), (b), (d), (e), (g), (h), (i), (j), (k), (l), (m), (n), (s), and (t).

### (Item 6B)

The complex of any one of item 5B, wherein the antibody is selected from the group consisting of:

(a) an antibody comprising amino acid sequences of a heavy chain set forth in SEQ ID NO: 158 and a light chain set forth in SEQ ID NO: 160;
(b) an antibody comprising amino acid sequences of a heavy chain set forth in SEQ ID NO: 126 and a light chain set forth in SEQ ID NO: 128;
(c) an antibody comprising amino acid sequences of a heavy chain set forth in SEQ ID NO: 130 and a light chain set forth in SEQ ID NO: 132;
(d) an antibody comprising amino acid sequences of a heavy chain set forth in SEQ ID NO: 134 and a light chain set forth in SEQ ID NO: 136;
(e) an antibody comprising amino acid sequences of a heavy chain set forth in SEQ ID NO: 138 and a light chain set forth in SEQ ID NO: 140;
(f) an antibody comprising amino acid sequences of a heavy chain set forth in SEQ ID NO: 142 and a light chain set forth in SEQ ID NO: 144;
(g) an antibody comprising amino acid sequences of a heavy chain set forth in SEQ ID NO: 146 and a light chain set forth in SEQ ID NO: 148;
(h) an antibody comprising amino acid sequences of a heavy chain set forth in SEQ ID NO: 150 and a light chain set forth in SEQ ID NO: 152;
(i) an antibody comprising amino acid sequences of a heavy chain set forth in SEQ ID NO: 154 and a light chain set forth in SEQ ID NO: 156;
(j) an antibody comprising amino acid sequences of a heavy chain set forth in SEQ ID NO: 162 and a light chain set forth in SEQ ID NO: 164;
(k) an antibody comprising amino acid sequences of a heavy chain set forth in SEQ ID NO: 166 and a light chain set forth in SEQ ID NO: 168;
(l) an antibody comprising amino acid sequences of a heavy chain set forth in SEQ ID NO: 170 and a light chain set forth in SEQ ID NO: 172;
(m) an antibody comprising amino acid sequences of a heavy chain set forth in SEQ ID NO: 174 and a light chain set forth in SEQ ID NO: 176;
(n) an antibody comprising amino acid sequences of a heavy chain set forth in SEQ ID NO: 178 and a light chain set forth in SEQ ID NO: 180;
(o) an antibody comprising amino acid sequences of a heavy chain set forth in SEQ ID NO: 182 and a light chain set forth in SEQ ID NO: 184;
(p) an antibody comprising amino acid sequences of a heavy chain set forth in SEQ ID NO: 186 and a light chain set forth in SEQ ID NO: 188;
(q) an antibody comprising amino acid sequences of a heavy chain set forth in SEQ ID NO: 190 and a light chain set forth in SEQ ID NO: 192;
(r) an antibody comprising amino acid sequences of a heavy chain set forth in SEQ ID NO: 194 and a light chain set forth in SEQ ID NO: 196;
(s) an antibody comprising amino acid sequences of a heavy chain set forth in SEQ ID NO: 198 and a light chain set forth in SEQ ID NO: 200;
(t) an antibody comprising amino acid sequences of a heavy chain set forth in SEQ ID NO: 202 and a light chain set forth in SEQ ID NO: 204; and
(u) a mutant of an antibody selected from (a) to (t) comprising at least one substitution, addition, or deletion.

### (Item 6B-1)

The complex of any one of item 6B, wherein the antibody is selected from the group consisting of (a), (b), (d), (e), (g), (h), (i), (j), (k), (l), (m), (n), (s), and (t).

### (Item 7B)

The complex of any one of items 1B to 6B, wherein the complex exhibits an IC₅₀ of about 0.5 nM or less in Glypican-1 positive cells.

### <Composition>

### (Item 10)

A composition for preventing or treating Glypican-1 positive cancer, comprising the complex of any one of items 1B to 7B.

### (Item 2C)

The composition of item 1C, wherein cancer cells with the Glypican-1 positive cancer express a high level of glypican-1 on a cell surface.

### (Item 3C)

The composition of item 2C, wherein the cancer cells with the Glypican-1 positive cancer have an anti-Glypican-1 antibody binding capacity of about 15000 or greater in an assay using QIFIKIT®.

### (Item 4C)

The composition of item 3C, wherein the Glypican-1 positive cancer is selected from esophageal cancer, pancreatic cancer, cervical cancer, lung cancer, head and neck cancer, breast cancer, uterine leiomyosarcoma, prostate cancer, and any combination thereof.

### (Item 5C)

The composition of item 3C or 4C, wherein the Glypican-1 positive cancer is esophageal cancer, which comprises esophageal cancer at a lymph node metastasis site, squamous cell carcinoma, and/or adenocarcinoma.

### (Item 6C)

The composition of any one of item 5C, wherein the esophageal cancer comprises squamous cell carcinoma.

### <Detection Agent and Diagnostic Method>

### (Item 1D)

A detection agent for identifying esophageal cancer, comprising the antibody or fragment thereof of any one of items 1A to 8A.

### (Item 2D)

The detection agent of item 1D, wherein the antibody or fragment thereof is labeled.

### (Item 3D)

The detection agent of item 1D or 2D, wherein the esophageal cancer is Glypican-1 positive.

### (Item 4D)

The detection agent of any one of items 1D to 3D, wherein the esophageal cancer comprises esophageal cancer at a lymph node metastasis site, squamous cell carcinoma, and/or adenocarcinoma.

### (Item 5D)

The detection agent of any one of items 1D to 4D, wherein the esophageal cancer comprises squamous cell carcinoma.

### (Item 6D)

The detection agent of any one of items 1D to 5D for administration to a patient determined to have developed Glypican-1 positive esophageal cancer.

### (Item 7D)

A method of using expression of Glypican-1 in a target sample as an indicator of esophageal cancer, the method comprising:

contacting the detection agent of any one of items 1D to 6D with the target sample;

measuring an amount of expression of Glypican-1 in the target sample; and

comparing amounts of expression of Glypican-1 in the target sample and a normal sample.

### (Item 8D)

A diagnostic drug for determining whether a subject is in need of therapy of esophageal cancer, comprising the antibody or antigen binding fragment thereof of any one of items 1A to 8A.

### <Companion Reagent>

### (Item 1E)

A companion reagent for determining whether a subject is in need of cancer therapy with a Glypican-1 inhibitor, comprising the antibody or antigen binding fragment thereof of any one of items 1A to 8A or the detection agent of any one of items 1D to 6D, wherein the reagent is contacted with a target sample, and an amount of expression of Glypican-1 in the target sample is measured, wherein the amount of expression of Glypican-1 in the target sample exceeding an amount of expression of Glypican-1 in a normal sample indicates that the target is in need of therapy with a Glypican-1 inhibitor.

### (Item 2E)

A composition for preventing or treating malignant tumor comprising the complex of any one of items 1B to 7B, wherein a subject having the malignant tumor has a higher expression of Glypican-1 than a normal individual.

### <Syngenic Nonhuman Animals>

### (Item 1F)

A nonhuman animal to which cells that express a cancer antigen are grafted, wherein the cancer antigen is syngenic with the nonhuman animal.

### (Item 2F)

The nonhuman animal of item 1F, wherein the cancer antigen is Glypican-1.

### (Item 3F)

The nonhuman animal of item 1F or 2F, wherein the cells are selected from the group consisting of an LLC cell strain, a 4T1 cell strain, an MH-1 cell strain, a CT26 cell strain, an MC38 cell strain, and a B1610 cell strain, which have been modified to express Glypican-1.

### (Item 4F)

The nonhuman animal of any one of items 1F to 3F, wherein the nonhuman animal is a mouse, and the cells are of an LLC cell strain modified to express mouse Glypican-1.

The present invention is intended so that one or more of the features described above can be provided not only as the explicitly disclosed combinations, but also as other combinations thereof. Additional embodiments and advantages of the present invention are recognized by those skilled in the art by reading and understanding the following detailed description as needed.

### Advantageous Effects of Invention

The present invention provides an antibody that binds to Glypican-1 with better specificity than conventional antibodies. It was found that such an antibody can be utilized as a detection agent used for diagnosis/companion therapy of esophageal cancer, and a complex of the antibody and a drug is very effective in treating esophageal cancer. Surprisingly, it was found that a complex of the antibody and a drug is effective on Glypican-1 positive cancer such as pancreatic cancer and cervical cancer in addition to esophageal cancer.

### BRIEF DESCRIPTION OF DRAWINGS

FIG. 1 depicts a schematic diagram of an ADC used in an ADC assay using an anticancer agent binding secondary antibody.

FIG. 2 shows results of anticancer agent sensitivity of TE14 cells. The vertical axis indicates the ratio of the viable cell count with respect to the control, and the horizontal axis indicates the concentration of anticancer agent.

FIG. 3 shows a table summarizing ADC 1050, K_{D} measured by Biacore, K_{D} measured by FACS, mGPC1 intersection, and epitope of each clone.

FIG. 4 shows results of ADC assays in LK2, TE8, and TE14 cells.

FIG. 5 shows a table summarizing the results obtained in FIG. 4.

FIG. 6 depicts a schematic diagram of a structure of an exemplary antibody-drug conjugate (ADC).

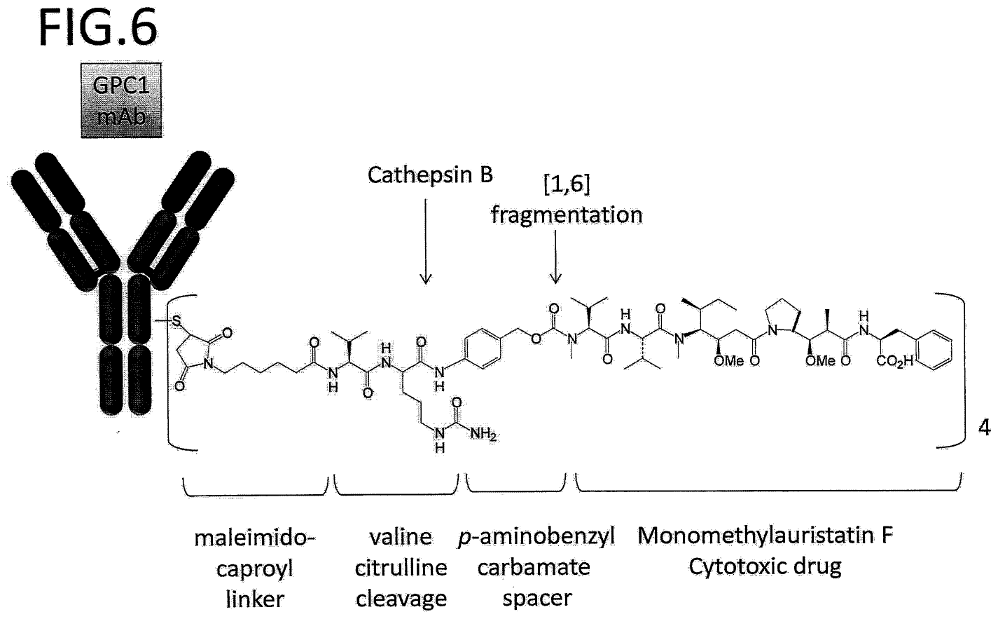

FIG. 7 shows results of analyzing binding affinity between 01a033 and 01a033 ADC's Glypican-1. The vertical axis indicates OD (450 nm to 630 nm) and the horizontal axis indicates the antibody concentration.

FIG. 8 shows the GPC1 antibody binding capacity of esophageal cancer cell strain TE4, TE5, TE6, TE8, TE9, TE10, TE11, TE11, TE14, and TE15 cells.

FIG. 9 shows data from analyzing the expression of GPC1 in various cancer cell strains by FACS. The figure shows the reactivity of an isotype control antibody and the reactivity of a clone 01a033 antibody. The arrows indicate the reactivity of a clone 01a033 antibody.

FIG. 10 shows results of an internalization assay. The results were measured at 0, 1, 2, 3, 4, 6, 12, and 24 hours. The cell strain used was TE8.

FIG. 11 shows % internalized at 4° C. or 37° C. at 0, 1, 2, 3, 4, 6, 12, and 24 hours of 01a033. The vertical axis indicates % internalized and the horizontal axis indicates time.

FIG. 12 shows results of cell growth suppression effects of GPC1 ADC on TE4, TE5, TE6, TE8, TE9, TE10, TE11, TE11, TE14, TE15, and LK2 cells. The vertical axis of each graph indicates the growth rate compared to no treatment (%) and the horizontal axis indicates the antibody concentration.

FIG. 13 shows results of cell growth suppression effects of GPC1 ADC on BxPC3, HeLa, T3M4, and ME180 cells. The vertical axis of each graph indicates the growth rate compared to no treatment (%), and the horizontal axis indicates the antibody concentration.

FIG. 14 is a table summarizing IC50 deduced from the results obtained in FIGS. 12 and 13.

FIG. 15 shows the rate of change in the body weight obtained by a single dose toxicity test. The vertical axis indicates the rate of change in the body weight, and the horizontal axis indicates the number of days after treatment.

FIG. 16 shows results of hematological inspection in a safety test for a single dose. White blood cells (WBC), hemoglobin (Hb), and platelets (Plt) were evaluated.

FIG. 17 shows results of hematological inspection in a safety test for a single dose. Alanine aminotransferase (ALT), amylase (Amy), and chromium (Cr) were evaluated.

FIG. 18 schematically depicts an in vivo efficacy test of GPC1 ADC using a pancreatic cancer cell strain. The error bar indicates mean±SEM.

FIG. 19 shows results of antitumor effect in vivo of GPC1 ADC using a pancreatic cancer cell, strain. The vertical axis indicates the tumor volume (mm³), and the horizontal axis indicates the number of days after treatment.

FIG. 20 shows the tumor weight (mg) on day 36 after treatment in an in vivo efficacy test of GPC1 ADC using a pancreatic cancer cell strain. The p value is determined by Scheffe's method following one-way ANOVA,

FIG. 21 shows the rate of change in the body weight of a mouse in an in vivo efficacy test of GPC1 ADC using a pancreatic cancer cell strain. The vertical axis indicates the rate of change in the body weight, and the horizontal axis indicates the number of days after treatment. The error bar indicates mean±SEM.

FIG. 22 indicates results of an immunohistochemical staining of tumor tissue with administration of a GPC1 ADC. After BxPC3 subcutaneous tumorigenesis, (1) PBS, (2) 10 mg/kg of control ADC, and (3) 1 mg/kg, 3 mg/kg, and 10 mg/kg of GPC1-ADC were administered to the tail vein once, and the tumor was extracted after 24 hours. The black dots indicate cells arrested at the G2/M phase.

FIG. 23 shows results of analysis of the GPC1 expression by immunohistochemical staining in BxPC3 and PDX graft models. After deparaffinization of a section of a paraffin embedded tissue, the section was dehydrated with alcohol. Immunohistochemical staining on GPC1 was performed by using an anti-GPC1 antibody (Atlas Antibodies: HPA030571) and ChemMate Envision kit HRP 500T (Dako: K5007).

FIG. 24 schematically depicts an in vivo efficacy test on GPC1 ADC using pancreatic cancer PDX.

FIG. 25 shows results of an anti-tumor effect in vivo of GPC1 ADC using pancreatic cancer PDX. The vertical axis indicates the tumor volume (mm³), and the horizontal axis indicates the number of days after treatment. The error bar indicates mean±SEM.

FIG. 26 shows the tumor weight (mg) on day 28 after treatment in an in vivo efficacy test of GPC1 ADC using pancreatic cancer PDX. The p value is determined by Scheffe's method following one-way ANOVA.

FIG. 27 shows the rate of change in the body weight of a mouse in an in vivo efficacy test of GPC1 ADC using pancreatic cancer PDX. The vertical axis indicates the rate of change in the body weight, and the horizontal axis indicates the number of days after treatment. The error bar indicates mean±SEM.

FIG. 28 schematically depicts an in vivo efficacy test on GPC1 ADC using a cervical cancer cell strain.

FIG. 29 shows results of an antitumor effect in vivo of GPC1 ADC using a cervical cancer cell strain. The vertical axis indicates the tumor volume (mm³), and the horizontal axis indicates the number of days after treatment. The error bar indicates mean±SEM.

FIG. 30 shows the tumor weight (mg) on day 32 after treatment in an in vivo efficacy test on GPC1 ADC using a cervical cancer cell strain. The p value is determined by Scheffe's method following one-way ANOVA.

FIG. 31 shows the rate of change in the body weight of a mouse in an in vivo efficacy test on a GPC1 ADC using a cervical cancer cell strain. The vertical axis indicates the rate of change in the body weight, and the horizontal axis indicates the number of days after treatment. The error bar indicates mean±SEM.

FIG. 32 indicates the results of immunohistochemical staining of tumor tissue administered with GPC1 ADC. After ME180 subcutaneous tumorigenesis, (1) PBS, (2) 10 mg/kg of control ADC, and (3) 10 mg/kg of GPC1-ADC were administered to the tail vein once, and the tumor was extracted after 24 hours. The black dots indicate cells arrested at the G2/M phase.

FIG. 33 shows results for antitumor effect of ADC and unlabeled antibody on TE4. The vertical axis indicates the rate of growth relative to no treatment (%), and the horizontal axis indicates ADC or antibody concentration.

FIG. 34 shows results for antitumor effect of ADC and unlabeled antibody on TE5. The vertical axis indicates the rate of growth relative to no treatment (%), and the horizontal axis indicates ADC or antibody concentration.

FIG. 35 shows results for antitumor effect of ADC and unlabeled antibody on TE6. The vertical axis indicates the rate of growth relative to no treatment (%), and the horizontal axis indicates ADC or antibody concentration.

FIG. 36 shows results for antitumor effect of ADC and unlabeled antibody on TE8. The vertical axis indicates the rate of growth relative to no treatment (%), and the horizontal axis indicates ADC or antibody concentration.

FIG. 37 shows results for antitumor effect of ADC and unlabeled antibody on TE9. The vertical axis indicates the rate of growth relative to no treatment (%), and the horizontal axis indicates ADC or antibody concentration.

FIG. 38 shows results for antitumor effect of ADC and unlabeled antibody on TE10. The vertical axis indicates the rate of growth relative to no treatment (%), and the horizontal axis indicates ADC or antibody concentration.

FIG. 39 shows results for antitumor effect of ADC and unlabeled antibody on TE11. The vertical axis indicates the rate of growth relative to no treatment (%), and the horizontal axis indicates ADC or antibody concentration.

FIG. 40 shows results for antitumor effect of ADC and unlabeled antibody on TE14. The vertical axis indicates the rate of growth relative to no treatment (%), and the horizontal axis indicates ADC or antibody concentration.

FIG. 41 shows results for antitumor effect of ADC and unlabeled antibody on TE15. The vertical axis indicates the rate of growth relative to no treatment (%), and the horizontal axis indicates ADC or antibody concentration.

FIG. 42 shows results for antitumor effect of ADC and unlabeled antibody on HeLa. The vertical axis indicates the rate of growth relative to no treatment (%), and the horizontal axis indicates ADC or antibody concentration.

FIG. 43 shows results for antitumor effect of ADC and unlabeled antibody on ME180. The vertical axis indicates the rate of growth relative to no treatment (%), and the horizontal axis indicates ADC or antibody concentration.

FIG. 44 schematically depicts the test of Example 12 (in vivo antitumor effect of an unlabeled anti-GPC1 antibody on a GPC1 positive cell strain).

FIG. 45 shows results for in vivo antitumor effect of an unlabeled anti-GPC1 antibody on GPC1 positive cell strain. The vertical axis of the left graph indicates the tumor volume (mm³), and the horizontal axis indicates the number of days after treatment. The right graph shows the tumor weight (mg) on day 31 after treatment.

FIG. 46 shows comparison of results of hematological analysis, in a safety test with a control IgG vs anti-GPC1 antibody in male mice (n=4). The left column in the table indicates the endpoints, white blood cells (WBC), lymphocytes (Ly), monocytes (Mo), granulocytes (Gr), red blood cells (RBC), hemoglobin (Hb), hematocrit value (Hct), and platelets (Plt). The p value was calculated by Student's t-test. The p value is determined by Student's t-test.

FIG. 47 shows comparison of results of hematological analysis in a safety test with a control IgG vs anti-GPC1 antibody in female mice (n=0.4). The left column in the table indicates the endpoints. White blood cells (WBC), lymphocytes (Ly), monocytes (Mo), granulocytes (Gr), red blood cells (RBC), hemoglobin (Hb), hematocrit value (Hct), and platelets (Plt) were evaluated. The p-value is determined by Student's t-test.

FIG. 48 shows comparison of results of hematological analysis in a safety test with a control IgG vs anti-GPC1 antibody in male mice (n=4). The left column in the table indicates the endpoints. Albumin (ALb), alkaline phosphatase (ALP), alanine aminotransferase (ALT), amylase (Amy), total bilirubin (T-Bil), blood urea nitrogen (BUN), calcium (Ca), phosphorous (P), chromium (Cr), glutamine (Glu), sodium (Na), potassium (K), total protein (TP), and globulin (Glob) were evaluated.

FIG. 49 shows comparison of results of hematological analysis in a safety test with a control IgG vs anti-GPC1 antibody in female mice (n=4). The left column in the table indicates the endpoints. Albumin (ALb), alkaline phosphatase (ALP), alanine aminotransferase (ALT), amylase (Amy), total bilirubin (T-Bil), blood urea nitrogen (BUN), calcium (Ca), phosphorous (P), chromium (Cr), glutamine (Glu), sodium (Na), potassium (K), total protein (TP), and globulin (Glob) were evaluated.

FIG. 50 shows results for growth suppression effect of clones 01a033, 01a002, and 02a010 on DU145 cells and MDA-MB231 cells. The vertical axis of each graph indicates the rate of growth compared to no treatment (%), and the horizontal axis indicates the antibody concentration.

FIG. 51 shows results for growth suppression effect of ADC of clones other than clones 01a033, 01a002, and 02a010 on DU145 cells. The vertical axis of each graph indicates the rate of growth compared to no treatment (%), and the horizontal axis indicates the antibody concentration.

FIG. 52 shows results for growth suppression effect of ADC of clones other than clones 01a033, 01a002, and 02a010 on DU145 cells. The vertical axis of each graph indicates the rate of growth compared to no treatment (%), and the horizontal axis indicates the antibody concentration.

FIG. 53 shows a table of IC₅₀ values of ADC of each clone against TE14 cells and EC₅₀ thereof against DU145 cells.

FIG. 54 shows the growth suppression effect against LLC-control-7 and LLC-mGPC1-16 cells. The vertical axis of each graph indicates the rate of growth compared to no treatment (%), and the horizontal axis indicates the antibody concentration.

FIG. 55 schematically depicts the test in Example 17.

FIG. 56 shows results for an in vivo antitumor effect of GPC1 ADC in a mouse grafted with LLC-mGPC1-16 cells. The vertical axis indicates the tumor volume (mm³), and the horizontal axis indicates the number of days after treatment. The error bar indicates mean±SEM (n=5).

FIG. 57 shows the tumor weight (mg) on day 24 after treatment in an in vivo efficacy test of GPC1 ADC in a mouse grafted with LLC-mGPC1-16 cells.

FIG. 58 shows the change in body weight of a mouse in an in vivo efficacy test of GPC1 ADC in a mouse grafted with LLC-mGPC1-16 cells. The error bar indicates mean±SEM (n=5).

### DESCRIPTION OF EMBODIMENTS

The present invention is described hereinafter. Throughout the entire specification, a singular expression should be understood as encompassing the concept thereof in plural form, unless specifically noted otherwise. Thus, singular articles (e.g., “a”, “an”, “the”, and the like in the case of English) should also be understood as encompassing the concept thereof in plural form, unless specifically noted otherwise. Further, the terms used herein should be understood to be used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

### Definitions

First, the terms and common technologies used herein are described.

As used herein, “Glypican-1”, “GPC-1”, or “GPC1” are terms that are used interchangeably, which are glycosylphosphatidylinositol (GPI) anchored cell surface proteoglycans having heparan sulfate. Glypican-1 is understood to be associated with cell adhesion, migration, lipoprotein metabolism, growth factor activity regulation, and suppression of blood coagulation. Glypican-1 is considered to bind to several fibroblast growth factors (FGF) such as FGF-1, FGF-2, and FGF-7. Glypican-1 is understood to function as an extracellular chaperone of VEGF165 and assist in the recovery of receptor binding capacity after oxidation. Currently, 6 types of Glypicans, i.e., Glypican-1 to Glypican-6, are known. Meanwhile, in relation to cancer, Glypican family members are not necessarily recognized as a cancer marker. The members appear to be unrelated to one another. Glypican-1 is registered as accession number P35052 in UniProt (see http://www.uniprot.org/uniprot/P35052), and registered as NP 002072.2 (progenitor amino acid sequence) and NM_002081.2 (mRNA) in NCBI, and as X54232.1 (mRNA), BC051279.1 (mRNA), and AC110619.3 (genomic) in EMBL, GenBank, and DDBJ. Such information can all be utilized herein, which is incorporated herein by reference. For Glypican-1, see David G et al., J Cell Biol. 1990 December; 111 (6 Pt 2): 3165-76; Haecker U et al., Nat Rev Mol Cell Biol. 2005 July; 6(7): 530-41; Aikawa T et al., J Clin Invest. 2008 January; 118(1): 89-99; Matsuda K, et al., Cancer Res. 2001 Jul. 15; 61(14): 5562-9, and the like. SEQ ID NO: 1 is a representative example of the nucleic acid sequence (full length) of human Glypican-1, and SEQ ID NO: 2 is a representative example of the amino acid sequence. SEQ ID NO: 3 is a representative example of the nucleic acid sequence (full length) of mouse Glypican-1, and SEQ ID NO: 4 is a representative example of the amino acid sequence. When used for the objectives herein, it is understood that “Glypican-1”, “GPC-1”, or “GPC1” can be used herein as not only as a protein (or a nucleic acid encoding the same) having an amino acid sequence set forth in a specific sequence number of accession number, but also as a functionally active analog or derivative thereof, a functionally active fragment thereof, homolog thereof, or a mutant encoded by a nucleic acid hybridizing to a nucleic acid encoding the protein under highly stringent conditions or low stringent conditions, as long as it is in alignment with the specific objective of the invention.

As used herein, “derivative”, “analog”, or “mutant” preferably includes, without intending to be limiting, molecules comprising a substantially homologous region in a target protein (e.g., Glypican-1 or antibody). In various embodiments, such a molecule is at least 30%, 40%, 50%, 60%, 70%, 80%, 90%, 95%, or 99% identical compared to a sequence aligned by a computer homology program that is known in the art throughout an amino acid sequence of the same size, or a nucleic acid encoding such a molecule can hybridize with a sequence encoding a constituent protein under (highly) stringent conditions, moderately stringent conditions, or non-stringent conditions. This is a product from modifying a protein by an amino acid substitution, deletion, and addition, respectively, and refers to a protein whose derivative still exhibits, although not necessarily to the same degree, the biological function of the original protein. For example, the biological function of such a protein can be found by a suitable and available in vitro assay that is described herein or known in the art. As used herein, “functionally active” refers to a polypeptide, a fragment, or a derivative having a structural function, regulating function, or biochemical function of a protein such as biological activity in accordance with the embodiment associated with the polypeptide, fragment or derivative of the invention.

In the present invention, humans are mainly discussed with regard to Glypican-1. Meanwhile, many animals other than humans such as chimpanzees (Pantroglodytes) (K7B6W5), rhesus monkeys (Macaca mulatta). (F6VPW9), mice (Mus musculus) (Q9QZF2), rats (Rattus norvegicus) (P35053), and chickens (Gallus gallus) (F1P150) are known to express Glypican-1 proteins. Thus, it is understood that these animals, especially mammals, are also within the scope of the invention. Preferably, the functional domain of Glypican-1, such as the extracellular domain (about 500 amino acids, including 12 cysteine residues) and the hydrophobic region of the C-terminus (GPI-anchor domain) are conserved.

As used herein, a fragment of Glypican-1 is a polypeptide comprising any region of Glypican-1, which does not necessarily have a biological function of a naturally-occurring Glypican-1, as long as the fragment functions as the objective (e.g., marker or therapeutic target) of the invention.

Thus, a typical nucleic acid sequence of Glypican-1 can be:

(a) a polynucleotide having the base sequence set forth in SEQ ID NO: 1 or a fragment sequence thereof;
(b) a polynucleotide encoding a polypeptide consisting of the amino acid sequence set forth in SEQ ID NO: 2 or a fragment thereof;
(c) a polynucleotide encoding a modified polypeptide having one or more amino acids with a mutation selected from the group consisting of substitution, addition, and deletion in the amino acid sequence set forth in SEQ ID NO: 2 or a fragment thereof, the modified polypeptide having biological activity;
(d) a polynucleotide, which is a splice mutant or allelic mutant of the base sequence set forth in SEQ ID NO: 1 or a fragment thereof;
(e) a polynucleotide encoding a species homolog of a polypeptide consisting of the amino acid sequence set forth in SEQ ID NO: 2 or a fragment thereof;
(f) a polynucleotide encoding a polypeptide having biological activity and hybridizing to a polynucleotide of any one of (a) to (e) under stringent conditions; or
(g) a polynucleotide encoding a polypeptide which consists of a base sequence that has at least 70%, at least 80%, at least 90%, at least 95%, at least 96%, at least 97%, at least 98%, or at least 99% identity to a polynucleotide of any one of (a) to (e) or a complementary sequence thereof and has biological activity. In this regard, biological activity typically refers to activity of Glypican-1 or being identifiable from other proteins in the same organism as a marker.

The amino acid sequence of Glypican-1 can be:

(a) a polypeptide consisting of the amino acid sequence set forth in SEQ ID NO: 2 or a fragment thereof;
(b) a polypeptide having one or more amino acids with a mutation selected from the group consisting of substitution, addition, and deletion in the amino acid sequence set forth in SEQ ID NO: 2, and having biological activity;
(c) a polypeptide encoded by a splice mutant or allelic mutant of the base sequence set forth in SEQ ID NO: 1;
(d) a polypeptide, which is a species homolog of the amino acid sequence set forth in SEQ ID NO: 2; or
(e) a polypeptide having an amino acid sequence with at least 70%, at least 80%, at least 90%, at least 95%, at least 96%, at least 97%, at least 98%, or at least 99% identity to a polypeptide of any one of (a) to (d) and having biological activity. In this regard, biological activity typically refers to activity of Glypican-1 or being identifiable from other proteins in the same organism as a marker (e.g., including a region that can function as a specific epitope when used as an antigen).

In relation to the present invention, a “substance that binds to Glypican-1”, “Glypican-1 binding agent” or “Glypican-1 interaction molecule” is a molecule or a substance that at least temporarily binds to Glypican-1. It is preferably and advantageously capable of displaying (e.g., labeled or in a labelable state) that the substance or molecule is bound for detection purposes, and is advantageously further bound to a therapeutic agent. Examples of a substance that binds to Glypican-1 include antibodies, bindable peptides, peptidomimetics, and the like. Preferably, a substance that binds to Glypican-1 has intracellular invasion (internalization) activity. As used herein, a “binding protein” or “binding peptide” with respect to Glypican-1 refers to any protein or peptide that binds to Glypican-1 including, but not limited to, antibodies directed to Glypican-1 (e.g., polyclonal antibody or monoclonal antibody), antibody fragments, and function equivalents.

As used herein, “protein”, “polypeptide”, “oligopeptide”, and “peptide” are used in the same meaning, referring to a polymer of amino acids of any length. Such a polymer may be straight, branched, or cyclic. Amino acids may be naturally-occurring, non-naturally occurring, or altered amino acids. These terms can also encompass those assembled into a complex of a plurality of polypeptide chains. These terms also encompass naturally-occurring or artificially-altered amino acid polymers. Examples of such an alteration include disulfide bond formation, glycosylation, lipidation, acetylation, phosphorylation, and any other manipulation or alteration (e.g., conjugation with a label component). This definition also encompasses, for example, polypeptides comprising one or more analogs of amino acids (e.g., including non-naturally-occurring amino acids or the like), peptide-like compounds (e.g., peptoid), and other alterations known in the art. As used herein, “amino acid” is a general term for organic compounds with an amino group and a carboxyl group. When the antibody according to an embodiment of the invention comprises a “specific amino acid sequence”, any of the amino acids in the amino acid sequence may be chemically modified. Further, any of the amino acids in the amino acid sequence may be forming a salt or a solvate. Further, any of the amino acids in the amino acid sequence may have an L form or a D form. Even for such cases, the protein according to an embodiment of the invention is considered as comprising the “specific amino acid sequence” described above. Examples of known chemical modifications applied to an amino acid comprised in a protein in vivo include modifications of the N-terminus (e.g., acetylation, myristylation, and the like), modifications of the C-terminus (e.g., amidation, addition of glycosylphosphatidylinositol and the like), modifications of a side chain (e.g., phosphorylation, glycosylation, and the like) and the like. The modifications may be naturally-occurring or non-naturally-occurring, as long as the objective of the present invention is met.

As used herein, “polynucleotide”, “oligonucleotide”, and “nucleic acid” are used in the same meaning, referring to a polymer of nucleotides of any length. These terms also encompass “oligonucleotide derivative” and “polynucleotide derivative”. The “oligonucleotide derivative” and “polynucleotide derivative” are interchangeably used and refer to an oligonucleotide or polynucleotide comprising a derivative of a nucleotide or an oligonucleotide or having a bond between nucleotides that is different from normal bonds. Specific examples of such oligonucleotides include: 2′-O-methyl-ribonucleotide; oligonucleotide derivatives with a phosphodiester bond in an oligonucleotide converted into phosphorothioate bond; oligonucleotide derivatives with a phosphodiester bond in an oligonucleotide converted into an N3′-P5′ phosphoramidate bond; oligonucleotide derivatives with a ribose and a phosphodiester bond in an oligonucleotide converted into a peptide nucleic acid bond; oligonucleotide derivatives with a uracil in an oligonucleotide substituted with a C-5 propynyl uracil; oligonucleotide derivatives with uracil in an oligonucleotide substituted with a C-5 thiazole uracil; oligonucleotide derivatives with a cytosine in an oligonucleotide substituted with a C-5 propynyl cytosine; oligonucleotide derivatives with a cytosine in an oligonucleotide substituted with a phenoxazine-modified cytosine; oligonucleotide derivatives with a ribose in DNA substituted with a 2′-O-propylribose; oligonucleotide derivatives with a ribose in an oligonucleotide substituted with a 2′-methoxyethoxy ribose; and the like. Unless noted otherwise, specific nucleic acid sequences are intended to encompass sequences that are explicitly set forth, as well as their conservatively altered variants (e.g., degenerate codon substitutes) and complementary sequences. Specifically, a degenerate codon substitute can be achieved by making a sequence in which the third position of one or more selected (or all) codons is substituted with a mixed base and/or deoxyinosine residue (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). As used herein, “nucleic acid” is also interchangeably used with gene, cDNA, mRNA, oligonucleotide, and polynucleotide. As used herein, “nucleotide” may be naturally occurring or non-naturally-occurring.

As used herein, “gene” refers to an agent that defines a genetic trait. A “gene” may refer to a “polynucleotide”, “oligonucleotide”, or “nucleic acid”.

As used herein, “homology” of genes refers to the degree of identity of two or more genetic sequences with respect to one another, and having “homology” generally refers to having a high degree of identity or similarity. Therefore, the identity or similarity of sequences is higher when homology of two genes is high. Whether two types of genes have homology can be found by direct comparison of sequences or by a hybridization method under stringent conditions for nucleic acids. When two genetic sequences are directly compared, the genes are homologous typically if DNA sequences are at least 50% identical, preferably at least 70% identical, and more preferably at least 80%, 90%, 95%, 96%, 97%, 98%, or 99% identical between the genetic sequences. Thus, as used herein, “homolog” or “homologous gene product” refers to a protein in another species, preferably mammal, exerting the same biological function as a protein constituent of a complex, which will be further described herein. Such a homolog is also known as “ortholog gene product”. It is understood that such a homolog, homologous gene product, ortholog gene product, or the like can also be used, as long as they are in alignment with the objective of the invention.

Amino acids may be mentioned herein by either their commonly known three letter symbols or their one character symbols recommended by the IUPAC-IUB Biochemical Nomenclature Commission. Similarly, nucleotides may be mentioned by their commonly recognized one character codes. Comparison of similarity, identity, and homology of an amino acid sequence and a base sequence is calculated herein by using a sequence analysis tool BLAST with default parameters. For example, identity can be searched using BLAST 2.2.28 (published on Apr. 2, 2013) of the NCBI. Herein, values for identity generally refer to a value obtained when aligned under the default conditions using BLAST. However, when a higher value is obtained by changing a parameter, the highest value is considered the value of identity. When identity is evaluated in a plurality of regions, the highest value thereamong is considered the value of identity. Similarity is a value calculated by taking into consideration a similar amino acid in addition to identity.

In one embodiment of the invention, “several” may be, for example, 10, 8, 6, 5, 4, 3 or 2, or a value less than any one of the values. It is known that a polypeptide with one or several amino acid residue deletions, additions, insertions, or substitutions by other amino acids maintains its biological activity (Mark et al., Proc Natl Acad Sci USA. 1984 September; 81(18): 5662-5666, Zoller et al., Nucleic Acids Res. 0.1982 Oct. 25; 10(20): 6487-6500, Wang et al., Science. 1984 Jun. 29; 224 (4656): 1431-1433.) An antibody with a deletion or the like can be made, for example, by site-directed mutagenesis, random mutagenesis, biopanning using an antibody phage library, or the like. For example, KOD-Plus-Mutagenesis Kit (TOYOBO CO., LTD.) can be used for site-directed mutagenesis. An antibody with the same activity as the wild-type can be selected from mutant antibodies introduced with a deletion or the like by performing various characterizations in FACS analysis, ELISA, or the like.

In one embodiment of the invention, “90% or greater” may be, for example, 90, 95, 96, 97, 98, 99 or 100% or greater, or within the range of any two such values. For the “homology”, the percentage of the number of homologous amino acids in two or a plurality of amino acid sequences may be calculated in accordance with a method that is known in the art. Before calculating the percentage, amino acid sequences in a group of amino acid sequences to be compared are aligned. A space is introduced in a portion of amino acid sequences when it is necessary to maximize the percentage of the same amino acids. An alignment method, method of calculating the percentage, comparison method, and computer programs associated therewith have been well known in the art (e.g., BLAST, GENETYX, and the like). As used herein, “homology” can be represented by a value measured with BLAST of the NCBI, unless specifically noted otherwise. Blastp can be used in the default setting for an algorithm for comparing amino acid sequences with BLAST. Results of measurement are expressed in a numerical form as Positives or Identities.

As used herein, “polynucleotide which hybridizes under a stringent condition” refers to commonly used, well-known conditions in the art. Such a polynucleotide can be obtained by using a method such as colony hybridization, plaque hybridization, or southern blot hybridization while using a polynucleotide selected from the polynucleotides of the inventions as a probe. Specifically, the polynucleotide refers to a polynucleotide that can be identified by using a filter with immobilized DNA from a colony or plaque and performing hybridization at 65° C. in the presence of 0.7 to 1.0 M NaCl, and then using an SSC (saline-sodium citrate) solution with 0.1 to 2× concentration (composition of an SSC solution with 1× concentration is 150 mM sodium chloride and 15 mM sodium citrate) to wash the filter under the condition of 65° C. For “stringent condition”, the following are examples of conditions that can be used. (1) low ionic strength and a high temperature are used for washing (e.g., 0.015 M sodium chloride/0.0015 M sodium citrate/0.1% sodium dodecyl sulfate at 50° C.), (2) a denaturing agent such as formamide is used in hybridization (e.g., 50% (v/v) formamide, 0.1% bovine serum albumin/0.1% ficoll/0.1% polyvinyl pyrrolidone/50 mM sodium phosphate buffer with a pH of 6.5, 750 mM sodium chloride, and 75 mM sodium citrate at 42° C.), and (3) a solution comprising 20% formamide, 5×SSC, 50 mM sodium phosphate (pH 7.6), 5×Denhardt's solution, 10% dextran sulfate, and 20 mg/ml denatured sheared salmon sperm DNA, is incubated overnight at 37° C. and then a filter is washed with 1×SSC at about 37 to 50° C. The formamide concentration may be 50% or greater. Washing time can be 5, 15, 30, 60, or 120 minutes or longer. A plurality of elements such as temperature and salt concentration are conceivable as elements affecting the stringency of hybridization reactions. Ausubel et al., Current Protocols in Molecular Biology, Wiley Interscience Publishers, (1995) can be referred for details. “Highly stringent condition”, for example, is 0.0015 M sodium chloride, 0.0015 M sodium citrate, and 65-68° C. or 0.015 M sodium chloride, 0.0015 M sodium citrate, 50% formamide, and 42° C. Hybridization can be performed in accordance with the method described in experimental publications such as Molecular Cloning 2nd ed., Current Protocols in Molecular Biology, Supplement 1-38, DNA Cloning 1: Core Techniques, A Practical Approach, Second Edition, Oxford University Press (1995). In this regard, a sequence comprising only an A sequence or only a T sequence is preferably excluded from a sequence that hybridizes under stringent conditions. A moderately stringent condition can be readily determined by those skilled in the art based on, for example, the length of a DNA, and is shown in Sambrook et al., Molecular Cloning: A Laboratory Manual, Third Ed., Vol. 1, 7.42-7.45 Cold Spring Harbor Laboratory Press, 2001, including, for a nitrocellulose filters, use of hybridization conditions of a pre-wash solution of 1.0 mM EDTA (pH 8.0), 0.5% SDS, and 5×SSC, and about 50% formamide and 2×SSC-6×SSC at about 40-50° C. (or other similar hybridization solutions such as a Stark's solution in about 50% formamide at about 42° C.) and washing conditions of 0.5×SSC, 0.1% SDS at about 60° C. Thus, the polypeptides used herein encompass polypeptides encoded by a nucleic acid molecule that hybridizes under highly or moderately stringent conditions to a nucleic acid molecule encoding a polypeptide described herein in particular.

As used herein, a “purified” substance or biological agent (e.g., nucleic acid, protein, or the like) refers to a substance or a biological agent having at least a part of an agent naturally accompanying the substance or biological agent removed. Thus, the purity of a biological agent in a purified biological agent is generally higher than the purity in the normal state of the biological agent (i.e., concentrated). The term “purified” as used herein refers to the presence of preferably at least 75% by weight, more preferably at. least 85% by weight, still more preferably at least 95% by weight, and most preferably at least 98% by weight of a biological agent of the same type. The substance or biological agent used herein is preferably a “purified” substance. An “isolated” substance or biological agent (e.g., nucleic acid, protein, or the like) as used herein refers to a substance or biological agent having agents that naturally accompany the substance or biological agent. substantially removed. The term “isolated” as used herein varies depending on the objective. Thus, the term does not necessarily have to be represented by purity. However, when necessary, the term refers to the presence of preferably at least 75% by weight, more preferably at least 85% by weight, still more preferably at least 95% by weight, and most preferably at least 98% by weight. of a biological agent of the same type. The substance used herein is preferably an “isolated” substance or biological agent.

As used herein, a “corresponding” amino acid, nucleic acid, or moiety refers to an amino acid or a nucleotide which has or is expected to have, in a certain polypeptide molecule or polynucleotide molecule (e.g., Glypican-1), similar action as a given amino acid, nucleotide, or moiety in a benchmark polypeptide or a polynucleotide for comparison, and, particularly for enzyme molecules, refers to an amino acid which is present at a similar position in an active site and makes a similar contribution to catalytic activity and refers to a corresponding moiety in a complex molecule (e.g., heparan sulfate or the like). For example, for an antisense molecule, it can be a similar moiety in an ortholog corresponding to a specific moiety of the antisense molecule. A corresponding amino acid can be a specific amino acid subjected to, for example, cysteination, glutathionylation, S—S bond formation, oxidation (e.g., oxidation of methionine side chain), formylation, acetylation, phosphorylation, glycosylation, myristylation, or the like. Alternatively, a corresponding amino acid can be an amino acid responsible for dimerization. Such a “corresponding” amino acid or nucleic acid may be a region or a domain over a certain range. Thus, it is referred herein as a “corresponding” region or domain in such a case. Such a corresponding region or domain is useful for designing a complex molecule in the present invention.

As used herein, a “corresponding” gene (e.g., polynucleotide sequence or molecule) refers to a gene (e.g., polynucleotide sequence or molecule) of a certain species which has or is expected to have similar action as a given gene in a benchmark species for comparison. If there is a plurality of genes having such action, the corresponding gene refers to a gene having the same evolutionary origin. Hence, a gene corresponding to a certain gene may be an ortholog of such a gene. Thus, for each human Glypican-1, a corresponding Glypican-1 can be found in other animals (especially mammals). Such a corresponding gene can be identified by using a technology that is well known in the art. For example, a corresponding gene in a certain animal (e.g., mouse) can be found by searching a database comprising sequences of the animal from using the sequence of SEQ ID NO: 1, 2, or the like as a query sequence, as a benchmark gene of the corresponding gene (e.g., Glypican-1).

As used herein, “fragment” refers to a polypeptide or polynucleotide with a sequence length of 1 to n−1 with respect to the full length polypeptide or polynucleotide (with length n). The length of a fragment can be appropriately changed in accordance with the objective. Examples of the lower limit of such a length include 3, 4, 5, 6, 7, 8, 9, 10, 15, 20, 25, 30, 40, 50 and more amino acids for a polypeptide. Lengths represented by an integer that is not specifically mentioned herein (e.g., 11 and the like) can also be suitable as a lower limit. Further, examples of the length include 5, 6, 7, 8, 9, 10, 15, 20, 25, 30, 40, 50, 75, 100, and more nucleotides for polynucleotide. Lengths represented by an integer that is not specifically mentioned herein (e.g., 11 and the like) can also be suitable as a lower limit. As used herein, such a fragment is understood to be within the scope of the present invention, for example when a full length version functions as a marker or a target molecule, as long as the fragment itself also functions as a marker or a target molecule.

According to the present invention, the term “activity” as used herein refers to a function of a molecule in the broadest sense. Activity generally encompasses, but is not intended to be limited to, biological function, biochemical function, physical function, and chemical function of a molecule. Examples of activity include enzymatic activity, ability to interact with another molecule, ability to activate, promote, stabilize, inhibit, suppress, or destabilize a function of another molecule, stability, and ability to localize at a specific position in a cell. When applicable, the term is also directed to a function of a protein complex in the broadest sense.

As used herein, “biological function”, with regard to a gene or a nucleic acid molecule or polypeptide related thereto, refers to a specific function that the gene, nucleic acid molecule, or polypeptide can have in vivo. Examples thereof include, but are not limited to, production of a specific antibody, enzymatic activity, impartation of resistance, and the like. Examples thereof include, but are not limited to, functions of Glypican-1 involved in apoptosis of GPC-1 positive tumor cells (e.g., esophageal cancer cells), cleavage of caspase-3, phosphorylation of AKT, and the like. As used herein, biological function can be exerted by “biological activity”. As used herein, “biological activity” refers to activity that a certain agent (e.g., polynucleotide, protein, or the like) can have in vivo, including activity exerting a variety of functions (e.g., transcription promoting activity) such as the activity of activating or deactivating a molecule from interaction with another molecule. When two agents interact, the biological activity thereof can be understood as the bond between the two molecules and the biological change resulting therefrom, e.g., the two molecules are bound when precipitation of one of the molecules with an antibody results in co-precipitation of the other molecule. Thus, one method of determination includes observing such co-precipitation. If an agent is for example an enzyme, the biological activity thereof encompasses the enzymatic activity thereof. Another example includes binding of a ligand to a corresponding receptor when an agent is a ligand. Such biological activity can be measured by a technology that is well known in the art. Thus, “activity” refers to various measurable indicators that indicate or reveal the bond (either directly or indirectly) or affect a response (i.e., having a measurable effect in response to some exposure or stimulation). Examples thereof include the affinity of a compound that directly binds to the polypeptide or polynucleotide of the invention, the amount of proteins upstream or downstream after some stimulation or event, and a scale of another similar function.

As used herein, “expression” of a gene, a polynucleotide, a polypeptide, or the like refers to the gene or the like being subjected to a certain action in vivo to be converted into another form. Preferably, expression refers to a gene, a polynucleotide, or the like being transcribed and translated into a form of a polypeptide. However, transcription to make an mRNA is also one embodiment of expression. Thus, “expression product” as used herein encompasses such a polypeptide and protein, and mRNA. More preferably, such a polypeptide form can be a form which has undergone post-translation processing. For example, the expression level of Glypican-1 can be determined by any method. Specifically, the expression level of Glypican-1 can be found by evaluating the amount of mRNA of Glypican-1, the amount of Glypican-1 protein, and the biological activity of Glypican-1 protein. Such a measurement value can be used in companion diagnosis. The amount of protein or mRNA of Glypican-1 can be determined by the method described in detail in other parts of the specification or other methods known in the art.

As used herein, “functional equivalent” refers to any entity having the same function of interest but a different structure relative to the original target entity. Thus, it is understood that a functional equivalent of “Glypican-1” or an antibody thereof encompasses mutants and variants (e.g., amino acid sequence variants and the like) of Glypican-1 or antibody thereof that are not Glypican-1 or antibody thereof itself, which have the biological action of Glypican-1 or antibody thereof or can change, upon action, into Glypican-1 or the antibody thereof itself, or a mutant or variant of Glypican-1 or the antibody thereof (e.g., including nucleic acids encoding Glypican-1 or an antibody thereof itself and mutants and variants of Glypican-1 or antibody thereof, and vectors, cells, and the like comprising such a nucleic acid). It is understood, even without specifically mentioning, that a functional equivalent of Glypican-1 or an antibody thereof can be used in the same manner as Glypican-1 or antibody thereof. A functional equivalent can be found by searching a database or the like. As used herein, “search” refers to utilizing a certain nucleic acid base sequence electronically, biologically, or by another method to find another nucleic acid base sequence having a specific function and/or property. Examples of electronic search include, but are not limited to, BLAST (Altschul et al., J. Mol. Biol. 215: 403-410 (1990)), FASTA (Pearson & Lipman, Proc. Natl. Acad. Sci., USA 85: 2444-2448 (1988)), Smith and Waterman method (Smith and Waterman, J. Mol. Biol. 147: 195-197 (1981)), Needleman and Wunsch method (Needleman and Wunsch, J. Mol. Biol. 48: 443-453 (1970)) and the like. Examples of biological search include, but are not limited to, stringent hybridization, a macroarray with a genomic DNA applied to a nylon membrane or the like or a microarray with a genomic DNA applied to a glass plate (microarray assay), PCR, in situ hybridization, and the like. Herein, a gene used in the present invention is intended to include corresponding genes identified by such electronic search or biological search.

As a functional equivalent of the invention, it is possible to use an amino acid sequence with one or more amino acid insertions, substitutions, or deletions, or addition to one or both ends. As used herein, “one or more amino acid insertions, substitutions, or deletions, or addition to one or both ends in an amino acid sequence” refers to an alteration with a substitution of a plurality of amino acids or the like to the extent that can occur naturally by a well-known technical method such as site-directed mutagenesis or natural mutation. An altered amino acid sequence can have, for example, 1 to 30, preferably 1 to 20, more preferably 1 to 9, still more preferably 1 to 5, and especially preferably 1 to 2 amino acid insertions, substitutions, or deletions, or additions to one or both ends. Preferably, an altered amino acid sequence may be an amino acid sequence having one or more (preferably 1 or several, or 1, 2, 3 or 4) conservative substitutions in the amino acid sequence of Glypican-1. “Conservative substitution” refers herein to a substitution of one or more amino acid residues with other chemically similar amino acid residue so as not to substantially alter a function of a protein. Examples thereof include substitutions of a hydrophobic residue with another hydrophobic residue, substitutions of a polar residue with another polar residue having the same charge, and the like. Functionally similar amino acids that can be substituted in this manner are known in the art for each amino acid. Specific examples include alanine, valine, isoleucine, leucine, proline, tryptophan, phenylalanine, methionine, and the like for nonpolar (hydrophobic) amino acids, and glycine, serine, threonine, tyrosine, glutamine, asparagine, cysteine, and the like for polar (neutral) amino acids. Examples of positively charged (basic) amino acids include arginine, histidine, lysine, and the like. Further, examples of a negatively-charged (acidic) amino acid include aspartic acid, glutamic acid, and the like.

As used herein, “inhibitor” refers to a substance or agent that inhibits biological action of a receptor or a cell against a target entity (e.g., receptor or cell). A Glypican-1 inhibitor of the invention is an agent that can temporarily or permanently reduce or eliminate a function of a target Glypican-1, a cell expressing Glypican-1, or the like. Examples of such an agent include, but are not limited to, antibodies, antigen binding fragments thereof, derivatives, functional equivalents, antisense nucleic acids, RNAi agents such as siRNAs, other nucleic acid forms, and the like. In one embodiment of the invention, an “anti-Glypican-1 antibody” includes antibodies capable of binding to Glypican-1. While the production method of such an anti-Glypican-1 antibody is not particularly limited, the antibody can be produced by, for example, immunizing a mammal or avian with Glypican-1. An “antigen binding fragment” of an anti-Glypican-1 antibody refers to a fragment of an antibody retaining the ability to bind to Glypican-1. Examples of such an antigen binding fragment include, but are not limited to, single chain antibodies, scFvs, Fab fragments, F(ab′)₂ fragments, and the like.

It is understood that a “functional equivalent” of an “antibody to Glypican-1 (anti-Glypican-1 antibody) or an antigen binding fragment thereof” includes, for antibodies, antibodies having binding activity of Glypican-1 and fragments thereof themselves, as well as chimeric antibodies, humanized antibodies, human antibodies, multifunctional antibodies, bispecific or oligospecific antibodies, single chain antibodies, scFvs, diabodies, sc(FV)2 (single chain (Fv)2), scFv-Fcs, and the like.

An anti-Glypican-1 antibody according to one embodiment of the invention is preferably an anti-Glypican-1 antibody that specifically binds to a specific epitope of Glypican-1 from the viewpoint of especially strong suppression of growth of malignant tumors.

An anti-Glypican-1 antibody according to one embodiment of the invention can be a monoclonal antibody. A monoclonal antibody can act more efficiently on Glypican-1 compared to polyclonal antibodies. It is preferable to immunize chickens with Glypican-1 from the viewpoint of efficiently producing anti-Glypican-1 monoclonal antibodies.

The antibody class of the anti-Glypican-1 antibody according to one embodiment of the invention is not particularly limited. For example, the class may be IgM, IgD, IgG, IgA, IgE, or IgY.

The anti-Glypican-1 antibody according to one embodiment of the present invention may be an antibody fragment having antigen binding activity (hereinafter, also referred to as “antigen binding fragment”). In such a case, there is an effect of improved stability, antibody production efficiency, or the like.

The anti-Glypican-1 antibody according to one embodiment of the invention may be a fusion protein. The fusion protein may comprise a polypeptide or oligopeptide bound to the N or C-terminus of an anti-Glypican-1 antibody. The oligopeptide in this regard may be an His-tag. The fusion protein may also be fused to a mouse, human, or chicken antibody partial sequence. Such fusion proteins are also encompassed as one form of the anti-Glypican-1 antibody according to the present embodiment.

The anti-Glypican-1 antibody according to one embodiment of the invention may be, for example, an antibody obtained via the step of immunizing an organism with a purified Glypican-1, Glypican-1-expressing cell, or Glypican-1 containing lipid membrane. It is preferable that a Glypican-1-expressing cell is used for immunization from the viewpoint of enhancing a therapeutic effect against Glypican-1 positive cancer.

The anti-Glypican-1 antibody according to one embodiment of the invention may be an antibody having a CDR set of an antibody obtained via the step of immunizing an organism with a purified Glypican-1, Glypican-1-expressing cell, or Glypican-1 containing lipid membrane. It is preferable that a Glypican-1-expressing cell is used for immunization from the viewpoint of enhancing a therapeutic effect against Glypican-1 positive cancer. A CDR set, is a set of heavy chain CDRs 1, 2, and 3 and light chain CDRs 1, 2, and 3.

“Glypican-1 expressing cell” in one embodiment of the invention may be obtained, for example, by introducing a polynucleotide encoding Glypican-1 into a cell and having the Glypican-1 expressed. Glypican-1 in this regard encompasses Glypican-1 fragments. Further, “Glypican-1-containing lipid membrane” in one embodiment of the invention may be obtained, for example, by mixing Glypican-1 and a lipid bilayer. Glypican-1 in this regard encompasses Glypican-1 fragments. Further, the anti-Glypican-1 antibody according to one embodiment of the invention is preferably an antibody obtained via the step of immunizing a chicken with an antigen or an antibody having a CDR set of such an antibody from the viewpoint of enhancing a therapeutic effect against Glypican-1 positive cancer.

The anti-Glypican-1 antibody of the invention may have any binding capacity as long as the objective can be accomplished. For example, it is sufficient for the anti-Glypican-1 antibody of the invention to have internalization activity and/or ADC activity even if the antibody has low affinity to Glypican-1. However, the antibody preferably has a strong binding capacity for the purpose of diagnosis and companion reagent. For example, the K_{D} value (kd/ka) can be 1.0×10⁻⁷ (M) or less, 1.0×10⁻⁸ (M) or less, 1.0×10⁻⁹ (M) or less, or 1.0×10⁻¹⁰ or less. Preferably, the binding capacity of an anti-Glypican-antibody for the purpose of diagnosis and companion reagent is K_{D} value (kd/ka) of 1.0×10⁻⁸ (M) or less.

The anti-Glypican-1 antibody according to one embodiment of the invention may be an antibody that binds to a wild-type or mutant Glypican-1. Mutant Glypican-1 includes mutants due to individual differences in the DNA sequences. The amino acid sequence of wild-type or mutant Glypican-1 is preferably 80% or more, more preferably 90% or more, more preferably 95% or more, and especially preferably 98% or more homologous to the amino acid sequence set forth in SEQ ID NO: 2.

As used herein, an “antibody” includes a molecule capable of specifically binding to a specific epitope on an antigen or a population thereof. An antibody may be a polyclonal antibody or a monoclonal antibody. Antibodies can have various forms such as one or more forms selected from the group consisting of full length antibodies (antibodies with a Fab region and an Fc region), Fv antibodies, Fab antibodies, F(ab′)2 antibodies, Fab′ antibodies, diabodies, single stranded (single chain) antibodies (e.g., scFv), sc(Fv)2 (single chain (Fv)2), scFv-Fc, dsFv, multispecific antibodies (e.g., oligospecific antibodies and bispecific antibodies), diabodies, peptides or polypeptides with an antigen binding property, chimeric antibodies (e.g., mouse-human chimeric antibodies, chicken-human chimeric antibodies, and the like), mouse antibodies, chicken antibodies, humanized antibodies, human antibodies, and an equivalent thereof. Antibodies also encompass modified and unmodified antibodies. Modified antibodies may be formed by an antibody binding to various molecules such as polyethylene glycol. A modified antibody can be obtained by applying chemical modification to an antibody using a known approach. Such an antibody can also be covalently bound, or fused by recombination, to an enzyme such as alkaline phosphatase, horseradish peroxidase, or a galactosidase. The anti-Glypican-1 antibodies or the like used herein can be of any origin, type, shape, or the like, as long as the antibodies bind to a Glypican-1 protein. Specifically, known antibodies such as a nonhuman animal antibody (e.g., a mouse antibody, a rat antibody, or a camel antibody), a human antibody, a chimeric antibody, or a humanized antibody can be used. In the present invention, a monoclonal or polyclonal antibody can be utilized, but a monoclonal antibody is preferable. It is preferable that an antibody binds specifically to a Glypican-1 protein. Further, antibodies encompass modified and unmodified antibodies. Modified antibodies may be formed by an antibody binding to various molecules such as polyethylene glycol. A modified antibody can be obtained by applying a chemical modification to an antibody by using a known approach.

“Polyclonal antibody” in one embodiment of the invention can be produced, for example, by administering an immunogen comprising an antigen of interest to mammals (e.g., rat, mouse, rabbit, cow, monkey, or the like), birds or the like in order to induce production of a polyclonal antibody specific to the antigen. An immunogen may be administered by injection of one or more immunizing agents and, when desired, an adjuvant. An adjuvant may be used to increase immune responses and may comprise a Freund's adjuvant (complete or incomplete), mineral gel (aluminum hydroxide or the like), surfactant (lysolecithin or the like), or the like. Immunization protocols are known in the art and, in some cases, may be implemented by any method that induces an immune response in accordance with the selected host organism (Tanpakushitsu Jikken Handobukku [Protein experiment handbook], Yodosha (2003): 86-91).

“Monoclonal antibody” in one embodiment of the invention encompasses individual antibodies constituting a population that are antibodies corresponding to substantially a single epitope except for antibodies having a mutation that can occur naturally in small amounts. Further, individual antibodies constituting a population may be antibodies that are substantially the same except for antibodies having a mutation that can occur naturally in small amounts. Monoclonal antibodies are highly specific, which are different from common polyclonal antibodies that typically include different antibodies corresponding to different epitopes. In addition to their specificity, monoclonal antibodies are useful in that they can be synthesized from a hybridoma culture which is not contaminated with other immunoglobulins. The description “monoclonal” may indicate a characteristic of being obtained from substantially homogeneous antibody. population. However, such a description does not mean that antibodies must be produced by a specific method. For example, monoclonal antibodies may be made by a method similar to the hybridoma method described in “Kohler G, Milstein C., Nature. 1975 Aug. 7; 256 (5517): 495-497”. Alternatively, monoclonal antibodies may be made by a method similar to the recombinant method described in U.S. Pat. No. 4,816,567. Monoclonal antibodies may also be isolated from a phage antibody library using a method similar to the technique that is described in, “Clackson et al., Nature. 1991 Aug. 15; 352 (6336): 624-628.” or “Marks et al., J Mol Biol. 1991 Dec. 5; 222(3): 581-597”. Monoclonal antibodies may also be made by the method described in “Tanpakushitsu Jikken Handobukku [Protein experiment handbook], Yodosha (2003): 92-96”.

Antibodies can be mass-produced by using any approach that is known in the art. Examples of representative antibody mass production system construction and antibody manufacture include the following. Specifically, an H chain antibody expression vector and L chain antibody expression vector are transfected into CHO cells. The cells are cultured using a selection reagent G418 and Zeocin and cloned by limiting dilution. After cloning, clones stably expressing antibodies are selected by ELISA. The culture is expanded with the selected CHO cells, and the culture supernatant comprising antibodies are collected. Antibodies can be purified from the collected culture supernatant by Protein A or Protein G purification.

“Fv antibody” in one embodiment of the invention is an antibody comprising an antigen recognition site. This region comprises a dimer of one heavy chain variable domain non-covalently bound to one light chain variable domain. In this configuration, three CDRs of each variable domain can interact with one another to form an antigen binding site on the surface of a VH-VL dimer.

“Fab antibody” in one embodiment of the invention is, for example, a fragment obtained by treating an antibody comprising an Fab region and an Fc region with proteinase papain, which is an antibody in which about half of the N-terminus side of the H chain is bound to the entire L chain via some disulfide bonds. Fabs can be obtained, for example, by treating the anti-Glypican-1 antibody according to the embodiments of the invention comprising an Fab region and an Fc region with proteinase papain.

“F(ab′)2 antibody” in one embodiment of the invention is a fragment obtained by treating an antibody comprising an Fab region and an Fc region with proteinase pepsin, which is an antibody comprising two sites corresponding to Fabs. F(ab′)2 can be obtained, for example, by treating the anti-Glypican-1 antibody according to an embodiments of the invention comprising an Fab region and an Fc region with proteinase pepsin. For example, F(ab′)2 can be made by binding the following Fab′ with a thioether bond or a disulfide bond.

“Fab′ antibody” in one embodiment of the invention is an antibody obtained, for example, by cleaving a disulfide bond at a hinge region of F(ab′)2. For example, this can be obtained through treating F(ab′)2 with a reducing agent dithiothreitol.

“scFv antibody” in one embodiment of the invention is an antibody comprising VH and VL that are linked with a suitable peptide linker. scFv antibodies can be produced, for example, by obtaining a cDNA encoding VH and VL of the anti-Glypican-1 antibody according to an embodiment of the invention, constructing a polynucleotide encoding VH-peptide linker-VL, incorporating the polynucleotide into a vector, and using a cell for expression.

“Diabody” in one embodiment of the invention is an antibody having divalent antigen binding activity. Divalent antigen binding activity can be configured to be identical or configured such that one of them has a different antigen binding activity. A diabody can be produced, for example, by constructing a polynucleotide encoding scFv such that the length of the amino acid sequence of a peptide linker is 8 residues or less, incorporating the resulting polynucleotide into a vector, and using a cell for expression.

“dsFv” in one embodiment of the invention is an antibody in which a polypeptide introduced with cysteine residues in VH and VL is bound via a disulfide bond between the cysteine residues. The position to which cysteine residues are introduced can be selected based on steric structure prediction of an antibody in accordance with the method demonstrated by Reiter et al (Reiter et al., Protein Eng. 1994 May; 7(5): 697-704).

“Peptide or polypeptide with antigen binding affinity” in one embodiment of the invention is an antibody comprised of antibody VH, VL or CDR1, 2 or 3 thereof. A peptide comprising a plurality of CDRs can be bound directly or via a suitable peptide linker.

The production method of the aforementioned Fv antibody, Fab antibody, F(ab′)² antibody, Fab′ antibody, scFv antibody, diabody, dsFv antibody, and peptide or polypeptide with antigen binding affinity (hereinafter, also referred to as “Fv antibodies and the like”) is not particularly limited. Fv antibodies can be produced, for example, by incorporating a DNA encoding a region of the Fv antibodies and the like in the anti-Glypican-1 antibody according to an embodiment of the invention into an expression vector and using an expression cell. Further, Fv antibodies may be produced by a chemical synthesis method such as Fmoc (fluorenylmethyloxycarbonyl) or tBOC (t-butyloxycarbonyl) method. It should be noted that the antigen binding fragment according to one embodiment of the invention may be one or more types of the Fv antibodies and the like described above.

“Chimeric antibody” in one embodiment of the invention is, for example, a variable region of an antibody linked to a constant region of an antibody between xenogenic organisms and can be constructed by a genetic engineering technology. A mouse-human chimeric antibody can be made by, for example, the method described in “Roguska et al., Proc Natl Acad Sci USA. 1994 Feb. 1; 91(3): 969-973.” For example, the basic method of making a mouse-human chimeric antibody links a mouse leader sequence and a variable region sequence in a cloned cDNA with a sequence encoding a human antibody constant region already present in an expression vector of a mammalian cell. After linking the mouse leader sequence and variable region sequence in a cloned cDNA with the sequence encoding a human antibody constant region, the resultant sequence may be linked to a mammalian cell expression vector. A fragment of a human antibody constant region can be from any human antibody H chain constant region and human antibody L chain constant region. Examples of human H chain fragment include Cγ1, Cγ2, Cγ3, and Cγ4, and examples of L chain fragment include Cλ and Cκ.

“Humanized antibody” in one embodiment of the invention is, for example, an antibody, which has one or more CDRs from nonhuman species, a framework region (FR) from a human immunoglobulin, and a constant region from human immunoglobulin and binds to a desired antigen. Antibodies can be humanized using various approaches known in the art (Almagro et al., Front Biosci. 2008 Jan. 1; 13: 1619-1633). Examples thereof include CDR grafting (Ozaki et al., Blood. 1999 Jun. 1; 93(11): 3922-3930), Re-surfacing (Roguska et al., Proc Natl Acad Sci USA. 1994 Feb. 1; 91(3): 969-973), FR shuffle (Damschroder et al., Mol Immunol. 2007 April; 44(11): 3049-3060. Epub 2007 Jan. 22.) and the like. An amino acid residue of a human FR region may be substituted with a corresponding residue from a CDR donor antibody in order to alter (preferably in order to improve) the antigen bond. The FR substitution can be performed by a method that is well known in the art (Riechmann et al., Nature. 1988 Mar. 24; 332 (6162): 323-327.) For example, an FR residue that is important for antigen binding may be identified by modeling an interaction between a CDR and an FR residue. Further, an abnormal FR residue at a specific position may be identified by sequence comparison.

“Human antibody” in one embodiment of the invention is, for example, an antibody in which a region comprising a variable region and constant region of a heavy chain and variable region and constant region of a light chain constituting the antibody is derived from a gene encoding a human immunoglobulin. Main production methods include a method using a transgenic mouse for making human antibodies, phage display method, and the like. A method using a transgenic mouse for making human antibodies produces human antibodies with diverse antigen binding capacities instead of mouse antibodies if a functional human Ig gene is introduced into an endogenous Ig knockout mouse. Furthermore, this mouse can be immunized to obtain human monoclonal antibodies by a conventional hybridoma method. This can be made, for example, by the method described in “Lonberg et al., Int Rev Immunol. 1995; 13(1): 65-93.” The phage display method is a system that typically expresses an exogenous gene as a fusion protein such that phage infectivity is not lost on the N-terminus side of a coat protein (g3p, g10p, or the like) of fibrous phage such as an E. coli virus M13 or T7. Antibodies can be made, for example, by the method described in “Vaughan et al., Nat Biotechnol. 1996 March; 14(3): 309-314”.

Antibodies may also be prepared by grafting a heavy chain CDR or light chain CDR of the anti-Glypican-1 antibody according to an embodiment of the invention onto any antibody by CDR-grafting (Ozaki et al., Blood. 1999 Jun. 1; 93(11): 3922-3930). Alternatively, antibodies can be obtained by linking a DNA encoding a heavy chain CDR or light chain CDR of the anti-Glypican-1 antibody according to an embodiment of the invention and a DNA encoding a region excluding a heavy chain CDR or light chain CDR of a known antibody derived from a human or a nonhuman organism to a vector in accordance with a known method in the art and then using a known cell for expression. In doing so, a known method in the art (e.g., method of allowing amino acid residues of an antibody to randomly mutate and screening for antibodies with high reactivity, phage display method, or the like) may be used to optimize the region excluding a heavy chain CDR or light chain CDR in order to enhance the efficiency of anti-Glypican-1 antibody acting upon a target antigen. Further, an FR region may be optimized by using, for example, FR shuffle (Damschroder et al., Mol Immunol. 2007 April; 44(11): 3049-3060. Epub 2007 Jan. 22.) or a method of replacing a vernier zone amino acid residue or packaging residue (Japanese Laid-Open Publication No. 2006-241026 or Foote et al., J Mol Biol. 1992 Mar. 20; 224(2): 487-499).

“Heavy chain” in one embodiment of the invention is typically the main constituent element of a full-length antibody. A heavy chain is generally bound to a light chain by a disulfide bond and non-covalent bond. A region called a variable region (VH), which has an amino acid sequence that is not constant, even among antibodies in the same class of the same species, is present in a domain on the N-terminus side of a heavy chain. VH is generally known to greatly contribute to the specificity and affinity to an antigen. For example, “Reiter et al., J Mol Biol. 1999 Jul. 16; 290(3): 685-98.” describes that a molecule with only a VH, when made, bound to an antigen with specificity and high level of affinity. Furthermore, “Wolfson W, Chem Biol. 2006 December; 13(12): 1243-1244.” describes that there are antibodies having only a heavy chain without a light chain among camel antibodies.

“CDR (complementarity determining region)” in one embodiment of the invention is a region that is actually in contact with an antigen to form a binding site in an antibody. CDRs are generally located on an Fv (variable region: including heavy chain variable region (VH) and light chain variable region (VL)) of an antibody. Further, CDRs generally have CDR1, CDR2, and CDR3 consisting of about 5 to 30 amino acid residues. In addition, CDRs of a heavy chain are particularly known for their contribution to binding of an antibody to an antigen. Among the CDRs, CDR3 is known to contribute the most in binding of an antibody to an antigen. For example, “Willy et al., Biochemical and Biophysical Research Communications Volume 356, Issue 1, 27 Apr. 2007, Pages 124-128” describes that a heavy chain CDR3 was altered to elevate the binding capacity of an antibody. An Fv region other than the CDRs is called a framework region (FR), consisting of FR1, FR2, FR3, and FR4, which are conserved relatively well among antibodies (Kabat et. al., “Sequence of Proteins of Immunological Interest” US Dept. Health and Human Services, 1983.) Specifically, it is understood that a factor characterizing the reactivity of an antibody is in CDRs, especially heavy chain CDRs.

A plurality of methods for defining CDRs and determining the positions thereof have been reported. For example, the Kabat definition (Sequences of Proteins of Immunological Interest, 5th ed., Public Health Service, National Institutes of Health, Bethesda, Md. (1991)) or the Chothia definition (Chothia et al., J. Mol. Biol., 1987; 196: 901-917) may be used. One embodiment of the invention uses the Kabat definition as an optimal example, but the definition is not necessarily limited thereto. Further, the definitions may be determined in some cases after considering both the Kabat definition and the Chothia definition. For example, an overlapping portion of CDR according to each of the definitions, or a portion comprising CDRs according to both of the definitions can be deemed the CDR. A specific example of such a method is the method of Martin et al using Oxford Molecular's AbM antibody modeling software, which is a proposal combining the Kabat definition and the Chothia definition (Proc. Natl. Acad. Sci. USA, 1989; 86: 9268-9272). Such CDR information can be used to produce a mutant that can be used in the present invention. Such an antibody mutant comprises one or several (e.g., 2, 3, 4, 5, 6, 7, 8, 9, or 10) substitutions, additions, or deletions in the framework of the original antibody. However, a mutant can be produced such that the CDR does not comprise a mutation.

As used herein, “antigen” refers to any substrate that can be specifically bound by an antibody molecule. As used herein, “immunogen” refers to an antigen, which can initiate lymphocyte activation that leads to an antigen specific immune response. As used herein, “epitope” or “antigen determinant” refers to a site in an antigen molecule to which an antibody or a lymphocyte receptor binds. A method of determining an epitope is well known in the art. Such an epitope can be determined by those skilled in the art by using a well-known and conventional technology when a primary sequence of an amino acid or a nucleic acid is provided. It is understood that the antibody of the invention having other sequences can be similarly used, as long as the epitope is the same.

It is understood that antibodies with any specificity may be used as the antibody used herein, as long as false positive reactions are reduced. Thus, the antibodies used herein may be a polyclonal antibody or a monoclonal antibody.

As used herein, “means” refers to anything that can be a tool for accomplishing an objective (e.g., detection, diagnosis, or therapy). As used herein, “selective recognition means” in particular refers to means capable of recognizing a certain subject differently from others.

As used herein, “marker (substance or gene)” refers to a substance that can be an indicator for tracking whether a target is in or at risk of being in a certain state (e.g., diseased state, disorder state, level of or presence of malignant state, or the like). Examples of such a marker include genes, gene products, metabolites, enzymes, and the like. In the present invention, detection, diagnosis, preliminary detection, prediction, or prediagnosis of a certain state (e.g., state of a disease such as cancer) can be materialized by using an agent or means specific to a marker associated with such a state, or a composition, kit, or system comprising the same or the like. As used herein, “expression product” (also referred to as a gene product) refers to a protein or mRNA encoded by a gene. It was found herein that a gene product (Glypican-1), which has not been proven for therapy of GPC-1 positive cancer such as esophageal cancer, can be used as a therapeutic target of GPC-1 positive cancer such as esophageal cancer.

“Cancer” targeted by the present invention includes one or more types selected from the group consisting of lung cancer, esophageal cancer, gastric cancer, liver cancer, pancreatic cancer, renal cancer, adrenal cancer, bile duct cancer, breast cancer, colon cancer, small intestine cancer, ovarian cancer, uterine cancer, bladder cancer, prostate cancer, ureteral cancer, renal pelvis cancer, ureteral cancer, penile cancer, testicular cancer, cerebral tumor, cancer of the central nervous system, cancer of the peripheral nervous system, head and neck cancer, glioma, glioblastoma multiform, skin cancer, melanoma, thyroid cancer, salivary gland cancer, malignant lymphoma, carcinoma, sarcoma, leukemia, and hematologic malignancy. Ovarian cancer in this regard includes, for example, ovarian serous adenocarcinoma and ovarian clear cell adenocarcinoma. Uterine cancer includes, for example, endometrial cancer and cervical cancer. Head and neck cancer includes, for example, oral cavity cancer, pharyngeal cancer, nasal cavity cancer, paranasal cancer, salivary gland cancer, and thyroid cancer. Lung cancer includes, for example, non-small-cell lung cancer and small cell lung cancer. Malignant tumor may be PD-L1 positive.

As used herein, “esophageal cancer” is used in the normal sense and used broadly in the sense to include cancer in the esophagus. Esophageal cancer includes, but is not limited to, squamous cell carcinoma as well as adenocarcinoma, esophageal cancer at a lymph node metastasis site, and the like. It is understood that about half the incidences of esophageal cancer in Japanese patients develop near the center of the esophagus in the chest, and then ¼ of the incidences develops in the lower portion of the esophagus. It is understood that the present invention targets both types of esophageal cancers. Although not wishing to be bound by any theory, the present invention is expected to be usable as an indicator for all esophageal cancers including squamous cell carcinoma as well as adenocarcinoma, and esophageal cancer at a lymph node metastasis site.

As used herein, “pancreatic cancer” refers to a malignant tumor that has developed from the pancreas, but generally refers to pancreatic ductal adenocarcinoma, and encompasses tumors of other parts of the pancreas.

As used herein, “cervical cancer” is a type of uterine cancer. Uterine cancer includes cervical cancer and endometrial cancer. Cervical cancer develops from the portion called the cervix at the uterine entrance.

As used herein, “lung cancer” is a malignant tumor from epithelial cells developing in the lung, including small cell lung cancer, non-small cell lung cancer, and the like. Non-small cell lung cancer includes adenocarcinoma, squamous cell carcinoma, and large cell cancer.

As used herein, “head and neck cancer” is a tumor of the head and neck, referring to tumor developed in a portion from the face to the neck, such as the nose, mouth, throat, upper jaw, lower jaw, or ear.

As used herein, “breast cancer” is a tumor developed in the mamma, including ductal carcinoma, lobular carcinoma, and the like.

As used herein, “subject (person)” refers to a target subjected to diagnosis, detection, therapy, or the like of the invention (e.g., an organism such as a human or a cell, blood, serum, or the like extracted from an organism).

As used herein, “sample” refers to any substance obtained from a subject or the like. For example, serum and the like are encompassed thereby. Those skilled in the art can appropriately select a preferred sample based on the descriptions herein.

As used herein, “agent” is used broadly and may be any substance or other elements (e.g., energy, radiation, heat, electricity, and other forms of energy) as long as the intended objective can be achieved. Examples of such a substance include, but are not limited to, protein, polypeptide, oligopeptide, peptide, polynucleotide, oligonucleotide, nucleotide, nucleic acid (including, for example, DNAs such as cDNA and genomic DNA and RNAs such as mRNA), polysaccharide, oligosaccharide, lipid, organic small molecule (e.g., hormone, ligand, information transmitting substance, organic small molecule, molecule synthesized by combinatorial chemistry, small molecule that can be used as medicine (e.g., small molecule ligand and the like)) and composite molecule thereof. A substance that binds to Glypican-1 can also be such an agent. Typical examples of an agent specific to a polynucleotide include, but are not limited to, a polynucleotide having complementarity with a certain sequence homology (e.g., 70% or greater sequence identity) to the sequence of the polynucleotide, polypeptide such as a transcription factor that binds to a promoter region, and the like. Typical examples of an agent specific to a polypeptide include, but are not limited to, an antibody directed specifically to the polypeptide or a derivative or analog thereof (e.g., single chain antibody), a specific ligand or receptor when the polypeptide is a receptor or ligand, a substrate when the polypeptide is an enzyme, and the like.

As used herein, “diagnosis” refers to identifying various parameters associated with a disease, disorder, condition (e.g., malignant tumor), or the like in a subject to determine the current or future state of such a disease, disorder, or condition. The condition in the body can be investigated by using the method, apparatus, or system of the invention. Such information can be used to select and determine various parameters of a formulation or method for the treatment or prevention to be administered, disease, disorder, or condition in a subject, or the like. As used herein, “diagnosis” when narrowly defined refers to diagnosis of the current state, but when broadly defined includes “early diagnosis”, “predictive diagnosis”, “prediagnosis”, and the like. Since the diagnostic method of the invention in principle can utilize what comes out from a body and can be conducted away from a medical practitioner such as a physician, the present invention is industrially useful. In order to clarify that diagnosis can be conducted away from a medical practitioner such as a physician, the term as used herein may be particularly called “assisting” “predictive diagnosis, prediagnosis, or diagnosis”.

As used herein, “detecting drug (agent)” or “inspection drug (agent)” broadly refers to all agents capable of detecting or inspecting a target of interest.

As used herein, “diagnostic drug (agent)” broadly refers to all agents capable of diagnosing a condition of interest (e.g., disease such as esophageal cancer or the like).

As used herein, “therapy” refers to the prevention of exacerbation, preferably maintaining of the current state, more preferably alleviation, and still more preferably elimination of a disease or disorder (e.g., esophageal cancer) when such a condition has developed, including being capable of exerting a prophylactic effect or an effect of improving a disease of a patient or one or more symptoms accompanying the disease. Preliminary diagnosis with suitable therapy may be referred to as “companion therapy” and a diagnostic drug therefor may be referred to as “companion diagnostic agent”.

As used herein, “therapeutic drug (agent)” broadly refers to all agents capable of treating a condition of interest (e.g., diseases such as esophageal cancer or the like). In one embodiment of the invention, “therapeutic drug” may be a pharmaceutical composition comprising an active ingredient and one or more pharmacologically acceptable carriers. A pharmaceutical composition can be manufactured, for example, by mixing an active ingredient and the carriers by any method known in the technical field of pharmaceuticals. Further, usage mode of a therapeutic drug is not limited, as long as it is used for therapy. A therapeutic drug may be the active ingredient alone or a mixture of an active ingredient and any ingredient. Further, the shape of the carriers is not particularly limited. For example, the carrier may be a solid or liquid (e.g., buffer solution). It should be noted that a therapeutic drug of esophageal cancer includes a drug (prophylactic drug) for preventing esophageal cancer or a growth inhibitor for esophageal cancer cells.

As used herein, “prevention (prophylaxis)” refers to the action of taking a measure against a disease or disorder (e.g., esophageal cancer) from being in such a condition prior to being in such a condition. For example, it is possible to use the agent of the invention to perform diagnosis, and optionally use the agent of the invention to prevent or take measures to prevent esophageal cancer or the like.

As used herein, “prophylactic drug (agent)” broadly refers to all agents capable of preventing a condition of interest (e.g., diseases such as esophageal cancer or the like).

As used herein, “interaction”, for two substances, refers to applying a force (e.g., intermolecular force (Van der Waals force), hydrogen bond, hydrophobic interaction, or the like) between one substance and the other substance. Generally, two substances that have interacted are in a conjugated or bound state. The detection, inspection, and diagnosis in the invention can be materialized by utilizing such an interaction.

As used herein, the term “bond (binding)” refers to a physical or chemical interaction between two substances or between combinations thereof. A bond includes an ionic bond, non-ionic bond, hydrogen bond, Van der Waals bond, hydrophobic interaction, and the like. A physical interaction (bond) can be direct or indirect. Indirect physical interaction (bond) is mediated by or is due to an effect of another protein or compound. A direct bond refers to an interaction, which does not occur through or due to an effect of another protein or compound and does not substantially involve another intermediate.

Thus, an “agent” (or detection agent or the like) that “specifically” interacts (or binds) to a biological agent such as a polynucleotide or a polypeptide as used herein encompasses agents with affinity to the biological agent such as a polynucleotide or polypeptide that is typically similar or higher, preferably significantly (e.g., statistically significantly) higher, than affinity to other unrelated polynucleotides or polypeptides (especially those with less than 30% identity). Such affinity can be measured, for example, by hybridization assay, binding assay, or the like.

As used herein, “specific” interaction (or binding) of a first substance or agent with a second substance or agent refers to the first substance or agent interacting with (or binding to) the second substance or agent at a higher affinity than with substances or agents other than the second substance or agent (especially other substances or agents in a sample comprising the second substance or agent). Examples of an interaction (or bond) specific to a substance or agent include, but are not limited to, hybridization in a nucleic acid, antigen-antibody reaction in a protein, enzyme-substrate reaction, other nucleic acid-protein reactions, protein-lipid interaction, nucleic acid-lipid interaction, and the like. Thus, if substances or agents are both nucleic acids, a first substance or agent “specifically interacting” with a second substance or agent encompasses the first substance or agent having at least partial complementarity to the second substance or agent. Further, examples of a first substance or agent “specifically” interacting with (or binding to) a second substance or agent if substances or agents are both proteins include, but are not limited to, interaction by an antigen-antibody reaction, interaction by a receptor-ligand reaction, enzyme-substrate interaction, and the like. If two types of substances or agents include a protein and a nucleic acid, a first substance or agent “specifically” interacting with (or binding to) a second substance or factor encompasses an interaction (or a bond) between an antibody and an antigen thereof. Such a specific interaction or binding reaction can be utilized to detect or quantify a target in a sample.

As used herein, “detection” or “quantification” of polynucleotide or polypeptide expression can be accomplished using a suitable method including, for example, an immunological measuring method and measurement of mRNAs, including a bond to or interaction with a detection agent, inspection agent, or diagnostic agent. Examples of a molecular biological measuring method include Northern blot, dot blot, PCR, and the like. Examples of an immunological measurement method include ELISA using a microtiter plate, RIA, fluorescent antibody method, luminescence immunoassay (LIA), immunoprecipitation (IP), single radial immunodiffusion (SRID), turbidimetric immunoassay (TIA), Western blot, immunohistochemical staining, and the like. Further, examples of a quantification method include ELISA, RIA, and the like. This may also be performed using a gene analysis method with an array (e.g., DNA array, protein array). DNA arrays are outlined extensively in (Ed. by Shujurisha, Saibo Kogaku Bessatsu “DNA Maikuroarei to Saishin PCR ho” [Cellular engineering, Extra issue, “DNA Microarrays and Latest PCR Methods”]. Protein arrays are discussed in detail in Nat Genet. 2002 December; 32 Suppl: 526-532. Examples of a method of analyzing gene expression include, but are not limited to, RT-PCR, RACE, SSCP, immunoprecipitation, two-hybrid system, in vitro translation, and the like, in addition to the methods described above. Such additional analysis methods are described in, for example, Genomu Kaiseki Jikkenho Nakamura Yusuke Labo Manyuaru [Genome analysis experimental method Yusuke Nakamura Lab Manual], Ed. by Yusuke Nakamura, Yodosha (2002) and the like. The entirety of the descriptions therein is incorporated herein by reference.

As used herein, “amount of expression” refers to the amount of polypeptide, mRNA, or the like that is expressed in a cell, tissue, or the like of interest. Examples of such an amount of expression include the amount of expression of the polypeptide of the invention at a protein level assessed by any suitable method including an immunological measurement method such as ELISA, RIA, fluorescent antibody method, Western blot, and immunohistochemical staining by using the antibody of the invention, and the amount of expression of the polypeptide used in the present invention at an mRNA level assessed by any suitable method including a molecular biological measuring method such as Northern blot, dot blot, and PCR. “Change in amount of expression” refers to an increase or decrease in the amount of expression of the polypeptide used in the present invention at a protein level or mRNA level assessed by any suitable method including the aforementioned immunological measuring method or molecular biological measuring method. A variety of detection or diagnosis based on a marker can be performed by measuring the amount of expression of a certain marker.

As used herein, “decrease” or “suppression” of activity or expression product (e.g., protein, transcript (RNA or the like)) or synonyms thereof refers to a decrease in the quantity, quality, or effect of a specific activity, transcript, or protein, or activity that decreases the same. Among decrease, “elimination” refers to activity, expression product, or the like being less than the detection limit and is especially referred to as “elimination”. As used herein, “elimination” is encompassed by “decrease” or “suppression”.

As used herein, “increase” or “activation” of activity or expression product (e.g., protein, transcript (RNA or the like)) or synonyms thereof refers to an increase in the quantity, quality, or effect of a specific activity, transcript, or protein, or activity that increases the same.

As used herein, “label” refers to an entity (e.g., substance, energy, electromagnetic wave, or the like) for distinguishing a molecule or substance of interest from others. Such a method of labeling includes RI (radioisotope) method, fluorescence method, biotin method, chemiluminescent method, and the like. When a plurality of markers of the invention or agents or means for capturing the same are labeled by a fluorescence method, labeling is performed with fluorescent substances having different fluorescent emission maximum wavelengths. It is preferable that the difference in fluorescent emission maximum wavelengths is 10 nm or greater. When labeling a ligand, any label that does not affect the function can be used. However, Alexa™ Fluor is desirable as a fluorescent substance. Alexa™ Fluor is a water-soluble fluorescent dye obtained by modifying coumarin, rhodamine, fluorescein, cyanine, or the like. This is a series compatible with a wide range of fluorescence wavelengths. Relative to other fluorescent dyes for the corresponding wavelength, Alexa™ Fluor is very stable, bright, and has a low pH sensitivity. Combinations of fluorescent dyes with fluorescence maximum wavelength of 10 nm or greater include a combination of Alexa™ 555 and Alexa™ 633, combination of Alexa™ 488 and Alexa™ 555, and the like. When a nucleic acid is labeled, any label can be used that can bind to a base portion thereof. However, it is preferable to use a cyanine dye (e.g., Cγ3, Cγ5 or the like of the CyDye™ series), rhodamine 6G reagent, 2-acetylaminofluorene (AAF), AAIF (iodine derivative of AAF), or the like. Examples of a fluorescent substance with a difference in fluorescent emission maximum wavelengths of 10 nm or greater include a combination of Cγ5 and a rhodamine 6G reagent, a combination of Cγ3 and fluorescein, a combination of a rhodamine 6G reagent and fluorescein, and the like. The present invention can utilize such a label to alter a target of interest to be detectable by the detecting means to be used. Such alteration is known in the art. Those skilled in the art can appropriately carry out such a method in accordance with the label and target of interest.

As used herein, “tag” refers to a substance for distinguishing a molecule by a specific recognition mechanism such as receptor-ligand, or more specifically, a substance serving the role of a binding partner for binding a specific substance (e.g., having a relationship such as biotin-avidin or biotin-streptavidin). A tag can be within the scope of “label”. Accordingly, a specific substance to which a tag is bound can distinguish the specific substance by contacting a substrate, to which a binding partner of a tag sequence is bound. Such a tag or label is well known in the art. Typical examples of tag sequences include, but are not limited to, myc tag, His tag, HA, Avi tag, and the like.

Such a tag may be bound to the detection agent, inspection agent, or diagnostic agent of the invention.

As used herein, “in vivo” refers to inside of a living organism. In a specific context, “in a living organism” refers to the position where a substance of interest should be disposed.

As used herein, “in vitro” refers to a state where a portion of a living organism is extracted or freed “outside of a living organism” (e.g., into a test tube) for various research purposes. This is a term that is an antonym of in vivo.

As used herein, “ex vivo” refers to a series of operations of a certain treatment performed outside the body, but the sample is intended to be returned into the body later. In the present invention, this can include an embodiment envisioned to treat a certain cell in an organism with an agent of the invention and return the cell to the patient.

As used herein, “kit” refers to a unit generally providing portions to be provided (e.g., inspection drug, diagnostic drug, therapeutic drug, antibody, label, manual, and the like) in two or more separate sections. This form of a kit is preferred when a composition that should not be provided in a mixed state and is preferably mixed immediately before use for safety reasons or the like is intended to be provided. Such a kit advantageously comprises an instruction or manual describing how the provided portions (e.g., inspection drug, diagnostic drug, or therapeutic drug) are used or how a reagent should be handled. When the kit is used herein as a reagent kit, the kit generally comprises an instruction describing how to use an inspection drug, diagnostic drug, therapeutic drug, antibody, and the like.

As used herein, “instruction” is a document explaining the method of use of the present invention for a physician or other users. The instruction has a description of the detection method of the invention, method of use of a diagnostic agent, or instruction to administer a medicament or the like. Further, an instruction may have a description instructing oral administration or administration to the esophagus (e.g., by injection or the like) as a site of administration. The instruction is prepared in accordance with a format specified by the regulatory agency of the country in which the present invention is practiced (e.g., the Ministry of Health, Labor and Welfare in Japan, Food and Drug Administration (FDA) in the U.S., or the like), with an explicit description showing approval by the regulatory agency. The instruction is a so-called package insert and is typically provided in, but not limited to, paper media. The instructions can also be provided in a form such as electronic media (e.g., web sites provided on the Internet or emails).

As used herein, “intracellular invasion (internalize/internalization) refers to intake of a substance bound to an antigen by a cell by endocytosis or phagocytosis mediated by the substance bound to an antigen on a cell surface. A substance bound to Glypican-1 having such activity (e.g., anti-Glypican-1 antibody) can make an active ingredient of interest undergo intracellular invasion into a cell expressing Glypican-1 on the cell surface to exert a desired effect due to the active ingredient of interest in Glypian-1 expressing cells. Examples of the active ingredient of interest include, but are not limited to, agents with cytotoxic activity, anticancer agents, contrast media, siRNAs, antisense nucleic acids, ribozymes, and the like.

As used herein, “antibody-drug conjugate (ADC)” refers to an antibody chemically linked to one or more active ingredients of interest, or an antigen binding fragment thereof. In a preferred embodiment, ADC is operably linked via a linker. As used herein, “operably linked” refers to a relationship where a linked substance can operate in a predicted manner. Examples of active ingredients of interest that can be contained in an ADC include, but are not limited to, agents with cytotoxic activity, anticancer agents, contrast media, siRNAs, antisense nucleic acids, ribozymes, and the like. A linker that can be used include cleavable linkers and non-cleavable linkers. Examples of cleavable linkers include, but are not limited to, linkers with a sequence cleaved by a protease, acid labile linkers, disulfide linkers, and the like. Examples of non-cleavable linkers include, but are not limited to, MCC linkers and the like.

As used herein, “cytotoxic activity” refers to, for example, inducing a pathological change to a cell, or inducing cell depth by not only direct trauma, but also any structural or functional damage to cells such as cleavage of DNA or formation of a dimer of a base, cleavage of a chromosome, damage to the cell division system, or reduction in various enzymatic activity to directly or indirectly block the function of cells. Therefore, examples agent having cytotoxic activity” include, but are not limited to, alkylating agents, tumor necrosis factor inhibitors, intercalators, microtubule inhibitors, kinase inhibitors, proteasome inhibitors, topoisomerase inhibitors, and the like.

As used herein, “half maximal inhibitory concentration (ICH)” refers to a concentration of a compound required for killing 50% of cells. IC₅₀ is measured herein using the method described in Example 7.

### PREFERRED EMBODIMENTS

The preferred embodiments of the invention are explained hereinafter. It is understood that the embodiments provided hereinafter are provided to facilitate better understanding of the present invention, so that the scope of the invention should not be limited by the following descriptions. Thus, it is apparent that those skilled in the art can refer to the descriptions herein to make appropriate modifications within the scope of the invention. It is also understood that the following embodiments of the invention can be used individually or as a combination.

(Antibodies)

The inventors have found new clones of an antibody that specifically binds to human Glypican-1 as shown in Example 1. Thus, in one aspect, the present invention provides an anti-human Glypican-1 antibody or antigen binding fragment thereof, wherein the antibody is selected from the group consisting of:

(a) an antibody comprising amino acid sequences of heavy chain CDR1, heavy chain CDR2, and heavy chain CDR3 set forth in SEQ ID NO: 53, 54, and 55, respectively, and light chain CDR1, light chain CDR2, and light chain CDR3 set forth in SEQ ID NO: 56, 57, and 58, respectively (clone K090-01a033);
(b) an antibody comprising amino acid sequences of heavy chain CDR1, heavy chain CDR2, and heavy chain CDR3 set forth in SEQ ID NOs: 5, 6, and 7, respectively, and light chain CDR1, light chain CDR2, and light chain CDR3 set forth in SEQ ID NOs: 8, 9, and 10, respectively (clone K090-01a002);
(c) an antibody comprising amino acid sequences of heavy chain CDR1, heavy chain CDR2, and heavy chain CDR3 set forth in SEQ ID NOs: 11, 12, and 13, respectively, and light chain CDR1, light chain CDR2, and light chain CDR3 set forth in SEQ ID NOs: 14, 15, and 16, respectively (clone K090-01a007);
(d) an antibody comprising amino acid sequences of heavy chain CDR1, heavy chain CDR2, and heavy chain CDR3 set forth in SEQ ID NOs: 17, 18, and 19, respectively, and light chain CDR1, light chain CDR2, and light chain CDR3 set forth in SEQ ID NOs: 20, 21, and 22, respectively (clone K090-01a016);
(e) an antibody comprising amino acid sequences of heavy chain CDR1, heavy chain CDR2, and heavy chain CDR3 set forth in SEQ ID NOs: 23, 24, and 25, respectively, and light chain CDR1, light chain CDR2, and light chain CDR3 set forth in SEQ ID NOs: 26, 27, and 28, respectively (clone K090-01a017);
(f) an antibody comprising amino acid sequences of heavy chain CDR1, heavy chain CDR2, and heavy chain CDR3 set forth in SEQ ID NOs: 29, 30, and 31, respectively, and light chain CDR1, light chain CDR2, and light chain CDR3 set forth in SEQ ID NOs: 32, 33, and 34, respectively (clone K090-01a021);
(g) an antibody comprising amino acid sequences of heavy chain CDR1, heavy chain CDR2, and heavy chain CDR3 set forth in SEQ ID NOs: 35, 36, and 37, respectively, and light chain CDR1, light chain CDR2, and light chain CDR3 set forth in SEQ ID NOs: 38, 39, and 40, respectively (clone K090-01a026);
(h) an antibody comprising amino acid sequences of heavy chain CDR1, heavy chain CDR2, and heavy chain CDR3 set forth in SEQ ID NOs: 41, 42, and 43, respectively, and light chain CDR1, light chain CDR2, and light chain CDR3 set forth in SEQ ID NOs: 44, 45, and 46, respectively (clone K090-01a009);
(i) an antibody comprising amino acid sequences of heavy chain CDR1, heavy chain CDR2, and heavy chain CDR3 set forth in SEQ ID NOs: 47, 48, and 49, respectively, and light chain CDR1, light chain CDR2, and light chain CDR3 set forth in SEQ ID NOs: 50, 51, and 52, respectively (clone K090-01a030);
(j) an antibody comprising amino acid sequences of heavy chain CDR1, heavy chain CDR2, and heavy chain CDR3 set forth in SEQ ID NOs: 59, 60, and 61, respectively, and light chain CDR1, light chain CDR2, and light chain CDR3 set forth in SEQ ID NOs: 62, 63, and 64, respectively (clone K090-01a042);
(k) an antibody comprising amino acid sequences of heavy chain CDR1, heavy chain CDR2, and heavy chain. CDR3 set forth in SEQ ID NOs: 65, 66, and 67, respectively, and light chain CDR1, light chain CDR2, and light chain CDR3 set forth. in SEQ ID NOs: 68, 69, and 70, respectively (clone K090-02a002);
(l) an antibody comprising amino acid sequences of heavy chain CDR1, heavy chain CDR2, and heavy chain CDR3 set forth in SEQ ID NOs: 71, 72, and 73, respectively, and light chain CDR1, light chain CDR2, and light chain CDR3 set forth in SEQ ID NOs: 74, 75, and 76, respectively (clone K090-02a006);
(m) an antibody comprising amino acid sequences of heavy chain CDR1, heavy chain CDR2, and heavy chain CDR3 set forth in SEQ ID NOs: 77, 78, and 79, respectively, and light chain CDR1, light chain CDR2, and light chain CDR3 set forth in SEQ ID NOs: 80, 81, and 82, respectively 0.15 (clone K090-02a010);
(n) an antibody comprising amino acid sequences of heavy chain CDR1, heavy chain CDR2, and heavy chain CDR3 set forth in SEQ ID NOs: 83, 84, and 85, respectively, and light chain CDR1, light chain CDR2, and light chain CDR3 set forth in SEQ ID NOs: 86, 87, and 88, respectively (clone K090-02a014);
(o) an antibody comprising amino acid sequences of heavy chain CDR1, heavy chain CDR2, and heavy chain CDR3 set forth in SEQ ID NOs: 89, 90, and 91, respectively, and light chain CDR1, light chain CDR2, and light chain CDR3 set. forth in SEQ ID NOs: 92, 93, and 94, respectively (clone K090-02a022);
(p) an antibody comprising amino acid sequences of heavy chain CDR1, heavy chain CDR2, and heavy chain CDR3 set forth in SEQ ID NOs: 95, 96, and 97, respectively, and light chain CDR1, light chain CDR2, and light chain CDR3 set forth in SEQ ID NOs: 98, 99, and 100, respectively (clone K090-02a034);
(q) an antibody comprising amino acid sequences of heavy chain CDR1, heavy. chain CDR2, and heavy chain CDR3 set forth in SEQ ID NOs: 101, 102, and 103, respectively, and light chain CDR1, light chain CDR2, and light chain CDR3 set forth in SEQ ID NOs: 104, 105, and 106, respectively (clone K090-02a035);
(r) an antibody comprising amino acid sequences of heavy chain CDR1, heavy chain. CDR2, and heavy chain CDR3 set forth in SEQ ID NOs: 107, 108, and 109, respectively, and light chain CDR1, light chain CDR2, and light chain CDR3 set forth in SEQ ID NOs: 110, 111, and 112, respectively (clone K090-02b006); and
(s) a mutant of an antibody selected from (a) to (r) comprising at least one substitution, addition, or deletion in a CDR moiety

In a preferred embodiment, the antibody or antigen binding fragment thereof that can be used in the present invention has sequences of heavy chain CDRs and light chain CDRs of the following clone: 01a033, 01a002, 02a010, 02a002, 02a014, 02b006, 01a042, 01a017, 01a026, 01a016, 01a030, or 01a009. These clones exhibited excellent EC50 values against TE14 cells as well as DU1.45 cells.

In a certain embodiment, the antibody of the invention is selected from the group consisting of:

(a) an antibody comprising amino acid sequences of a heavy chain set forth in SEQ ID NO: 158 and a light chain set forth in SEQ ID NO: 160 (clone K090-01a033);
(b) an antibody comprising amino acid sequences of a heavy chain set forth in SEQ ID NO: 126 and a light chain set forth in SEQ ID NO: 128 (clone K090-01a002);
(c) an antibody comprising amino acid sequences of a heavy chain set forth in SEQ ID NO: 130 and a light chain set forth in SEQ ID NO: 132 (clone K090-01a007);
(d) an antibody comprising amino acid sequences of a heavy chain set forth in SEQ ID NO: 134 and a light chain set forth in SEQ ID NO: 136 (clone K090-01a016);
(e) an antibody comprising amino acid sequences of a heavy chain set forth in SEQ ID NO: 138 and a light chain set forth in SEQ ID NO: 140 (clone K090-01a017);
(f) an antibody comprising amino acid sequences of a heavy chain set forth in SEQ ID NO: 142 and a light chain set forth in SEQ ID NO: 144 (clone K090-01a021);
(g) an antibody comprising amino acid sequences of a heavy chain set forth in SEQ ID NO: 146 and a light chain set forth in SEQ ID NO: 148 (clone K090-01a026);
(h) an antibody comprising amino acid sequences of a heavy chain set forth in SEQ ID NO: 150 and a light chain set forth in SEQ ID NO: 152 (clone K090-01a009);
(i) an antibody comprising amino acid sequences of a heavy chain set forth in SEQ ID NO: 154 and a light chain set forth in SEQ ID NO: 156 (clone K090-01a030);
(j) an antibody comprising amino acid sequences of a heavy chain set forth in SEQ ID NO: 162 and a light chain set forth in SEQ ID NO: 164 (clone K090-01a042);
(k) an antibody comprising amino acid sequences of a heavy chain set forth in SEQ ID NO: 166 and a light chain set forth in SEQ ID NO: 168 (clone K090-02a002);
(l) an antibody comprising amino acid sequences of a heavy chain set forth in SEQ ID NO: 170 and a light chain set forth in SEQ ID NO: 172 (clone K090-02a006);
(m) an antibody comprising amino acid sequences of a heavy chain set forth in SEQ ID NO: 174 and a light chain set forth in SEQ ID NO: 176 (clone K090-02a010);
(n) an antibody comprising amino acid sequences of a heavy chain set forth in SEQ ID NO: 178 and a light chain set forth in SEQ ID NO: 180 (clone K090-02a014);
(o) an antibody comprising amino acid sequences of a heavy chain set forth in SEQ ID NO: 182 and a light chain set forth in SEQ ID NO: 184 (clone K090-02a022);
(p) an antibody comprising amino acid sequences of a heavy chain set forth in SEQ ID NO: 186 and a light chain set forth in SEQ ID NO: 188 (clone K090-02a034);
(q) an antibody comprising amino acid sequences of a heavy chain set forth in SEQ ID NO: 190 and a light chain set forth in SEQ ID NO: 192 (clone K090-02a035);
(r) an antibody comprising amino acid sequences of a heavy chain set forth in SEQ ID NO: 194 and a light chain set forth in SEQ ID NO: 196 (clone K090-02b006); and
(s) a mutant of an antibody selected from (a) to (r) comprising at least one substitution, addition, or deletion.

In a preferred embodiment, the antibody or antigen binding fragment thereof that can be used in the present invention has sequences of heavy chains and light chains of the following clone: 01a033, 01a002, 02a010, 02a002, 02a014, 02b006, 01a042, 01a017, 01a026, 01a016, 01a030, or 01a009. These clones exhibited excellent EC50 values against TE14 cells as well as DU145 cells.

In another embodiment, the mutant of the antibody described above can have an amino acid sequence that is at least about 50%, about 60%, about 70%, about 80%, about 90%, about 95%, about 98%, or about 99% identical to the amino acid sequence of the original antibody. The mutant of the antibody described above can comprise at least one substitution, addition, or deletion in a framework.

The antibody that can be used in the present invention preferably binds to human Glypican-1 with a binding affinity at a binding constant of about 10 nM or less, but it is understood that the binding affinity can be weaker than the above binding affinity if the antibody has internalization activity and desired ADC activity.

Those skilled in the art understand that an antibody is useful as an antibody used in an ADC or an antibody for diagnosis/detection if the antibody has internalization activity of at least about 30% with respect to GPC-1 positive cells (e.g., TE8 cells). This is because drug efficacy can be observed when any antibody having internalization activity of at least 30% is used in an ADC. Therefore, in a specific embodiment, the antibody of the invention has an internalization activity of about 30% or greater, about 40% or greater, about 50% or greater, about 60% or greater, about 70% or greater, about 80% or greater, or about. 90% or greater with respect to GPC-1 positive cells (e.g., TE8 cells) after 6 hours from starting incubation with the antibody or antigen fragment thereof in an internalization assay. In another embodiment, the antibody of the invention has an internalization activity of about 30% or greater, about 40% or greater, about 50% or greater, about 60% or greater, about 70% or greater, about 80% or greater, or about 90% or greater with respect to GPC-1 positive cells (e.g., TE8 cells) after 2 hours from starting incubation with the antibody or antigen fragment thereof in an internalization assay. In another embodiment, the antibody of the invention has internalization activity of preferably about 50% or greater and more preferably about 60% or greater with respect to GPC-1 positive cells (e.g., TE8 cells) after 6 hours from starting incubation with the antibody or antigen fragment thereof in an internalization assay. In another embodiment, the antibody of the invention has internalization activity of preferably about 50% or greater and more preferably about 60% or greater with respect to GPC-1 positive cells (e.g., TE8 cells) after 2 hours from starting incubation with the antibody or antigen fragment thereof in an internalization assay. For example, internalization activity can be measured by an assay using TE8 cells as described in the Examples. A value obtained by subtracting the percentage of residual Glypican-1 on the cell surface from 100% was used as the % internalized herein.

In still another embodiment, the antibody of the invention has an internalization activity of at least about 60% or greater with respect to Glypican-1 positive cells expressing Glypican-1 at a high level on the cell surface. Expression of Glypican-1 at a high level means having anti-Glypican-1 antibody binding capacity of about 15000 or greater by using clone 01a033 in an assay using QIFIKIT®. The measurement method of antibody binding capacity is described in detail in Example 5.

In still another embodiment, the antibody of the invention can be an antibody selected from a monoclonal antibody, a polyclonal antibody, a chimeric antibody, a humanized antibody, a human antibody, a multifunctional antibody, a bispecific or oligospecific antibody, a single chain antibody, an scFV, a diabody, an sc(Fv)2 (single chain (Fv)2), and an scFv-Fc.

The antibody of the invention has high intracellular invasion (internalization) activity with respect to Glypican-1 positive cells. Such activity was not found in conventional anti-Glypican-1 antibodies. Therefore, the present invention can be used in various therapeutic applications that were not envisioned in conventional art by utilizing high internalization activity of the antibody of the invention. Therefore, in a specific embodiment, the present invention provides a pharmaceutical composition comprising the anti-Glypican-1 antibody described above. In still another embodiment, the pharmaceutical composition of the invention can be for intercellular invasion of an active ingredient. Examples of the active ingredient include, but are not limited to, agents with cytotoxic activity, anticancer agents, contrast media, siRNAs, antisense nucleic acids, ribozymes, and the like.

A siRNA is an RNA molecule having a double stranded RNA moiety consisting of about 15 to about 40 bases. An siRNA has a function of cleaving an mRNA of a target gene with a sequence that is complementary to an antisense strand of the siRNA, and suppressing the expression of the target gene. For example, the siRNA used herein is an RNA comprising a double stranded RNA moiety consisting of a sense RNA strand consisting of a sequence that is homologous to a contiguous RNA sequence in an mRNA encoding Glypican-1 in a Glypican-1 positive cell or an mRNA encoding a gene affecting cell viability, and an antisense RNA strand consisting of a sequence that is complementary to the sense RNA sequence.

Design and manufacture of such siRNA and mutant siRNA described below are within the technical competence of those skilled in the art. Those skilled in the art can appropriately select any contiguous RNA regions of mRNA that is a transcription product in a target cell to make a double stranded RNA corresponding to the region within the ordinary practice. Those skilled in the art can also appropriately select an siRNA sequence having a stronger RNAi effect from an mRNA sequence that is a transcription product of the sequence using a known method.

For the preparation of siRNA, conditions such as (1) absence of 4 or more consecutive G or C, (2) absence of 4 or more consecutive A or T, or (3) less than 9 bases of G or C can be added. The length of a double stranded RNA moiety, as bases, is 15 to 40 bases, preferably 15 to 30 bases, more preferably 15 to 25 bases, still more preferably 18 to 23 bases, and most preferably 19 to 21 bases. It is understood that the upper and lower limits thereof are not limited to such specific numbers. The limits can be any combination of the listed numbers. The terminal structure of a sense or antisense strand of siRNA is not particularly limited, but can be appropriately selected depending on the objective. For example, the structure can have a blunt end or a cohesive end (overhang), and a structural type with a protruding 3′ terminus is preferred. An siRNA with an overhang consisting of several bases, preferably 1 to 3 bases, and more preferably 2 bases at the 3′ terminus of a sense RNA strand and an antisense RNA strand is preferred for often having a significant effect of suppressing the expression of a target gene. The type of bases of an overhang is not particularly limited, which can be either a base constituting an RNA or a base constituting a DNA. Examples of a preferred overhang sequence include dTdT (2 bp of deoxy T) at the 3′ end and the like. Examples of preferred siRNA include, but are not limited to, siRNA with dTdT (2 bp of deoxy T) at the 3′ terminus of all siRNA sense/antisense strands.

Furthermore, it is also possible to use an siRNA in which one to several nucleotides are deleted, substituted, inserted, and/or added at one or both of the sense strand and antisense strand of the siRNA described above. One to several bases as used herein is not particularly limited, but is preferably 1 to 4 bases, more preferably 1 to 3 bases, and most preferably 1 to 2 bases. Specific examples of such mutations include, but are not limited to, mutations resulting in 0 to 3 bases at the 3′ overhang portion, mutations that change the base sequence of the 3′-overhang portion to another base sequence, mutations resulting in the lengths of the sense RNA strand and antisense RNA strand being different by 1 to 3 bases due to insertion, addition, or deletion of bases, mutations substituting a base in the sense strand and/or antisense strand with another base, and the like. However, it is necessary that the sense strand and antisense strand can hybridize in such mutant siRNAs, and these mutant siRNAs have the ability to suppress gene expression that is equivalent to that of siRNAs without any mutation.

An siRNA can also be a molecule with a structure in which one end is closed, such as an siRNA with a hairpin structure (Short Hairpin RNA; shRNA). An shRNA is an RNA comprising a sense strand RNA of a specific sequence of a target gene, an antisense strand RNA consisting of a sequence complementary to the sense strand sequence, and a linker sequence for connecting the two strands, wherein the sense strand portion hybridizes with the antisense strand portion to form a double-stranded RNA portion.

In order to make the siRNA of the invention, a known method, such as a method using chemical synthesis or a method using a gene recombination technique, can be appropriately used. With a method using synthesis, a double-stranded RNA can be synthesized based on sequence information by using a common method. With a method using a gene recombination technique, an siRNA can be made by constructing an expression vector encoding a sense strand sequence and an antisense strand sequence and introducing the vector into a host cell, and then obtaining each of sense strand RNA and antisense strand RNA produced by transcription. It is also possible to make a desired double-stranded RNA by expressing an shRNA forming a hairpin structure, comprising a sense strand of a specific sequence of a target gene, an antisense strand consisting of a sequence complementary to the sense strand sequence, and a linker sequence for linking the two strands.

For an siRNA, all or some of nucleic acids constituting the siRNA can be a naturally-occurring or modified nucleic acid, as long as it has activity to suppress the expression of a target gene. A modified nucleic acid refers to a nucleic acid, which has a modification at a nucleoside (base moiety, sugar moiety) and/or an inter-nucleoside binding site and has a structure that is different from that of a naturally occurring nucleic acid.

In one embodiment, an active ingredient that is intracellularly migrated by the pharmaceutical composition of the invention can be an antisense nucleic acid. A technology that is well known to those skilled in the art can be used as a method of utilizing an antisense nucleic acid. An antisense nucleic acid can inhibit the expression of a target gene by inhibiting various processes such as transcription, splicing, or translation (Hirashima and Inoue, Shin-seikagaku Jikken Kouza 2 [New Biochemical Experiment Course 2] Kakusan IV Idenshi no Fukusei to Hatsugen [Replication and Expression of Gene of Nucleic Acid IV], Ed. by the Japanese Biochemical Society, Tokyo Kagaku Dojin, 1993, 319-347).

In one embodiment, designing an antisense sequence that is complementary to an untranslated region in the vicinity of the 5′ terminus of mRNA encoding a gene in a target cell is considered effective for inhibiting the translation of the gene. A sequence that is complementary to a 3′ untranslated region or a coding region can also be used. In this manner, the antisense nucleic acid used in the present invention also encompasses nucleic acids comprising an antisense sequence of the sequence of not only the translated region, but also the untranslated region of a gene. An antisense nucleic acid to be used is linked downstream of a suitable promoter, and preferably a sequence comprising a transcription termination signal is linked to the 3′ side. A nucleic acid prepared in this manner can be transformed into a cell by using a known method. The sequence of an antisense nucleic acid is preferably a sequence that is complementary to a gene to be transformed or a portion thereof. However, such a sequence does not need to be fully complementary, as long as the gene expression can be effectively suppressed. A transcribed RNA preferably has complementarity that is 90% or greater, and most preferably 95% or greater, with respect to a transcript of a target gene. In order to effectively inhibit the expression of a target gene using an antisense nucleic acid, it is preferable that the length of the antisense nucleic acid is at least 12 bases and less than 25 bases, but the antisense nucleic acid of the invention is not necessarily limited to this length. For example, the length may be 11 bases or less, 100 bases or more, or 500 bases or more. An antisense nucleic acid may be comprised of only DNA, but may comprise a nucleic acid other than DNAs, such as a locked nucleic acid (LNA). As one embodiment, an antisense nucleic acid used in the invention can be an LNA containing antisense nucleic acid comprising LNA at the 5′ terminus or LNA at the 3′ terminus. In an embodiment using the antisense nucleic acid in the present invention, the antisense sequence can be designed using the method described in, for example, Hirashima and Inoue, Shin-seikagaku Jikkenn Kouza 2 [New Biochemical Experiment Course 2] Kakusan IV Idenshi no Fukusei to Hatsugen [Replication and Expression of Gene of Nucleic Acid IV], Ed. by the Japanese Biochemical Society, Tokyo Kagaku Dojin, 1993, 319-347.

In one embodiment, an active ingredient that is intracellularly migrated by the pharmaceutical composition of the invention can be a ribozyme or DNA encoding a ribozyme. A ribozyme refers to an RNA molecule having catalytic activity. While there are ribozymes with various activities, a study focusing on ribozymes particularly as an enzyme for cleaving an RNA has made it possible to design a ribozyme that site-specifically cleaves an RNA. There are ribozymes with a size of 400 nucleotides or more as in M1 RNA included in RNase P and group I intron ribozymes, but there are also those with an active domain of about 40 nucleotides called hammerhead or hair-pin ribozymes (Makoto Koizumi and Eiko Otsuka, Protein, Nucleic Acid and Enzyme, 1990, 35, 2191).

Hairpin ribozymes are also useful for the objective of the invention. Such a ribozyme is found, for example, in the minus strand of a tobacco ringspot virus satellite RNA (Buzayan J M, Nature, 1986, 323, 349). It is demonstrated that target specific RNA-cleaving ribozymes can also be created from hairpin ribozymes (Kikuchi, Y. & Sasaki, N., Nucl. Acids Res, 1991, 19, 6751., Yo Kikuchi, Kagaku to Seibutsu [Chemistry and Biology], 1992, 30, 112).

(Complex)

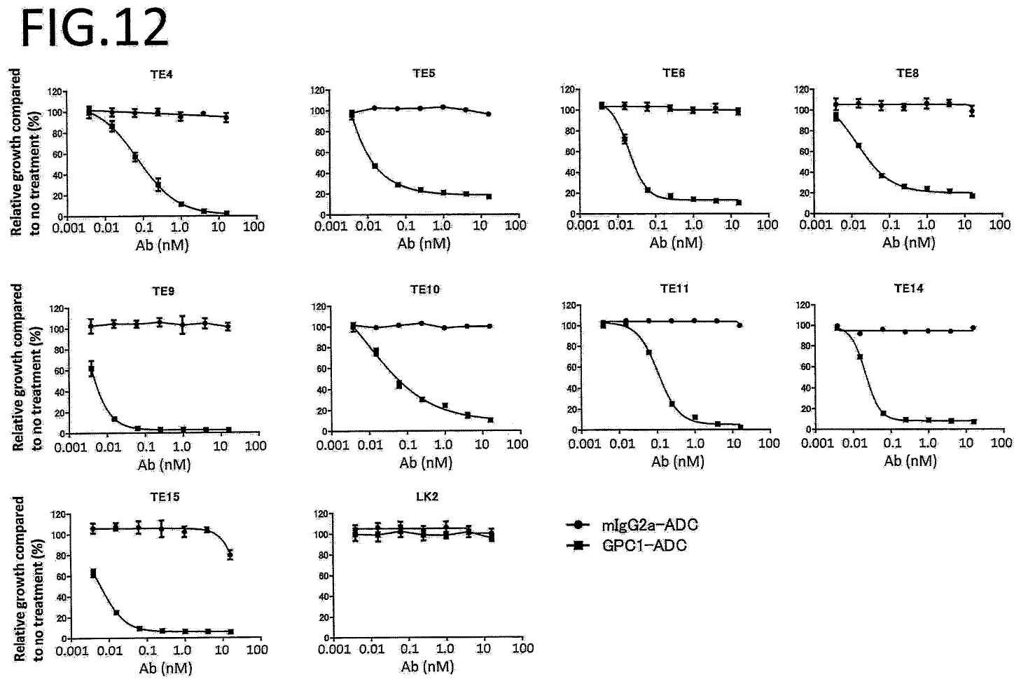

In another aspect, the present invention provides a complex of a substance that binds to Glypican-1 and an agent having efficacy such as cytotoxic activity. As shown in the Examples, Glypican-1 is highly expressed only in specific cells. Thus, the complex of the invention can specifically act on Glypican-1 positive cells. More specifically, the complex of the invention targets Glypican-1 positive cells expressing Glypican-1 at a high level on a cell surface. Thus, the complex of the invention does not exhibit an effect of suppressing cell growth against lung cancer derived LK2 cell strains expressing Glypican-1 at a low level (FIG. 12). Expression of Glypican-1 at a high level means having anti-Glypican-1 antibody binding capacity of about 15000 or greater using clone 01a033 in an assay using QIFIKIT®. A measurement method of antibody binding capacity is described in detail in Example 5.

In some embodiments, a substance that binds to Glypican-1 can bind to an agent with toxic activity in any binding mode. For example, a substance that binds to Glypican-1 can covalently or non-covalently link to an agent having toxic activity. In a preferred embodiment, a substance that binds to Glypican-1 can be linked to an agent having cytotoxic activity via a linker. In such a case, the substance that bind to Glypican-1 is operably linked to the agent having cytotoxic activity. Therefore, a linker used in the complex of the invention can be any linker, as long as the substance that binds to Glypican-1 is operably linked to the agent having cytotoxic activity.

In a specific embodiment, it is preferable that a linker is stable in blood, but is cleaved after intracellular invasion. For example, a linker can be designed so that the linker has a cleavable site, which is not degraded by an enzyme that is present in blood, but is cleaved by an enzyme that is present only in cells. Examples of such a linker include linkers having intralysosomal enzyme cleaved sequence (valine-citrulline (Val-Citr)). Those skilled in the art can design an appropriate linker having a suitable enzyme cleaved site using a well-known method.

In still another specific embodiment, the linker used in the complex of the invention can comprise a cathepsin cleaved sequence. Such a linker has, for example, the MC-Val-Citr-PAB-portion of the structure (MC-Val-Citr-PAB-agent)

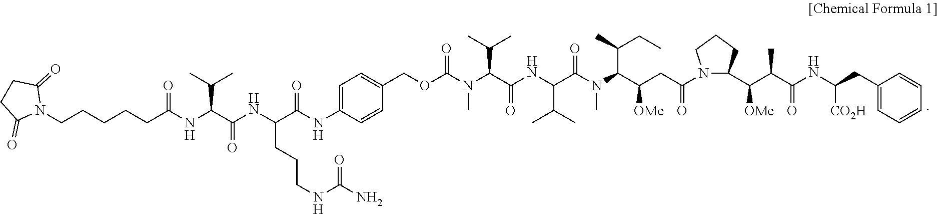 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)C(NC(=O)[C@H](C(C)C)N(C)C(=O)OCc1ccc(NC(=O)C(CCCNC(N)=O)NC(=O)[C@@H](NC(=O)CCCCCN2C(=O)CCC2=O)C(C)C)cc1)C(C)C]

In another embodiment, a linker used in the complex of the invention can be an acid labile linker. An acid labile linker responsive to a pH can be used by utilizing an acidic pH in the endosome after intracellular invasion. Examples of such a linker include hydrazone and the like.

In still another embodiment, a linker used in the complex of the invention can be a disulfide linker. Examples of such a linker include N-succinimidyl4-(2-pyridylthio)butanoate (SPDB), N-succinimidyl4-(2-pyridylthio)pentanoate (SPP), and the like.

A linker used in the complex of the invention can be a noncleavable linker. Examples of noncleavable linkers include, but are not limited to, maleimidomethylcyclohexane-1-carboxylate (MCC linker) and the like.

The agent can be any agent known to have cytotoxicity. Examples thereof include auristatin (monomethyl auristatin-E (MMAE), monomethyl auristatin-F (MMAF), and the like), maytansinoids (DM1 and DM4), calicheamicin, duocarmycin, pyrrolobenzothiazepine (PBD), and topoisomerase inhibitors. Those skilled in the art can appropriately select an agent that is used in a complex, depending on the sensitivity of the targeted cancer to an agent. The agent sensitivity with respect to cancer is well known in the art. Even if sensitivity of a specific cancer to a specific agent were not known, those skilled in the art can readily find the sensitivity of an agent with respect to a cancer cell strain by using a cell strain of a targeted cancer as in Example 2 or the like.

The complex of the invention can exhibit cytotoxic activity by invasion into cells. Therefore, in a certain embodiment, a substance that binds to Glypican-1 can have activity for intracellular invasion into a target cell. In another embodiment, cytotoxic activity can be exhibited by an agent having cytotoxic activity itself having cell permeability.

In some embodiments, the complex of the invention exhibits an 1050 of about 0.5 nM (5.0×10⁻¹⁰ M) or less in Glypican-1 positive cells. In another embodiment, the complex of the invention exhibits an 1050 of about 0.1 nM (1.0×10⁻¹⁰ M) or less in Glypican-1 positive cells. In still another embodiment, the complex of the invention exhibits an 1050 of about 0.05 nM (5.0×10⁻¹¹ M) or less in Glypican-1 positive cells. In a specific embodiment, the complex of the invention exhibits an ICH of about 0.03 nM (3.0×10⁻¹¹ M) or less in Glypican-1 positive cells. In a more specific embodiment, the complex of the invention exhibits an IC₅₀ of about 0.02 nM (2.0×10⁻¹¹ M) or less in Glypican-1 positive cells.

In a specific embodiment, a substance that binds to Glypican-1 can be an antibody or an antigen binding fragment thereof.

In a specific embodiment, a substance that binds to Glypican-1 can be an antibody or an antigen binding fragment thereof, wherein an epitope of the antibody can be:

(a) positions 33 to 61 of SEQ ID NO: 2;
(b) positions 339 to 358 and/or 388 to 421 of SEQ ID NO: 2;
(c) positions 430 to 530 of SEQ ID NO: 2;
(d) positions 33 to 61, 339 to 358, and/or 388 to 421 of SEQ ID NO: 2;
(e) positions 339 to 358, 388 to 421, and/or 430 to 530 of SEQ ID NO: 2; or
(f) positions 33 to 61, 339 to 358, 388 to 421, and/or 430 to 530 of SEQ ID NO: 2. More preferably, an epitope of the antibody can be or comprise:
(a) positions 33 to 61 of SEQ ID NO: 2;
(b) positions 339 to 358 and 388 to 421 of SEQ ID NO: 2;
(c) positions 33 to 61, 339 to 358, and 388 to 421 of SEQ ID NO: 2; or
(d) positions 33 to 61, 339 to 358, 388 to 421, and 430 to 530 of SEQ ID NO: 2.

In another specific embodiment, an antibody used in the complex of the invention is selected from the group consisting of:

(a) an antibody comprising amino acid sequences of heavy chain CDR1, heavy chain CDR2, and heavy chain CDR3 set forth in SEQ ID NOs: 53, 54, and 55, respectively, and light chain CDR1, light chain CDR2, and light chain CDR3 set forth in SEQ ID NOs: 56, 57, and 58, respectively (clone K090-01a033);
(b) an antibody comprising amino acid sequences of heavy chain CDR1, heavy chain CDR2, and heavy chain CDR3 set forth in SEQ ID NOs: 5, 6, and 7, respectively, and light chain CDR1, light chain CDR2, and light chain CDR3 set forth in SEQ ID NOs: 8, 9, and 10, respectively (clone K090-01a002);
(c) an antibody comprising amino acid sequences of heavy chain CDR1, heavy chain CDR2, and heavy chain CDR3 set forth in SEQ ID NOs: 11, 12, and 13, respectively, and light chain CDR1, light chain CDR2, and light chain CDR3 set forth in SEQ ID NOs: 14, 15, and 16, respectively (clone K090-01a007);
(d) an antibody comprising amino acid sequences of heavy chain CDR1, heavy chain CDR2, and heavy chain CDR3 set forth in SEQ ID NOs: 17, 18, and 19, respectively, and light chain CDR1, light chain CDR2, and light chain CDR3 set forth in SEQ ID NOs: 20, 21, and 22, respectively (clone K090-01a016);
(e) an antibody comprising amino acid sequences of heavy chain CDR1, heavy chain CDR2, and heavy chain CDR3 set forth in SEQ ID NOs: 23, 24, and 25, respectively, and light chain CDR1, light chain CDR2, and light chain CDR3 set forth in SEQ ID NOs: 26, 27, and 28, respectively (clone K090-01a017);
(f) an antibody comprising amino acid sequences of heavy chain CDR1, heavy chain CDR2, and heavy chain CDR3 set forth in SEQ ID NOs: 29, 30, and 31, respectively, and light chain CDR1, light chain CDR2, and light chain CDR3 set forth in SEQ ID NOs: 32, 33, and 34, respectively (clone K090-01a021);
(g) an antibody comprising amino acid sequences of heavy chain CDR1, heavy chain CDR2, and heavy chain CDR3 set forth in SEQ ID NOs: 35, 36, and 37, respectively, and light chain CDR1, light chain CDR2, and light chain CDR3 set forth in SEQ ID NOs: 38, 39, and 40, respectively (clone K090-01a026);
(h) an antibody comprising amino acid sequences of heavy chain CDR1, heavy chain CDR2, and heavy chain CDR3 set forth in SEQ ID NOs: 41, 42, and 43, respectively, and light chain CDR1, light chain CDR2, and light chain CDR3 set forth in SEQ ID NOs: 44, 45, and 46, respectively (clone K090-01a009);
(i) an antibody comprising amino acid sequences of heavy chain CDR1, heavy chain CDR2, and heavy chain CDR3 set forth in SEQ ID NOs: 47, 48, and 49, respectively, and light chain CDR1, light chain CDR2, and light chain CDR3 set forth in SEQ ID NOs: 50, 51, and 52, respectively (clone K090-01a030);
(j) an antibody comprising amino acid sequences of heavy chain CDR1, heavy chain CDR2, and heavy chain CDR3 set forth in SEQ ID NOs: 59, 60, and 61, respectively, and light chain CDR1, light chain CDR2, and light chain CDR3 set forth in SEQ ID NOs: 62, 63, and 64, respectively (clone K090-01a042);
(k) an antibody comprising amino acid sequences of heavy chain CDR1, heavy chain CDR2, and heavy chain CDR3 set forth in SEQ ID NOs: 65, 66, and 67, respectively, and light chain CDR1, light chain CDR2, and light chain CDR3 set forth in SEQ ID NOs: 68, 69, and 70, respectively (clone K090-02a002);
(l) an antibody comprising amino acid sequences of heavy chain CDR1, heavy chain CDR2, and heavy chain CDR3 set forth in SEQ ID NOs: 71, 72, and 73, respectively, and light chain CDR1, light chain CDR2, and light chain CDR3 set forth in SEQ ID NOs: 74, 75, and 76, respectively (clone K090-02a006);
(m) an antibody comprising amino acid sequences of heavy chain CDR1, heavy chain CDR2, and heavy chain CDR3 set forth in SEQ ID NOs: 77, 78, and 79, respectively, and light chain CDR1, light chain CDR2, and light chain CDR3 set forth in SEQ ID NOs: 80, 81, and 82, respectively (clone K090-02a010);
(n) an antibody comprising amino acid sequences of heavy chain CDR1, heavy chain CDR2, and heavy chain CDR3 set forth in SEQ ID NOs: 83, 84, and 85, respectively, and light chain CDR1, light chain CDR2, and light chain CDR3 set forth in SEQ ID NOs: 86, 87, and 88, respectively (clone K090-02a014);
(o) an antibody comprising amino acid sequences of heavy chain CDR1, heavy chain CDR2, and heavy chain CDR3 set forth in SEQ ID NOs: 89, 90, and 91, respectively, and light chain CDR1, light chain CDR2, and light chain CDR3 set forth in SEQ ID NOs: 92, 93, and 94, respectively (clone K090-02a022);
(p) an antibody comprising amino acid sequences of heavy chain CDR1, heavy chain CDR2, and heavy chain CDR3 set forth in SEQ ID NOs: 95, 96, and 97, respectively, and light chain CDR1, light chain CDR2, and light chain CDR3 set forth in SEQ ID NOs: 98, 99, and 100, respectively (clone K090-02a034);
(q) an antibody comprising amino acid sequences of heavy chain CDR1, heavy chain CDR2, and heavy chain CDR3 set forth in SEQ ID NOs: 101, 102, and 103, respectively, and light chain CDR1, light chain CDR2, and light chain CDR3 set forth in SEQ ID NOs: 104, 105, and 106, respectively (clone K090-02a035);
(r) an antibody comprising amino acid sequences of heavy chain CDR1, heavy chain CDR2, and heavy chain CDR3 set forth in SEQ ID NOs: 107, 108, and 109, respectively, and light chain CDR1, light chain CDR2, and light chain CDR3 set forth in SEQ ID NOs: 110, 111, and 112, respectively (clone K090-02b006);
(s) an antibody comprising amino acid sequences of heavy chain CDR1, heavy chain CDR2, and heavy chain CDR3 set forth in SEQ ID NOs: 113, 114, and 115, respectively, and light chain CDR1, light chain CDR2, and light chain CDR3 set forth in SEQ ID NOs: 116, 117, and 118, respectively (clone 4);
(t) an antibody comprising amino acid sequences of heavy chain CDR1, heavy chain CDR2, and heavy chain CDR3 set forth in SEQ ID NOs: 125, 126, and 127, respectively, and light chain CDR1, light chain CDR2, and light chain CDR3 set forth in SEQ ID NOs: 128, 129, and 130, respectively (clone 18); and
(u) a mutant of an antibody selected from (a) to (t) comprising at least one substitution, addition, or deletion.

In a preferred embodiment, the antibody or antigen binding fragment thereof that can be used in the present invention has sequences of heavy chain CDRs and light chain CDRs of the following clone: 01a033, 01a002, 02a010, 02a002, clone 18, 02a014, 02b006, 01a042, 01a017, 01a026, 01a016, 01a030, clone 4, or −01a009. These clones exhibited excellent EC50 values against TE14 cells as well as DU145 cells.

In still another embodiment, the antibody that is used in the complex of the invention is selected from the group consisting of:

(a) an antibody comprising amino acid sequences of a heavy chain set forth in SEQ ID NO: 158 and a light chain set forth in SEQ ID NO: 160 (clone K090-01a033);
(b) an antibody comprising amino acid sequences of a heavy chain set forth in SEQ ID NO: 126 and a light chain set forth in SEQ ID NO: 128 (clone K090-01a002);
(c) an antibody comprising amino acid sequences of a heavy chain set forth in SEQ ID NO: 130 and a light chain set forth in SEQ ID NO: 132 (clone K090-01a007);
(d) an antibody comprising amino acid sequences of a heavy chain set forth in SEQ ID NO: 134 and a light chain set forth in SEQ ID NO: 136 (clone K090-01a016);
(e) an antibody comprising amino acid sequences of a heavy chain set forth in SEQ ID NO: 0.138 and a light chain set forth in SEQ ID NO: 140 (clone K090-01a017);
(f) an antibody comprising amino acid sequences of a heavy chain set forth in SEQ ID NO: 142 and a light chain set forth in SEQ ID NO: 144 (clone K090-01a021);
(g) an antibody comprising amino acid sequences of a heavy chain set forth in SEQ ID NO: 146 and a light chain set forth in SEQ ID NO: 148 (clone K090-01a026);
(h) an antibody comprising amino acid sequences of a heavy chain set forth in SEQ ID NO: 150 and a light chain set forth in SEQ ID NO: 152 (clone K090-01a009);
(i) an antibody comprising amino acid sequences of a heavy chain set forth in SEQ ID NO: 154 and a light chain set forth in SEQ ID NO: 156 (clone K090-01a030);
(j) an antibody comprising amino acid sequences of a heavy chain set forth in SEQ ID NO: 162 and a light chain set forth in SEQ ID NO: 164 (clone K090-01a042);
(k) an antibody comprising amino acid sequences of a heavy chain set forth in SEQ ID NO: 166 and a light chain set forth in SEQ ID NO: 168 (clone K090-02a002);
(l) an antibody comprising amino acid sequences of a heavy chain set forth in SEQ ID NO: 170 and a light chain set forth in SEQ ID NO: 172 (clone K090-02a006);
(m) an antibody comprising amino acid sequences of a heavy chain set forth in SEQ ID NO: 174 and a light chain set forth in SEQ ID NO: 176 (clone K090-02a010);
(n) an antibody comprising amino acid sequences of a heavy chain set forth in SEQ ID NO: 178 and a light chain set forth in SEQ ID NO: 180 (clone K090-02a014);
(o) an antibody comprising amino acid sequences of a heavy chain set forth in SEQ ID NO: 182 and a light chain set forth in SEQ ID NO: 184 (clone K090-02a022);
(p) an antibody comprising amino acid sequences of a heavy chain set forth in SEQ ID NO: 186 and a light chain set forth in SEQ ID NO: 188 (clone K090-02a034);
(q) an antibody comprising amino acid sequences of a heavy chain set forth in SEQ ID NO: 190 and a light chain set forth in SEQ ID NO: 192 (clone K090-02a035);
(r) an antibody comprising amino acid sequences of a heavy chain set forth in SEQ ID NO: 194 and a light chain set forth in SEQ ID NO: 196 (clone K090-02b006);
(s) an antibody comprising amino acid sequences of a heavy chain set forth in SEQ ID NO: 198 and a light chain set forth in SEQ ID NO: 200 (clone 4);
(t) an antibody comprising amino acid sequences of a heavy chain set forth in SEQ ID NO: 202 and a light chain set forth in SEQ ID NO: 204 (clone 18); and
(u) a mutant of an antibody selected from (a) to (t) comprising at least one substitution, addition, or deletion.

In a preferred embodiment, the antibody or antigen binding fragment that can be used in the present invention has sequences of heavy chains and light chains of the following clone: 01a033, 01a002, 02a010, 02a002, clone 18, 02a014, 02b006, 01a042, 01a017, 01a026, 01a016, 01a030, clone 4, or 01a009. These clones exhibited excellent EC50 values against TE14 cells as well as DU145 cells.

In another embodiment, a mutant of the antibody described above can have an amino acid sequence that is at least about 50%, about 60%, about 70%, about 80%, about 90%, about 95%, about 98%, or about 99% identical to the amino acid sequence of the original antibody. The mutant of the antibody described above can comprise at least one substitution, addition, or deletion in a framework.

(Therapeutic Drug)

In another aspect, the present invention provides a composition for preventing or treating Glypican-1 positive cancer. The composition of the invention comprises the complex described above of a substance that binds to Glypican-1 and an agent having cytotoxic activity. More specifically, the target of prevention or treatment of the composition of the invention is a Glypican-1 positive cell expressing Glypican-1 at a high level on a. cell surface. Expression of Glypican-1 at a high level means having an anti-Glypican-1 antibody binding capacity of about 15000 or greater using clone 01a033 in an assay using QIFIKIT®. A measurement method of antibody binding capacity is described in detail in Example 5.

In some embodiments, a substance that binds to Glypican-1 can be an anti-Glypican-1 antibody or antigen binding fragment thereof having activity for intracellular invasion into Glypican-1 positive cells. An epitope of an anti-Glypican-1 antibody or antigen binding fragment thereof having activity for intracellular invasion into Glypican-1 positive cells can be:

(a) positions 33 to 61 of SEQ ID NO: 2;
(b) positions 339 to 358 and/or 388 to 421 of SEQ ID NO: 2;
(c) positions 430 to 530 of SEQ ID NO: 2;
(d) positions 33 to 61, 339 to 358, and/or 388 to 421 of SEQ ID NO: 2;
(e) positions 339 to 358, 388 to 421, and/or 430 to 530 of SEQ ID NO: 2; or
(f) positions 33 to 61, 339 to 358, 388 to 421, and/or 430 to 530 of SEQ ID NO: 2. More preferably, an epitope of an anti-glypican-1 antibody or antigen binding fragment thereof having activity for intracellular invasion into Glypican-1 positive cells can be or comprise:
(a) positions 33 to 61 of SEQ ID NO: 2;
(b) positions 339 to 358 and 388 to 421 of SEQ ID NO: 2;
(c) positions 33 to 61, 339 to 358, and 388 to 421 of SEQ ID NO: 2; or
(d) positions 33 to 61, 339 to 358, 388 to 421, and 430 to 530 of SEQ ID NO: 2.

In some embodiments, examples of anti-Glypican-1 antibody having activity for intracellular invasion into Glypican-1 positive cells include clone K090-01a002, K090-01a007, K090-01a016, K090-01a017, K090-01a021, K090-01a026, K090-01a009, K090-01a030, K090-01a033, K090-01a042, K090-02a002, K090-02a006, K090-02a010, K090-02a014, K090-02a022, K090-02a034, K090-02a035, K090-02b006, clone 4, and clone 18, and mutants of these antibodies comprising at least one substitution, addition, or deletion. These antibodies are described above in detail.

In some embodiments, Glypican-1 positive cancer is selected from esophageal cancer, pancreatic cancer, cervical cancer, lung cancer, head and neck cancer, breast cancer, uterine leiomyosarcoma, prostate cancer, and any combination thereof. Esophageal cancer can comprise esophageal cancer at a lymph node metastasis site, squamous cell carcinoma, and/or adenocarcinoma.

(Detection Agent)

In still another aspect, the present invention provides a detection agent for identifying esophageal cancer, comprising an anti-Glypican-1 antibody or fragment thereof. The inventors have created 18 new clones of an anti-Glypican-1 antibody, and found that these clones have intracellular invasion (internalization) activity. Glypican-1 positive cancer (e.g., esophageal cancer) can be specifically detected by utilizing such a property to specifically detect cells into which a labeled anti-Glypican-1 antibody has intracellularly migrated.

The new clones disclosed herein have been found to have intracellular invasion (internalization) activity that was not observed in conventional antibodies. Therefore, in one embodiment, an anti-Glypican-1 antibody can be labeled. In a preferred embodiment, the label is in an inactive state in blood but is activated to be identifiable upon invasion into a Glypican-1 positive cell (e.g., esophageal cancer cell). As a result, Glypican-1 positive cells (e.g., esophageal cancer cells) can be detected with high specificity.

In a specific embodiment, examples of labels include RI (radioisotope), fluorescent labels, biotins, chemiluminescent labels, and the like. When a plurality of labels are used for labeling by the fluorescence method, fluorescent substances having different fluorescent emission maximum wavelengths are used for labeling. It is preferable that the difference in fluorescent emission maximum wavelengths is 10 nm or greater. Since an antibody is labeled, any substance can be used as long as the function of the antibody (especially the binding capacity or internalization activity) is not affected, but Alexa™ Fluor is desirable as the fluorescent substance. Alexa™ Fluor is a water-soluble fluorescent dye obtained by modifying coumarin, rhodamine, fluorescein, cyanine, or the like. This is a series compatible with a wide range of fluorescence wavelengths. Relative to other fluorescent dyes for the corresponding wavelength, Alexa™ Fluor is very stable, bright, and has low pH sensitivity. Examples of combinations of fluorescent dyes with fluorescence maximum wavelength of 10 nm or greater include, but are not limited to, a combination of Alexa™ 555 and Alexa™ 633, combination of Alexa™ 488 and Alexa™ 555, and the like.

In some embodiments, esophageal cancer can comprise esophageal cancer at a lymph node metastasis site, squamous cell carcinoma, and/or adenocarcinoma. In still another embodiment, an agent can be administered to a patient determined to have developed Glypican-1 positive esophageal cancer.

In another aspect, the present invention provides a method of using expression of Glypican-1 in a target sample as an indicator of esophageal cancer, the method comprising: contacting the detection agent described above with the target sample; measuring an amount of expression of Glypican-1 in the target sample; and comparing amounts of expression of Glypican-1 in the target sample and a normal sample.

Since the new clones of anti-Glypican-1 antibodies disclosed herein can specifically detect esophageal cancer cells, the clones can also be provided as a diagnostic agent for determining whether a subject is in need of therapy of esophageal cancer.

The detection agent, inspection agent, or diagnostic agent of the invention can. be used as a detection kit, inspection kit, or diagnostic kit.

(Kit)

In one aspect, the present invention provides a kit for detection, inspection, and/or diagnosis for carrying out the method for detection, inspection, and/or diagnosis according to the present invention. The kit comprises the detection agent, inspection agent, and/or diagnostic agent of the invention. As an embodiment thereof, any embodiment described herein can be used alone or in combination.

(Companion Reagent)

In another aspect, the present invention provides a companion reagent for determining whether a subject is in need of cancer therapy with a Glypican-1 inhibitor, comprising a new clone of an anti-Glypican-1 antibody disclosed herein or the detection agent described above, wherein the reagent is contacted with a target sample, and an amount of expression of Glypican-1 in the target sample is measured, wherein the amount of expression of Glypican-1 in the target sample exceeding an amount of expression of Gypican-1 in a normal sample indicates that the target is in need of therapy with the Glypican-1 inhibitor. If a test is conducted on whether esophageal cancer is Glypican-1 positive in advance using a companion diagnostic drug, the therapeutic efficacy of esophageal cancer therapy targeting Glypican-1 can be examined. If the examination yields a result of Glypican-1 positive, the esophageal cancer therapy targeting Glypican-1 can be determined to be effective. In one embodiment of the invention, “companion diagnosis” comprises diagnosis that is conducted in order to assist optimal dosing by testing and predicting the individual difference in the efficacy and side effects of an agent for the patients.

In another aspect, the present invention provides a composition for preventing or treating Malignant tumor comprising a complex of a substance that binds to Glypican-1 and an agent having cytotoxic activity, wherein a subject having the malignant tumor has a higher expression of Glypican-1 than a normal individual. In some embodiments, whether Glypican-1 is more highly expressed than in normal individuals can be determined by using the detection agent or diagnostic agent described above.

### (Syngenic Nonhuman Animals)

In still another embodiment, the present invention provides a nonhuman animal (syngenic nonhuman animal) to which cells that express a cancer antigen are grafted, wherein the cancer antigen is syngenic with the nonhuman animal. Since the antigen expressed from grafted cells and proteins expressed in normal cells are proteins originating from the same animal for such an animal, the efficacy of an anticancer agent and toxicity to normal cells can be evaluated equally.

In some embodiments, cells expressing a cancer antigen are of cancer cell derived cell strain. In a specific embodiment, cells expressing a cancer antigen are of cancer cell-derived cell strain. In a specific embodiment, the cell strain can be a cell strain modified to express a cancer antigen given that the cancer antigen is syngenic with the nonhuman animal, or a cell strain naturally expressing the cancer antigen. In a preferred embodiment, a cancer antigen can evaluate the efficacy and/or toxicity of an anticancer agent with respect to Glypican-1 positive cancer (e.g., esophageal cancer, pancreatic cancer, cervical cancer, lung cancer, head and neck cancer, breast cancer, uterine leiomyosarcoma, or prostate cancer) by using a cell expressing Glypican-1. Examples of cell strains that can be used include, but are not limited to, LLC (mouse lung cancer cell strain), 4T1 (mouse breast cancer cell strain), MH-1 (mouse ovarian cancer cell strain), CT26 (mouse colon cancer cell strain), MC38 (mouse colon cancer cells strain), B1610 (mouse melanoma cell strain), and the like. In a preferred embodiment, a cell strain that can be used is an LLC cell strain.

In some embodiment, a nonhuman animal is a nonhuman mammal, preferably a rodent (e.g., mouse, rat, hamster, or the like). In a preferred embodiment, a nonhuman animal is a mouse (syngenic mouse).

In a specific embodiment in the present invention, a cell strain modified to express mouse Glypican-1 (e.g., LLC cell strain), can be used. In a preferred embodiment, the nonhuman animal is a mouse, and the cells are of an LLC cell strain modified to express mouse Glypican-1.

As used herein, “or” is used when “at least one or more” of the listed matters in the sentence can be employed. When explicitly described herein as “within the range of two values”, the range also includes the two values themselves.

Reference literatures such as scientific literatures, patents, and patent applications cited herein are incorporated herein by reference to the same extent that the entirety of each document is specifically described.

As described above, the present invention has been described while showing preferred embodiments to facilitate understanding. The present invention is described hereinafter based on Examples. The above descriptions and the following Examples are not provided to limit the present invention, but for the sole purpose of exemplification. Thus, the scope of the present invention is not limited to the embodiments and Examples specifically described herein and is limited only by the scope of claims.

### EXAMPLES

The Examples are described hereinafter. The animals used in the following Examples were handled based on the Declaration of Helsinki while complying with the guidelines stipulated by the National Institutes of Biomedical Innovation, Health and Nutrition when needed. For reagents, the specific products described in the Examples were used. However, the reagents can be substituted with an equivalent product from another manufacturer (Sigma-Aldrich, Wako Pure Chemical, Nacalai Tesque, R & D Systems, USCN Life Science INC, or the like).

### Example 1: Antibody Production

(Materials and Methods)

In order to produce an mAb to human Glypican-1 (GPC1), 4 to 6 week old mice with an MRL or C3H background were immunized with a recombinant human GPC1 protein (R&D systems). After 4 to 5 intraperitoneal immunizations, lymphocytes were collected from the spleen and inguinal lymph node of the immunized mice. Total RNA was isolated from lymphocytes by using a reagent for RNA extraction (ISOGEN, NIPPN GENE CO., LTD.) First strand cDNA was synthesized with SuperScript III Reverse Transcriptase (Thermo Fisher Scientific Inc.) Immunoglobulin V_{H} and V_{L} genes were amplified by PCR from the resulting cDNA and inserted into a pSCCA5-E8d vector (MBL). PCR primers were designed in accordance with IMGT (http://www.imgt.org/). E. coli (DH12S, Thermo Fisher Scientific Inc.) was transformed with a plasmid DNA comprising a cDNA library. The number of independent transformants was estimated to be about 1.5×10⁸. Next, the transformants were infected with helper phage M13K07. A phage expressing the scFv form of an antibody on the surface was obtained from the culture supernatant of the liquid medium. The obtained phase was selected by a known panning method (Marks et al., 1991; Suzuki et al., 2007; Kurosawa et al., 2008). In summary, a human Glypican-1 protein conjugated to Dynabeads (VERITAS) was used as an antigen in the first to third panning. Next, cell strain TE14 that expresses GPC1 on the surface was used for the 4th to 5th panning. The selected phages were screened by ELISA using a human GPC1 immobilized 96-well plate, and then were further screened by cell-ELISA using a cell strain stably transformed with human GPC1. Lastly, reactivity of the selected phages to human GPC1 was studied by flow cytometry (FC500, Beckman Coulter Inc.) by using human GPC1 transformants and TE14. 18 types of clones were obtained as a result.

To form a complete immunoglobulin (IgG) protein, the V_{H} and V_{L} genes of the obtained clones were incorporated into a genetically singular mouse IgG2a expression vector (Mammalian PowerExpress System, TOYOBO). The expression vector was straightened by an enzymatic reaction and transformed into a CHO-K1 cell strain by electroporation. After 10 days of culture, an antibody in an IgG form of the resulting clone was collected from the CHO culture supernatant and purified in an affinity column packed with nProteinA Sepharose 4 Fast Flow (GE healthcare). The reactivity of the purified antibody to human GPC1 was retested by flow cytometry using human GPC1 highly expressing cell strain TE14.

For clones #4, #17, and #18, antibodies were produced as described above based on sequence information obtained by the method described in International Publication No. WO 2015/098112, which is incorporated herein by reference.

### Example 2: ADC Assay Combining Anti-GPC-1 Antibody and MMAF Binding Secondary Antibody

(Materials and Methods)

(Examining Anticancer Agent Sensitivity of TE14 Cells)

2000 TE14 cells were added to a 96-well plate at 90 μl. The cells were cultured overnight at 37° C. in a 5% CO2 incubator. The next day, 10 μl of an anticancer agent concentrated 10-fold relative to the final concentration was added to each well, so that the total amount was 100 μl. The cells were cultured for 6 days at 37° C. in a 5% CO2 incubator. The cell viability was measured by using a CellTiter-Glo Luminescent Cell Viability Assay reagent to detect the amount of ATP. RPMI1640+10% FBS+1% PS was used as the medium. MMAE (model number: 474645-27-7, ALB Technology) and MMAF (model number: 745017-94-1, ALB Technology) were used as the anticancer agent.

(ADC Used in the Assay)

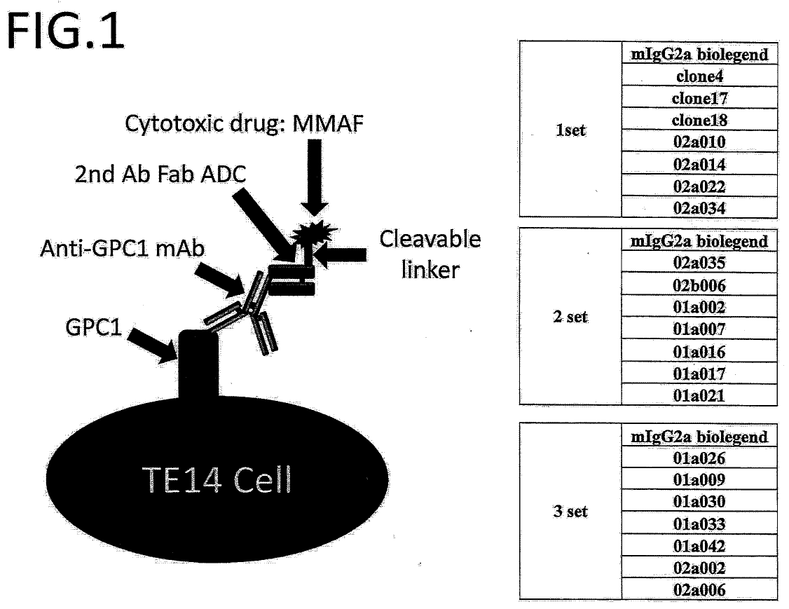

FIG. 1 depicts a schematic diagram of an ADC used in an assay. This Example used a secondary antibody (MORADEC) to the anti-GPC1 antibody made in Example 1, to which an anticancer agent (MMAF) was bound via a linker.

This Example used the following clone sets.

TABLE 1

| 1set | mIgG2a biolegend |
|---|---|
| | clone4 |
| | clone17 |
| | clone18 |
| | 02a010 |
| | 02a014 |
| | 02a022 |
| | 02a034 |
| 2 set | mIgG2a biolegend |
| | 02a035 |
| | 02b006 |
| | 01a002 |
| | 01a007 |
| | 01a016 |
| | 01a017 |
| | 01a021 |
| 3 set | mIgG2a biolegend |
| | 01a026 |
| | 01a009 |
| | 01a030 |
| | 01a033 |
| | 01a042 |
| | 02a002 |
| | 02a006 |

2000 TE14 cells were added to a 96-well plate at 80 μl. The cells were cultured overnight at 37° C. in a 5% CO2 incubator. The next day, 10 μl of an anticancer agent bound secondary antibody and 10 μl of a primary antibody concentrated 10-fold relative to the final concentration was added to each well, so that the total amount was 100 μl. The cells were cultured for 6 days at 37° C. in a 5% CO2 incubator. The cell viability was measured by using a CellTiter-Glo Luminescent Cell Viability Assay reagent to detect the amount of ATP. RPMI1640+10% FBS+1% PS was used as the medium. The final concentrations of the primary antibody were 0, 0.004, 0.0156, 0.0625, 0.25, and 1.0 nM. 2 μg/ml of Fab-aMFc-CL-MMAF (model number: AM202AF-50, Moradec) was used as the secondary antibody.

(Results)

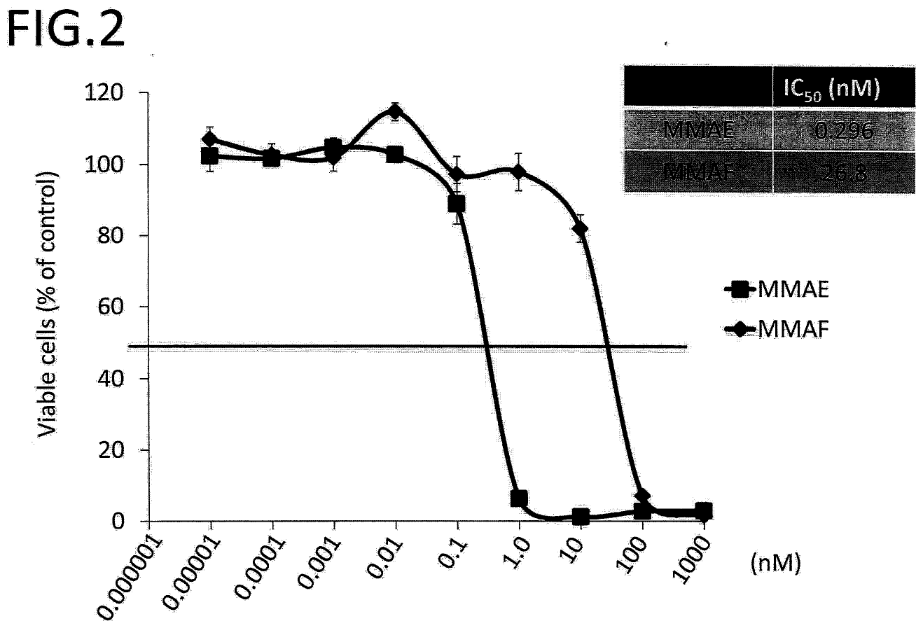

The results are shown in FIG. 2. The anticancer agents MMAE and MMAF exhibited an IC₅₀ of 0.926 nM and 26.8 nM, respectively. TE14 cells were confirmed to be anticancer sensitive.

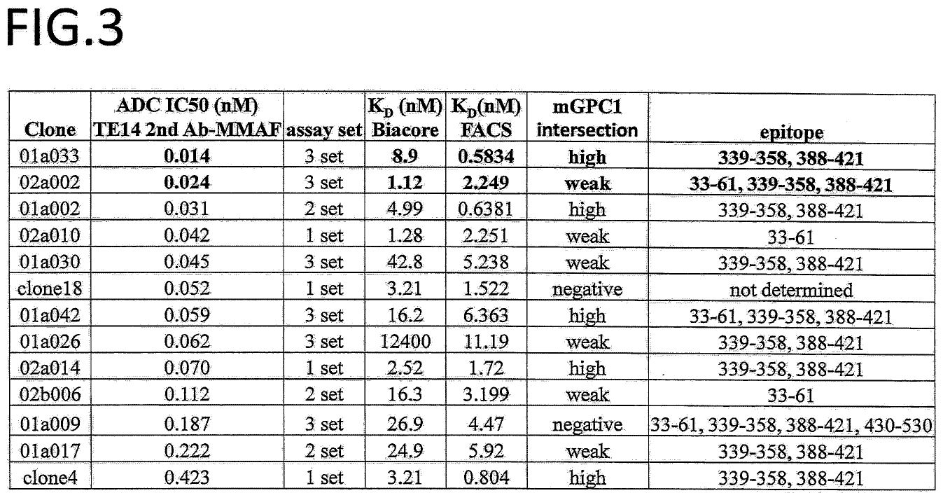

FIG. 3 shows results of an ADC assay of clones exhibiting an IC₅₀ of 0.5 nM or less. Although not wishing to be bound by any theory, this value is used herein as a baseline for evaluating an ADC as exhibiting a certain degree of activity. ADC using clone 01a033 exhibited the highest ADC activity. It is understood that a high ADC activity of 0.5 nM or less is exhibited due to having a high internalization activity. Thus, it is suggested that epitopes that are important for internalization are, as shown in FIG. 3, (a) positions 33 to 61 of SEQ ID NO: 2; (b) positions 339 to 358 and 388 to 421 of SEQ ID NO: 2; (c) positions 33 to 61, 339 to 358, and 388 to 42 of SEQ ID NO: 2; and (d) positions 33 to 61, 339 to 358, 388 to 421, and 430 to 530 of SEQ ID NO: 2.

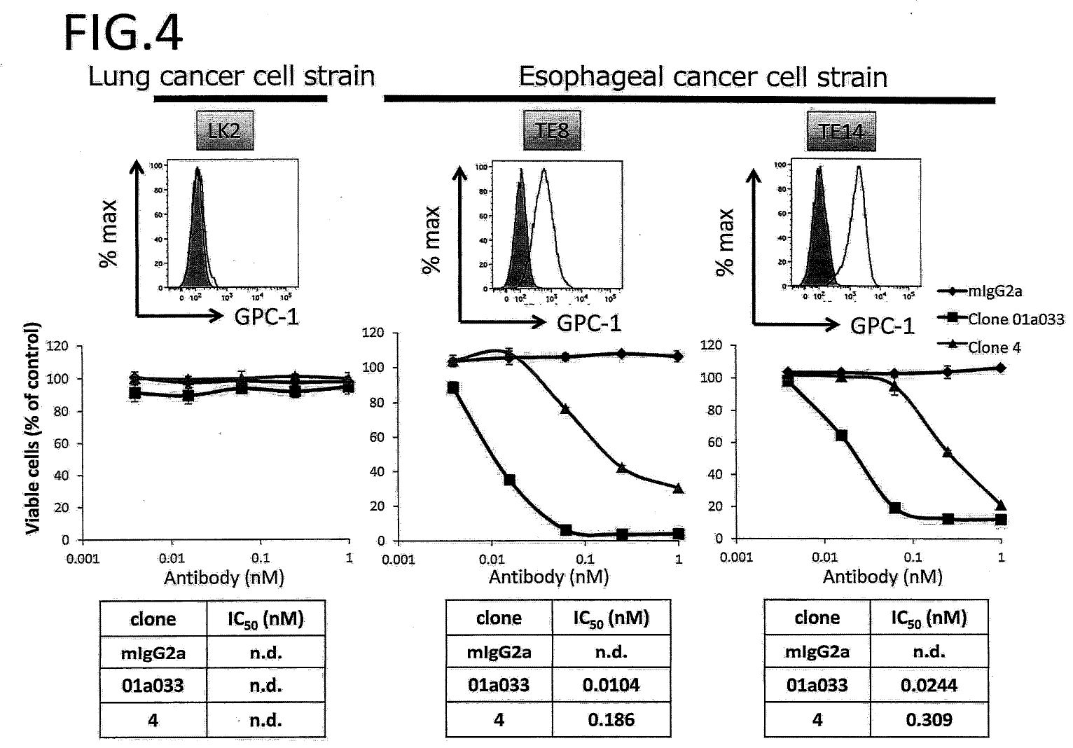

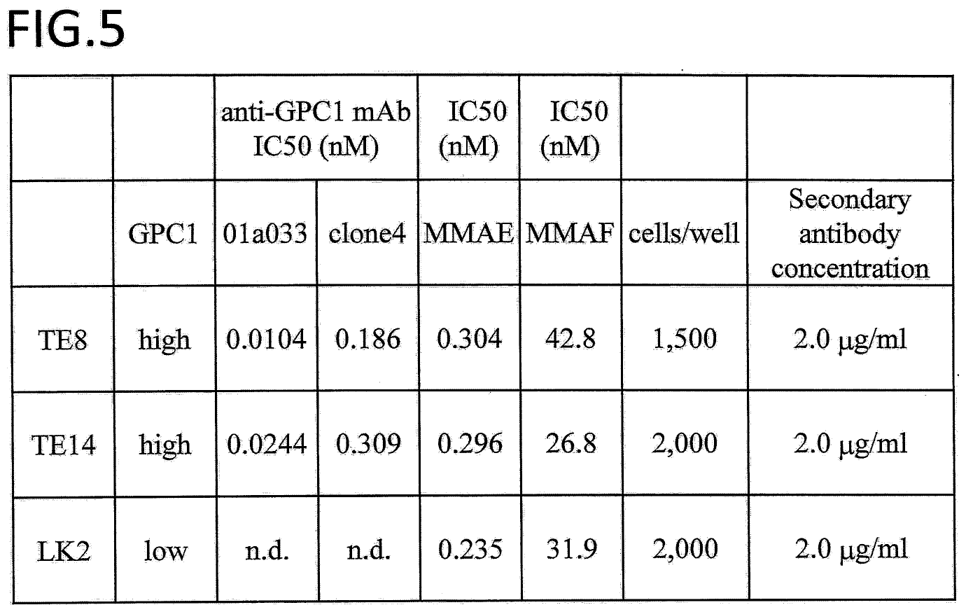

FIGS. 4 and 5 show results of conducting ADC assays using LK2, TE8, and TE14 cell strain. An antitumor effect due to an ADC of an anti-GPC-1 antibody was hardly exhibited with LK2, which is a lung cancer derived cell strain with low expression of Glypican-1, while a potent antitumor effect due to an ADC of an anti-GPC-1 antibody was exhibited with TE8 and TE14, which are esophageal cancer derived cell strains. An ADC of clone 01a033 exhibited a particularly potent antitumor effect on TE8 and TE14 while exhibiting high specificity.

### Example 3: Production of ADC

The ADC of the invention was produced as follows.

*140 ml of 1 mg/ml BioLegend mouse IgG2aκ isotype control MOPC173 (Part #92394, lot # B222287) and 70 ml of 2 mg/ml mouse anti-GPC-1 clone 01a033 (lot #160316) were obtained.

*First, conjugation was performed in a small scale. 2 ml of αGPC1 was concentrated to about 0.8 ml (4.4 mg/ml), and 2 ml of MOPC173 was concentrated to about 0.4 ml (4.4 mg/ml). Different reducing conditions were set using 50 μl of each mAb for each condition to achieve a final drug-antibody ratio (DAR) of about 4. mAb was conjugated to maleimidocaproyl-valine-citrulline-p-aminobenzoyloxy carbonyl-monomethyl auristatin-F (MC-vc-PAB-MMAF). The conjugation was performed using a maleimide-cysteine based method of first reducing an inter-strand disulfide bond of mAb by TCEP at 37° C., and then binding a maleimide moiety of a drug to the reduced cysteine. The profile of a complex was analyzed by hydrophobic interaction chromatography (HIC).

*Next, optimal conditions for conjugation in a large scale determined in small scale were used. First, 138 ml of MOPC173 was concentrated to a 28 ml (4.88 ml/ml) stock, and 68 ml of αGPC1 was concentrated to a 26.2 ml (5.14 mg/ml) stock. mAb was reduced with TCEP at 37° C., and then reacted with drug-linker-MC-vc-PAB-MMAF.

*The ADC was desalinated with a Sephadex G50 column, and unreacted toxins were removed, and then the buffer was exchanged with PBS. The ADC was then filtered and sterilized, and diluted to 1 mg/ml with a sterilized PBS buffer. The ADC was fractionated into a 1 ml tube and then stored at 4° C.

*The drug-antibody ratio (DAR) determined by the ratio of A₂₄₈ₙₘ:A₂₈₀ₙₘ was 3.8 for MOPC173-CL-MMAF, and 4.1 for αGPC1-CL-MMAF.

*Size-exclusion chromatography (SEC) and hydrophobic interaction chromatography (HIC) were used to analyze unconjugated mAb and two ADCs. According to the analysis of unconjugated mAb before and after concentration by SEC and HIC, the properties of mAb did not change even when the mAb was concentrated to about 5 mg/ml. MOPC173 was present as a monomer, and αGPC1 was also present mostly as a monomer, but a small aggregation peak was observed. Both mAbs conjugated well with MC-vc-PAB-MMAF. The HIC profile of ADC exhibited excellent peaks of DAR=2, 4, 6, and 8, and enables peak integration method for determining DAR. Figure depicts a schematic diagram of a structure of an exemplary ADC.

### Example 4: Antigen Affinity Analysis of an Unlabeled Antibody and GPC1 ADC

(Materials and Methods)

(Buffer Preparation)

The buffer used in this Example was prepared as follows.

*Coating buffer: PBS (−).

*Dilution buffer: 10% Block Ace was used as the diluent. 10% Block Ace was prepared by dissolving 4 g of Block Ace powder into 100 mL of MiliQ, and then diluting 10-fold with PBS (−). *Blocking buffer: A buffer was prepared by dissolving 4 g of Block Ace powder into 100 mL of MiliQ, and then diluting 4-fold with PBS (−). A solution prepared by dissolving 4 g of Block Ace powder into 100 mL of MiliQ was used as the stock solution (100% solution).

*Washing buffer: 1 pack of PBS-T (Sigma, P3563) was dissolved in 1 L of ultrapure water (0.05% Tween 20/PBS).

(Primary Antibody Preparation)

The primary antibodies used in this Example were prepared. GPC1-CL-MMAF and anti-GPC1 monoclonal antibodies (01a033) were diluted stepwise to 100, 20, 4, 0.8, 0.16, 0.032, 0.0064, 0.00128, 0.000256, 0.0000512, and 0.00001024 nM with a dilution buffer. Recombinant human Glypican-1 proteins (CF, model number: 4519-GP-050, volume: 50 μg) were used as the antigen.

(Methodology)

Recombinant human GPC1 was diluted to 2.5 μg/mL with PBS (−) and added to 64 wells of Immuno 96 well MicroWell Solid Plate (Maxisorp, Nunc) at 50 μL/well, and the plate was sealed. The plate was shaken, and liquid was confirmed to spread throughout the plate. Next, the following procedure was followed.

*Incubate (4° C. overnight)

*Wash (200 μL/well×3)

*Add blocking buffer to the 96-well microplate at 200 μL/well.

*React for 1 hour at 37° C.

*Wash (200 μL/well, three times)

*Dilute (1) GPC1-CL-MMAF and (2) anti-GPC1 monoclonal antibody (01a033) with a dilution buffer in a 10-fold dilution series from 100 nM, and add the mixture to the 96-well microplate at 100 μL/well.

*React for 1 hour at 37° C.

*Wash (200 μL/well×3)

*Dilute Anti-Mouse IgG (H+L) Antibody, Human Serum Adsorbed and Peroxidase labeled (KPL, cat. No.: 474-1806) with a dilution buffer (10% Block Ace) to 0.2 μg/ml (prepare a 5000-fold dilution upon use), and add the mixture to the 96-well microplate at 100 μL/well.

*React for 1 hour at 37° C.

*Wash (200 μL/well, three times)

*Return TMB (SurModics, TMBW-1000-01) to normal temperature before use, and add the TMB to the 96-well microplate at 100 μL/well.

*Agitate (protected from light, room temperature)

*Discontinue at 50 μL/well of 1N-sulfuric acid.

*Measure absorbance at 450 nm.

*Analyze data with GraphPad Prism 6.04.

(Results)

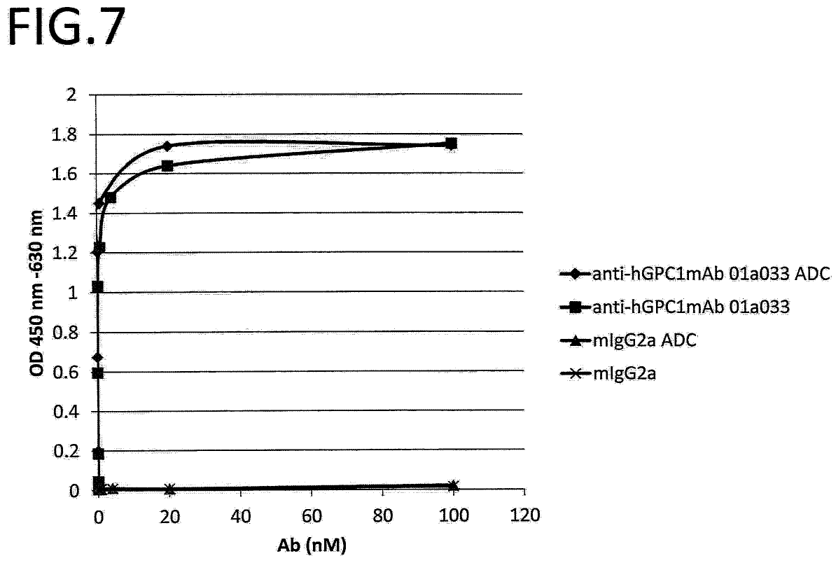

Results are shown in FIG. 7. Controls mIgG2a and mIgG2a ADCs did not exhibit any affinity to an antigen, whereas 01a033 and 01a033 ADCs exhibited high affinity. Further, the affinity levels of 01a033 and 01a033 ADCs are about the same, indicating that the binding capacity of the antibody itself is not compromised when a drug is conjugated.

### Example 5: Analysis of Antibody Binding Capacity

The amount of antigen per one cell of Glypican-1 expressed on the cell surface of various cell strains was quantified as Antibody Binding Capacity (ABC) by using QIFIKIT®. Measurement was taken in accordance with the instruction manual of QIFIKIT® by using anti-Glypican-1 antibody clone 01a033 as the antibody. 1×10⁵ cells and 10 μg/ml of clone 01a033 were used.

(Results)

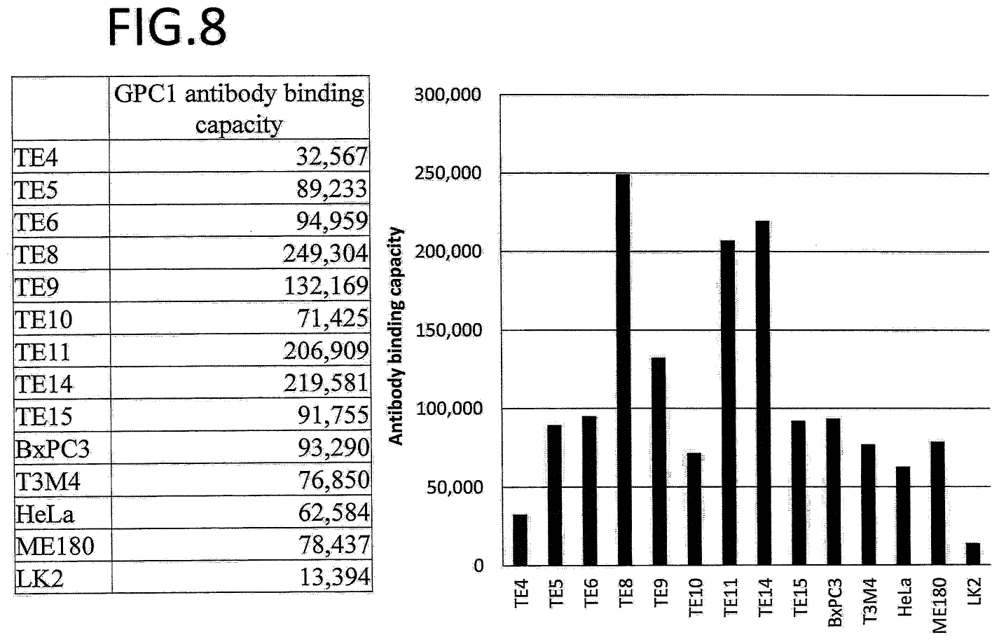

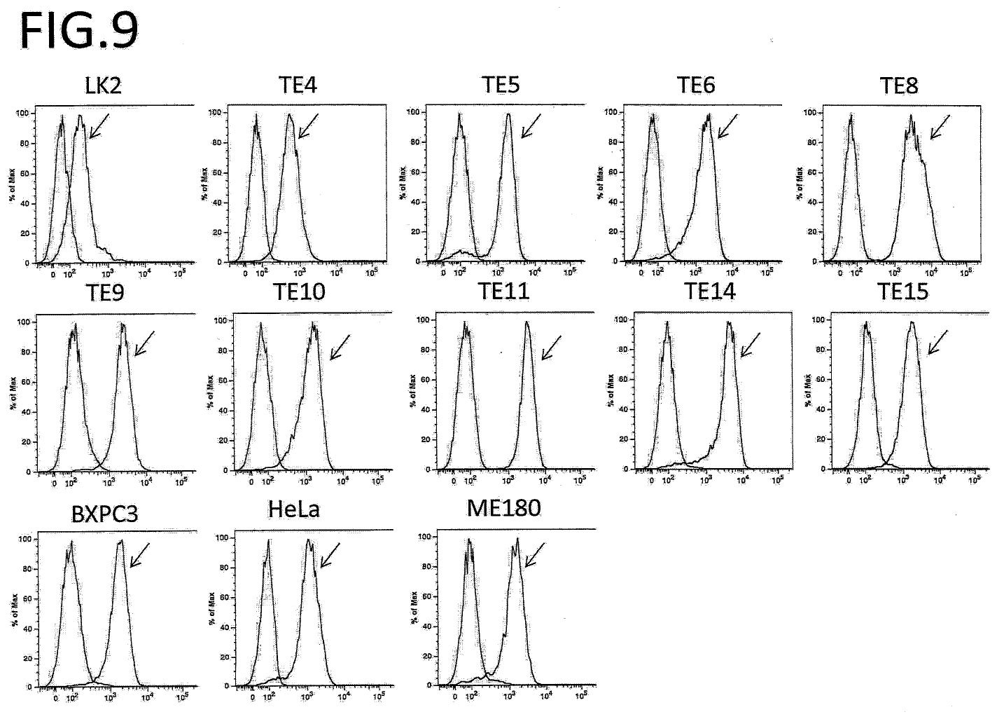

As is apparent from FIG. 8, GPC1 was highly expressed on the surface of esophageal cancer cell strains TE4, TE5, TE6, TE8, TE9, TE10, TE11, TE14, and TE15 cells. Human lung squamous cell carcinoma derived cell strain LK2 cells exhibited low expression. Human pancreatic cancer cell strains BxPC3 cells and T3M4 cells, and cervical cancer cell strains HeLa cells and ME180 cells also exhibited the same degree of expression as esophageal cancer cell strains. FIG. 9 shows data from analyzing the reactivity of GPC1 and an isotype control antibody or a clone 01a033 antibody in various cancer cell strains by FACS. It can be seen that the reactivity with a clone 01a033 antibody is low and the amount of expression of GPC1 is low in LK2 cells.

### (Example 6: Internalization Assay on GPC1 ADC (01a033))

This Example studied the internalization (intracellular invasion) activity using various GPC1 positive cell strains for GPC1 ADC (01a033) MMAF.

(Materials and Methods)

Detection was performed on cells treated with GPC1 ADC (01a033) MMAF with anti-GPC1 mAb (biotin-labeled 02b006) and PE-labeled streptavidin.

TE8, HeLa, ME180, BxPC3, and T3M4 cell strains were detached and collected from a 10 cm plate using 0.02% EDTA. Cells were suspended in RPMI1640+10% FBS+100 U/ml penicillin+100 μg/ml streptomycin, and two 1.5 ml tubes were created at 1.0×10⁶ cells/0.9 ml. To lower the internalization activity, the tubes were left standing on ice for 1 hour. 1 mg/ml of GPC1 ADC (01a033) MMAF was diluted to 24 μg/mL (160 nM) with ice-cooled RPMI1640+10% FBS+100 U/ml penicillin+100 μg/ml streptomycin. 100 μl of the ADC was added and suspended in the cell suspension and incubated on ice for 30 minutes to stain the antigens on the cell surface. After 30 minutes, the cell suspension was dispensed into 7 tubes each at 100 μl each. The cell suspension was separated into 4° C. incubation group and 37° C. incubation group in 14 tubes. The incubation times were 0.0 h, 1 h, 2 h, 3 h, 4 h, 6 h, 12 h, and 24 h. After reaching the incubation time, the cells were centrifuged at 1500 rpm at 4° C. for 5 minutes to remove the supernatant. The cells were washed with 100 μl of ice cooled PBS+0.2% BSA and subjected to centrifugation to wash the cells. Washing operation was performed three times in total. 50 μL of biotin-labeled anti-GPC1 antibody 02b006 at a concentration of 1 μg/ml was added and suspended. The antigens on the cell surface were stained for 30 minutes on ice (GPC1 that was not internalized is stained). After reaching the incubation time, the cells were centrifuged at 1500 rpm at 4° C. for 5 minutes to remove the supernatant. The cells were washed with 100 μl of ice cooled PBS+0.2% BSA and subjected to centrifugation to wash the cells. Washing operation was performed three times in total. Streptavidin-PE (PharmMingen, #554061) was diluted 200-fold with ice-cooled PBS+0.2% BSA. 50 μL was added to suspend the cells. The suspension was allowed to react under protection from light for 30 minutes on ice. After reaching the incubation time, the cells were centrifuged at 1500 rpm at 4° C. for 5 minutes to remove the supernatant. The cells were washed with 100 μl of ice cooled PBS+0.2% BSA and subjected to centrifugation to wash the cells. Washing operation was performed three times in total. 150 μL of ice-cooled PBS+0.2% BSA was added for measurement with FACS Canto II and analysis with FlowJo ver

(Results)

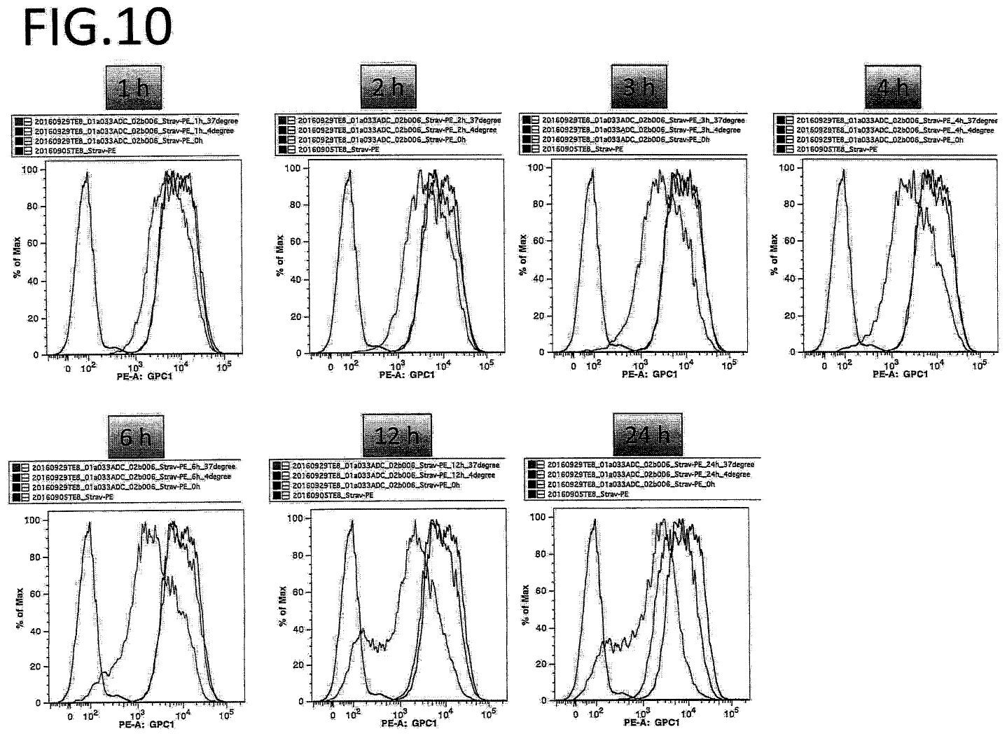

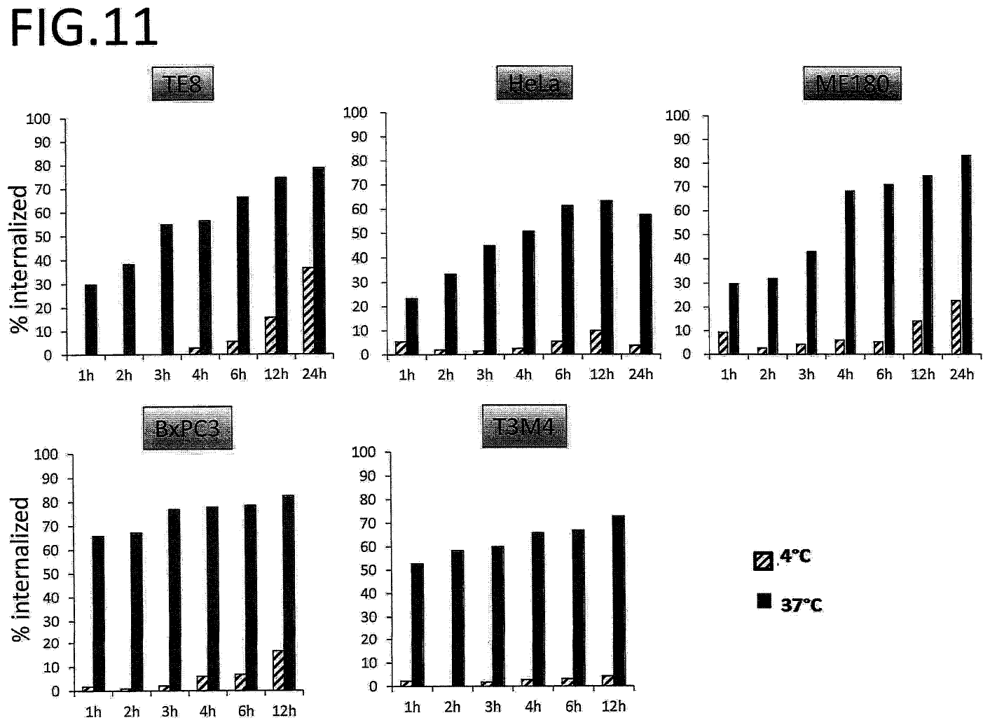

The results are shown in FIGS. 10 and 11. FIG. 10 shows histograms of the amount of Glypican-1 remaining on the TE8 cell surface for each elapsed time after addition of clone 01a033 ADC. The blue line is the result of staining with clone 02b006 in the absence of 01a033 ADC. This state would exhibit the maximum reactivity (100% of Glypican-1 remains on the cell surface). Since treatment at 4° C. stops the physiological function of the cells, this is a condition under which endocytosis is suppressed and internalization does not occur, whereas 37° C. is a temperature that is the most suitable for internalization. It can be seen from the orange color that Glypican-1 on the cell surface decreases with the passage of incubation time at 37° C. with addition of antibodies. Meanwhile, the residual amount of Glypican-1 on the cell surface hardly changed at 4° C. as shown in green. FIG. 11 shows a value found by subtracting the percentage of residual Glypican-1 on the cell surface from 100% as % internalized. FIG. 11 clearly shows that the anti-Glypican-1 antibody of the invention has high internalization activity against various cell strains including esophageal cancer, pancreatic cancer, and cervical cancer derived cell strains.

### Example 7: In Vitro ADC Assay Using Anti-GPC1 Antibody

(Materials and Methods)

Preparation of a conjugate of mouse anti-Glypican-1 mAb (01a033), mouse IgG2a (biolegend #400224) as a control, and mc-vc-PAB-MMAF was commissioned to MORADEC. An anticancer agent was conjugated to a cysteine residue via a cleavable linker. The DAR (drug-antibody ratio) was as follows. GPC1-CL-MMAF DAR=3.8, mouse IgG2a-CL-MMAF DAR=4.1.

Specifically, the following assay was conducted.

(1) The cells were seeded on Thermo Fisher Scientific's 96-well white plate (model no.: 136101) (90 μl cell suspension). RPMI1640+10% FBS+100 U/ml penicillin+100 μg/ml streptomycin was used as the medium. The cells were seeded in 60 wells, and 100 μL of medium was added to 36 wells, and cultured in a 5% CO2 incubator at 37° C.
(2) The next day, 10 μL of ADC was added to the cells (total amount: 100 μL).
(3) After 144 hours of culture, a CellTiter-Glo™ Luminescence Cell Viability Assay reagent (Promega) was added and mixed at 100 μL/well.
(4) Measurements were taken with a plate reader.
(5) Analysis was conducted with GraphPad Prism 6.

The IC₅₀ values were calculated from the following equation (Hossain M M, Hosono-Fukao T, Tang R, Sugaya N, van Kuppevelt T H, Jenniskens G J, Kimata K, Rosen S D, Uchimura K (2010) Direct detection of HSulf-1 and HSulf-2 activities on extracellular heparan sulphate and their inhibition by PI-88. Glycobiology 20(2): 175-186.)

IC50=10\hat{ }(Log [A][B]×(50−C)/(D−C)+Log [B])

A: High concentration straddling 50%
B: Low concentration straddling 50%
C: Inhibition rate at B
D: Inhibition rate at A

(Results)

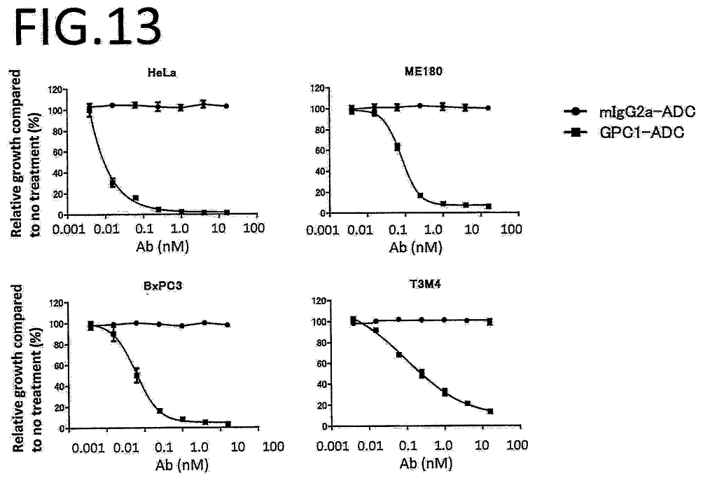

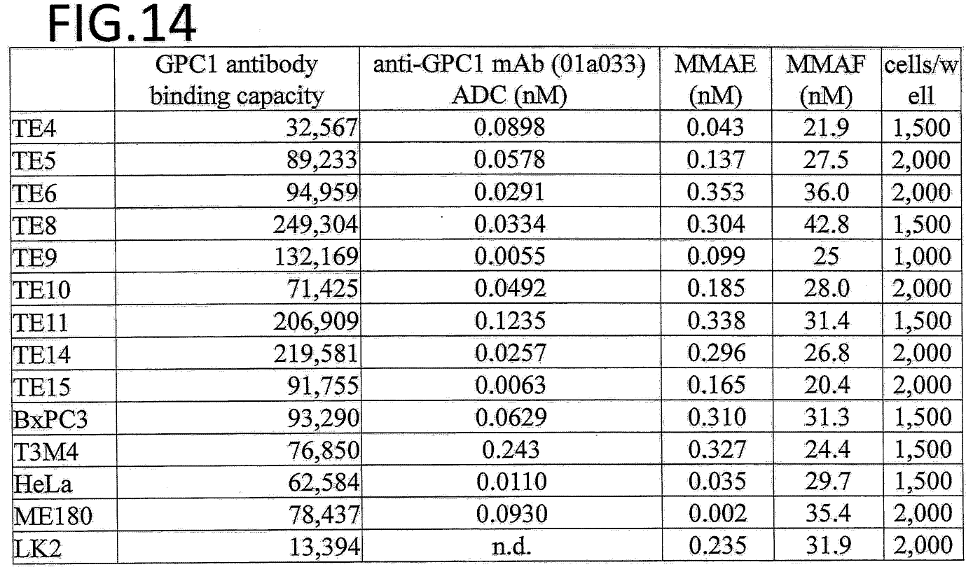

FIGS. 12 and 13 show results for antitumor effect on each cell strain. As shown, GPC1 ADC exhibits an effect of suppressing cell growth against GPC1 positive esophageal cancer cell strains. Interestingly, an antitumor effect was hardly exhibited against LK2 cells (human lung squamous cell carcinoma derived cell strain) that hardly express GPC1 (FIG. 12). This indicates that ADC exerts an antitumor effect specifically against GPC1 positive cells. Surprisingly, it was revealed that GPC1 ADC also exhibits an effect of suppressing cell growth on pancreatic cancer derived cell strains BxPC3 cells and T3M4 cells and cervical cancer derived cell strains HeLa cells and ME180 cells (FIG. 13). This indicates that the ADC of the invention also exerts the same effect of suppressing cell growth on GPC1 positive cancer (e.g., pancreatic cancer, cervical cancer, and the like) highly expressing GPC1 in the same manner as esophageal cancer. Meanwhile, the ADC of the invention did not exhibit an effect of suppressing cell growth against LK2 cell strains that express GPC1 at a low level. FIG. 14 is a table summarizing ICH deduced from the results obtained from FIGS. 12 and 13.

### Example 8: Safety Test on GPC1 ADC

(Materials and Methods)

A single dose (1 ml) of control PBS, control ADC (50 mg/kg), anti-GPC1 ADC (3 mg/kg), anti-GPC1 ADC (15 mg/kg), and anti-GPC1 ADC (50 mg/kg) was intraperitoneally administered on day 1 to a group of 4 male and 4 female C57BL/6J mice (8W). The mice were dissected after 7 days from administration.

Endpoints

*Blood collection (Blood cell count, biochemical test)

(Results)

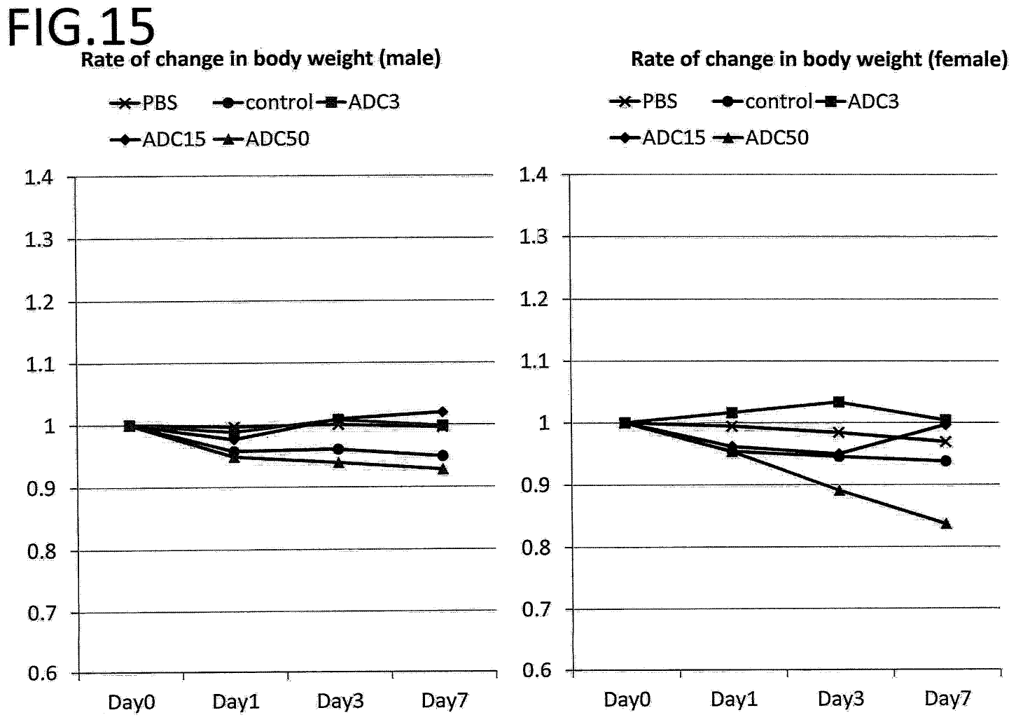

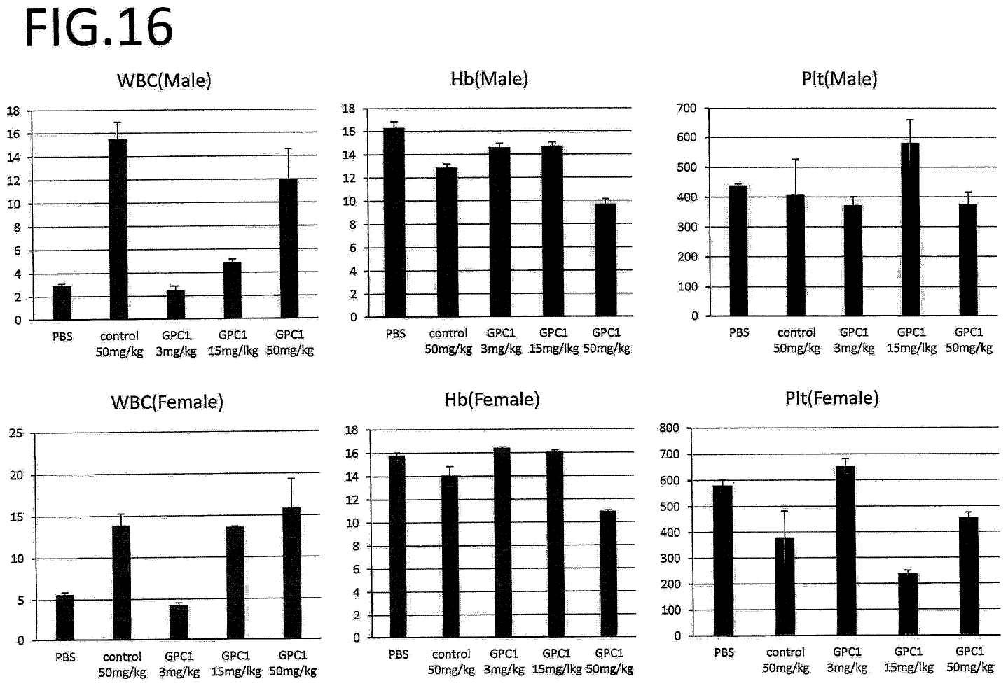

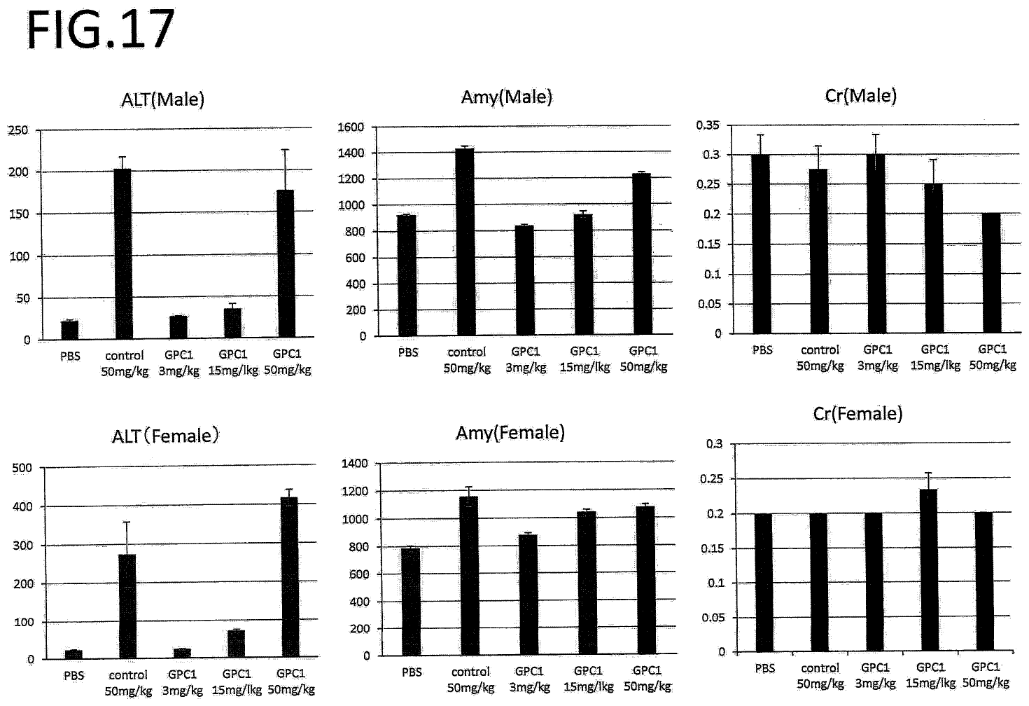

The results are shown in FIG. 15. Significant change in the body weight was not observed in mice other than the group of mice administered with extremely large dose such as 50 mg/kg of ADC. As described in detail in the following Examples, sufficient antitumor effect is exhibited even with administration of the GPC1 ADC of the invention at 1 mg/kg. Thus, it is understood that an antitumor effect is achieved without exhibiting toxicity within the normal range of dosages. FIG. 16 shows results of a hematological test. While an increase in the while blood cells was observed in a high dose administration group, this was not GPC1-ADC specific. Mild anemia was observed only in the 50 mg/kg ADC administration group. Abnormal platelet count specific to each group was not observed. FIG. 17 shows results of a hematological biochemical test. An abnormality in the liver function was found in the high dose administration group, but this was not GPC1-ADC specific. Specific abnormality was not observed for amylase. Significant toxicity due to ADC administration was not observed from the results of the hematological test.

### Example 9: In Vivo Efficacy Test on GPC1 ADC Using Pancreatic Cancer Cell Strain

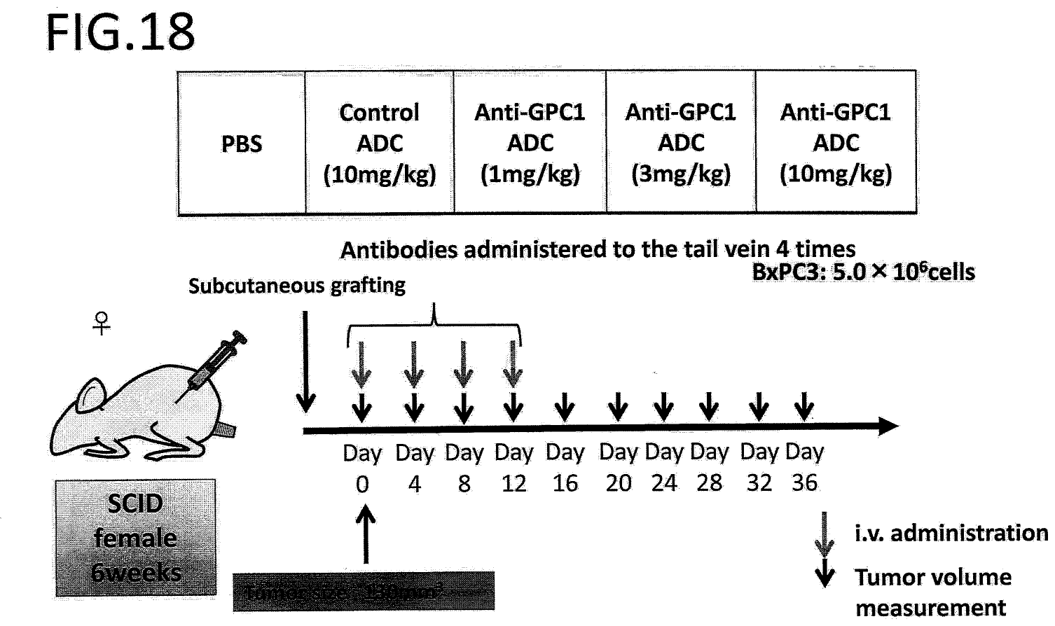

FIG. 18 schematically shows the test in this Example. Specifically, 5.0×10⁶ cells of the BxPC3 cell strain were subcutaneously grafted into female SCID mice. When the tumor size reached about 130 mm³ after grafting, 5. administration of PBS as a. control, control ADC (10 mg/kg), anti-GPC1 ADC (1 mg/kg), anti-GPC1 ADC (3 mg/kg), and anti-GPC1 ADC (10 mg/kg) was started, which were intravenously administered on day 0, day 4, day 8, and day 12, with the day starting the administration considered 0 day. The tumor volume and body weight were measured on day 0, day 4, day 8, day 12, day 16, day 20, day 24, day 28, day 32, and day 36.

After subcutaneous tumorigenesis of BxPC3, (1) PBS, (2) control ADC at 10 mg/kg, and (3) GPC1-ADC at 1 mg/kg, 3 mg/kg, and 10 mg/kg were administered once into the tail vein. The tumor was extracted after 24 hours. After deparaffinization of a section of a paraffin embedded tissue, the slice was dehydrated with alcohol.

Immunohistochemical staining on Phospho-Histon H3 (Ser10) was performed using an anti-Phospho-Histon H3 (Ser10) antibody (Cell Signaling Technology: #9701) and ChemMate Envision kit HRP 500T (Dako: K5007).

(Results)

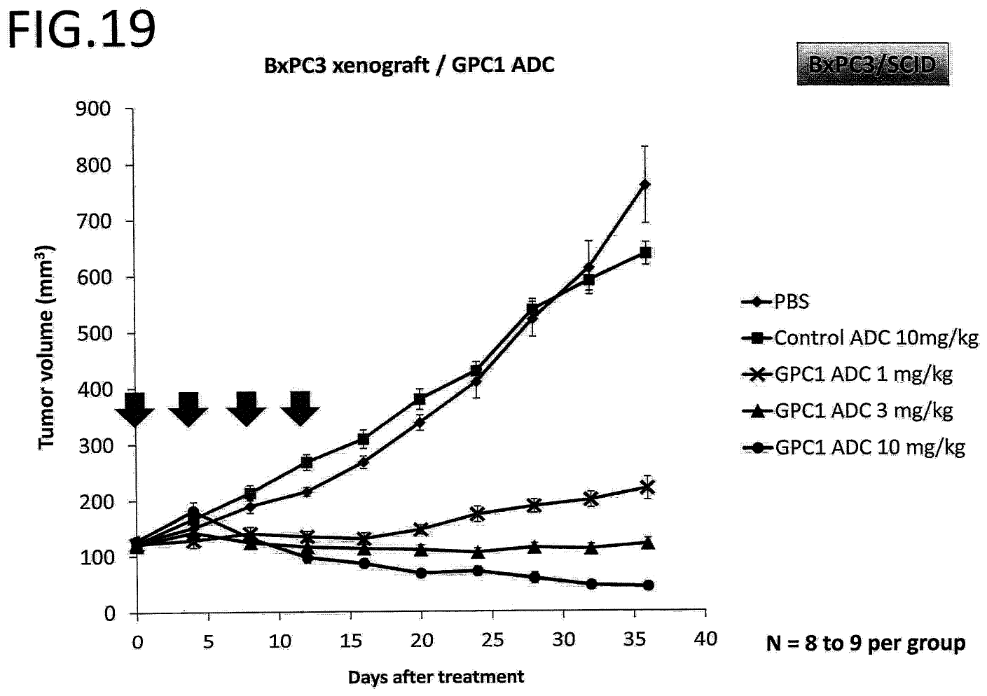

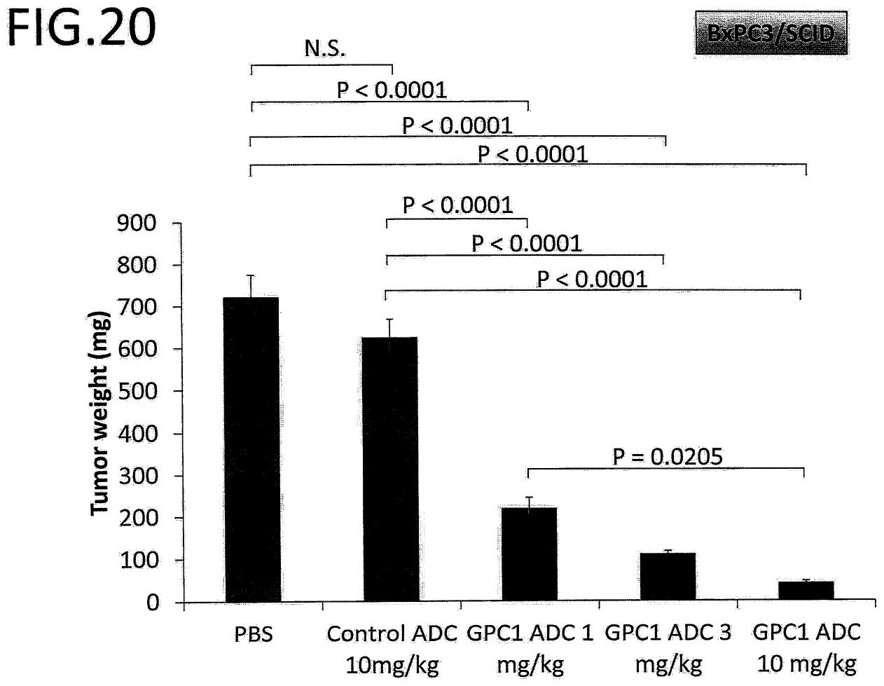

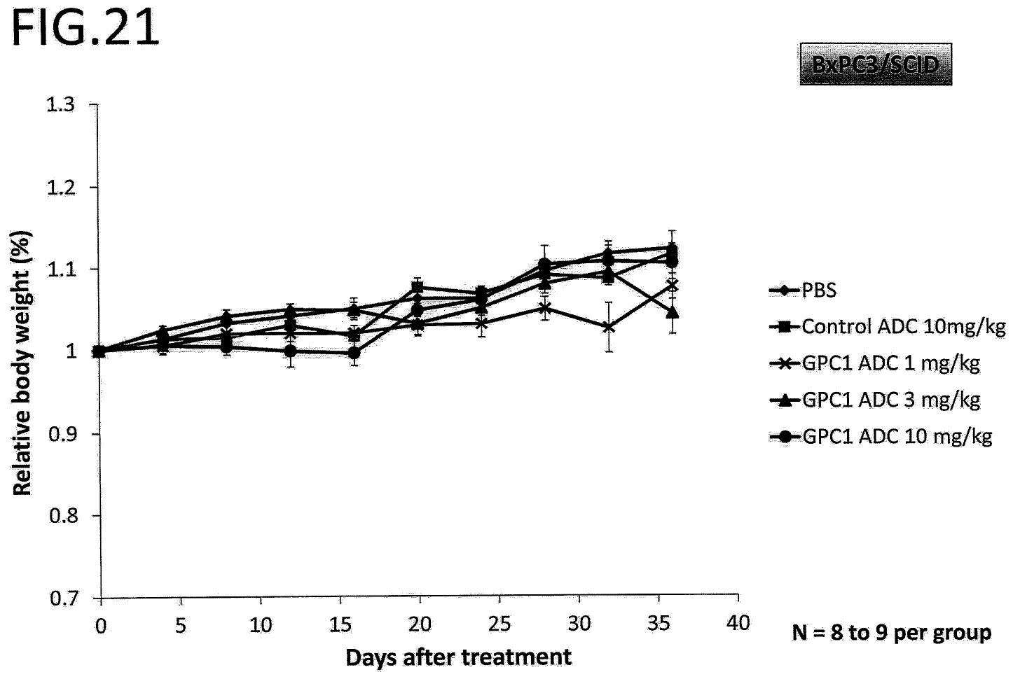

The results are shown in FIGS. 19 to 21. An antitumor effect was not exhibited in the PBS administration group and control ADC administration group, whereas a significant antitumor effect was exhibited in all groups administered with anti-GPC1 ADC in mice grafted with pancreatic cancer cell strain, BxPC3 cell strain (FIGS. 19 and 20). Surprisingly, not only was the tumor growth suppressed, but also the tumor volume was significantly reduced in the groups administered with anti-GPC1 ADC at 3 mg/kg and 10 mg/kg (FIG. 19). This revealed that the ADC of the invention exhibits a very high antitumor effect. Further, a significant change in the body weight was not observed in the anti-GPC1 ADC administration groups from any dosage tested. Thus, it is understood that toxicity due to anti-GPC1 ADC administration is low (FIG. 21).

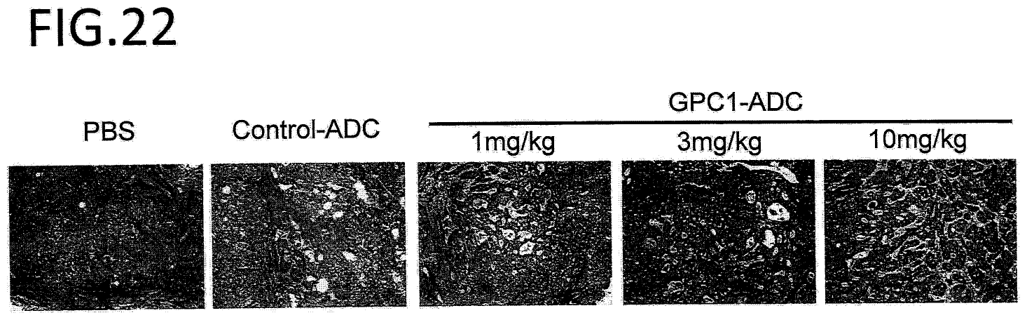

As shown in FIG. 22, administration of GPC1 ADC resulted in G2/M phase arrest in tumor tissue of a pancreatic cancer cell strain grafted model. This indicates that cell division of pancreatic cancer is suppressed by uptake of the GPC1 ADC of the invention into pancreatic cancer cells, then cleavage of a linker moiety to free MMAF in the cytoplasm, and arrest of cell cycle at the G2/M phase via tubulin polymerization inhibition. The results of FIG. 22 show that the GPC1 ADC of the invention can suppress or stop cell growth in tumor tissue.

### Example 10: In Vivo Efficacy Test on GPC1 ADC Using Pancreatic Cancer PDX

Pancreatic cancer PDX (Patient-derived tumor xenograft) refers to residual tissue from pathological analysis of cancer tissue upon pancreatic cancer surgery from a pancreatic cancer patient. This Example used a pancreatic cancer model created by subcutaneously grafting in superimmunodeficient mice such as NOG mice. While human tumor hardly survives in SCID mice after grafting, NOG mice have more severe immunodeficiency than SCID mouse, so that human tumor is more likely to survive. For this reason, a PDX model has an environment that is closer to human tumor than a mouse model to which a cell strain is grafted. Thus, information obtained with a PDX model is useful in evaluating drug efficacy.

(Examination of GPC1 Expression in Pancreatic Cancer PDX)

After deparaffinization of a section of a paraffin embedded tissue, the slice was dehydrated with alcohol. Immunohistochemical staining on GPC1 was performed by using an anti-GPC1 antibody (Atlas Antibodies: HPA030571) and ChemMate Envision kit HRP 500T (Dako: K5007).

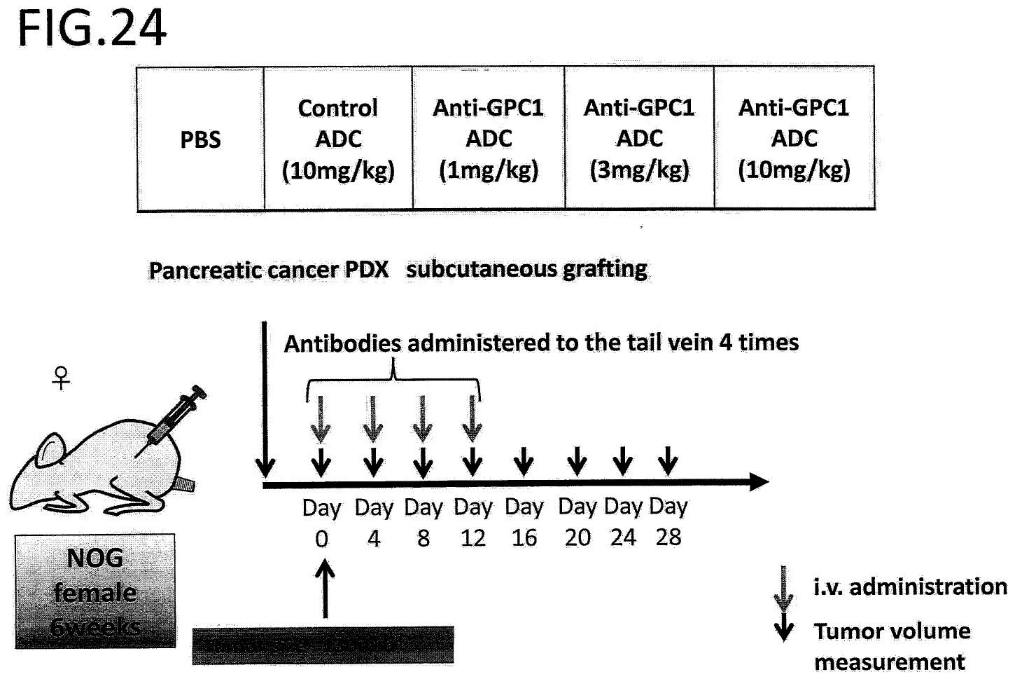

FIG. 24 schematically depicts the test in this Example. Specifically, pancreatic cancer PDX was subcutaneously grafted into 6 week old female NOG mice. When the tumor size reached about 130 mm³ after grafting, administration of PBS as a control, control ADC (10 mg/kg), anti-GPC1 ADC (1 mg/kg), anti-GPC1 ADC (3 mg/kg), and anti-GPC1 ADC (10 mg/kg) was started, which were intravenously administered on day 0, day 4, day 8, and day 12, with the day starting the administration considered 0 day. The tumor volume and body weight were measured on day 0, day 4, day 8, day 12, day 16, day 20, day 24, and day 28.

(Results)

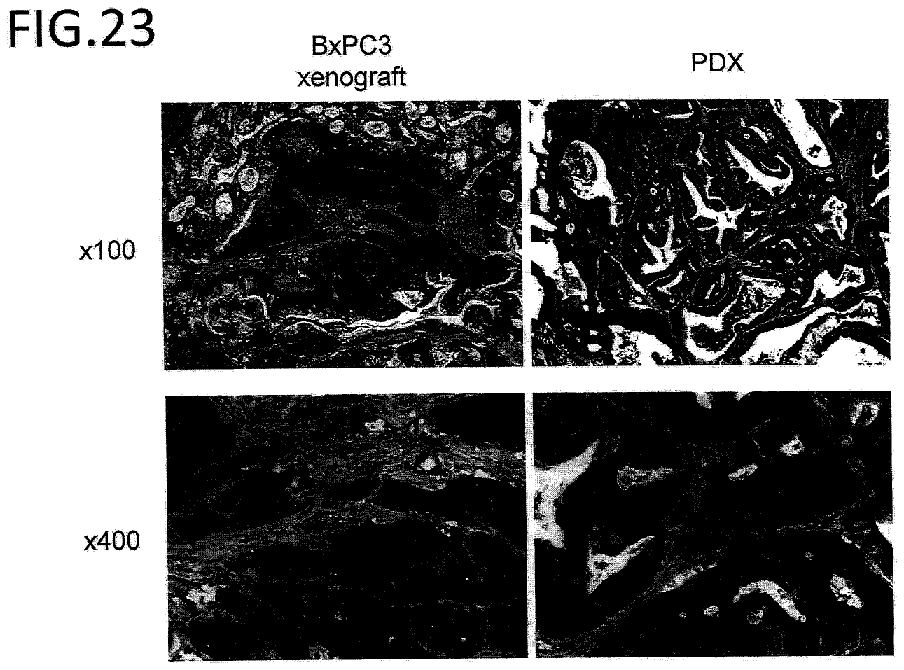

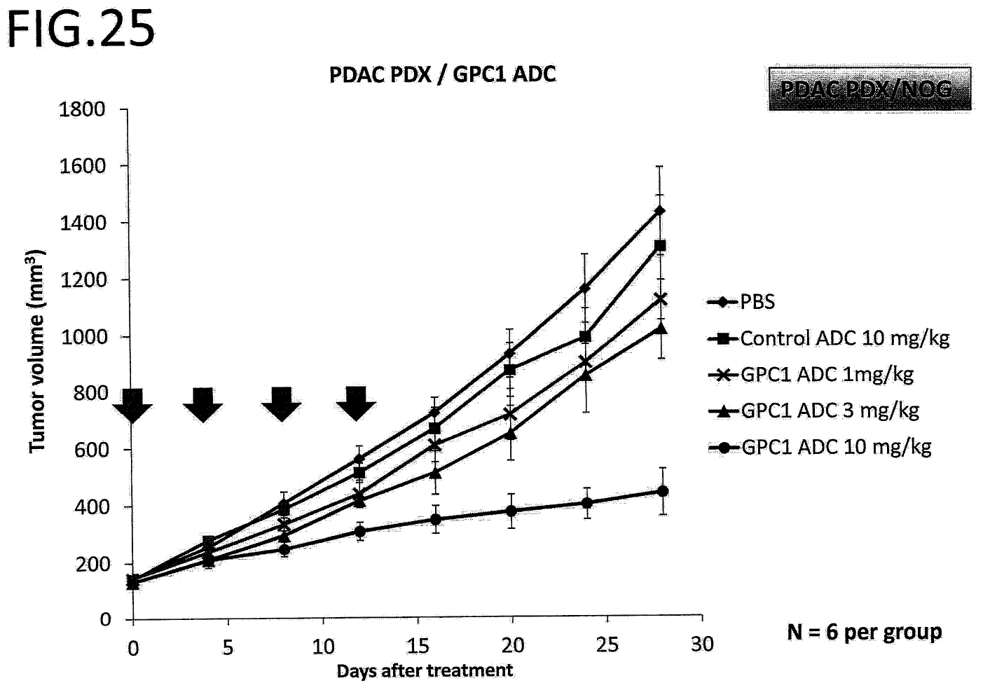

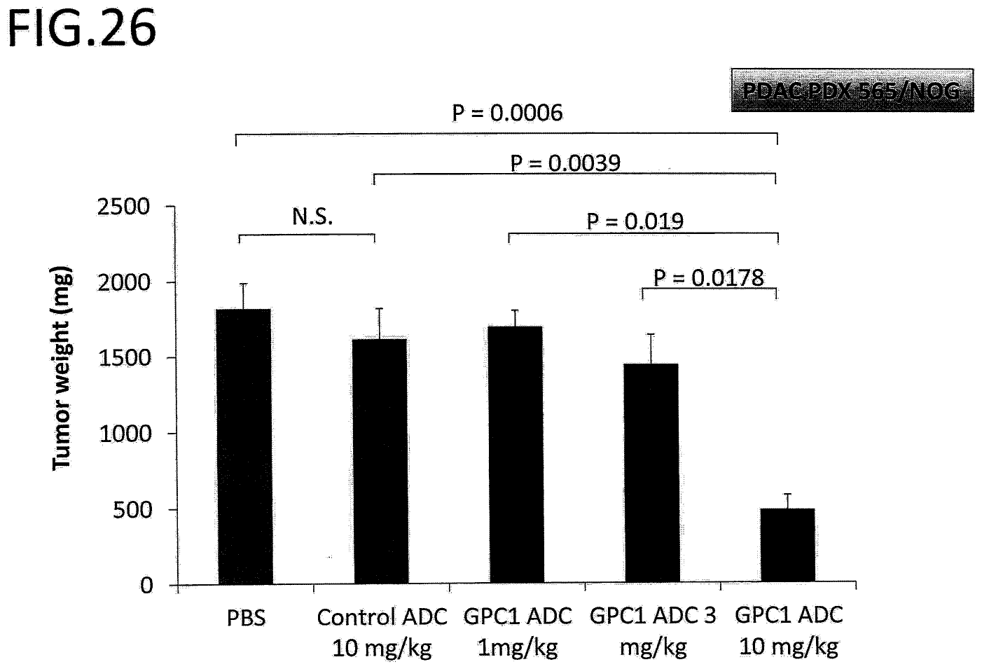

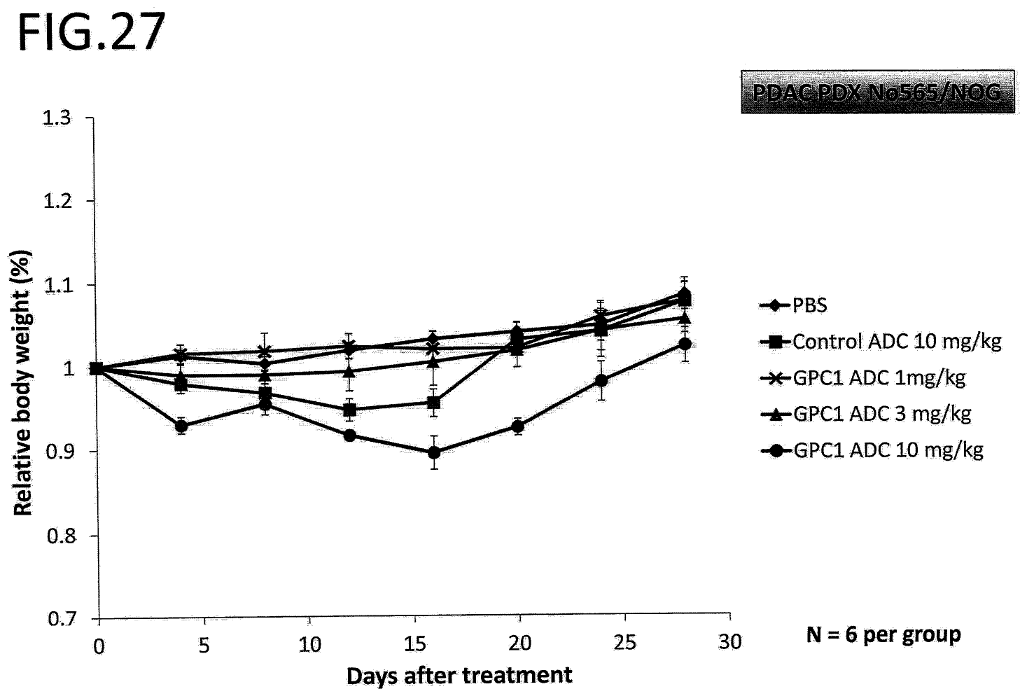

GPC1 was expressed in tumor tissue of the pancreatic cancer PDX used in this Example at an amount of expression that is close to tumor tissue in BxPC3 graft model (FIG. 23). FIGS. 25 and 26 show the results for antitumor effect on pancreatic cancer PDX due to anti-GPC1 ADC. While an anti-GPC1 ADC exhibited an antitumor effect in vivo in a model using pancreatic PDX, the efficacy was slightly lower for the pancreatic cancer PDX than BxPC3 graft model. It is conceivable that the sensitivity of the ADC agent MMAF itself was weaker than BxPC3 for the pancreatic cancer PDX. It is suggested that the efficacy is further enhanced if an agent other than MMAF is used. Further, the body weight slightly decreased on day 0.16 in the anti-GPC1 ADC (10 mg/kg) administration group in the anti-GPC1 ADC administration group, but the body weight recovered thereafter to the level at the start of measurement, indicating that the effect of an anti-GPC1 ADC on the change in body weight is reversible. Therefore, it is likely that toxicity due to administration of an anti-GPC1 ADC is low (FIG. 27).

(Example 11: In Vivo Efficacy Test on GPC1 ADC Using Cervical Cancer Cell Strain)

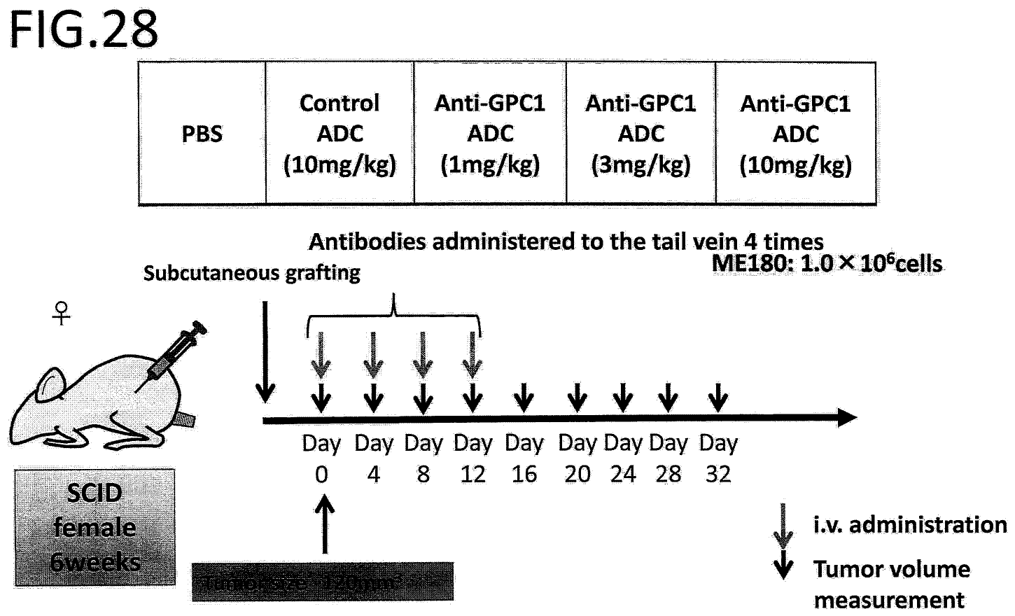

FIG. 28 schematically shows the test in this Example. Specifically, 1.0×10⁶ cells of ME180 cell strain were subcutaneously grafted into female SCID mice. When the tumor size reached about 120 mm³ after grafting, administration of PBS as a control, control ADC (10 mg/kg), anti-GPC1 ADC (1 mg/kg), anti-GPC1 ADC (3 mg/kg), and anti-GPC1 ADC (10 mg/kg) was started, which were intravenously administered on day 0, day 4, day 8, and day 12, with the day starting the administration considered 0 day. The tumor volume and body weight were measured on day 0, day 4, day 8, day 12, day 16, day 20, day 24, day 28, and day 32.

After subcutaneous tumorigenesis of ME180, (1) PBS, (2) control ADC at 10 mg/kg, and (3) GPC1-ADC at 10 mg/kg were administered once into the tail vein. The tumor was extracted after 24 hours. After deparaffinization of a section of a paraffin embedded tissue, the slice was dehydrated with alcohol. Immunohistochemical staining on Phospho-Histon H3 (Ser10) was performed by using an anti-Phospho-Histon H3 (Ser10) antibody (Cell Signaling Technology: #9701) and ChemMate Envision kit HRP 500T (Dako: K5007).

(Results)

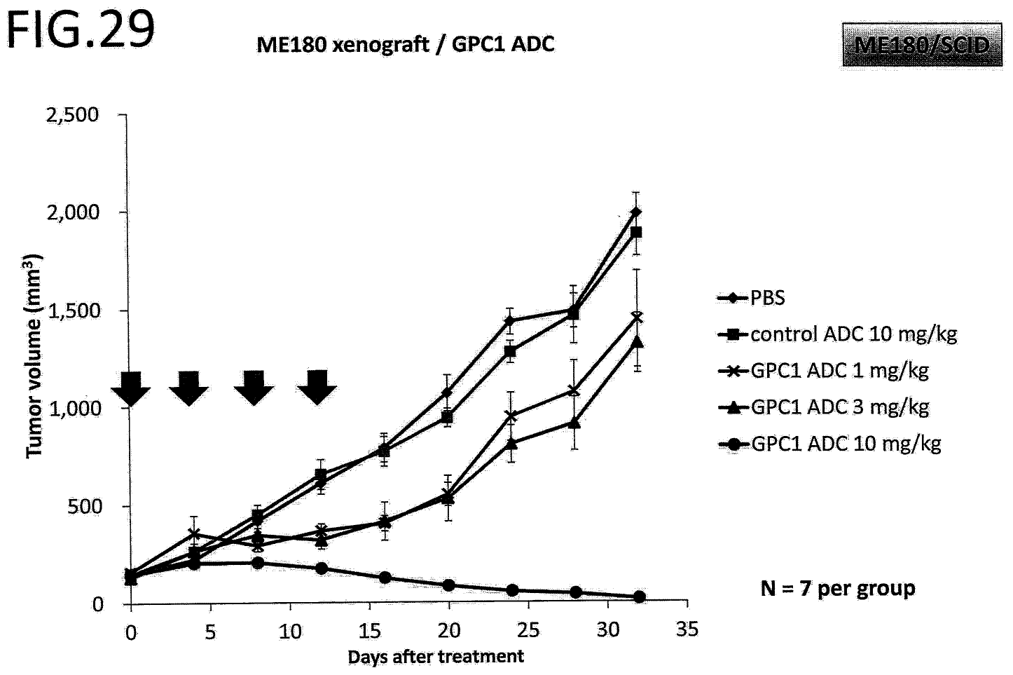

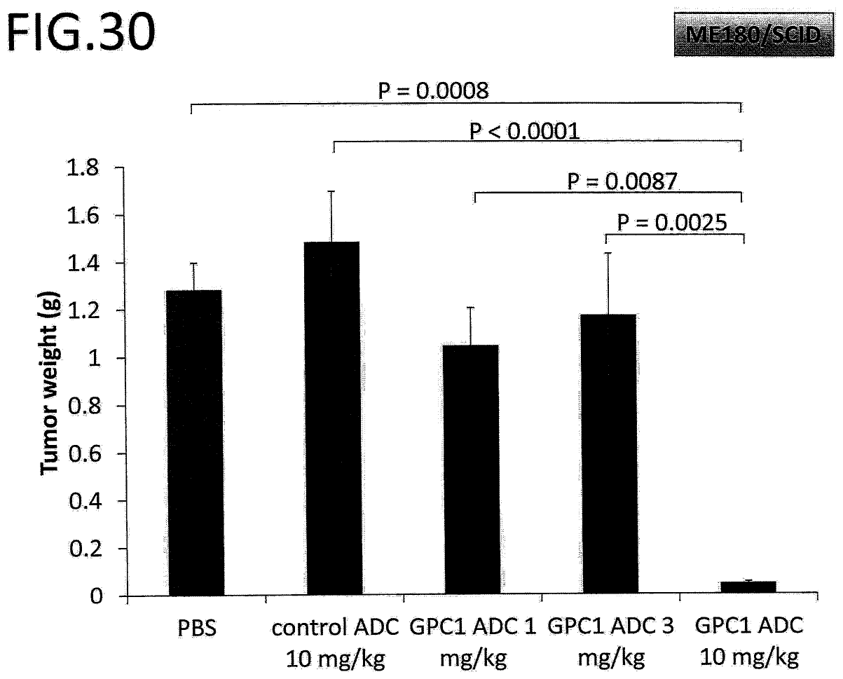

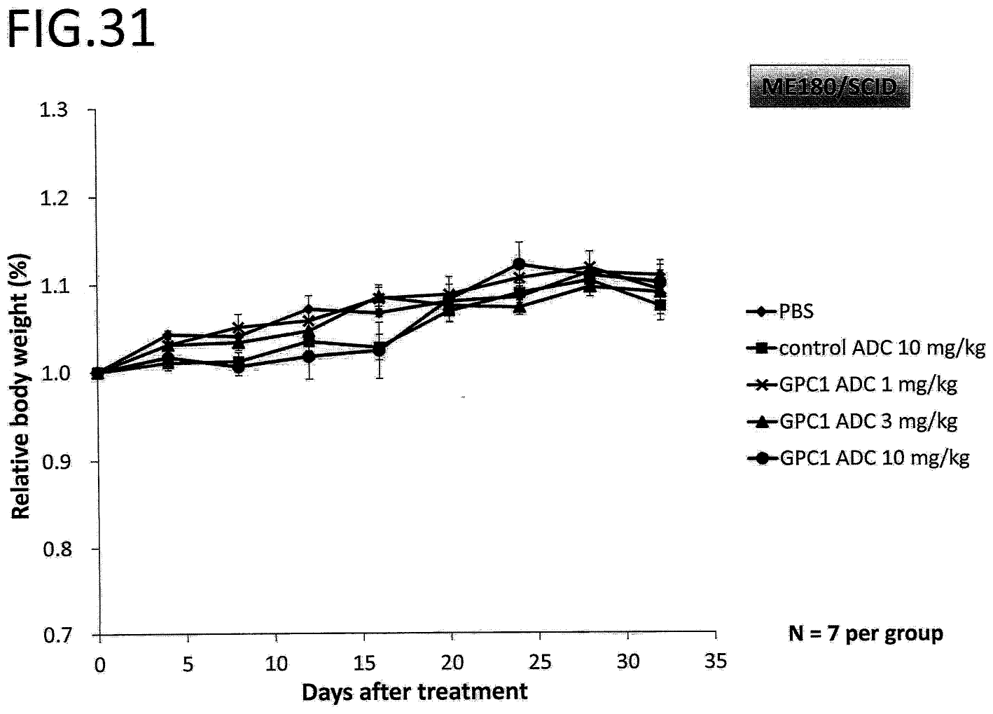

The results are shown in FIGS. 29 to 31. In mice grafted with cervical cancer cell strain ME 180, an antitumor effect was not exhibited in the PBS administration group and control ADC administration group, whereas a slight antitumor effect was exhibited in groups administered with an anti-GPC1 ADC at 1 mg/kg and 3 mg/kg, and a significant antitumor effect was exhibited in the group administered with anti-GPC1 ADC at 10 mg/kg (FIGS. 29 and 30). Surprisingly, not only was the tumor growth suppressed, but also the tumor volume was significantly reduced in the groups administered with anti-GPC1 ADC at 10 mg/kg (FIG. 29). This revealed that the ADC of the invention exhibits a very high antitumor effect. Further, a significant change in the body weight was not observed in the anti-GPC1 ADC administration groups from any dosage tested. Thus, it is understood that toxicity due to anti-GPC1 ADC administration is low (FIG. 31).

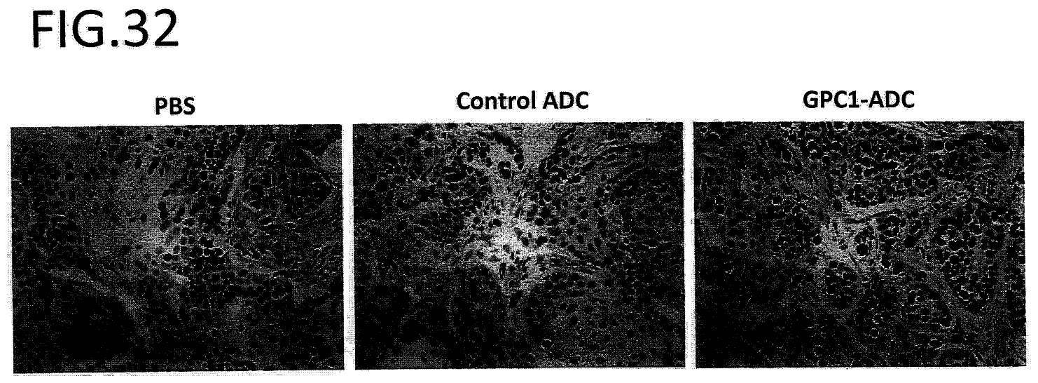

As shown in FIG. 32, administration of GPC1 ADC resulted in G2/M phase arrest in tumor tissue of a cervical cancer cell strain graft model. This indicates that cell division of cervical cancer is suppressed by uptake of the GPC1 ADC of the invention into pancreatic cancer cells, then cleavage of a linker moiety to free MMAF in the cytoplasm, and arrest of cell cycle at the G2/M phase via tubulin polymerization inhibition. The results of FIG. 32 show that the GPC1 ADC of the invention can suppress or stop cell growth in tumor tissue.

### Example 12: Examination of Antitumor Effect of Unlabeled Antibody

This Example examined the antitumor effect of unlabeled antibodies (antibodies that are not conjugated to an agent having cytotoxic activity).

(Materials and Methods)

2000 cells were added to a 96-well plate at 90 μl. The cells were cultured overnight at 37° C. in a 5% CO2 incubator. The next day, 10 μl of an unlabeled primary antibody concentrated 1.0-fold relative to the final concentration was added to each well, so that the total amount was 100 μl. The cells were cultured for 6 days at 37° C. in a 5% CO2 incubator. The cell viability was measured by using a CellTiter-Glo Luminescent Cell Viability Assay reagent to detect the amount of ATP.

(Results)

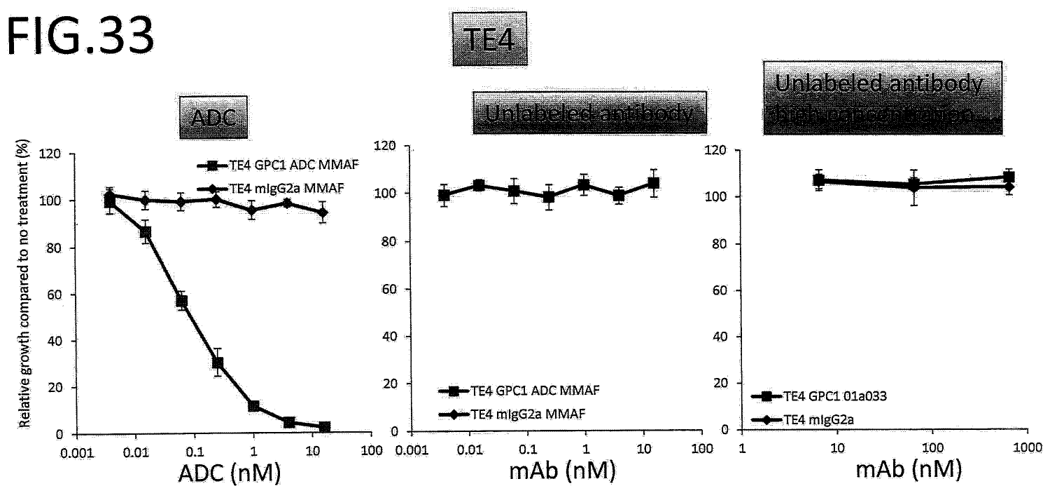

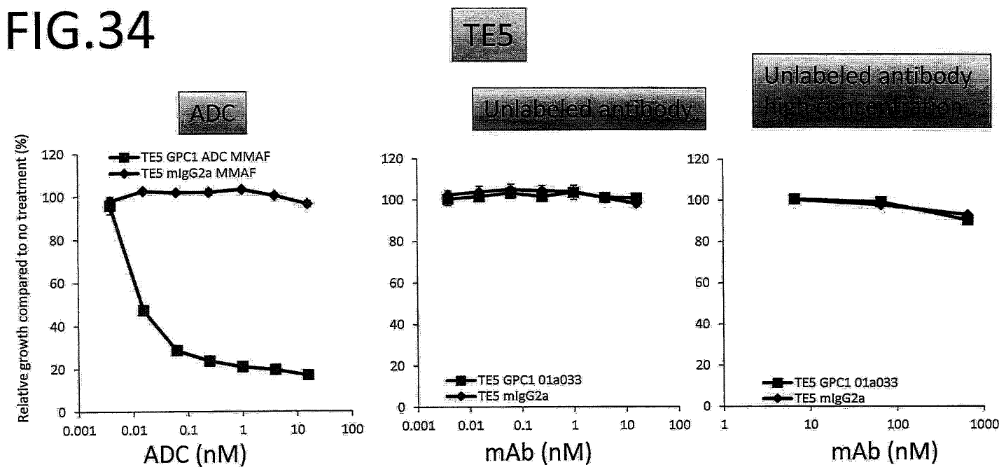

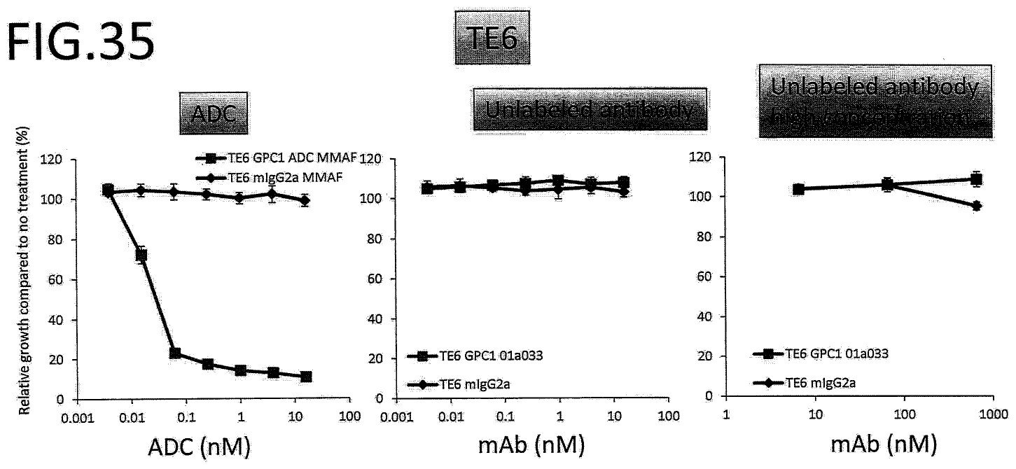

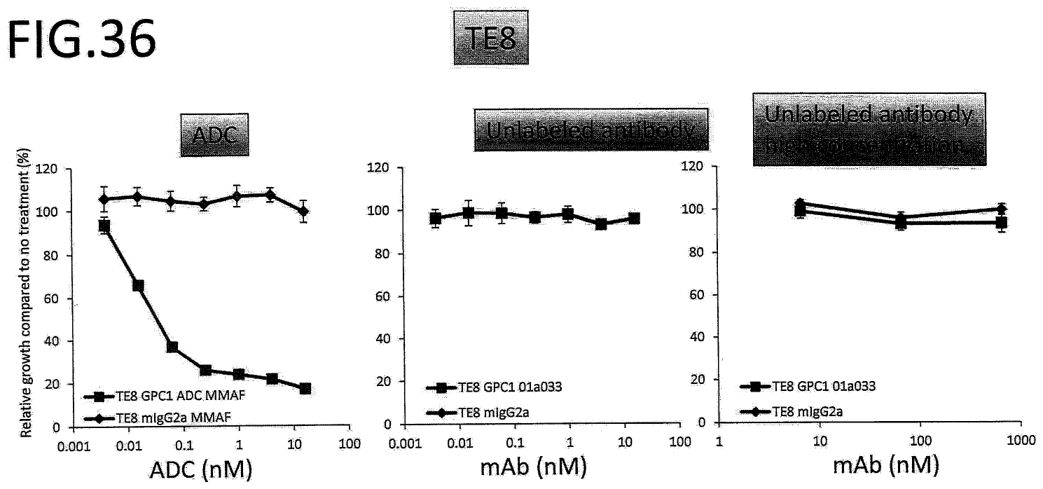

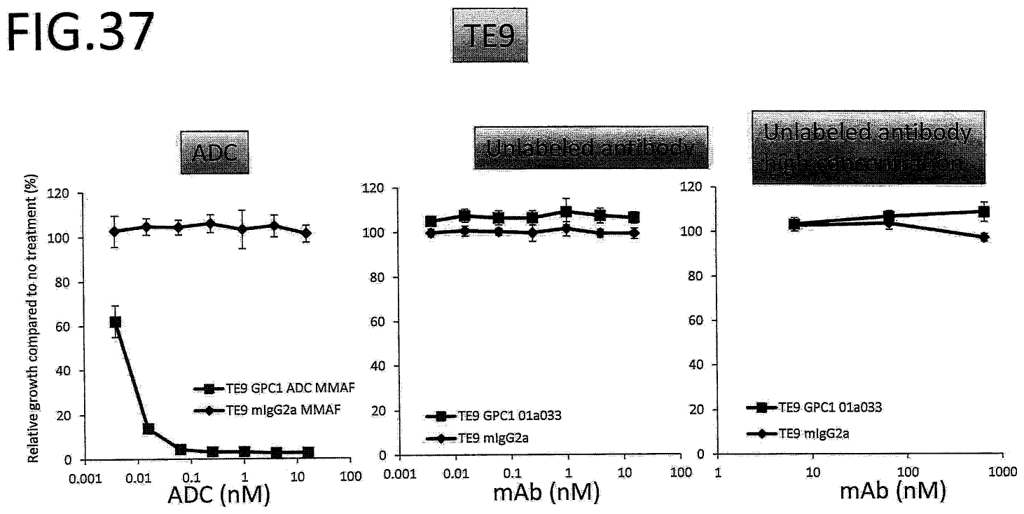

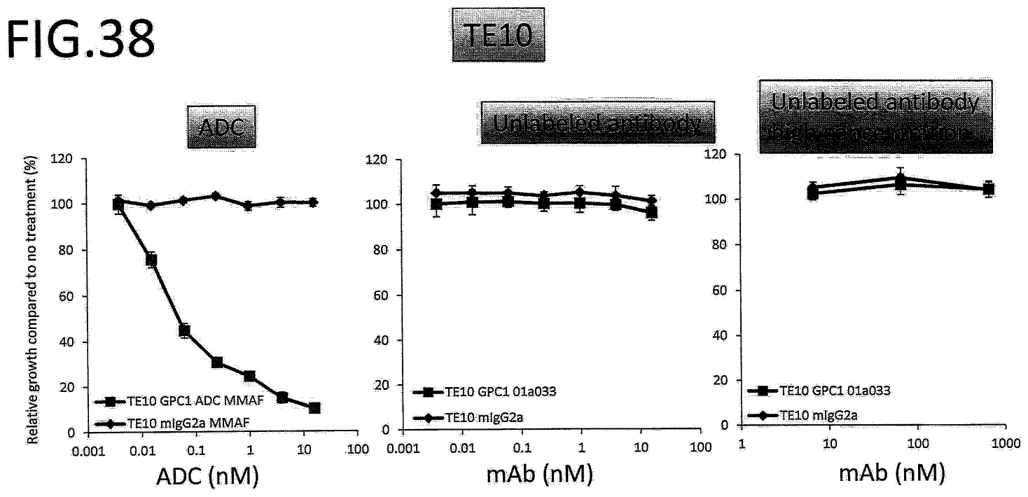

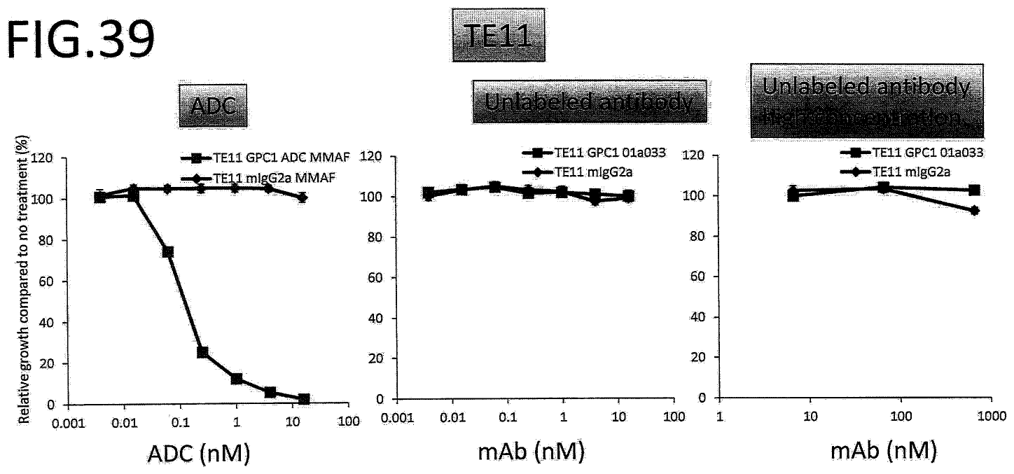

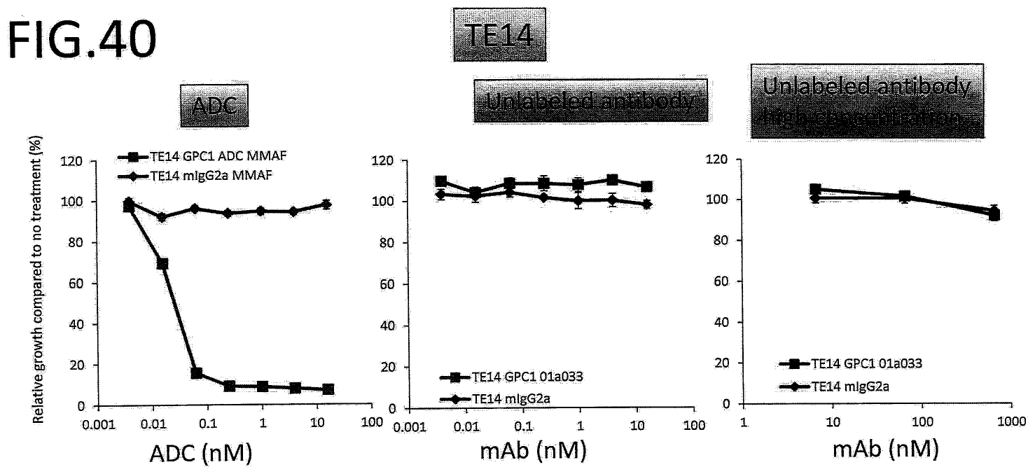

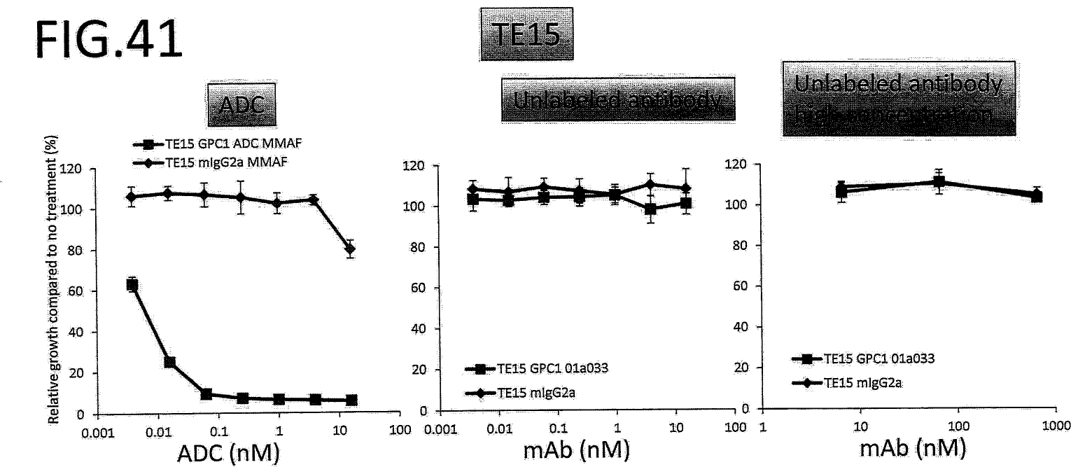

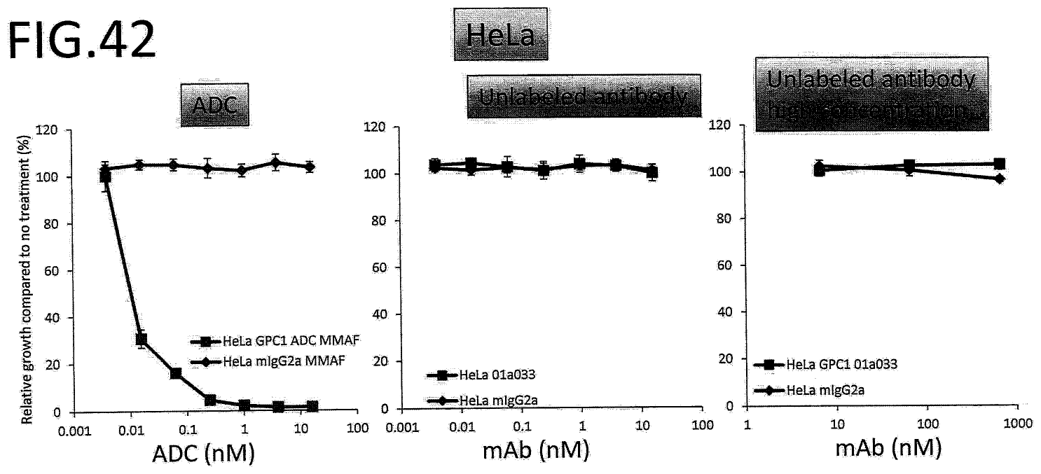

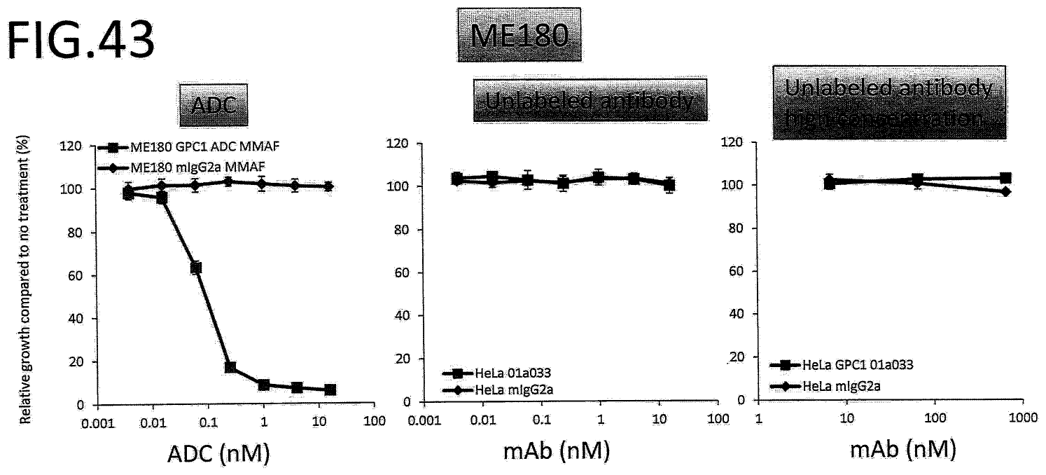

The results are shown in FIGS. 33 to 43. An antitumor effect in vitro due to 01a033 unlabeled antibody was not observed against any cell strain. Likewise, an antitumor effect was not observed with a higher concentration of antibodies. Therefore, it was revealed that a 01a033 antibody does not have an antitumor effect in itself, but exhibits an antitumor effect only as an ADC.

### Example 13: In Vivo Antitumor Effect of Unlabeled Anti-GPC1 Antibody on GPC1 Positive Cell Strain

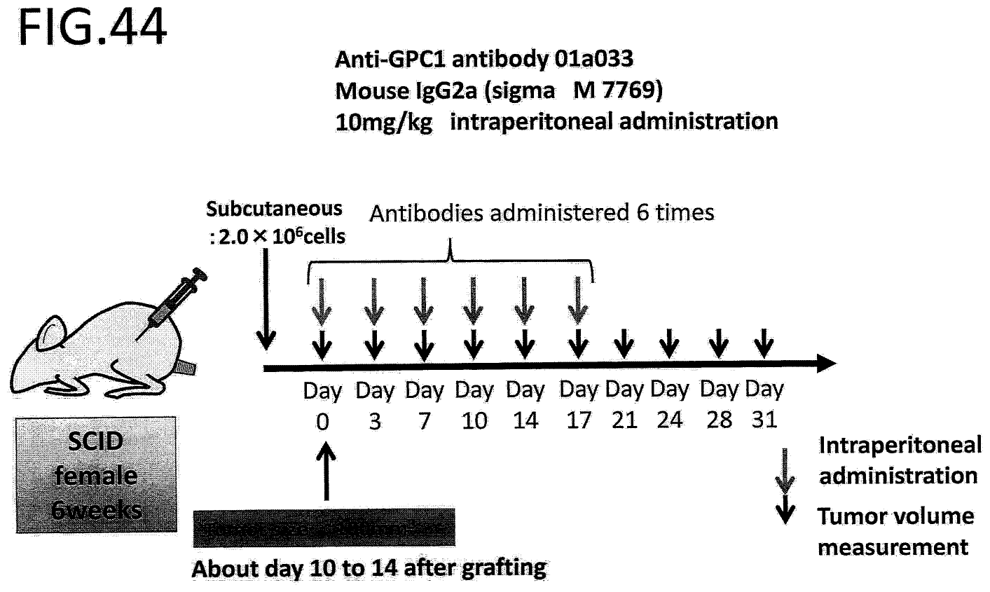

FIG. 44 schematically shows the test in this Example. Specifically, 2.0×10⁶ cells of TE14 cell strain were subcutaneously grafted into female SCID mice (6 week old, n=9). When the tumor size reached about 100 mm³ on about day 10 to 14 after grafting, administration of control mouse IgG2a (sigma, M7769) and anti-GPC1 antibody 01a033 each at 10 mg/kg was started, which were intraperitoneally administered on day 0, day 3, day 7, day 10, day 14, and day 17, with the day starting the administration considered 0 day. The tumor volume was measured on day 0, day 3, day 7, day 10, day 14, day 17, day 21, day 24, day 28, and day 31.

(Results)

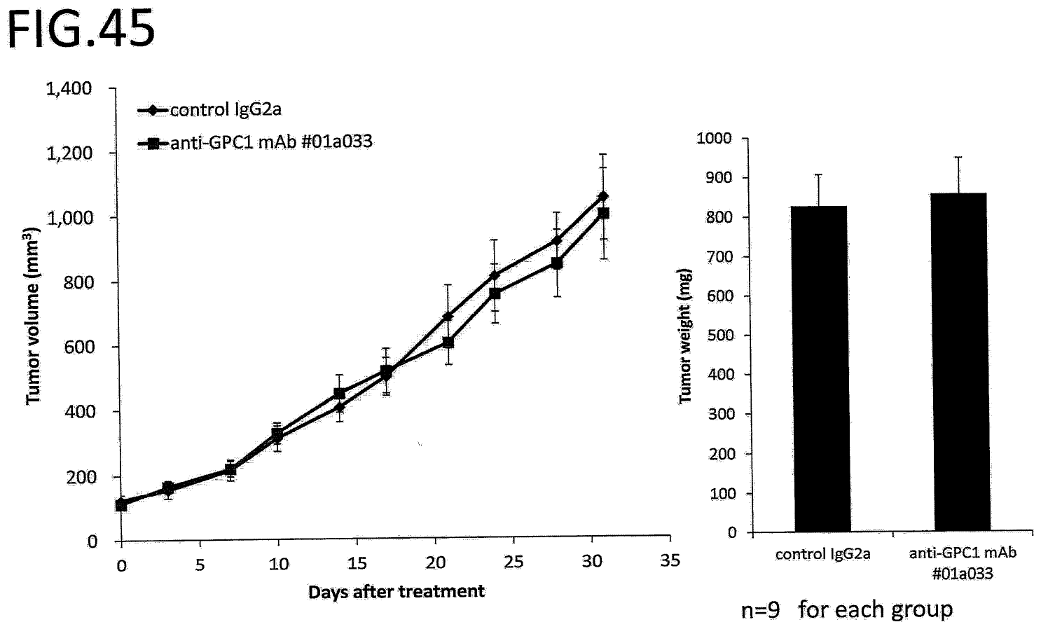

The results are shown in FIG. 45. As shown, it was revealed that the unlabeled anti-GPC1 antibody 01a033 that is not conjugated to an agent having cytotoxic activity exhibits nearly the same degree of tumor growth as control IgG, thus exhibiting no antitumor effect. This result shows that anti-GPC1 antibody 01a033 does not have ADCC activity. While Example 5 shows that anti-GPC1 antibody 01a033 has high internalization activity, the anti-GPC1 antibody 01a033 does not exhibit ADCC activity, potentially due to high internalization activity.

### Example 14: Safety Test on Anti-GPC1 Antibody 01a033 Using Mice

1 mg/body of each of control mouse IgG2a (Sigma, M7769) and anti-GPC1 antibody 01a033 was intraperitoneally administered to a group of 4 male and 4 female C57BL/6J mice (8w). The following items were evaluated on day 7.

Blood collection endpoints: WBC, RBC, Hb, Plt, T-Bil, ALT, ALP, Amy, BUN, Cr, Ca, P, TP, Alb, Na, K, Glob, and Glu, automatic blood cell counter: VetScan HMII, animal biochemical blood analyzer: VetScan VS2.

(Results)

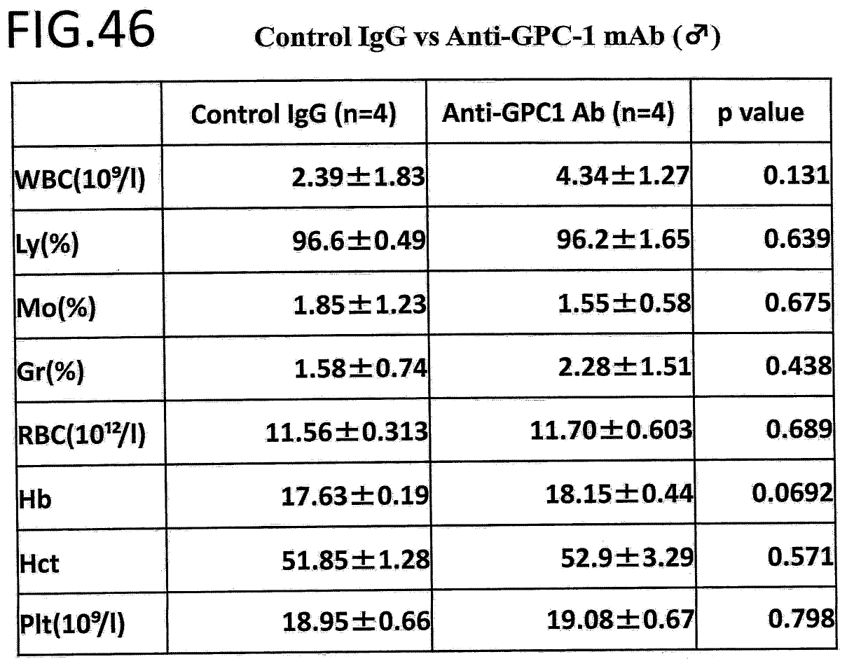

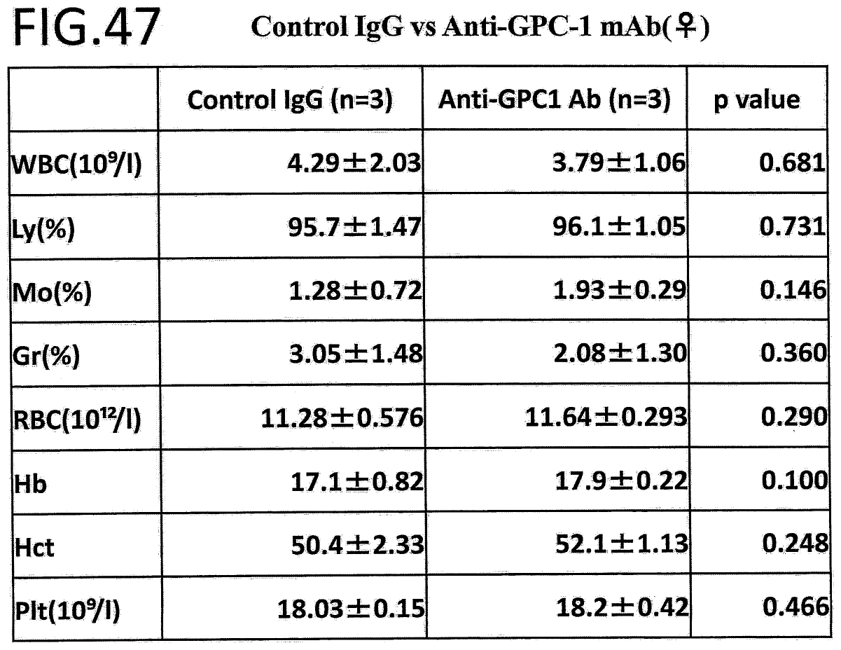

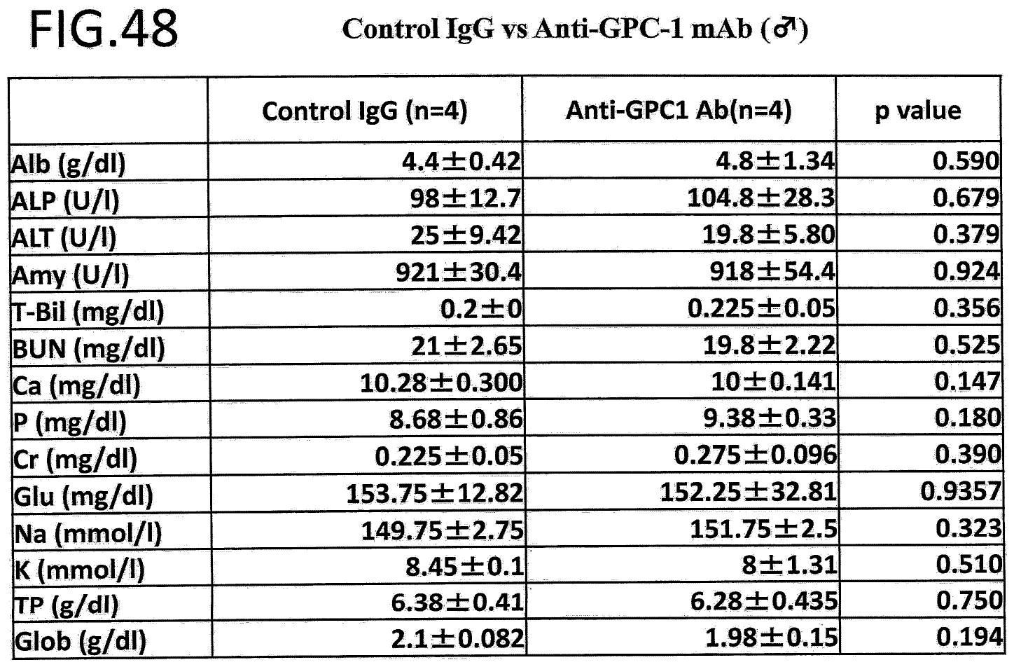

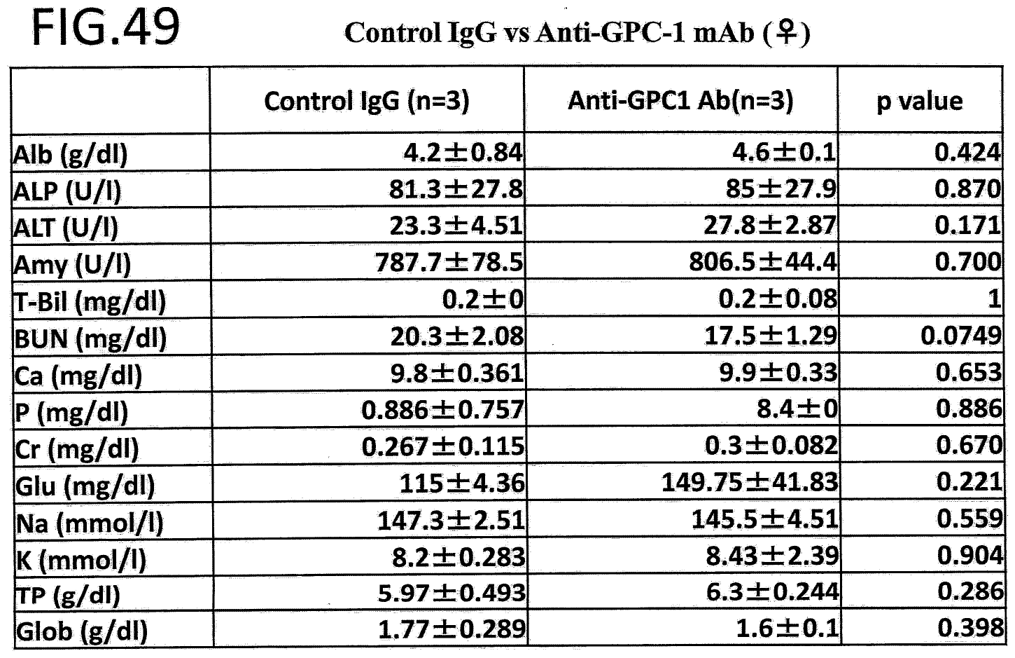

The results are shown in FIGS. 46 to 49. Toxicity was not found after administering unlabeled antibody of clone 01a033 used in ADC itself to mice.

### Example 15: ADC Assay Combining Anti-GPC-1 Antibody and MMAF Binding Secondary Antibody in DU145 Cells

(Materials and Methods)

2000 DU145 cells were added to a 96-well plate at 80 μl. The cells were cultured overnight at 37° C. in a 5% CO2 incubator. The next day, 10 μl each of anticancer agent binding secondary antibody (2 μg/ml) and primary antibody concentrated 10-fold relative to the final concentration was added to each well, so that the total amount was 100 μl. The cells were cultured for 3 days at 37° C. in a 5% CO2 incubator. The cell viability was measured by using a CellTiter-Glo Luminescent Cell Viability Assay reagent to detect the amount of ATP. RPMI1640+10% FBS+1% PS was used as the medium. The final concentrations of the primary antibodies were 0, 0.004, 0.0156, 0.0625, 0.25, 1.0, and 40 nM. 2 μg/ml of Fab-aMFc-CL-MMAF (model number AM202AF-50, Moradec) was used as the secondary antibody. The EC50 value was analyzed with GraphPad Prism 6. The same experiment was conducted using 4000 MDA-MB231 cells as the control.

(Results)

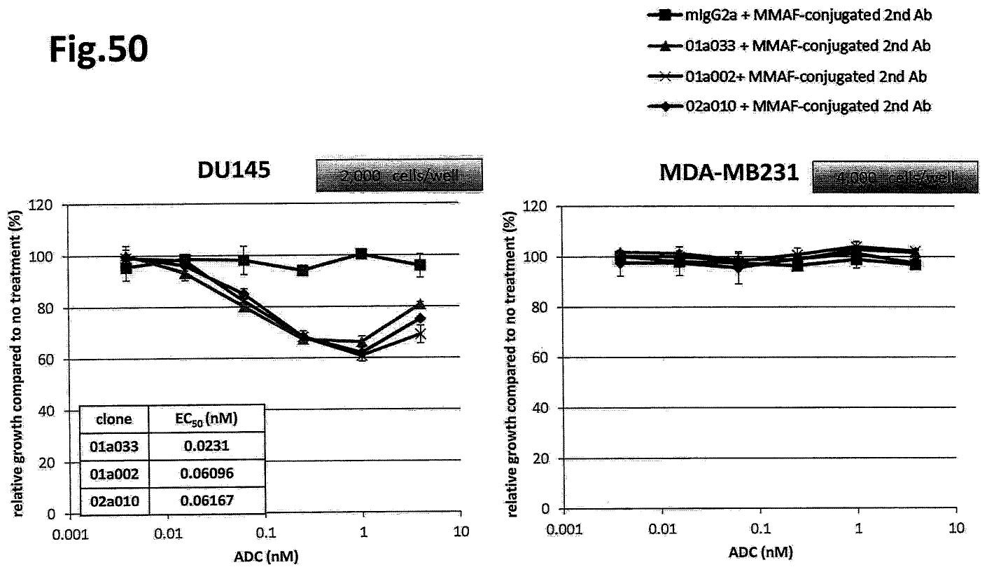

The results are shown in FIG. 50. Clones 01a033, 01a002, and 02a010 exhibited a high efficacy, i.e., high growth suppressing effect, against prostate cancer cell strain DU145 cells. None of the clones exhibited a growth suppressing effect against MDA-MD231 used as a control. Surprisingly, a higher EC₅₀ value than the antibody-drug complex (MIL-38, EC₅₀: 0.8572 nM) described in International Publication No. WO 2016/168885 was exhibited by using any of the clones (FIG. 50). In particular, the activity was 37-fold higher for clone 01a033 compared to MIL-38. A slight action attenuation was observed at high concentrations for all of the clones. Although not wishing to be bound by any theory, it is presumed that action attenuation at a high concentration (4.0 nM) is induced by excessive primary antibodies leading to a competitive reaction with an anticancer agent conjugated secondary antibodies. In view of the results in this Example, the antibody-drug complex of the invention exhibits a better effect of suppressing growth on DU145 cells than existing antibody-drug complexes (e.g., MIL-38).

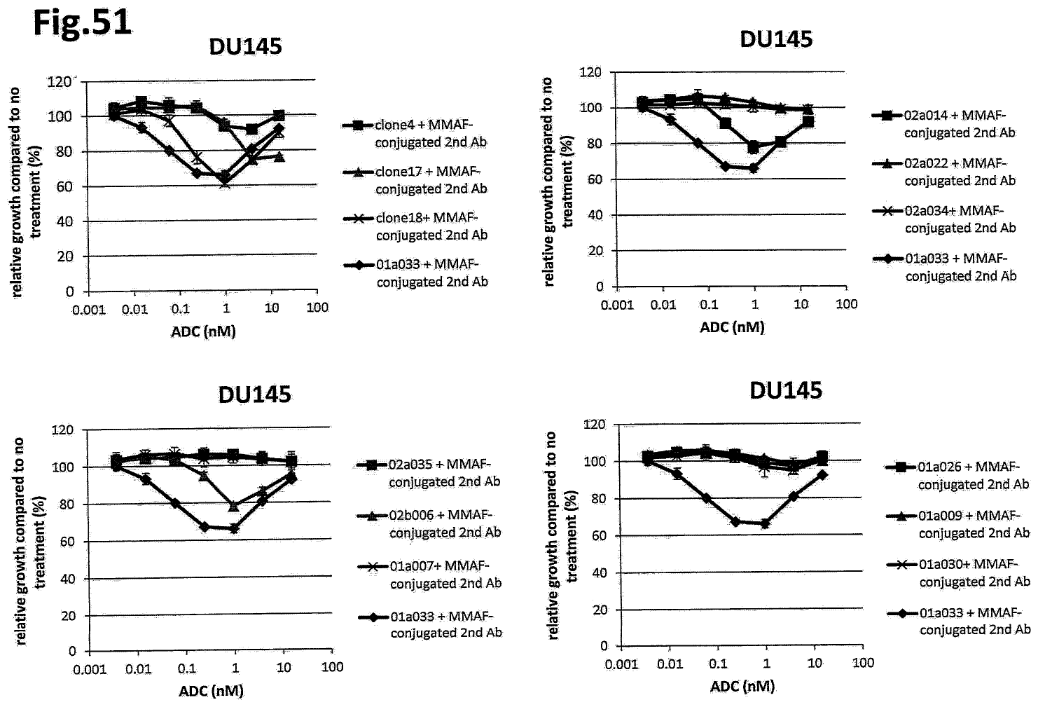

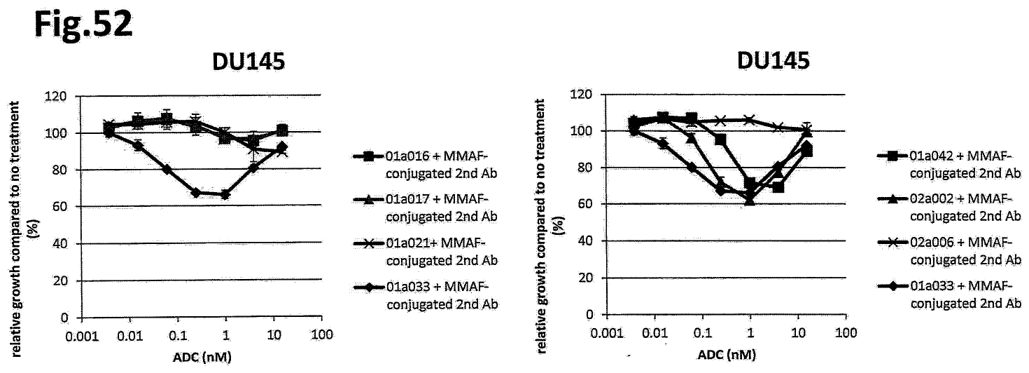

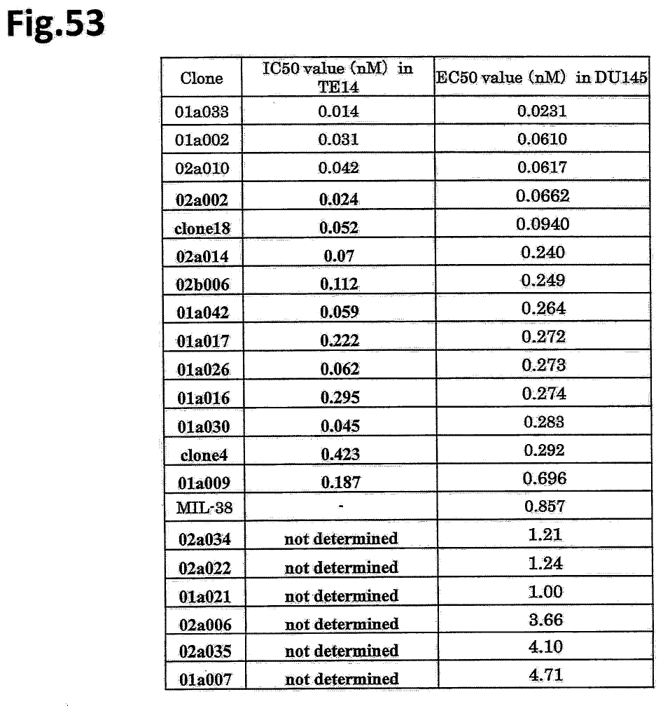

The same test was conducted for clones other than clones 01a033, 01a002, and 02a010. The EC₅₀ value for DU145 cells was calculated (FIGS. 51 to 53). For comparison, the EC₅₀ value of the antibody-drug complex (MIL-38) described in International Publication No. WO 2016/168885 for DU145 cells was used. A better EC₅₀ value than MIL-38 was exhibited for all clones except clones 02a034, 02a022, 01a021, 02a006, 02a035, and 01a007. These clones also exhibit good IC₅₀ value of 0.5 nM or less with respect to TE14, demonstrating that the clones are also effective against different cell strains.

### (Example 16: Establishment of Mouse GPC1 (mGPC1) Expressing LLC (Mouse Lung Cancer Cell Strain) and In Vitro ADC Assay Using Anti-GPC1 Antibody)

(Materials and Methods)

An mGPC1 expression vector (pcDNA3.1-mGPC1) and control vector (pcDNA3.1V5/His) were transfected using Lipofectamine® 2000 into LLC cell strain (mouse lung cancer cell strain). Cells subjected to gene transfer were selected by adding G418 to a medium, and the formed colony was picked up. The expression of mGPC1 was analyzed by FACS using anti-GPC1 antibody clone 01a033. LLC-mGPC1-16 cells were established as an mGPC1 stable expression strain, and LLC-control-7 was established as the control.

Preparation of a conjugate of mouse anti-glypican-1 mAb (01a033), mouse IgG2a (biolegend #400224) as a control, and mc-vc-PAB-MMAF was commissioned to MORADEC. An anticancer agent was conjugated to a cysteine residue via a cleavable linker. The DAR (drug-antibody ratio) was as follows. GPC1-CL-MMAF DAR=3.8, mouse IgG2a-CL-MMAF DAR=4.1.

Specifically, the following assay was conducted.

(1) 1000 each of LLC-control-7 cells that does not express mGPC1 and LLC-mGPC1-16 cells were seeded on a Thermo Fisher Scientific's 96-well white, plate (model no.: 136101) (90 μl cell suspension). RPMI1640+10% FBS+100 U/ml penicillin+100 μg/ml streptomycin was used as the medium. The cells were seeded in 60 wells and 100 μL of medium was added to 36 wells, and cultured in a 5% CO2 incubator at 37° C.
(2) The next day, 10 μL of ADC was added to the cells (total amount: 100 μL).
(3) After 72 hours of culturing, CellTiter-Glo™ Luminescence Cell Viability Assay reagent (Promega) was added and mixed at 100 μL/well.
(4) Measurements were taken with a plate reader.
(5) Analysis was conducted with GraphPad Prism 6.

The IC₅₀ values were calculated from the following equation (Hossain M M, Hosono-Fukao T, Tang R, Sugaya N, van Kuppevelt T H, Jenniskens G J, Kimata K, Rosen S D, Uchimura K (2010) Direct detection of HSulf-1 and HSulf-2 activities on extracellular heparan sulphate and their inhibition by PI-88. Glycobiology 2.0(2): 175-186.)

IC50=10\hat{ }(Log [A][B]×(50−C)/(D−C)+Log [B])

A: High concentration straddling 50%
B: Low concentration straddling 50%
C: Inhibition rate at B
D: Inhibition rate at A

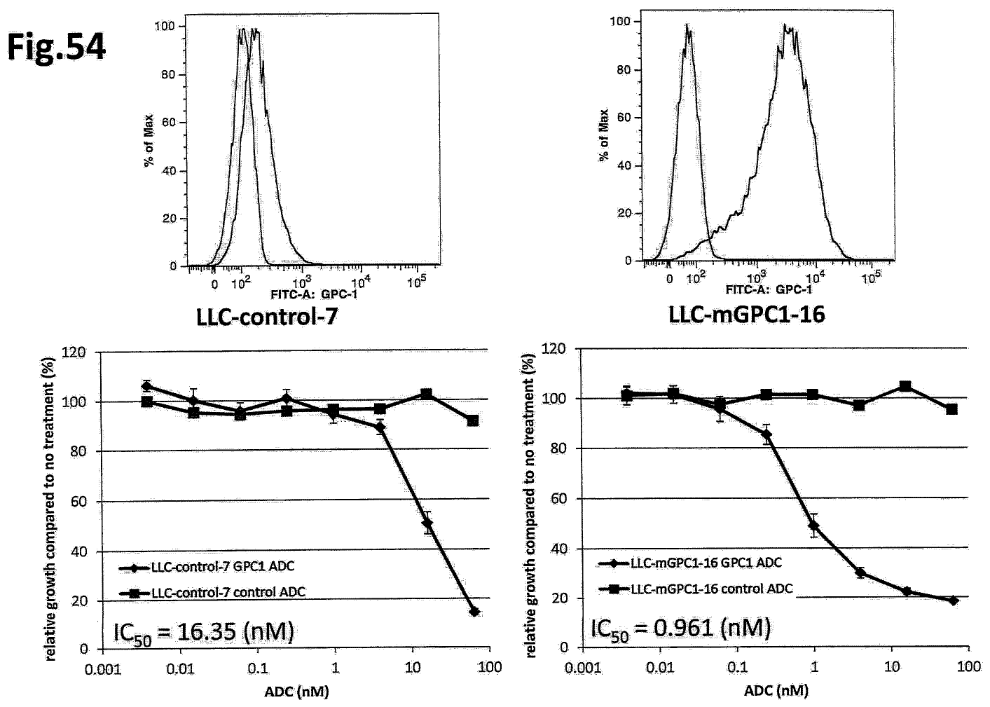

The results are shown in FIG. 54. Clone 01a033 exhibits a low effect of suppressing growth against LLC-control-7 that does not express mouse GPC-1, while exhibiting a 17-fold higher effect of suppressing growth relative to LLC-control-7 against LLC-mGPC1-16 that expressed mouse GPC-1. It was confirmed that a cell strain stably expressing mouse GPC1 was able to be established.

### Example 17: In Vivo Efficacy Test on GPC1 ADC with a Mouse Syngenic Model Using mGPC1 Expressing LLC Cell Strain

A mouse model (syngenic mouse) with GPC1 expressed in normal cells that is consistent with GPC1 expressed from cancer cells (i.e., both mouse GPC1) can be prepared by grafting the cell strain expressing mouse GPC1 established in Example 16 into mice. The efficacy and safety can be more accurately evaluated in an experiment using such a mouse model.

(Materials and Methods)

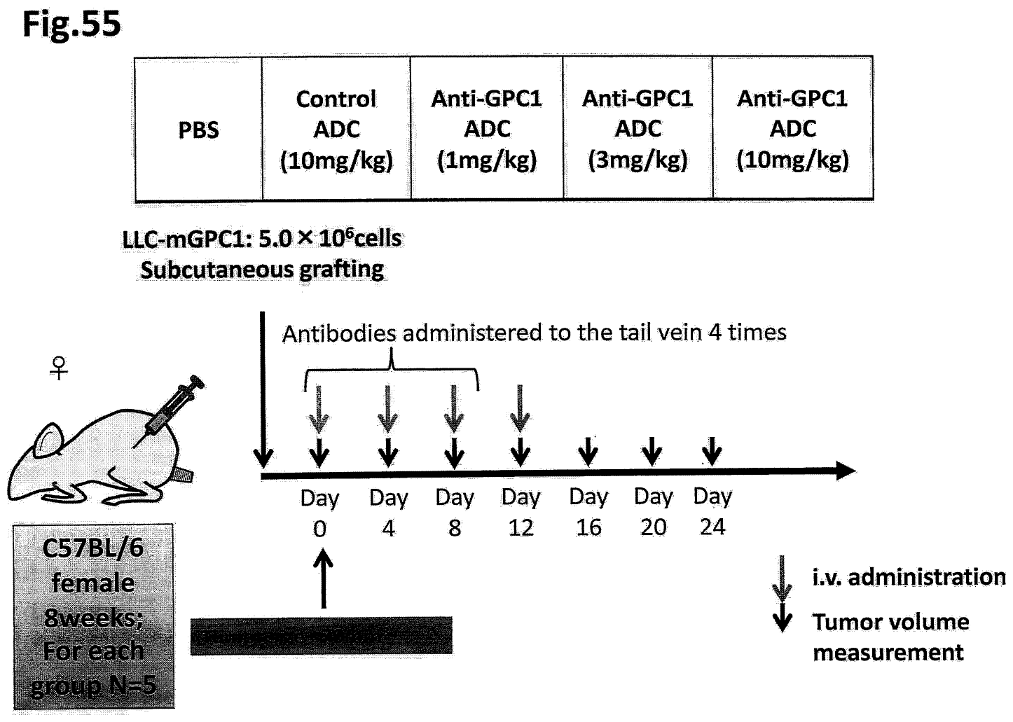

FIG. 55 schematically depicts the test in this Example. Specifically, 5.0×10⁶ cells of LLC-mGPC1-16 were subcutaneously grafted into female C57BL/6 mice. When the tumor size reached about 70. mm³ after grafting, administration of PBS as a control, control ADC (10 mg/kg), anti-GPC1 ADC (1 mg/kg), anti-GPC1 ADC (3 mg/kg), and anti-GPC1 ADC (10 mg/kg) was started, which were intravenously administered on day 0, day 4, day 8, and day 12, with the day starting the administration considered 0 day. The tumor volume and body weight were measured on day 0, day 4, day 8, day 12, day 16, day 20, and day 24.

(Results)

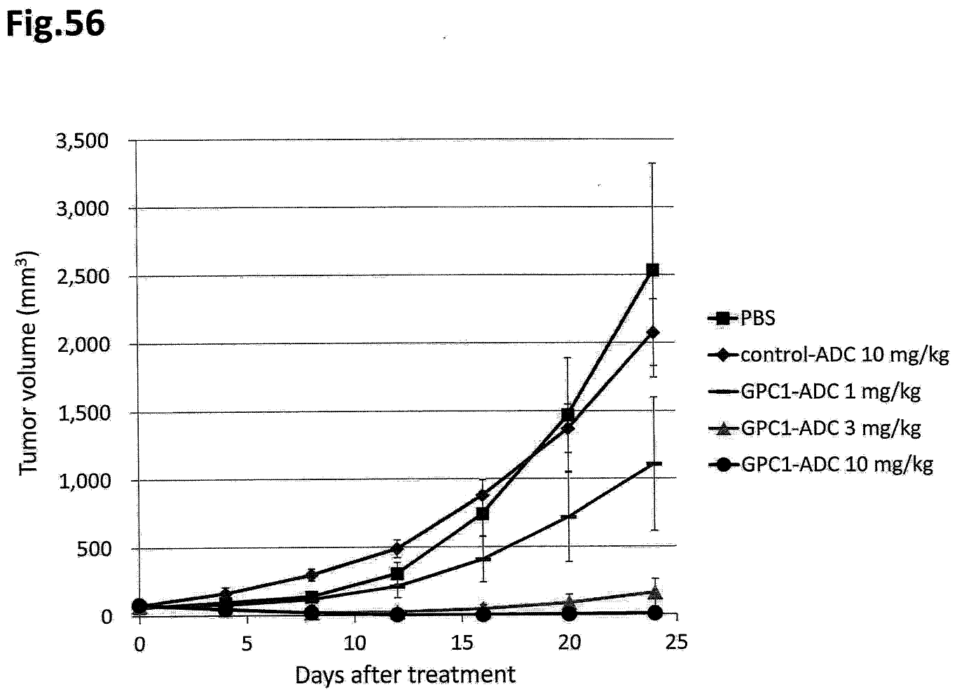

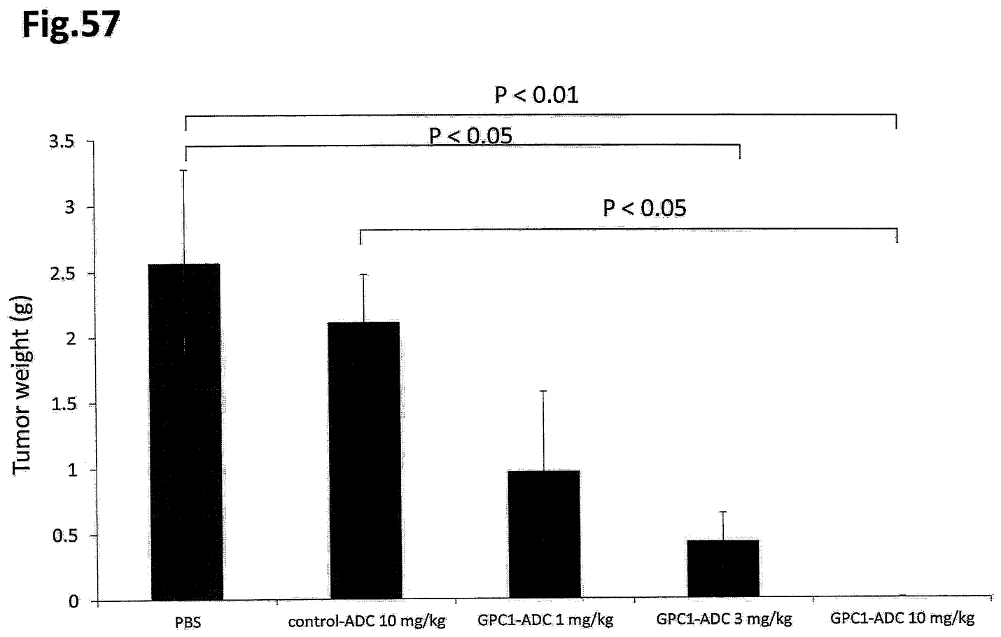

The results are shown in FIGS. 56 and 57. Tumor growth was able be significantly delayed compared to the control group in the group administered with anti-GPC1 ADC (1 mg/kg). Tumor hardly grew in the groups administered with anti-GPC1 ADC (3 mg/kg) and anti-GPC1 ADC (10 mg/kg). In this manner, clone 01a033 also resulted in a high antitumor effect against tumor expressing mouse GPC-1.

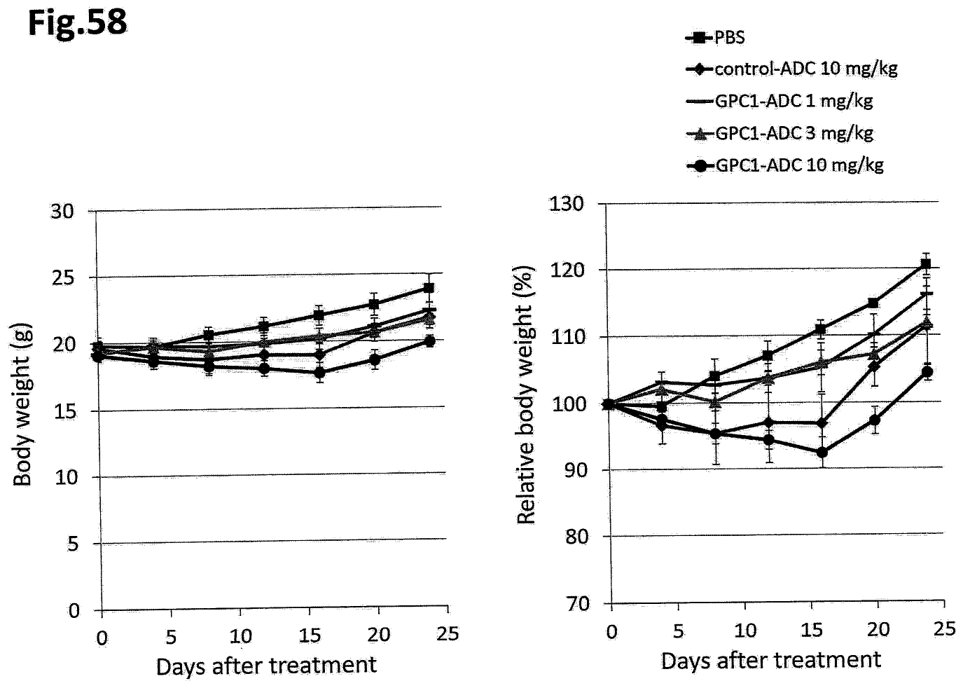

FIG. 58 shows the results for the change in body weight of syngenic mice. A transient decrease in body weight was observed in the group administered with a high concentration anti-GPC1 ADC (10 mg/kg), but the body weight tended to recover after 15 days from the start of administration. Significant toxicity was not observed.

### Examples 18: Application Example of Detection, Diagnosis, and Companion

(1) Cancer Detection/Diagnosis

Cancer can be detected by labeling anti-glypican-1 antibodies with radioisotope (RI) or the like and administering the labeled anti-glypican-1 antibodies to a patient to study the accumulation at a cancer site, thus enabling early diagnosis of cancer, determination of the site of metastasis, and determination of the therapeutic effect of an anticancer agent. Since the anti-glypican-1 antibody of the invention exhibits high internalization activity against cells expressing glypican-1, glypican-1 positive cancer cells can be specifically detected/diagnosed.

(2) Companion Reagent

A companion diagnostic drug of an antigen medicament targeting glypican-1 is prepared by using a new clone to establish sandwich ELISA and quantify glypican-1 in the cancer patient serum.

As described above, the present invention is exemplified by the use of its preferred embodiments. However, it is understood that the scope of the present invention should be interpreted solely based on the Claims. It is also understood that any patent, any patent application, and any references cited herein should be incorporated herein by reference in the same manner as the contents are specifically described herein.

The present application claims priority to Japanese Patent Application No. 2017-90054 (filed on Apr. 28, 2017). It is understood that the entire content of the specification of the application is incorporated herein by reference.

### INDUSTRIAL APPLICABILITY

A medicament for treating or preventing cancer has been provided. Technologies that can be used in industries (pharmaceutical, etc.) based on such a technology are provided.

### [Sequence Listing Free Text]

SEQ ID NO: 1: human glypican-1 nucleic acid sequence (NM_002081.2)
SEQ ID NO: 2: human glypican-1 amino acid sequence (P35052) SEQ ID NO: 3: mouse glypican-1 nucleic acid sequence (NM_016696.4)
SEQ ID NO: 4: mouse glypican-1 amino acid sequence (Q9QZF2)
SEQ ID NO: 5: amino acid sequence of heavy chain CDR1 of K090-01a002
SEQ ID NO: 6: amino acid sequence of heavy chain CDR2 of K090-01a002
SEQ ID NO: 7: amino acid sequence of heavy chain CDR3 of K090-01a002
SEQ ID NO: 8: amino acid sequence of light chain CDR1 of K090-01a002
SEQ ID NO: 9: amino acid sequence of light chain. CDR2 of K090-01a002
SEQ ID NO: 10: amino acid sequence of light chain CDR3 of K090-01a002
SEQ ID NO: 11: amino acid sequence of heavy chain CDR1 of K090-01a007
SEQ ID NO: 12: amino acid sequence of heavy chain CDR2 of K090-01a007
SEQ ID NO: 13: amino acid sequence of heavy chain CDR3 of K090-01a007
SEQ ID NO: 14: amino acid sequence of light chain CDR1 of K090-01a007
SEQ ID NO: 15: amino acid sequence of light chain CDR2 of K090-01a007
SEQ ID NO: 16: amino acid sequence of light chain CDR3 of K090-01a007
SEQ ID NO: 17: amino acid sequence of heavy chain CDR1 of K090-01a016
SEQ ID NO: 18: amino acid sequence of heavy chain CDR2 of K090-01a016
SEQ ID NO: 19: amino acid sequence of heavy chain CDR3 of K090-01a016
SEQ ID NO: 20: amino acid sequence of light chain CDR1 of K090-01a016
SEQ ID NO: 21: amino acid sequence of light chain CDR2 of K090-01a016
SEQ ID NO: 22: amino acid sequence of light chain CDR3 of K090-01a016
SEQ ID NO: 23: amino acid sequence of heavy chain CDR1 of K090-01a017
SEQ ID NO: 24: amino acid sequence of heavy chain CDR2 of K090-01a017
SEQ ID NO: 25: amino acid sequence of heavy chain CDR3 of K090-01a017
SEQ ID NO: 26: amino acid sequence of light chain CDR1 of K090-01a017
SEQ ID NO: 27: amino acid sequence of light chain CDR2 of K090-01a017
SEQ ID NO: 28: amino acid sequence of light chain CDR3 of K090-01a017
SEQ ID NO: 29: amino acid sequence of heavy chain CDR1 of K090-01a021
SEQ ID NO: 30: amino acid sequence of heavy chain CDR2 of K090-01a021
SEQ ID NO: 31: amino acid sequence of heavy chain CDR3 of K090-01a021
SEQ ID NO: 32: amino acid sequence of light chain CDR1 of K090-01a021
SEQ ID NO: 33: amino acid sequence of light chain CDR2 of K090-01a021
SEQ ID NO: 34: amino acid sequence of light chain CDR3 of K090-01a021
SEQ ID NO: 35: amino acid sequence of heavy chain CDR1 of K090-01a026
SEQ ID NO: 36: amino acid sequence of heavy chain CDR2 of K090-01a026
SEQ ID NO: 37: amino acid sequence of heavy chain CDR3 of K090-01a026
SEQ ID NO: 38: amino acid sequence of light chain CDR1 of K090-01a026
SEQ ID NO: 39: amino acid sequence of light chain CDR2 of K090-01a026
SEQ ID NO: 40: amino acid sequence of light chain CDR3 of K090-01a026
SEQ ID NO: 41: amino acid sequence of heavy chain CDR1 of K090-01a009
SEQ ID NO: 42: amino acid sequence of heavy chain CDR2 of K090-01a009
SEQ ID NO: 43: amino acid sequence of heavy chain CDR3 of K090-01a009
SEQ ID NO: 44: amino acid sequence of light chain CDR1 of K090-01a009
SEQ ID NO: 45: amino acid sequence of light chain CDR2 of K090-01a009
SEQ ID NO: 46: amino acid sequence of light chain CDR3 of K090-01a009
SEQ ID NO: 47: amino acid sequence of heavy chain CDR1 of K090-01a030
SEQ ID NO: 48: amino acid sequence of heavy chain CDR2 of K090-01a030
SEQ ID NO: 49: amino acid sequence of heavy chain CDR3 of K090-01a030.
SEQ ID NO: 50: amino acid sequence of light chain CDR1 of K090-01a030
SEQ ID NO: 51: amino acid sequence of light chain CDR2 of K090-01a030
SEQ ID NO: 52: amino acid sequence of light chain CDR3 of K090-01a030
SEQ ID NO: 53: amino acid sequence of heavy chain CDR1 of K090-01a033
SEQ ID NO: 54: amino acid sequence of heavy chain CDR2 of K090-01a033
SEQ ID NO: 55: amino acid sequence of heavy chain CDR3 of K090-01a033
SEQ ID NO: 56: amino acid sequence of light chain CDR1 of K090-01a033
SEQ ID NO: 57: amino acid sequence of light chain CDR2 of K090-01a033
SEQ ID NO: 58: amino acid sequence of light chain CDR3 of K090-01a033
SEQ ID NO: 59: amino acid sequence of heavy chain CDR1 of K090-01a042
SEQ ID NO: 60: amino acid sequence of heavy chain CDR2 of K090-01a042
SEQ ID NO: 61: amino acid sequence of heavy chain CDR3 of K090-01a042
SEQ ID NO: 62: amino acid sequence of light chain CDR1 of K090-01a042
SEQ ID NO: 63: amino acid sequence of light chain CDR2 of K090-01a042
SEQ ID NO: 64: amino acid sequence of light chain CDR3 of K090-01a042
SEQ ID NO: 65: amino acid sequence of heavy chain CDR1 of K090-02a002
SEQ ID NO: 66: amino acid sequence of heavy chain CDR2 of K090-02a002
SEQ ID NO: 67: amino acid sequence of heavy chain CDR3 of K090-02a002
SEQ ID NO: 68: amino acid sequence of light chain CDR1 of K090-02a002
SEQ ID NO: 69: amino acid sequence of light chain CDR2 of K090-02a002
SEQ ID NO: 70: amino acid sequence of light chain CDR3 of K090-02a002
SEQ ID NO: 71: amino acid sequence of heavy chain CDR1 of K090-02a006
SEQ ID NO: 72: amino acid sequence of heavy chain CDR2 of K090-02a006
SEQ ID NO: 73: amino acid sequence of heavy chain CDR3 of K090-02a006
SEQ ID NO: 74: amino acid sequence of light chain CDR1 of K090-02a006
SEQ ID NO: 75: amino acid sequence of light chain CDR2 of K090-02a006
SEQ ID NO: 76: amino acid sequence of light chain CDR3 of K090-02a006
SEQ ID NO: 77: amino acid sequence of heavy chain CDR1 of K090-02a010
SEQ ID NO: 78: amino acid sequence of heavy chain CDR2 of K090-02a010
SEQ ID NO: 79: amino acid sequence of heavy chain CDR3 of K090-02a010
SEQ ID NO: 80: amino acid sequence of light chain CDR1 of K090-02a010
SEQ ID NO: 81: amino acid sequence of light chain CDR2 of K090-02a010
SEQ ID NO: 82: amino acid sequence of light chain CDR3 of K090-02a010
SEQ ID NO: 83: amino acid sequence of heavy chain CDR1 of K090-02a014
SEQ ID NO: 84: amino acid sequence of heavy chain CDR2 of K090-02a014
SEQ ID NO: 85: amino acid sequence of heavy chain CDR3 of K090-02a014
SEQ ID NO: 86: amino acid sequence of light chain CDR1 of K090-02a014
SEQ ID NO: 87: amino acid sequence of light chain CDR2 of K090-02a014
SEQ ID NO: 88: amino acid sequence of light chain CDR3 of K090-02a014
SEQ ID NO: 89: amino acid sequence of heavy chain CDR1 of K090-02a022
SEQ ID NO: 90: amino acid sequence of heavy chain CDR2 of K090-02a022
SEQ ID NO: 91: amino acid sequence of heavy chain CDR3 of K090-02a022
SEQ ID NO: 92: amino acid sequence of light chain CDR1 of K090-02a022
SEQ ID NO: 93: amino acid sequence of light chain CDR2 of K090-02a022
SEQ ID NO: 94: amino acid sequence of light chain CDR3 of K090-02a022
SEQ ID NO: 95: amino acid sequence of heavy chain CDR1 of K090-02a034
SEQ ID NO: 96: amino acid sequence of heavy chain CDR2 of K090-02a034
SEQ ID NO: 97: amino acid sequence of heavy chain CDR3 of K090-02a034
SEQ ID NO: 98: amino acid sequence of light chain CDR1 of K090-02a034
SEQ ID NO: 99: amino acid sequence of light chain CDR2 of K090-02a034
SEQ ID NO: 100: amino acid sequence of light chain CDR3 of K090-02a034
SEQ ID NO: 101: amino acid sequence of heavy chain CDR1 of K090-02a035
SEQ ID NO: 102: amino acid sequence of heavy chain CDR2 of K090-02a035
SEQ ID NO: 103: amino acid sequence of heavy chain CDR3 of K090-02a035
SEQ ID NO: 104: amino acid sequence of light chain CDR1 of K090-02a035
SEQ ID NO: 105: amino acid sequence of light chain CDR2 of K090-02a035
SEQ ID NO: 106: amino acid sequence of light chain CDR3 of K090-02a035
SEQ ID NO: 107: amino acid sequence of heavy chain CDR1 of K090-02b006
SEQ ID NO: 108: amino acid sequence of heavy chain CDR2 of K090-02b006
SEQ ID NO: 109: amino acid sequence of heavy chain CDR3 of K090-02b006
SEQ ID NO: 110: amino acid sequence of light chain CDR1 of K090-02b006
SEQ ID NO: 111: amino acid sequence of light chain CDR2 of K090-02b006
SEQ ID NO: 112: amino acid sequence of light chain CDR3 of K090-02b006
SEQ ID NO: 113: amino acid sequence of heavy chain CDR1 of clone 4
SEQ ID NO: 114: amino acid sequence of heavy chain CDR2 of clone 4
SEQ ID NO: 115: amino acid sequence of heavy chain CDR3 of clone 4
SEQ ID NO: 116: amino acid sequence of light chain CDR1 of clone 4
SEQ ID NO: 117: amino acid sequence of light chain CDR2 of clone 4
SEQ ID NO: 118: amino acid sequence of light chain CDR3 of clone 4
SEQ ID NO: 119: amino acid sequence of heavy chain CDR1 of clone 18
SEQ ID NO: 120: amino acid sequence of heavy chain CDR2 of clone 18
SEQ ID NO: 121: amino acid sequence of heavy chain CDR3 of clone 18
SEQ ID NO: 122: amino acid sequence of light chain CDR1 of clone 18
SEQ ID NO: 123: amino acid sequence of light chain CDR2 of clone 18
SEQ ID NO: 124: amino acid sequence of light chain CDR3 of clone 18
SEQ ID NO: 125: nucleic acid sequence of heavy chain of K090-01a002
SEQ ID NO: 126: amino acid sequence of heavy chain of K090-01a002
SEQ ID NO: 127: nucleic acid sequence of light chain of K090-01a002
SEQ ID NO: 128: amino acid sequence of light chain of K090-01a002
SEQ ID NO: 129: nucleic acid sequence of heavy chain of K090-01a007
SEQ ID NO: 130: amino acid sequence of heavy chain of K090-01a007
SEQ ID NO: 131: nucleic acid sequence of light chain of K090-01a007
SEQ ID NO: 132: amino acid sequence of light chain of K090-01a007
SEQ ID NO: 133: nucleic acid sequence of heavy chain of K090-01a016
SEQ ID NO: 134: amino acid sequence of heavy chain of K090-01a016
SEQ ID NO: 135: nucleic acid sequence of light chain of K090-01a016
SEQ ID NO: 136: amino acid sequence of light chain of K090-01a016
SEQ ID NO: 137: nucleic acid sequence of heavy chain of K090-01a017
SEQ ID NO: 138: amino acid sequence of heavy chain of K090-01a017
SEQ ID NO: 139: nucleic acid sequence of light chain of K090-01a017
SEQ ID NO: 140: amino acid sequence of light chain of K090-01a017
SEQ ID NO: 141: nucleic acid sequence of heavy chain of K090-01a021
SEQ ID NO: 142: amino acid sequence of heavy chain of K090-01a021
SEQ ID NO: 143: nucleic acid sequence of light chain of K090-01a021
SEQ ID NO: 144: amino acid sequence of light chain of K090-01a021
SEQ ID NO: 145: nucleic acid sequence of heavy chain of K090-01a026
SEQ ID NO: 146: amino acid sequence of heavy chain of K090-01a026
SEQ ID NO: 147: nucleic acid sequence of light chain of K090-01a026
SEQ ID NO: 148: amino acid sequence of light chain of K090-01a026
SEQ ID NO: 149: nucleic acid sequence of heavy chain of K090-01a009
SEQ ID NO: 150: amino acid sequence of heavy chain of K090-01a009
SEQ ID NO: 151: nucleic acid sequence of light chain of K090-01a009
SEQ ID NO: 152: amino acid sequence of light chain of K090-01a009
SEQ ID NO: 153: nucleic acid sequence of heavy chain of K090-01a030
SEQ ID NO: 154: amino acid sequence of heavy chain of K090-01a030
SEQ ID NO: 155: nucleic acid sequence of light chain of K090-01a030
SEQ ID NO: 156: amino acid sequence of light chain of K090-01a030
SEQ ID NO: 157: nucleic acid sequence of heavy chain of K090-01a033
SEQ ID NO: 158: amino acid sequence of heavy chain of K090-01a033
SEQ ID NO: 159: nucleic acid sequence of light chain of K090-01a033
SEQ ID NO: 160: amino acid sequence of light chain of K090-01a033
SEQ ID NO: 161: nucleic acid sequence of heavy chain of K090-01a042
SEQ ID NO: 162: amino acid sequence of heavy chain of K090-01a042
SEQ ID NO: 163: nucleic acid sequence of light chain of K090-01a042
SEQ ID NO: 164: amino acid sequence of light chain of K090-01a042
SEQ ID NO: 165: nucleic acid sequence of heavy chain of K090-02a002
SEQ ID NO: 166: amino acid sequence of heavy chain of K090-02a002
SEQ ID NO: 167: nucleic acid sequence of light chain of K090-02a002
SEQ ID NO: 168: amino acid sequence of light chain of K090-02a002
SEQ ID NO: 169: nucleic acid sequence of heavy chain of K090-02a006
SEQ ID NO: 170: amino acid sequence of heavy chain of K090-02a006
SEQ ID NO: 171: nucleic acid sequence of light chain of K090-02a006.
SEQ ID NO: 172: amino acid sequence of light chain of K090-02a006
SEQ ID NO: 173: nucleic acid sequence of heavy chain of K090-02a010
SEQ ID NO: 174: amino acid sequence of heavy chain of K090-02a010
SEQ ID NO: 175: nucleic acid sequence of light chain of K090-02a010
SEQ ID NO: 176: amino acid sequence of light chain of K090-02a010
SEQ ID NO: 177: nucleic acid sequence of heavy chain of K090-02a014
SEQ ID NO: 178: amino acid sequence of heavy chain of K090-02a014
SEQ ID NO: 179: nucleic acid sequence of light chain of K090-02a014
SEQ ID NO: 180: amino acid sequence of light chain of K090-02a014
SEQ ID NO: 181: nucleic acid sequence of heavy chain of K090-02a022
SEQ ID NO: 182: amino acid sequence of heavy chain of K090-02a022
SEQ ID NO: 183: nucleic acid sequence of light chain of K090-02a022
SEQ ID NO: 184: amino acid sequence of light chain of K090-02a022
SEQ ID NO: 185: nucleic acid sequence of heavy chain of K090-02a034
SEQ ID NO: 186: amino acid sequence of heavy chain of K090-02a034
SEQ ID NO: 187: nucleic acid sequence of light chain of K090-02a034
SEQ ID NO: 188: amino acid sequence of light chain of K090-02a034
SEQ ID NO: 189: nucleic acid sequence of heavy chain of K090-02a035
SEQ ID NO: 190: amino acid sequence of heavy chain of K090-02a035
SEQ ID NO: 191: nucleic acid sequence of light chain of K090-02a035
SEQ ID NO: 192: amino acid sequence of light chain of K090-02a035
SEQ ID NO: 193: nucleic acid sequence of heavy chain of K090-02b006
SEQ ID NO: 194: amino acid sequence of heavy chain of K090-02b006
SEQ ID NO: 195: nucleic acid sequence of light chain of K090-02b006
SEQ ID NO: 196: amino acid sequence of light chain of K090-02b006
SEQ ID NO: 197: nucleic acid sequence of heavy chain of clone 4
SEQ ID NO: 198: amino acid sequence of heavy chain of clone 4
SEQ ID NO: 199: nucleic acid sequence of light chain of clone 4
SEQ ID NO: 200: amino acid sequence of light chain of clone 4
SEQ ID NO: 201: nucleic acid sequence of heavy chain of clone 18
SEQ ID NO: 202: amino acid sequence of heavy chain of clone 18
SEQ ID NO: 203: nucleic acid sequence of light chain of clone 18
SEQ ID NO: 204: amino acid sequence of light chain of clone 18

1. An anti-human glypican-1 antibody or antigen binding fragment thereof, wherein the antibody is selected from the group consisting of:
(a) an antibody comprising amino acid sequences of heavy chain CDR1, heavy chain CDR2, and heavy chain CDR3 set forth in SEQ ID NOs: 53, 54, and 55, respectively, and light chain CDR1, light chain CDR2, and light chain CDR3 set forth in SEQ ID NOs: 56, 57, and 58, respectively;
(b) an antibody comprising amino acid sequences of heavy chain CDR1, heavy chain CDR2, and heavy chain CDR3 set forth in SEQ ID NOs: 5, 6, and 7, respectively, and light chain CDR1, light chain CDR2, and light chain CDR3 set forth in SEQ ID NOs: 8, 9, and 10, respectively;
(c) an antibody comprising amino acid sequences of heavy chain CDR1, heavy chain CDR2, and heavy chain CDR3 set forth in SEQ ID NOs: 11, 12, and 13, respectively, and light chain CDR1, light chain CDR2, and light chain CDR3 set forth in SEQ ID NOs: 14, 15, and 16, respectively;
(d) an antibody comprising amino acid sequences of heavy chain CDR1, heavy chain CDR2, and heavy chain CDR3 set forth in SEQ ID NOs: 17, 18, and 19, respectively, and light chain CDR1, light chain CDR2, and light chain CDR3 set forth in SEQ ID NOs: 20, 21, and 22, respectively;
(e) an antibody comprising amino acid sequences of heavy chain CDR1, heavy chain CDR2, and heavy chain CDR3 set forth in SEQ ID NOs: 23, 24, and 25, respectively, and light chain CDR1, light chain CDR2, and light chain CDR3 set forth in SEQ ID NOs: 26, 27, and 28, respectively;
(f) an antibody comprising amino acid sequences of heavy chain CDR1, heavy chain CDR2, and heavy chain CDR3 set forth in SEQ ID NOs: 29, 30, and 31, respectively, and light chain CDR1, light chain CDR2, and light chain CDR3 set forth in SEQ ID NOs: 32, 33, and 34, respectively;
(g) an antibody comprising amino acid sequences of heavy chain CDR1, heavy chain CDR2, and heavy chain CDR3 set forth in SEQ ID NOs: 35, 36, and 37, respectively, and light chain CDR1, light chain CDR2, and light chain CDR3 set forth in SEQ ID NOs: 38, 39, and 40, respectively;
(h) an antibody comprising amino acid sequences of heavy chain CDR1, heavy chain CDR2, and heavy chain CDR3 set forth in SEQ ID NOs: 41, 42, and 43, respectively, and light chain CDR1, light chain CDR2, and light chain CDR3 set forth in SEQ ID NOs: 44, 45, and 46, respectively;
(i) an antibody comprising amino acid sequences of heavy chain CDR1, heavy chain CDR2, and heavy chain CDR3 set forth in SEQ ID NOs: 47, 48, and 49, respectively, and light chain CDR1, light chain CDR2, and light chain CDR3 set forth in SEQ ID NOs: 50, 51, and 52, respectively;
(j) an antibody comprising amino acid sequences of heavy chain CDR1, heavy chain CDR2, and heavy chain CDR3 set forth in SEQ ID NOs: 59, 60, and 61, respectively, and light chain CDR1, light chain CDR2, and light chain CDR3 set forth in SEQ ID NOs: 62, 63, and 64, respectively;
(k) an antibody comprising amino acid sequences of heavy chain CDR1, heavy chain CDR2, and heavy chain CDR3 set forth in SEQ ID NOs: 65, 66, and 67, respectively, and light chain CDR1, light chain CDR2, and light chain CDR3 set forth in SEQ ID NOs: 68, 69, and 70, respectively;
(l) an antibody comprising amino acid sequences of heavy chain CDR1, heavy chain CDR2, and heavy chain CDR3 set forth in SEQ ID NOs: 71, 72, and 73, respectively, and light chain CDR1, light chain CDR2, and light chain CDR3 set forth in SEQ ID NOs: 74, 75, and 76, respectively;
(m) an antibody comprising amino acid sequences of heavy chain CDR1, heavy chain CDR2, and heavy chain CDR3 set forth in SEQ ID NOs: 77, 78, and 79, respectively, and light chain CDR1, light chain CDR2, and light chain CDR3 set forth in SEQ ID NOs: 80, 81, and 82, respectively;
(n) an antibody comprising amino acid sequences of heavy chain CDR1, heavy chain CDR2, and heavy chain CDR3 set forth in SEQ ID NOs: 83, 84, and 85, respectively, and light chain CDR1, light chain CDR2, and light chain CDR3 set forth in SEQ ID NOs: 86, 87, and 88, respectively;
(o) an antibody comprising amino acid sequences of heavy chain CDR1, heavy chain CDR2, and heavy chain CDR3 set forth in SEQ ID NOs: 89, 90, and 91, respectively, and light chain CDR1, light chain CDR2, and light chain CDR3 set forth in SEQ ID NOs: 92, 93, and 94, respectively;
(p) an antibody comprising amino acid sequences of heavy chain CDR1, heavy chain CDR2, and heavy chain CDR3 set forth in SEQ ID NOs: 95, 96, and 97, respectively, and light chain CDR1, light chain CDR2, and light chain CDR3 set forth in SEQ ID NOs: 98, 99, and 100, respectively;
(q) an antibody comprising amino acid sequences of heavy chain CDR1, heavy chain CDR2, and heavy chain CDR3 set forth in SEQ ID NOs: 101, 102, and 103, respectively, and light chain CDR1, light chain CDR2, and light chain CDR3 set forth in SEQ ID NOs: 104, 105, and 106, respectively;
(r) an antibody comprising amino acid sequences of heavy chain CDR1, heavy chain CDR2, and heavy chain CDR3 set forth in SEQ ID NOs: 107, 108, and 109, respectively, and light chain CDR1, light chain CDR2, and light chain CDR3 set forth in SEQ ID NOs: 110, 111, and 112, respectively; and
(s) a mutant of an antibody selected from (a) to (r) comprising at least one substitution, addition, or deletion in a CDR moiety.

2. The anti-human glypican-1 antibody or antigen binding fragment thereof of claim 1, wherein the antibody is selected from the group consisting of:
(a) an antibody comprising amino acid sequences of a heavy chain set forth in SEQ ID NO: 158 and a light chain set forth in SEQ ID NO: 160;
(b) an antibody comprising amino acid sequences of a heavy chain set forth in SEQ ID NO: 126 and a light chain set forth in SEQ ID NO: 128;
(c) an antibody comprising amino acid sequences of a heavy chain set forth in SEQ ID NO: 130 and a light chain set forth in SEQ ID NO: 132;
(d) an antibody comprising amino acid sequences of a heavy chain set forth in SEQ ID NO: 134 and a light chain set forth in SEQ ID NO: 136;
(e) an antibody comprising amino acid sequences of a heavy chain set forth in SEQ ID NO: 138 and a light chain set forth in SEQ ID NO: 140;
(f) an antibody comprising amino acid sequences of a heavy chain set forth in SEQ ID NO: 142 and a light chain set forth in SEQ ID NO: 144;
(g) an antibody comprising amino acid sequences of a heavy chain set forth in SEQ ID NO: 146 and a light chain set forth in SEQ ID NO: 148;
(h) an antibody comprising amino acid sequences of a heavy chain set forth in SEQ ID NO: 150 and a light chain set forth in SEQ ID NO: 152;
(i) an antibody comprising amino acid sequences of a heavy chain set forth in SEQ ID NO: 154 and a light chain set forth in SEQ ID NO: 156;
(j) an antibody comprising amino acid sequences of a heavy chain set forth in SEQ ID NO: 162 and a light chain set forth in SEQ ID NO: 164;
(k) an antibody comprising amino acid sequences of a heavy chain set forth in SEQ ID NO: 166 and a light chain set forth in SEQ ID NO: 168;
(l) an antibody comprising amino acid sequences of a heavy chain set forth in SEQ ID NO: 170 and a light chain set forth in SEQ ID NO: 172;
(m) an antibody comprising amino acid sequences of a heavy chain set forth in SEQ ID NO: 174 and a light chain set forth in SEQ ID NO: 176;
(n) an antibody comprising amino acid sequences of a heavy chain set forth in SEQ ID NO: 178 and a light chain set forth in SEQ ID NO: 180;
(o) an antibody comprising amino acid sequences of a heavy chain set forth in SEQ ID NO: 182 and a light chain set forth in SEQ ID NO: 184;
(p) an antibody comprising amino acid sequences of a heavy chain set forth in SEQ ID NO: 186 and a light chain set forth in SEQ ID NO: 188;
(q) an antibody comprising amino acid sequences of a heavy chain set forth in SEQ ID NO: 190 and a light chain set forth in SEQ ID NO: 192;
(r) an antibody comprising amino acid sequences of a heavy chain set forth in SEQ ID NO: 194 and a light chain set forth in SEQ ID NO: 196; and
(s) a mutant of an antibody selected from (a) to (r) comprising at least one substitution, addition, or deletion.

3. The anti-human glypican antibody or antigen binding fragment thereof of claim 1, wherein the mutant comprises at least one substitution, addition, or deletion in a framework of the antibody.

4. The anti-human glypican antibody or antigen binding fragment thereof of claim 1, which binds to human glypican-1 at a binding constant of about 10 nM or less.

5. The anti-human glypican antibody or antigen binding fragment thereof of claim 1, having an internalization activity of about 30% or greater with respect to glypican-1 positive cells after 2 hours from starting incubation with the antibody or antigen fragment thereof in an internalization assay.

6. The anti-human glypican antibody or antigen binding fragment thereof of claim 1, having an internalization activity of about 50% or greater with respect to glypican-1 positive cells after 2 hours from starting incubation with the antibody or antigen fragment thereof in an internalization assay.

7. The anti-human glypican antibody or antigen binding fragment thereof of claim 1, having an internalization activity of about 60% or greater with respect to glypican-1 positive cells after 2 hours from starting incubation with the antibody or antigen fragment thereof in an internalization assay.

8. The anti-human glypican antibody or antigen binding fragment thereof of claim 1, wherein the antibody is an antibody selected from a monoclonal antibody, a polyclonal antibody, a chimeric antibody, a humanized antibody, a human antibody, a multifunctional antibody, a bispecific or oligospecific antibody, a single chain antibody, an scFV, a diabody, an sc(Fv)2 (single chain (Fv)2), and an scFv-Fc.

9.-10. (canceled)

11. A complex of an anti-glypican-1 antibody having activity for intracellular invasion into glypican-1 positive cells or antigen binding fragment thereof and an agent having cytotoxic activity.

12. The complex of claim 11, wherein the anti-glypican-1 antibody or antigen binding fragment thereof is operably linked to the agent having cytotoxic activity via a linker.

13. The complex of claim 12 or 13, wherein an epitope of the antibody comprises:
(a) positions 33 to 61 of SEQ ID NO: 2;
(b) positions 339 to 358 and/or 388 to 421 of SEQ ID NO: 2;
(c) positions 430 to 530 of SEQ ID NO: 2;
(d) positions 33 to 61, 339 to 358, and/or 388 to 421 of SEQ ID NO: 2;
(e) positions 339 to 358, 388 to 421, and/or 430 to 530 of SEQ ID NO: 2; or
(f) positions 33 to 61, 339 to 358, 388 to 421, and/or 430 to 530 of SEQ ID NO: 2.

14. The complex of claim 13, wherein the epitope of the antibody comprises:
(a) positions 33 to 61 of SEQ ID NO: 2;
(b) positions 339 to 358 and 388 to 421 of SEQ ID NO: 2;
(c) positions 33 to 61, 339 to 358, and 388 to 42 of SEQ ID NO: 2; or
(d) positions 33 to 61, 339 to 358, 388 to 421, and 430 to 530 of SEQ ID NO: 2.

15. The complex of claim 11, wherein the anti-glypican-1 antibody is selected from the group consisting of:
(a) an antibody comprising amino acid sequences of heavy chain CDR1, heavy chain CDR2, and heavy chain CDR3 set forth in SEQ ID NOs: 53, 54, and 55, respectively, and light chain CDR1, light chain CDR2, and light chain CDR3 set forth in SEQ ID NOs: 56, 57, and 58, respectively;
(b) an antibody comprising amino acid sequences of heavy chain CDR1, heavy chain CDR2, and heavy chain CDR3 set forth in SEQ ID NOs: 5, 6, and 7, respectively, and light chain CDR1, light chain CDR2, and light chain CDR3 set forth in SEQ ID NOs: 8, 9, and 10, respectively;
(c) an antibody comprising amino acid sequences of heavy chain CDR1, heavy chain CDR2, and heavy chain CDR3 set forth in SEQ ID NOs: 11, 12, and 13, respectively, and light chain CDR1, light chain CDR2, and light chain CDR3 set forth in SEQ ID NOs: 14, 15, and 16, respectively;
(d) an antibody comprising amino acid sequences of heavy chain CDR1, heavy chain CDR2, and heavy chain CDR3 set forth in SEQ ID NOs: 17, 18, and 19, respectively, and light chain CDR1, light chain CDR2, and light chain CDR3 set forth in SEQ ID NOs: 20, 21, and 22, respectively;
(e) an antibody comprising amino acid sequences of heavy chain CDR1, heavy chain CDR2, and heavy chain CDR3 set forth in SEQ ID NOs: 23, 24, and 25, respectively, and light chain CDR1, light chain CDR2, and light chain CDR3 set forth in SEQ ID NOs: 26, 27, and 28, respectively;
(f) an antibody comprising amino acid sequences of heavy chain CDR1, heavy chain CDR2, and heavy chain CDR3 set forth in SEQ ID NOs: 29, 30, and 31, respectively, and light chain CDR1, light chain CDR2, and light chain CDR3 set forth in SEQ ID NOs: 32, 33, and 34, respectively;
(g) an antibody comprising amino acid sequences of heavy chain CDR1, heavy chain CDR2, and heavy chain CDR3 set forth in SEQ ID NOs: 35, 36, and 37, respectively, and light chain CDR1, light chain CDR2, and light chain CDR3 set forth in SEQ ID NOs: 38, 39, and 40, respectively;
(h) an antibody comprising amino acid sequences of heavy chain CDR1, heavy chain CDR2, and heavy chain CDR3 set forth in SEQ ID NOs: 41, 42, and 43, respectively, and light chain CDR1, light chain CDR2, and light chain CDR3 set forth in SEQ ID NOs: 44, 45, and 46, respectively;
(i) an antibody comprising amino acid sequences of heavy chain CDR1, heavy chain CDR2, and heavy chain CDR3 set forth in SEQ ID NOs: 47, 48, and 49, respectively, and light chain CDR1, light chain CDR2, and light chain CDR3 set forth in SEQ ID NOs: 50, 51, and 52, respectively;
(j) an antibody comprising amino acid sequences of heavy chain CDR1, heavy chain CDR2, and heavy chain CDR3 set forth in SEQ ID NOs: 59, 60, and 61, respectively, and light chain CDR1, light chain CDR2, and light chain CDR3 set forth in SEQ ID NOs: 62, 63, and 64, respectively;
(k) an antibody comprising amino acid sequences of heavy chain CDR1, heavy chain CDR2, and heavy chain CDR3 set forth in SEQ ID NOs: 65, 66, and 67, respectively, and light chain CDR1, light chain CDR2, and light chain CDR3 set forth in SEQ ID NOs: 68, 69, and 70, respectively;
(l) an antibody comprising amino acid sequences of heavy chain CDR1, heavy chain CDR2, and heavy chain CDR3 set forth in SEQ ID NOs: 71, 72, and 73, respectively, and light chain CDR1, light chain CDR2, and light chain CDR3 set forth in SEQ ID NOs: 74, 75, and 76, respectively;
(m) an antibody comprising amino acid sequences of heavy chain CDR1, heavy chain CDR2, and heavy chain CDR3 set forth in SEQ ID NOs: 77, 78, and 79, respectively, and light chain CDR1, light chain CDR2, and light chain CDR3 set forth in SEQ ID NOs: 80, 81, and 82, respectively;
(n) an antibody comprising amino acid sequences of heavy chain CDR1, heavy chain CDR2, and heavy chain CDR3 set forth in SEQ ID NOs: 83, 84, and 85, respectively, and light chain CDR1, light chain CDR2, and light chain CDR3 set forth in SEQ ID NOs: 86, 87, and 88, respectively;
(o) an antibody comprising amino acid sequences of heavy chain CDR1, heavy chain CDR2, and heavy chain CDR3 set forth in SEQ ID NOs: 89, 90, and 91, respectively, and light chain CDR1, light chain CDR2, and light chain CDR3 set forth in SEQ ID NOs: 92, 93, and 94, respectively;
(p) an antibody comprising amino acid sequences of heavy chain CDR1, heavy chain CDR2, and heavy chain CDR3 set forth in SEQ ID NOs: 95, 96, and 97, respectively, and light chain CDR1, light chain CDR2, and light chain CDR3 set forth in SEQ ID NOs: 98, 99, and 100, respectively;
(q) an antibody comprising amino acid sequences of heavy chain CDR1, heavy chain CDR2, and heavy chain CDR3 set forth in SEQ ID NOs: 101, 102, and 103, respectively, and light chain CDR1, light chain CDR2, and light chain CDR3 set forth in SEQ ID NOs: 104, 105, and 106, respectively;
(r) an antibody comprising amino acid sequences of heavy chain CDR1, heavy chain CDR2, and heavy chain CDR3 set forth in SEQ ID NOs: 107, 108, and 109, respectively, and light chain CDR1, light chain CDR2, and light chain CDR3 set forth in SEQ ID NOs: 110, 111, and 112, respectively;
(s) an antibody comprising amino acid sequences of heavy chain CDR1, heavy chain CDR2, and heavy chain CDR3 set forth in SEQ ID NOs: 113, 114, and 115, respectively, and light chain CDR1, light chain CDR2, and light chain CDR3 set forth in SEQ ID NOs: 116, 117, and 118, respectively;
(t) an antibody comprising amino acid sequences of heavy chain CDR1, heavy chain CDR2, and heavy chain CDR3 set forth in SEQ ID NOs: 125, 126, and 127, respectively, and light chain CDR1, light chain CDR2, and light chain CDR3 set forth in SEQ ID NOs: 128, 129, and 130, respectively; and
(u) a mutant of an antibody selected from (a) to (t) comprising at least one substitution, addition, or deletion.

16. The complex of claim 15, wherein the anti-glypican-1 antibody is selected from the group consisting of:
(a) an antibody comprising amino acid sequences of a heavy chain set forth in SEQ ID NO: 158 and a light chain set forth in SEQ ID NO: 160;
(b) an antibody comprising amino acid sequences of a heavy chain set forth in SEQ ID NO: 126 and a light chain set forth in SEQ ID NO: 128;
(c) an antibody comprising amino acid sequences of a heavy chain set forth in SEQ ID NO: 130 and a light chain set forth in SEQ ID NO: 132;
(d) an antibody comprising amino acid sequences of a heavy chain set forth in SEQ ID NO: 134 and a light chain set forth in SEQ ID NO: 136;
(e) an antibody comprising amino acid sequences of a heavy chain set forth in SEQ ID NO: 138 and a light chain set forth in SEQ ID NO: 140;
(f) an antibody comprising amino acid sequences of a heavy chain set forth in SEQ ID NO: 142 and a light chain set forth in SEQ ID NO: 144;
(g) an antibody comprising amino acid sequences of a heavy chain set forth in SEQ ID NO: 146 and a light chain set forth in SEQ ID NO: 148;
(h) an antibody comprising amino acid sequences of a heavy chain set forth in SEQ ID NO: 150 and a light chain set forth in SEQ ID NO: 152;
(i) an antibody comprising amino acid sequences of a heavy chain set forth in SEQ ID NO: 154 and a light chain set forth in SEQ ID NO: 156;
(j) an antibody comprising amino acid sequences of a heavy chain set forth in SEQ ID NO: 162 and a light chain set forth in SEQ ID NO: 164;
(k) an antibody comprising amino acid sequences of a heavy chain set forth in SEQ ID NO: 166 and a light chain set forth in SEQ ID NO: 168;
(l) an antibody comprising amino acid sequences of a heavy chain set forth in SEQ ID NO: 170 and a light chain set forth in SEQ ID NO: 172;
(m) an antibody comprising amino acid sequences of a heavy chain set forth in SEQ ID NO: 174 and a light chain set forth in SEQ ID NO: 176;
(n) an antibody comprising amino acid sequences of a heavy chain set forth in SEQ ID NO: 178 and a light chain set forth in SEQ ID NO: 180;
(o) an antibody comprising amino acid sequences of a heavy chain set forth in SEQ ID NO: 182 and a light chain set forth in SEQ ID NO: 184;
(p) an antibody comprising amino acid sequences of a heavy chain set forth in SEQ ID NO: 186 and a light chain set forth in SEQ ID NO: 188;
(q) an antibody comprising amino acid sequences of a heavy chain set forth in SEQ ID NO: 190 and a light chain set forth in SEQ ID NO: 192;
(r) an antibody comprising amino acid sequences of a heavy chain set forth in SEQ ID NO: 194 and a light chain set forth in SEQ ID NO: 196;
(s) an antibody comprising amino acid sequences of a heavy chain set forth in SEQ ID NO: 198 and a light chain set forth in SEQ ID NO: 200;
(t) an antibody comprising amino acid sequences of a heavy chain set forth in SEQ ID NO: 202 and a light chain set forth in SEQ ID NO: 204; and
(u) a mutant of an antibody selected from (a) to (t) comprising at least one substitution, addition, or deletion.

17. The complex of claim 11, wherein the complex exhibits an IC₅₀ of about 0.5 nM or less in glypican-1 positive cells.

18. A method for preventing or treating glypican-1 positive cancer, comprising administering the complex of claim 11.

19. The method of claim 18, wherein cancer cells with the glypican-1 positive cancer express a high level of glypican-1 on a cell surface.

20. The method of claim 19, wherein the cancer cells with the glypican-1 positive cancer have an anti-glypican-1 antibody binding capacity of about 15000 or greater in an assay using QIFIKIT®.

21. The method of claim 20, wherein the glypican-1 positive cancer is selected from esophageal cancer, pancreatic cancer, cervical cancer, lung cancer, head and neck cancer, breast cancer, uterine leiomyosarcoma, prostate cancer, and any combination thereof.

22. The method of claim 20, wherein the glypican-1 positive cancer is esophageal cancer, which comprises esophageal cancer at a lymph node metastasis site, squamous cell carcinoma, and/or adenocarcinoma.

23. The method of claim 22, wherein the esophageal cancer comprises squamous cell carcinoma.

24.-37. (canceled)
